(19) United States
(12) Patent Application Publication
(10) Pub. No.: US 2023/0329101 A1
(43) Pub. Date: Oct. 12, 2023

(54) Organic Light Emitting Compound And Organic Light Emitting Device Including Same

(71) Applicant: P&H TECH Co., Ltd., Gyeonggi-do (KR)

(72) Inventors: Seo-Yong HYUN, Gyeonggi-do (KR); Seok-Keun YOON, Gyeonggi-do (KR); Gwan-hee PARK, Gyeonggi-do (KR)

(73) Assignee: P&H TECH Co., Ltd., Gyeonggi-do (KR)

(21) Appl. No.: 17/802,213

(22) PCT Filed: Mar. 12, 2021
(86) PCT No.: PCT/KR2021/003076

§ 371 Date: Aug. 25, 2022

(30) Foreign Application Priority Data
Mar. 24, 2020 (KR) 10-2020-0035483
Feb. 25, 2021 (KR) 10-2021-0025358

(51) Int. Cl. H10K 85/60; C07D 209/86; C09K 11/06; C07D 405/10; C07D 471/04

(52) U.S. Cl. CPC H10K 85/6572; C07D 209/86; C09K 11/06; H10K 50/84; C07D 405/10; H10K 85/6574; C07D 471/04; H10K 85/615

(57) ABSTRACT

The present invention relates to an organic light emitting compound adopted as a material for a light efficiency improving layer and the like provided in an organic light emitting device, and an organic light emitting device including the same, and when the organic light emitting compound according to the present invention is adopted as a material for a light efficiency improving layer provided in an organic light emitting device, the organic light emitting compound can be industrially usefully used for various display devices, lighting devices, and the like because it is possible to implement light emitting characteristics such as low voltage driving excellent color purity and excellent light emitting efficiency of the device.

### TECHNICAL FIELD

The present invention relates to an organic light emitting compound, and more specifically, to an organic light emitting compound which is characterized by being adopted as a material for a light efficiency improving layer (capping layer) provided in an organic light emitting device, and an organic light emitting device in which light emitting characteristics such as low-voltage driving, excellent color purity and excellent light emitting efficiency of the device are remarkably improved by adopting the same.

### BACKGROUND ART

Since an organic light emitting device has advantages in that the device can not only be formed on a transparent substrate, can but also be driven at a low voltage of 10 V or less compared to a plasma display panel or an inorganic electroluminescent (EL) display, and consume relatively low power and has excellent color tone, and can show three colors of green, blue and red, the organic light emitting device has recently attracted much attention as the next-generation display device.

However, in order for such an organic light emitting device to exhibit the characteristics as described above, first, a hole injection material, a hole transport material, a light emitting material, an electron transport material, an electron injection material, and the like, which are materials which form an organic layer in the device need to be supported by stable and efficient materials, but stable and efficient materials for an organic layer for an organic light emitting device have not been sufficiently developed to date.

Therefore, in order to implement a more stable organic light emitting device and achieve the high efficiency, long service life, large size, and the like of the device, additional improvements are required in terms of efficiency and service life characteristics, and in particular, there is an urgent need for the development of materials that constitute each organic layer of the organic light emitting device.

Recently, not only research to improve the characteristics of the organic light emitting device by changing the performance of each organic layer material, but also a technique of improving color purity and increasing light emitting efficiency by an optical thickness optimized between an anode and a cathode have been devised as one of the important factors to improve the device performance, and as an example of such a method, a capping layer may be used for electrodes to increase light efficiency and obtain excellent color purity.

### DISCLOSURE

### Technical Problem

Therefore, the present invention has been made in an effort to provide a novel organic light emitting compound which may be adopted for a light efficiency improving layer provided in an organic light emitting device to implement excellent light emitting characteristics such as a low-voltage driving, excellent color purity and improved light emitting efficiency of the device, and an organic light emitting device including the same.

### Technical Solution

In order to solve the problem, the present invention provides an organic light emitting compound represented by the following [Chemical Formula I].

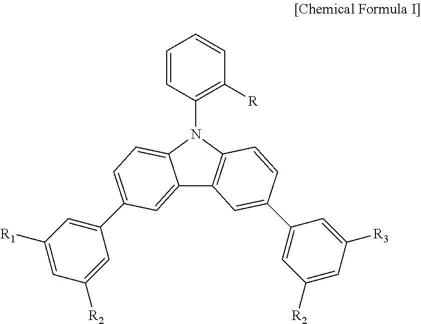

According to an exemplary embodiment of the present invention, [Chemical Formula I] may be an organic light emitting compound represented by the following [Chemical Formula I-1].

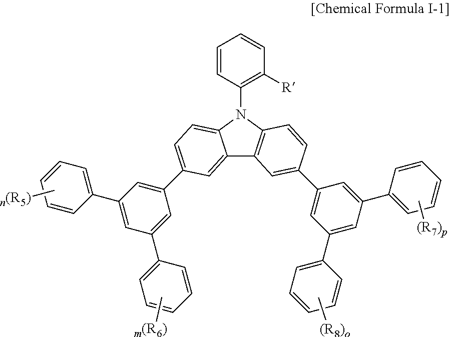

The specific structures of [Chemical Formula I] and [Chemical Formula I-1], compounds implemented by the chemical formulae, and R, R′, and R₁ to R₈ will be described below.

Further, to solve the problem, the present invention provides an organic light emitting device including a first electrode, a second electrode, and an organic layer having one or more layers disposed between the first electrode and the second electrode, in which a light efficiency improving layer (capping layer) formed on at least one side opposite to the organic layer at the top or bottom part of the first electrode and the second electrode is further included and the light efficiency improving layer includes the organic light emitting compound represented by [Chemical Formula I].

### Advantageous Effects

When the organic light emitting compound according to the present invention is adopted as a material for a light efficiency improving layer provided in an organic light emitting device, the organic light emitting compound can be usefully used for various display devices, lighting devices, and the like because it is possible to implement various light emitting characteristics such as low voltage driving, excellent color purity and excellent light emitting efficiency of the device.

### BEST MODE

Hereinafter, the present invention will be described in more detail.

The present invention relates to an organic light emitting compound capable of achieving light emitting characteristics such as low voltage driving, excellent color purity and excellent light emitting efficiency of an organic light emitting device.

The organic light emitting compound represented by [Chemical Formula I] according to the present invention has a structure in which a phenyl group is introduced into position Nos. 3, 6 and 9 of carbazole as shown in the following [Chemical Formula I] as a skeleton, in which substituents represented by R and R₁ to R₄ are introduced into specific positions of each phenyl group, and when the compound according to the present invention is applied to a light efficiency improving layer by characteristics of these skeletons and substituents, it is possible to implement an organic light emitting device having light emitting characteristics such as low-voltage driving, excellent color index and excellent light emitting efficiency.

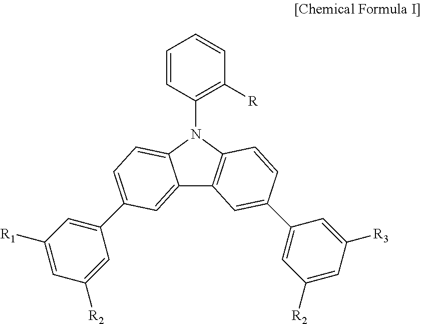

In [Chemical Formula I],

R is introduced into the ortho position of a phenyl group introduced into position No. 9 of carbazole, and is selected among deuterium, a cyano group, a halogen group, a substituted or unsubstituted alkyl group having 1 to 20 carbon atoms, a substituted or unsubstituted deuterated alkyl group having 1 to 20 carbon atoms, a substituted or unsubstituted halogenated alkyl group having 1 to 20 carbon atoms, a substituted or unsubstituted cycloalkyl group having 3 to 20 carbon atoms, a substituted or unsubstituted aryl group having 6 to 30 carbon atoms and a substituted or unsubstituted heteroaryl group having 3 to 30 carbon atoms.

R₁ to R₄ are the same as or different from each other, and are each independently selected among deuterium, a halogen group, a cyano group, a substituted or unsubstituted alkyl group having 1 to 20 carbon atoms, a substituted or unsubstituted cycloalkyl group having 3 to 20 carbon atoms, a substituted or unsubstituted deuterated alkyl group having 1 to 20 carbon atoms, a substituted or unsubstituted halogenated alkyl group having 1 to 20 carbon atoms and a substituted or unsubstituted aryl group having 6 to 30 carbon atoms.

According to an exemplary embodiment of the present invention, R₁ to R₄ are the same as or different from each other, and may be each independently a substituted or unsubstituted aryl group having 6 to 30 carbon atoms, more specifically a phenyl group which is each substituted.

Accordingly, [Chemical Formula I] according to the present invention may be an organic light emitting compound represented by the following [Chemical Formula I-1].

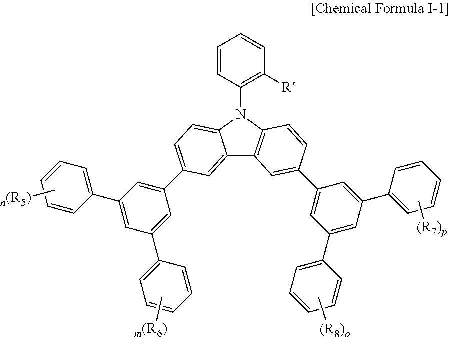

In [Chemical Formula I-1],

R′ is the same as the definition of R of [Chemical Formula I], R₅ to R₈ are the same as the definitions of R₁ to R₄ of [Chemical Formula I], n, m, o and p are each an integer from 1 to 5, and when n, m, o and p are each 2 or higher, a plurality of R₅ to R₈ are each the same as or different from each other.

In addition, according to an exemplary embodiment of the present invention, in [Chemical Formula I-1], R′ and R₅ to R₈ are the same as or different from each other, and are each independently selected among deuterium, a halogen group, a cyano group, a substituted or unsubstituted halogenated alkyl group having 1 to 20 carbon atoms, a substituted or unsubstituted deuterated alkyl group having 1 to 20 carbon atoms, a substituted or unsubstituted alkyl group having 1 to 20 carbon atoms and a substituted or unsubstituted aryl group having 6 to 30 carbon atoms.

Furthermore, according to an exemplary embodiment of the present invention, R′ and R₅ to R₈ may be each deuterium (D), a deuterated alkyl group (−CD₃), or a halogenated alkyl group (−CF₃).

Further, R′ and R₅ to R₈ may be each a substituted or unsubstituted aryl group having 6 to 30 carbon atoms, and may be preferably an unsaturated phenyl group, or a phenyl group which is substituted with any one selected among deuterium, a halogen group, a cyano group, a deuterated alkyl group (−CD₃), a halogenated alkyl group (−CF₃) and a phenyl group (Ph).

Meanwhile, the ‘substituted or unsubstituted’ means that R, R′ and R₁ to R₈ are each substituted with one or two or more substituents selected from the group consisting of deuterium, a halogen group, a cyano group, a silyl group, an alkyl group, a halogenated alkyl group, a deuterated alkyl group, a cycloalkyl group, an alkoxy group, an aryl group and a heterocyclic group, substituted with a substituent to which two or more substituents among the aforementioned substituents are linked, or have no substituent.

In the present invention, an example of the aforementioned substituents will be specifically described below, but is not limited thereto, and can be clearly confirmed in a specific compound according to the present invention.

In the present invention, the alkyl group may be straight-chained or branched, and the number of carbon atoms thereof is not particularly limited, but is preferably 2 to 20. Specific examples thereof include an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto. Further, the deuterated alkyl group and the halogenated alkyl group mean that the aforementioned alkyl group is substituted with one or more deuterium(s) and halogen group(s).

In the present specification, the aryl group may be monocyclic or polycyclic, and the number of carbon atoms thereof is not particularly limited, but is preferably 6 to 30. Examples of the monocyclic aryl group include a phenyl group, a biphenyl group, a terphenyl group, a stilbene group and the like, examples of the polycyclic aryl group include a naphthyl group, an anthracenyl group, a phenanthrenyl group, a pyrenyl group, a perylenyl group, a tetracenyl group, a chrysenyl group, a fluorenyl group, an acenapthacenyl group, a triphenylene group, a fluoranthrene group, and the like, but the scope of the present invention is not limited to these examples.

In the present invention, a heteroaryl group is a heterocyclic group including 0, N or S as a heteroatom, the number of carbon atoms thereof is not particularly limited, but is preferably 2 to 30, and specific examples thereof in the present invention include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, a triazole group, an acridyl group, a pyridazine group, a pyrazinyl group, a quinolinyl group, a quinazoline group, a quinoxalinyl group, a phthalazinyl group, a pyrido pyrimidinyl group, a pyrido pyrazinyl group, a pyrazino pyrazinyl group, an isoquinoline group, an indole group, a carbazole group, a benzoxazole group, a benzoimidazole group, a benzothiazole group, a benzocarbazole group, a benzothiophene group, a dibenzothiophene group, a benzofuranyl group, a dibenzofuranyl group, a phenanthroline group, a thiazolyl group, an isoxazolyl group, an oxadiazolyl group, a thiadiazolyl group, a benzothiazolyl group, a phenothiazinyl group, a phenoxazine group, a phenothiazine group, and the like, but are not limited thereto.

In the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In the present invention, specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

Specific examples of the halogen group, which is a substituent used in the present invention, include fluorine (F), chlorine (Cl), bromine (Br), and the like.

The organic light emitting compound represented by the above [Chemical Formula I] according to the present invention may be used as various organic layers in an organic light emitting device due to its structural specificity, and more specifically, may be used as a material for a light efficiency improving layer provided in the organic light emitting device.

Preferred specific examples of the organic light emitting compounds represented by [Chemical Formula I] and [Chemical Formula I-1] according to the present invention may be the following Compounds [1] to [171] and [1-1] to [1-207], but are not limited thereto.

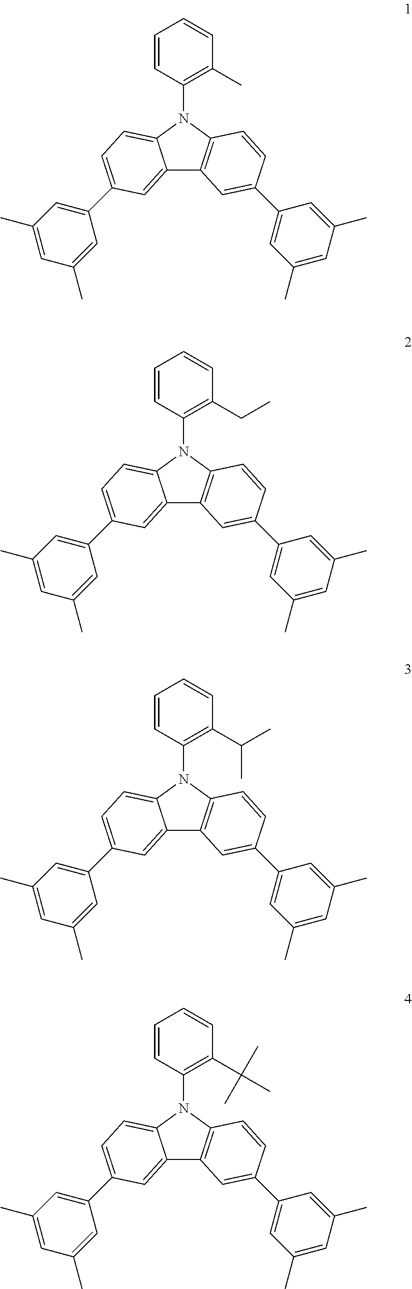

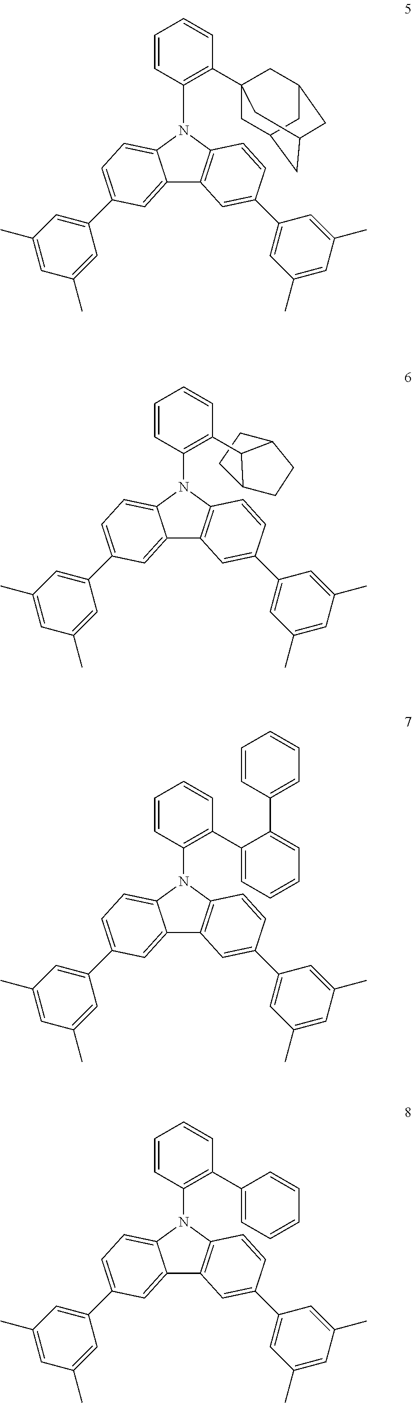

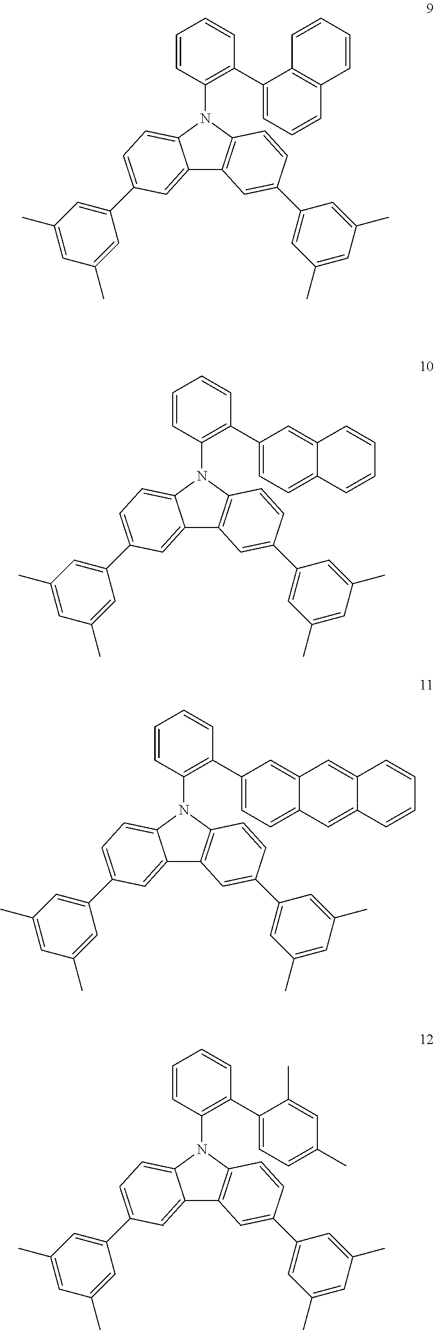

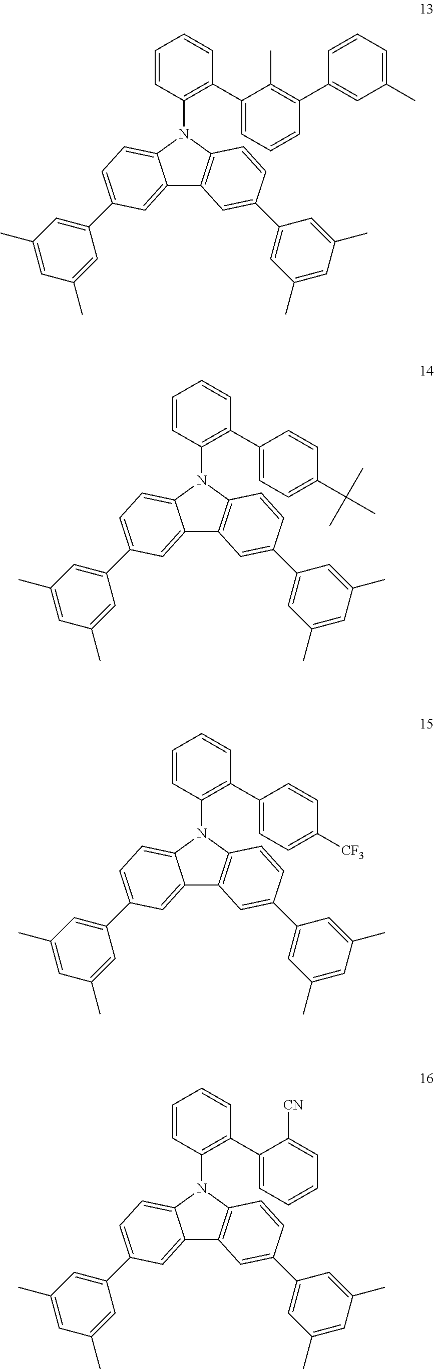

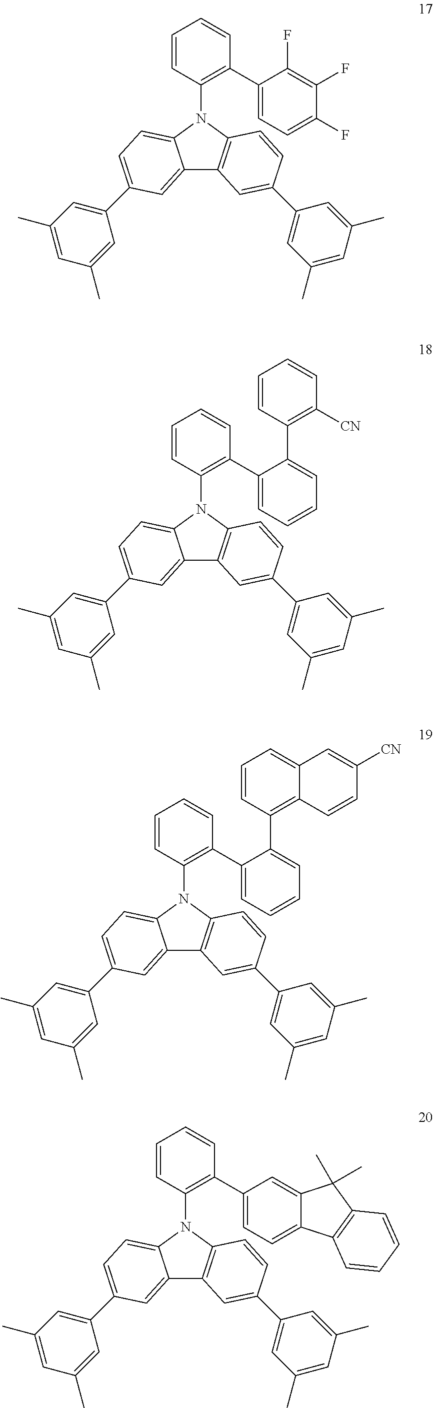

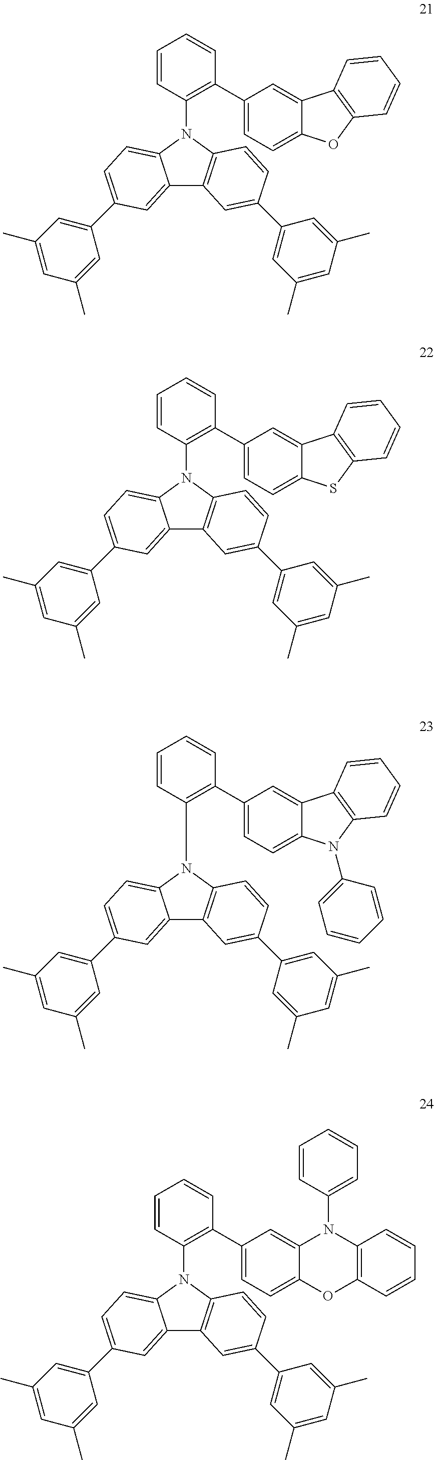 [SMILES: Cc1cc(C)cc(-c2ccc3c(c2)c2cc(-c4cc(C)cc(C)c4)ccc2n3-c2ccccc2-c2ccc3oc4ccccc4c3c2)c1 ; Cc1cc(C)cc(-c2ccc3c(c2)c2cc(-c4cc(C)cc(C)c4)ccc2n3-c2ccccc2-c2ccc3sc4ccccc4c3c2)c1 ; Cc1cc(C)cc(-c2ccc3c(c2)c2cc(-c4cc(C)cc(C)c4)ccc2n3-c2ccccc2-c2ccc3c(c2)c2ccccc2n3-c2ccccc2)c1 ; Cc1cc(C)cc(-c2ccc3c(c2)c2cc(-c4cc(C)cc(C)c4)ccc2n3-c2ccccc2-c2ccc3c(c2)N(c2ccccc2)c2ccccc2O3)c1]

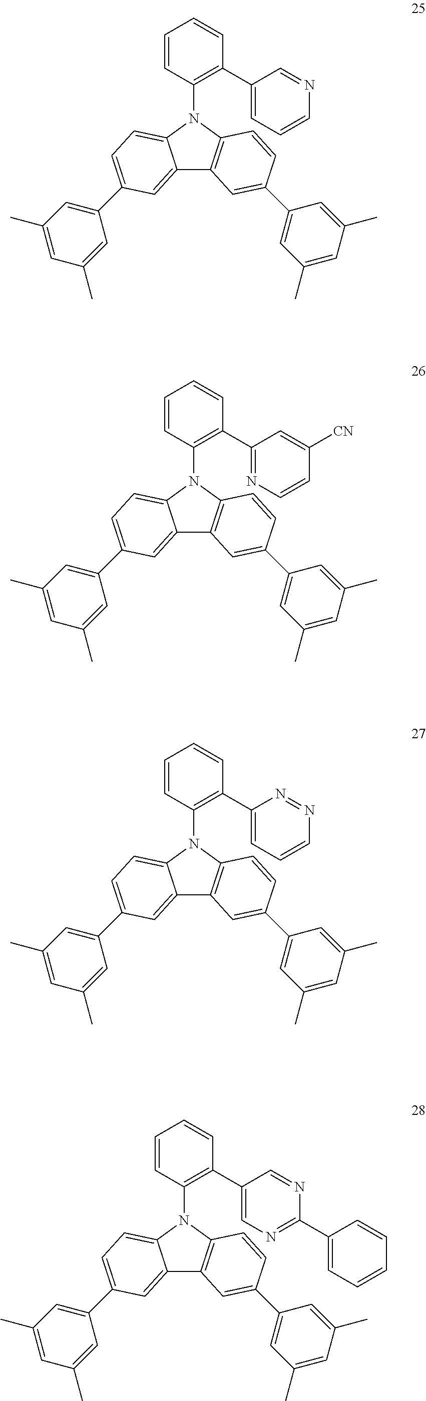

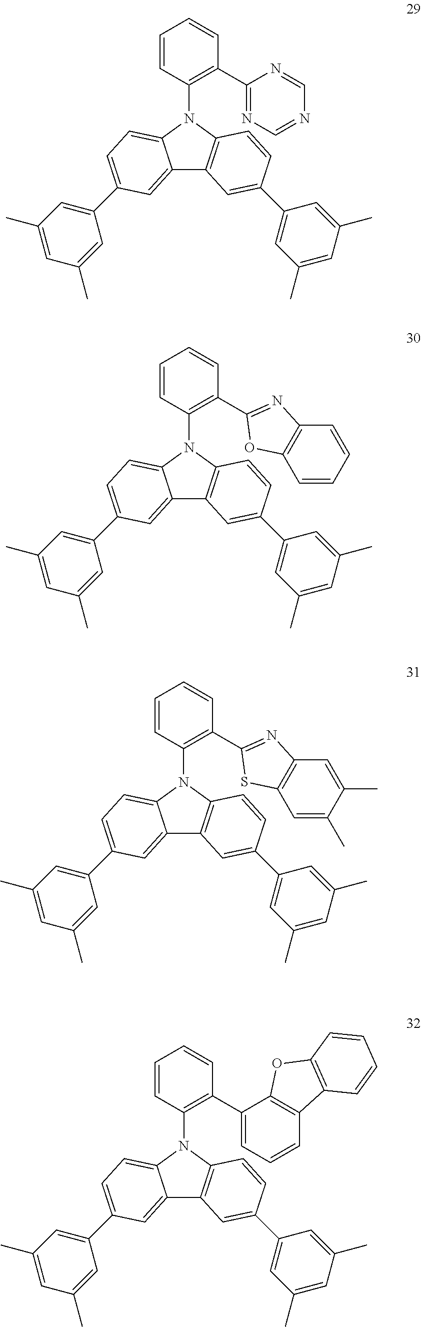

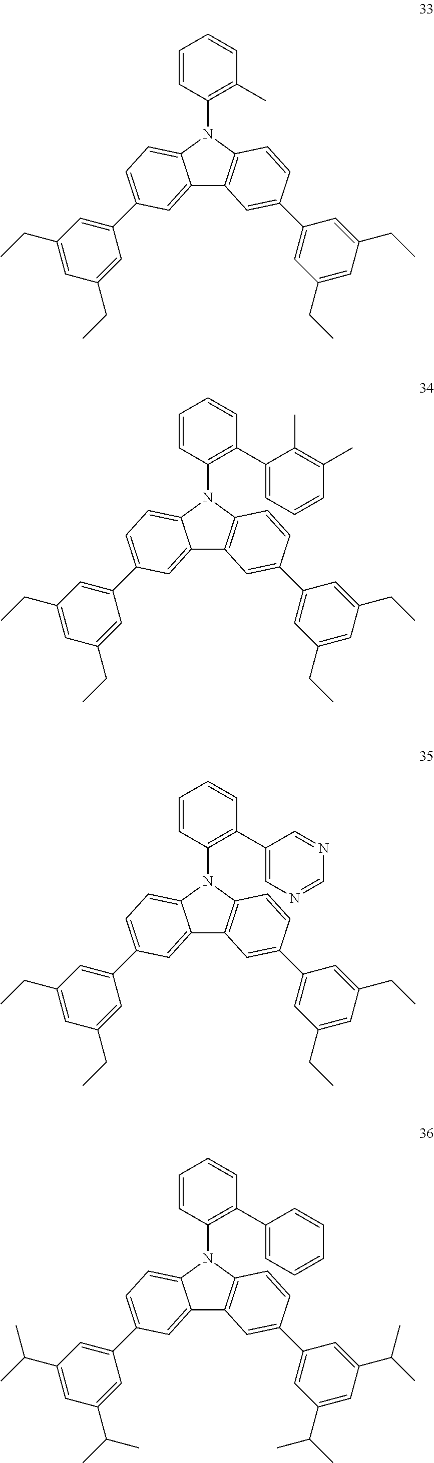

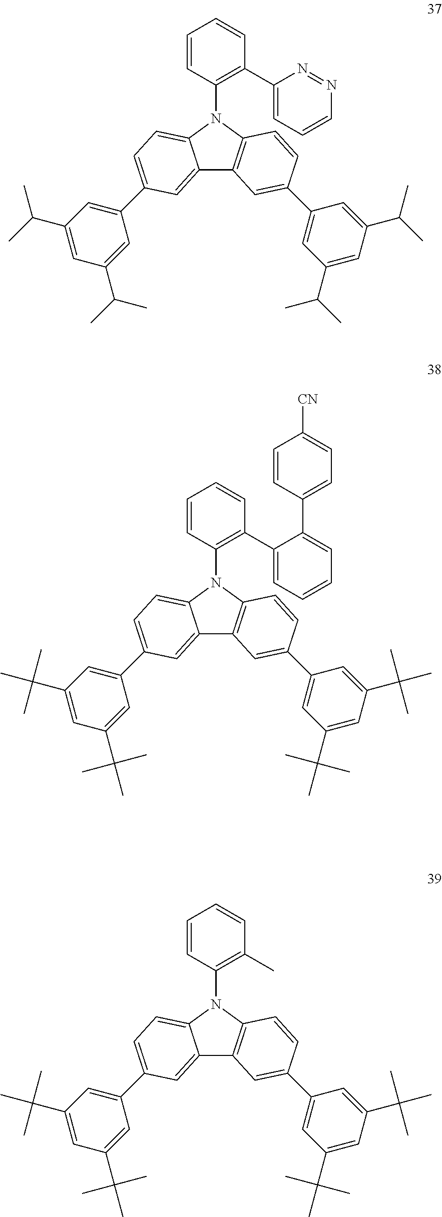

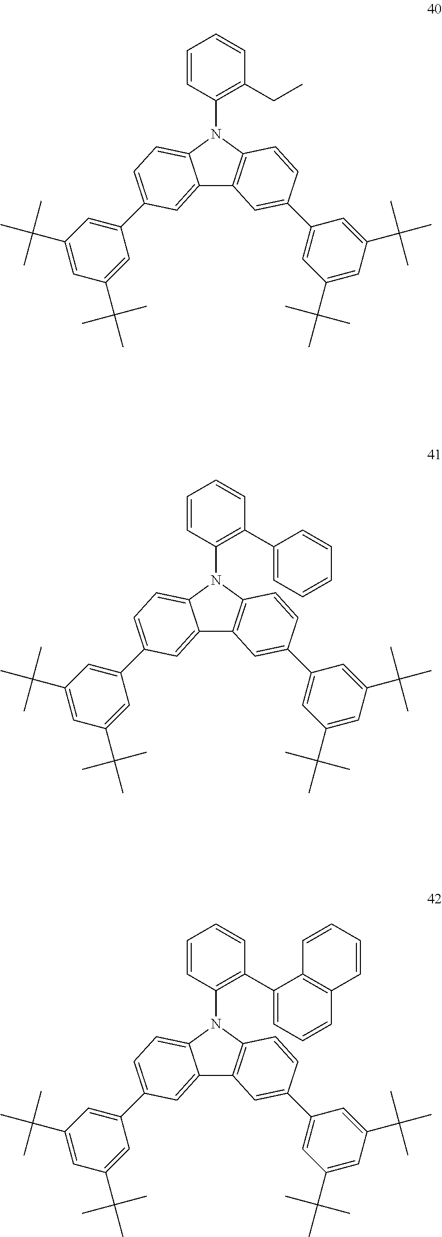

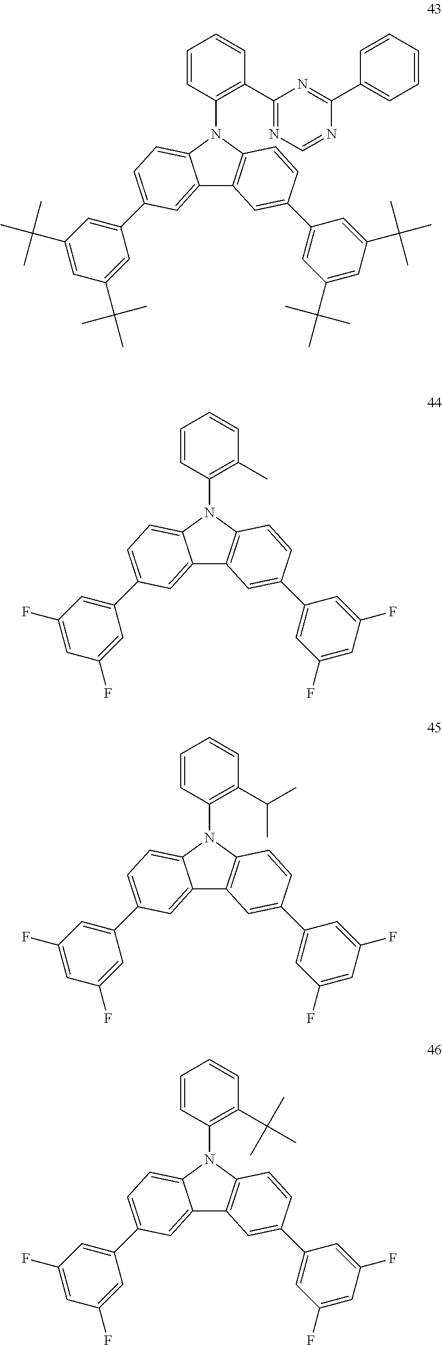 [SMILES: CC(C)(C)c1cc(-c2ccc3c(c2)c2cc(-c4cc(C(C)(C)C)cc(C(C)(C)C)c4)ccc2n3-c2ccccc2-c2ncnc(-c3ccccc3)n2)cc(C(C)(C)C)c1 ; Cc1ccccc1-n1c2ccc(-c3cc(F)cc(F)c3)cc2c2cc(-c3cc(F)cc(F)c3)ccc21 ; CC(C)c1ccccc1-n1c2ccc(-c3cc(F)cc(F)c3)cc2c2cc(-c3cc(F)cc(F)c3)ccc21 ; CC(C)(C)c1ccccc1-n1c2ccc(-c3cc(F)cc(F)c3)cc2c2cc(-c3cc(F)cc(F)c3)ccc21]

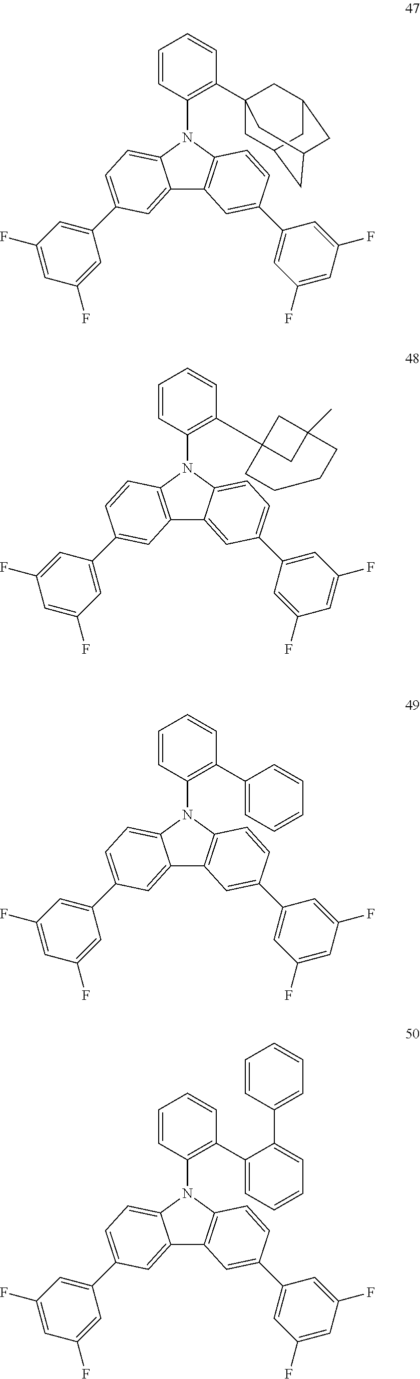 [SMILES: Fc1cc(F)cc(-c2ccc3c(c2)c2cc(-c4cc(F)cc(F)c4)ccc2n3-c2ccccc2C23CC4CC(CC(C4)C2)C3)c1 ; CC12CCCCC(c3ccccc3-n3c4ccc(-c5cc(F)cc(F)c5)cc4c4cc(-c5cc(F)cc(F)c5)ccc43)(C1)C2 ; Fc1cc(F)cc(-c2ccc3c(c2)c2cc(-c4cc(F)cc(F)c4)ccc2n3-c2ccccc2-c2ccccc2)c1 ; Fc1cc(F)cc(-c2ccc3c(c2)c2cc(-c4cc(F)cc(F)c4)ccc2n3-c2ccccc2-c2ccccc2-c2ccccc2)c1]

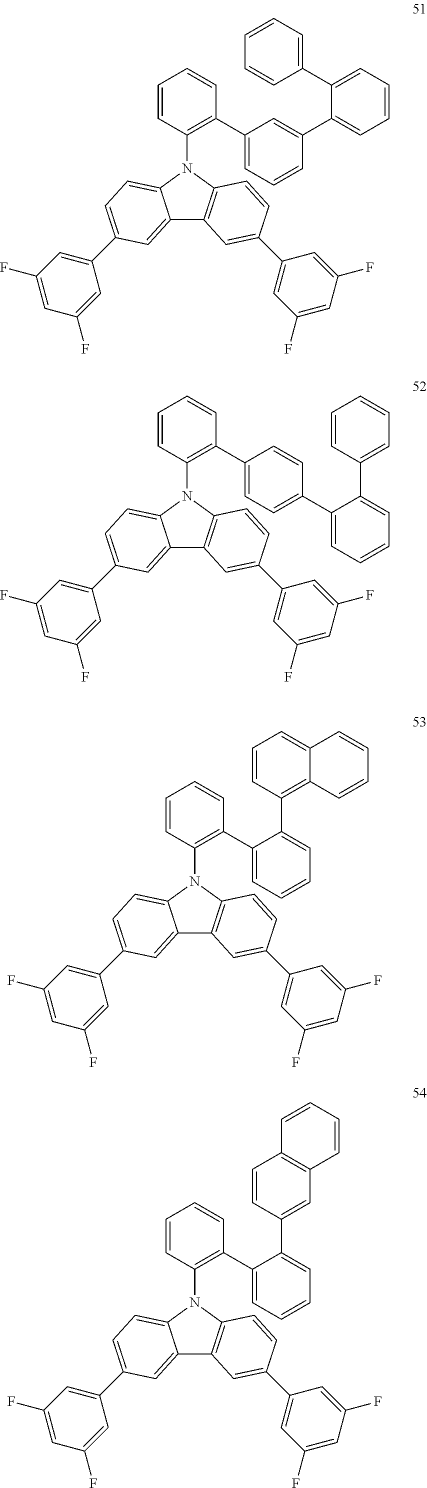

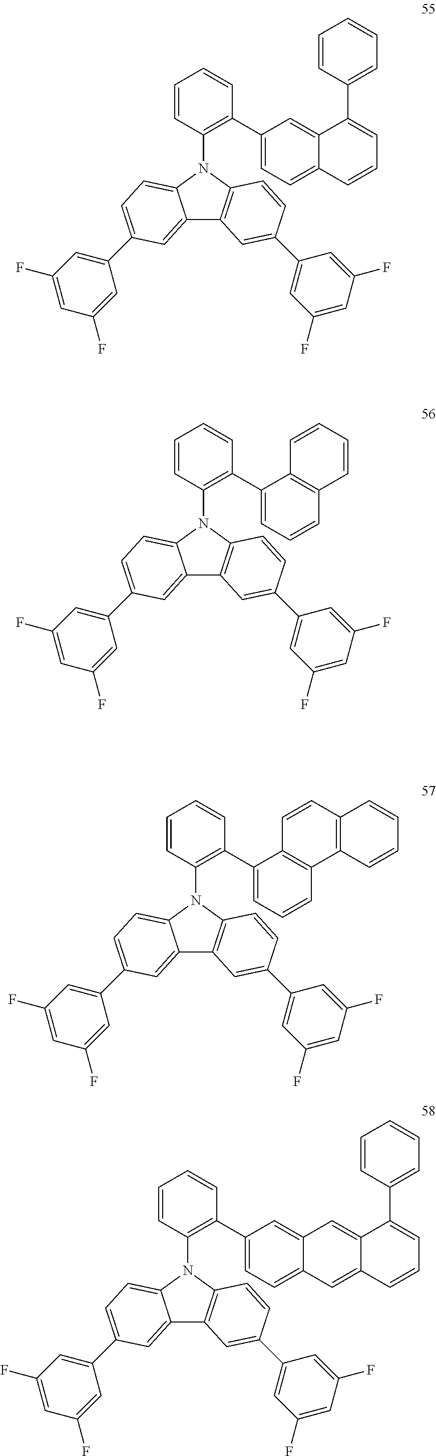 [SMILES: Fc1cc(F)cc(-c2ccc3c(c2)c2cc(-c4cc(F)cc(F)c4)ccc2n3-c2ccccc2-c2ccc3cccc(-c4ccccc4)c3c2)c1 ; Fc1cc(F)cc(-c2ccc3c(c2)c2cc(-c4cc(F)cc(F)c4)ccc2n3-c2ccccc2-c2cccc3ccccc23)c1 ; Fc1cc(F)cc(-c2ccc3c(c2)c2cc(-c4cc(F)cc(F)c4)ccc2n3-c2ccccc2-c2cccc3c2ccc2ccccc23)c1 ; Fc1cc(F)cc(-c2ccc3c(c2)c2cc(-c4cc(F)cc(F)c4)ccc2n3-c2ccccc2-c2ccc3cc4cccc(-c5ccccc5)c4cc3c2)c1]

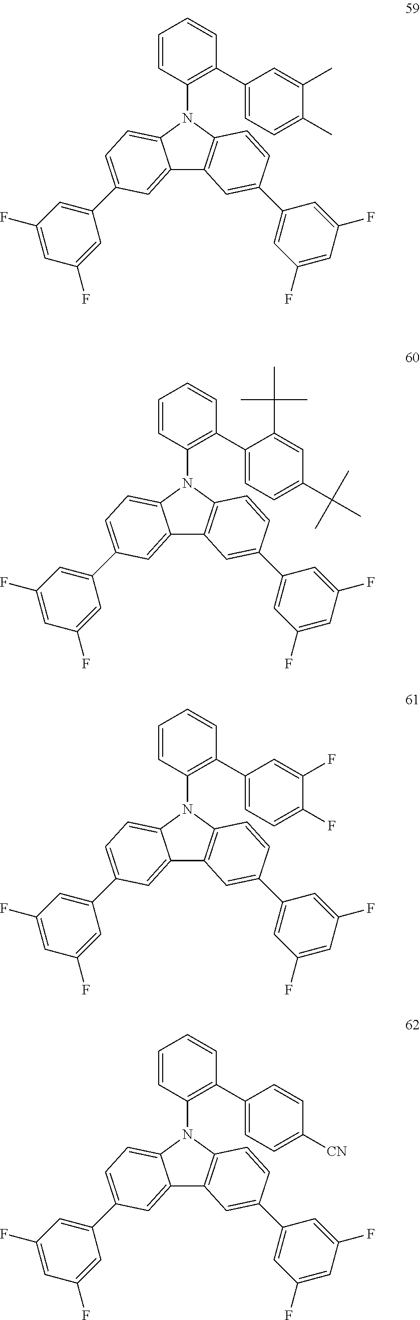 [SMILES: Cc1ccc(-c2ccccc2-n2c3ccc(-c4cc(F)cc(F)c4)cc3c3cc(-c4cc(F)cc(F)c4)ccc32)cc1C ; CC(C)(C)c1ccc(-c2ccccc2-n2c3ccc(-c4cc(F)cc(F)c4)cc3c3cc(-c4cc(F)cc(F)c4)ccc32)c(C(C)(C)C)c1 ; Fc1cc(F)cc(-c2ccc3c(c2)c2cc(-c4cc(F)cc(F)c4)ccc2n3-c2ccccc2-c2ccc(F)c(F)c2)c1 ; N#Cc1ccc(-c2ccccc2-n2c3ccc(-c4cc(F)cc(F)c4)cc3c3cc(-c4cc(F)cc(F)c4)ccc32)cc1]

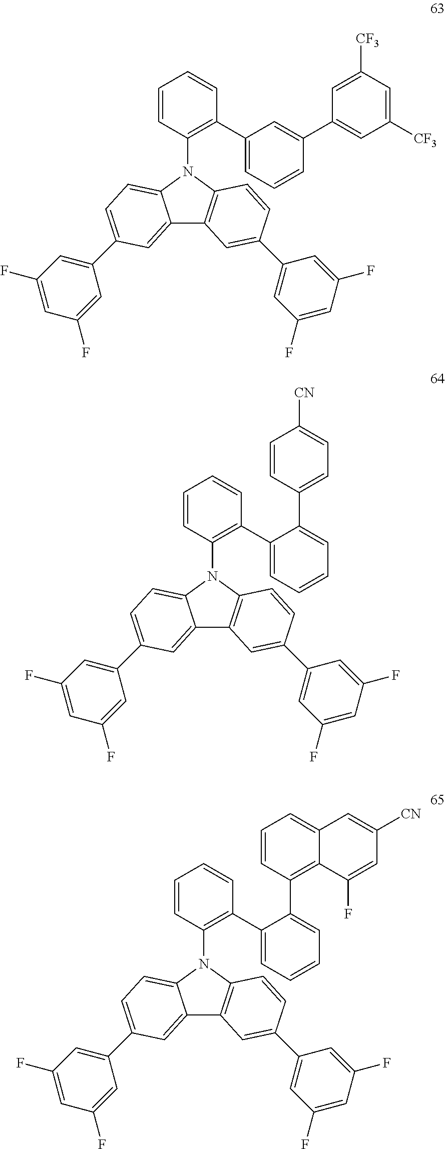

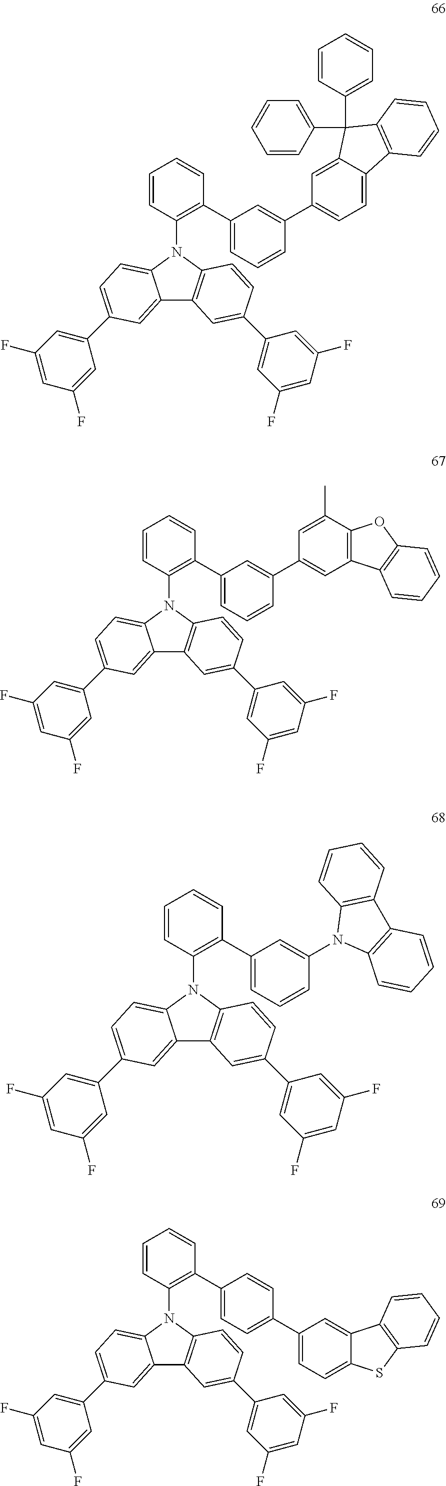 [SMILES: Fc1cc(F)cc(-c2ccc3c(c2)c2cc(-c4cc(F)cc(F)c4)ccc2n3-c2ccccc2-c2cccc(-c3ccc4c(c3)C(c3ccccc3)(c3ccccc3)c3ccccc3-4)c2)c1 ; Cc1cc(-c2cccc(-c3ccccc3-n3c4ccc(-c5cc(F)cc(F)c5)cc4c4cc(-c5cc(F)cc(F)c5)ccc43)c2)cc2c1oc1ccccc12 ; Fc1cc(F)cc(-c2ccc3c(c2)c2cc(-c4cc(F)cc(F)c4)ccc2n3-c2ccccc2-c2cccc(-n3c4ccccc4c4ccccc43)c2)c1 ; Fc1cc(F)cc(-c2ccc3c(c2)c2cc(-c4cc(F)cc(F)c4)ccc2n3-c2ccccc2-c2ccc(-c3ccc4sc5ccccc5c4c3)cc2)c1]

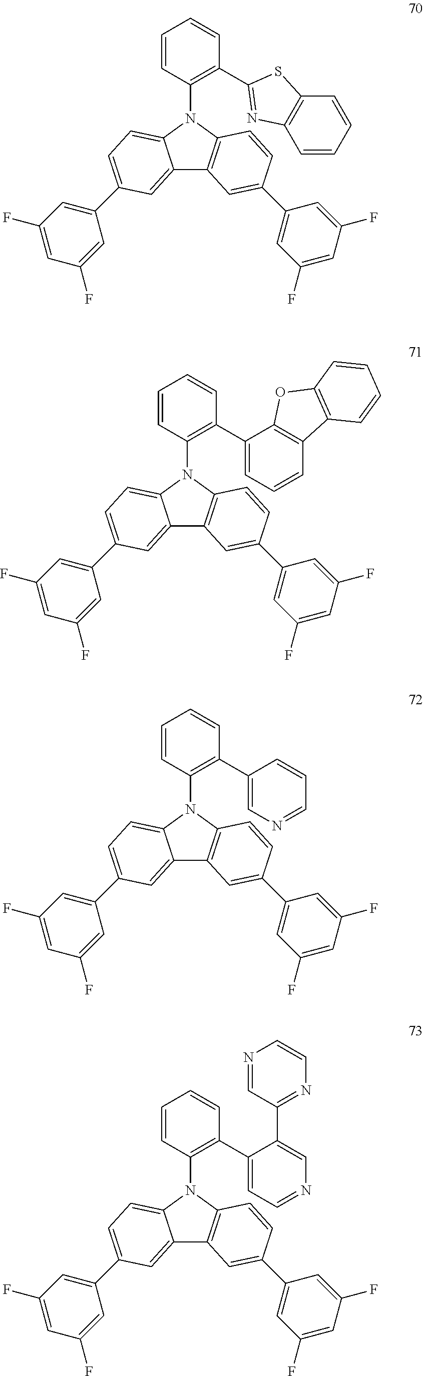 [SMILES: Fc1cc(F)cc(-c2ccc3c(c2)c2cc(-c4cc(F)cc(F)c4)ccc2n3-c2ccccc2-c2nc3ccccc3s2)c1 ; Fc1cc(F)cc(-c2ccc3c(c2)c2cc(-c4cc(F)cc(F)c4)ccc2n3-c2ccccc2-c2cccc3c2oc2ccccc23)c1 ; Fc1cc(F)cc(-c2ccc3c(c2)c2cc(-c4cc(F)cc(F)c4)ccc2n3-c2ccccc2-c2cccnc2)c1 ; Fc1cc(F)cc(-c2ccc3c(c2)c2cc(-c4cc(F)cc(F)c4)ccc2n3-c2ccccc2-c2ccncc2-c2cnccn2)c1]

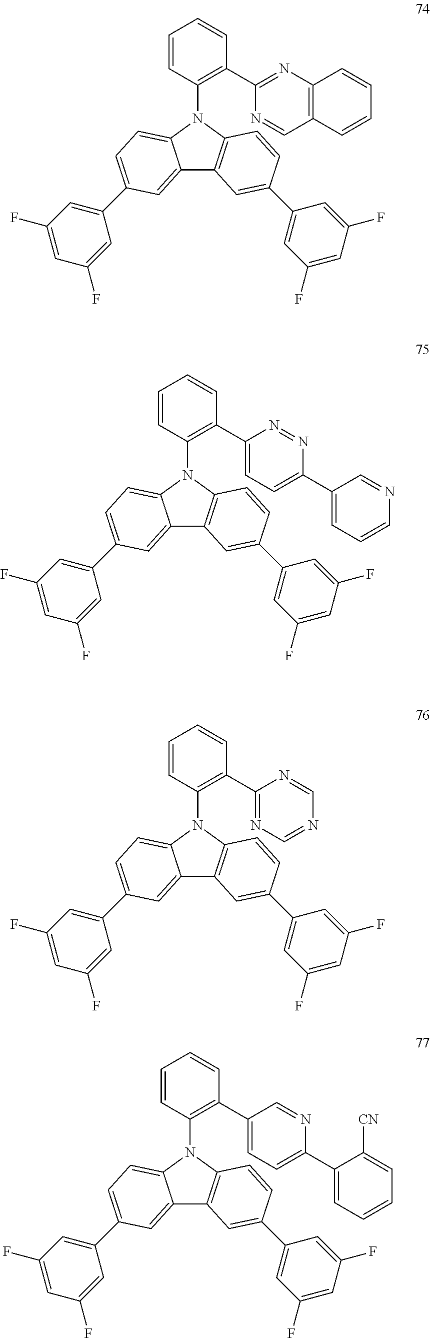 [SMILES: Fc1cc(F)cc(-c2ccc3c(c2)c2cc(-c4cc(F)cc(F)c4)ccc2n3-c2ccccc2-c2ncc3ccccc3n2)c1 ; Fc1cc(F)cc(-c2ccc3c(c2)c2cc(-c4cc(F)cc(F)c4)ccc2n3-c2ccccc2-c2ccc(-c3cccnc3)nn2)c1 ; Fc1cc(F)cc(-c2ccc3c(c2)c2cc(-c4cc(F)cc(F)c4)ccc2n3-c2ccccc2-c2ncncn2)c1 ; N#Cc1ccccc1-c1ccc(-c2ccccc2-n2c3ccc(-c4cc(F)cc(F)c4)cc3c3cc(-c4cc(F)cc(F)c4)ccc32)cn1]

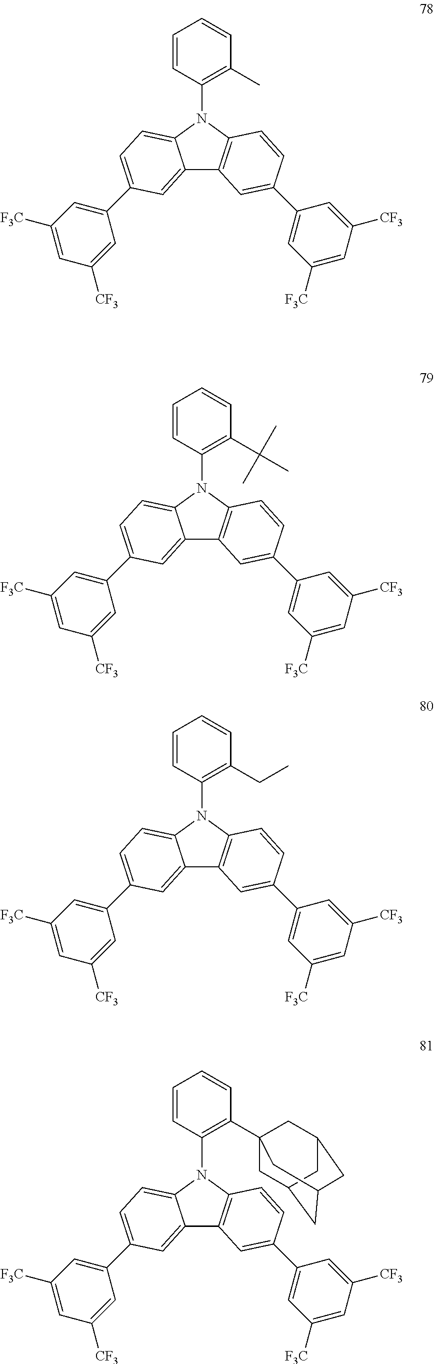 [SMILES: Cc1ccccc1-n1c2ccc(-c3cc(C(F)(F)F)cc(C(F)(F)F)c3)cc2c2cc(-c3cc(C(F)(F)F)cc(C(F)(F)F)c3)ccc21 ; CC(C)(C)c1ccccc1-n1c2ccc(-c3cc(C(F)(F)F)cc(C(F)(F)F)c3)cc2c2cc(-c3cc(C(F)(F)F)cc(C(F)(F)F)c3)ccc21 ; CCc1ccccc1-n1c2ccc(-c3cc(C(F)(F)F)cc(C(F)(F)F)c3)cc2c2cc(-c3cc(C(F)(F)F)cc(C(F)(F)F)c3)ccc21 ; FC(F)(F)c1cc(-c2ccc3c(c2)c2cc(-c4cc(C(F)(F)F)cc(C(F)(F)F)c4)ccc2n3-c2ccccc2C23CC4CC(CC(C4)C2)C3)cc(C(F)(F)F)c1]

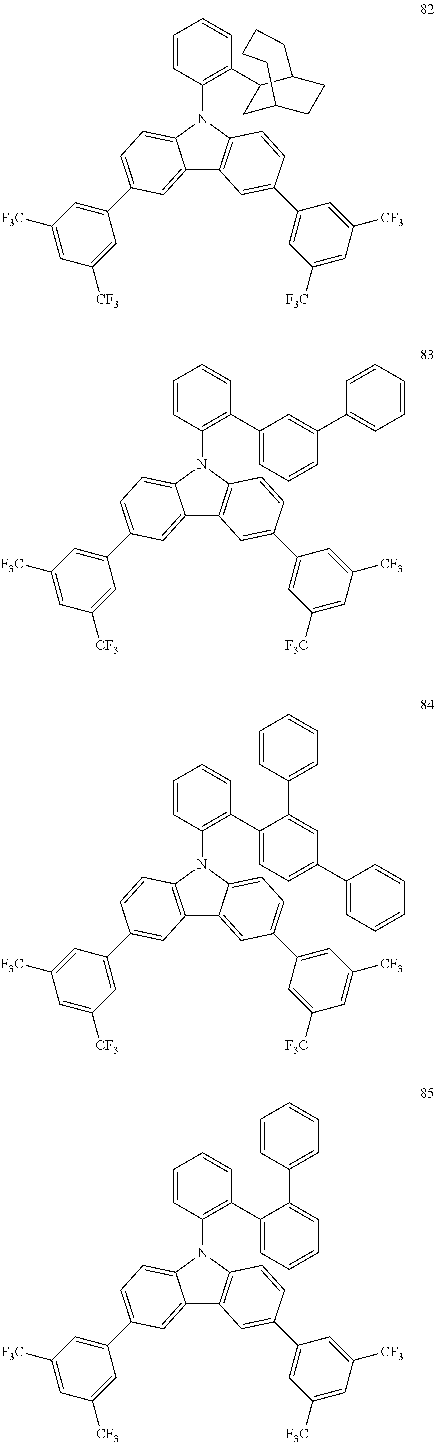 [SMILES: FC(F)(F)c1cc(-c2ccc3c(c2)c2cc(-c4cc(C(F)(F)F)cc(C(F)(F)F)c4)ccc2n3-c2ccccc2C2CC3CCCCC2CC3)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc(-c3ccccc3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2-c2ccccc2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccccc2-c2ccccc2)cc(C(F)(F)F)c1]

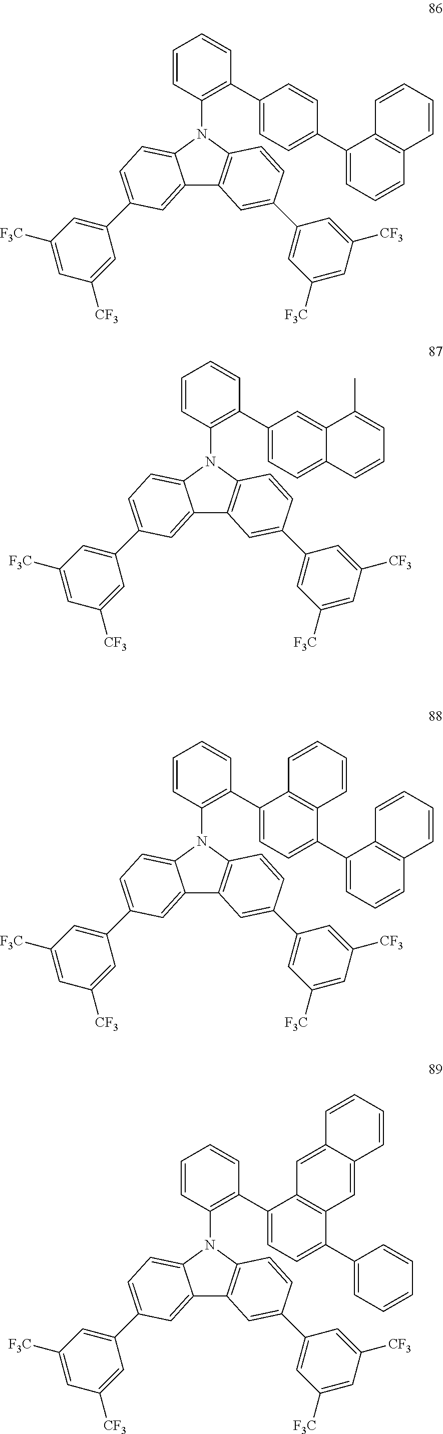 [SMILES: FC(F)(F)c1cc(-c2ccc3c(c2)c2cc(-c4cc(C(F)(F)F)cc(C(F)(F)F)c4)ccc2n3-c2ccccc2-c2ccc(-c3cccc4ccccc34)cc2)cc(C(F)(F)F)c1 ; Cc1cccc2ccc(-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)cc12 ; FC(F)(F)c1cc(-c2ccc3c(c2)c2cc(-c4cc(C(F)(F)F)cc(C(F)(F)F)c4)ccc2n3-c2ccccc2-c2ccc(-c3cccc4ccccc34)c3ccccc23)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)c3cc4ccccc4cc23)cc(C(F)(F)F)c1]

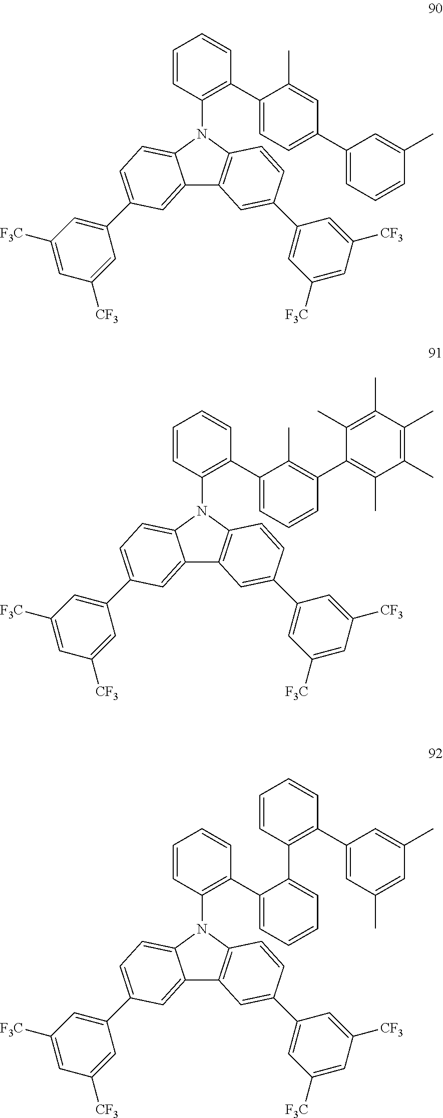 [SMILES: Cc1cccc(-c2ccc(-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)c(C)c2)c1 ; Cc1c(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cccc1-c1c(C)c(C)c(C)c(C)c1C ; Cc1cc(C)cc(-c2ccccc2-c2ccccc2-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c1]

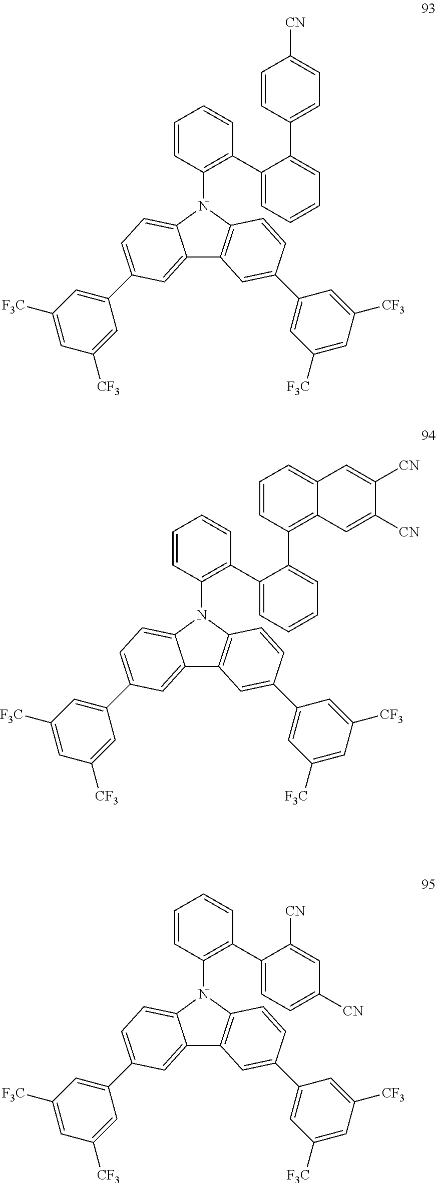 [SMILES: N#Cc1ccc(-c2ccccc2-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cc1 ; N#Cc1cc2cccc(-c3ccccc3-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)c2cc1C#N ; N#Cc1ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c(C#N)c1]

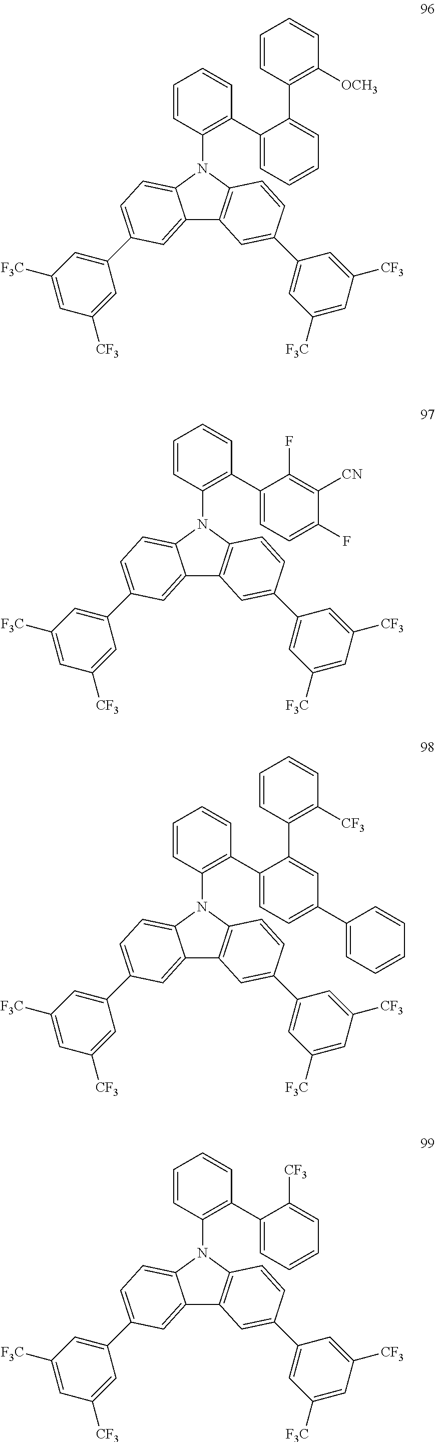 [SMILES: COc1ccccc1-c1ccccc1-c1ccccc1-n1c2ccc(-c3cc(C(F)(F)F)cc(C(F)(F)F)c3)cc2c2cc(-c3cc(C(F)(F)F)cc(C(F)(F)F)c3)ccc21 ; N#Cc1c(F)ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c1F ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2-c2ccccc2C(F)(F)F)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccccc2C(F)(F)F)cc(C(F)(F)F)c1]

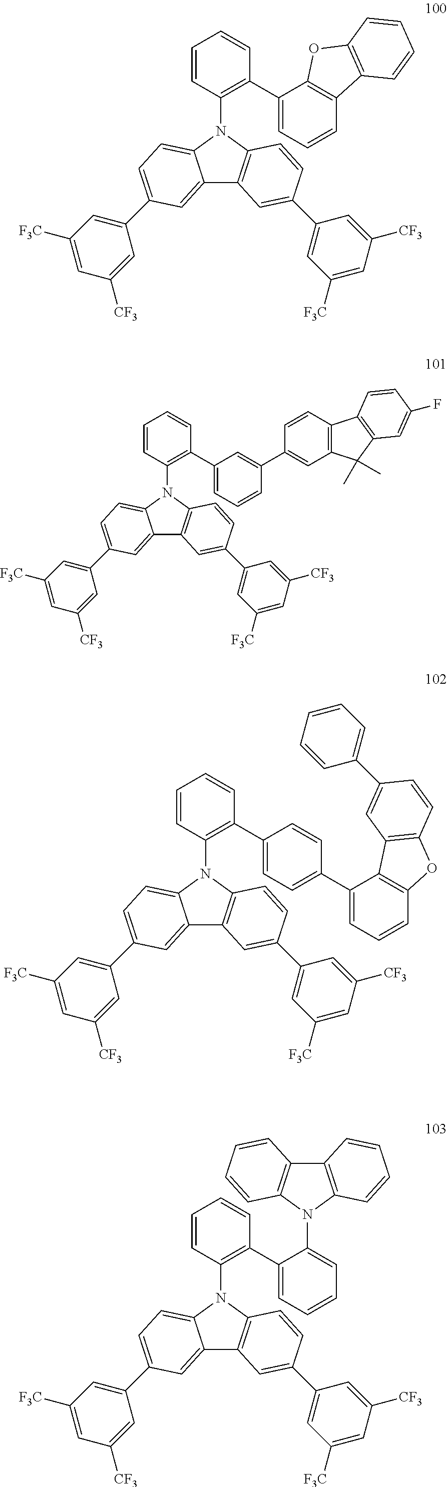

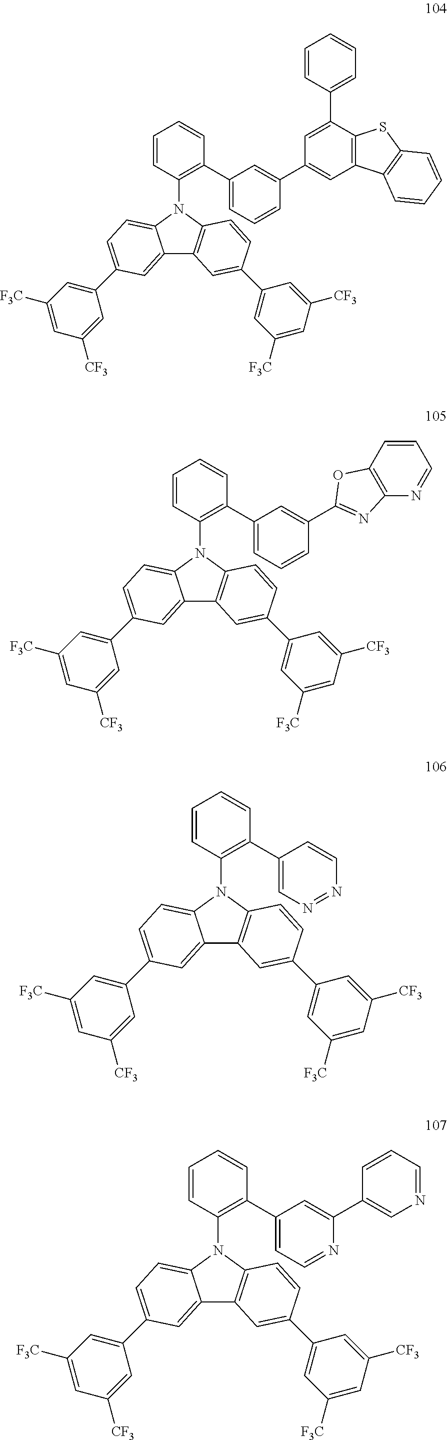 [SMILES: FC(F)(F)c1cc(-c2ccc3c(c2)c2cc(-c4cc(C(F)(F)F)cc(C(F)(F)F)c4)ccc2n3-c2ccccc2-c2cccc(-c3cc(-c4ccccc4)c4sc5ccccc5c4c3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc(-c3nc4ncccc4o3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccnnc2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccnc(-c3cccnc3)c2)cc(C(F)(F)F)c1]

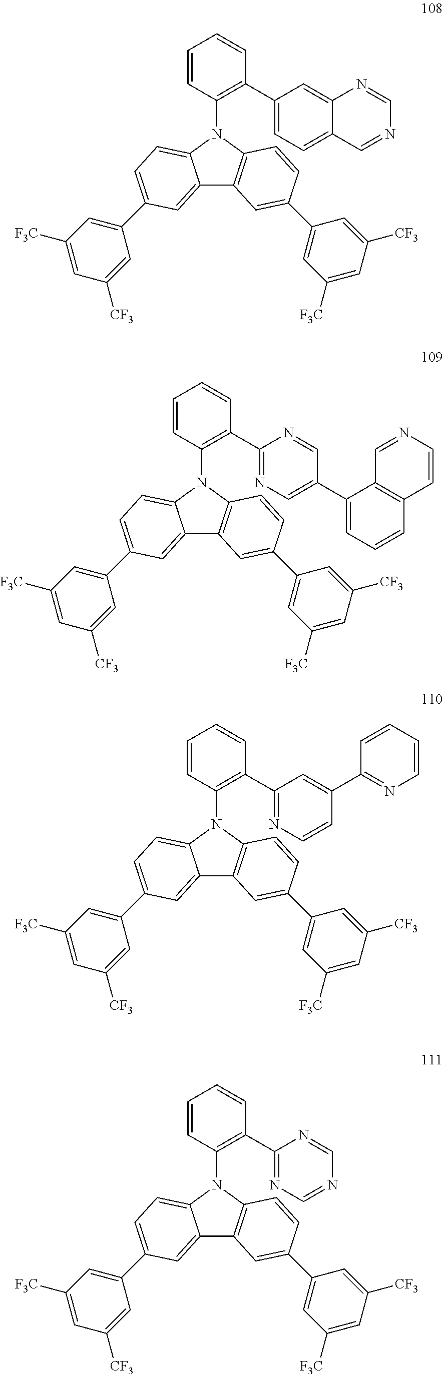 [SMILES: FC(F)(F)c1cc(-c2ccc3c(c2)c2cc(-c4cc(C(F)(F)F)cc(C(F)(F)F)c4)ccc2n3-c2ccccc2-c2ccc3cncnc3c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ncc(-c3cccc4ccncc34)cn2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cc(-c3ccccn3)ccn2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ncncn2)cc(C(F)(F)F)c1]

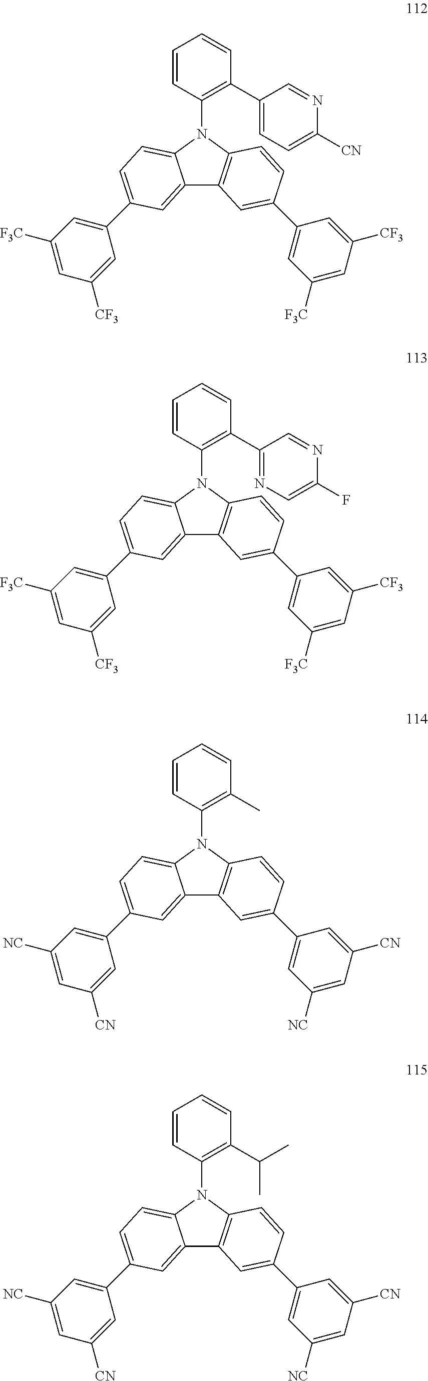 [SMILES: N#Cc1ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cn1 ; Fc1cnc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cn1 ; Cc1ccccc1-n1c2ccc(-c3cc(C#N)cc(C#N)c3)cc2c2cc(-c3cc(C#N)cc(C#N)c3)ccc21 ; CC(C)c1ccccc1-n1c2ccc(-c3cc(C#N)cc(C#N)c3)cc2c2cc(-c3cc(C#N)cc(C#N)c3)ccc21]

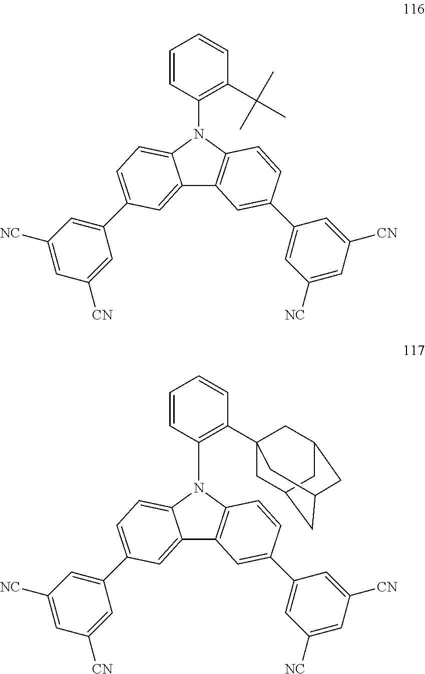 [SMILES: CC(C)(C)c1ccccc1-n1c2ccc(-c3cc(C#N)cc(C#N)c3)cc2c2cc(-c3cc(C#N)cc(C#N)c3)ccc21 ; N#Cc1cc(C#N)cc(-c2ccc3c(c2)c2cc(-c4cc(C#N)cc(C#N)c4)ccc2n3-c2ccccc2C23CC4CC(CC(C4)C2)C3)c1]

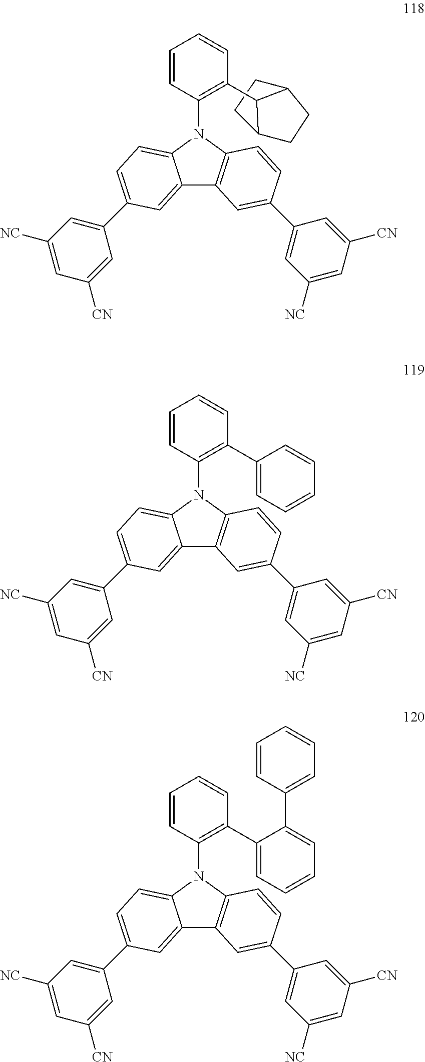 [SMILES: N#Cc1cc(C#N)cc(-c2ccc3c(c2)c2cc(-c4cc(C#N)cc(C#N)c4)ccc2n3-c2ccccc2C2C3CCC2CC3)c1 ; N#Cc1cc(C#N)cc(-c2ccc3c(c2)c2cc(-c4cc(C#N)cc(C#N)c4)ccc2n3-c2ccccc2-c2ccccc2)c1 ; N#Cc1cc(C#N)cc(-c2ccc3c(c2)c2cc(-c4cc(C#N)cc(C#N)c4)ccc2n3-c2ccccc2-c2ccccc2-c2ccccc2)c1]

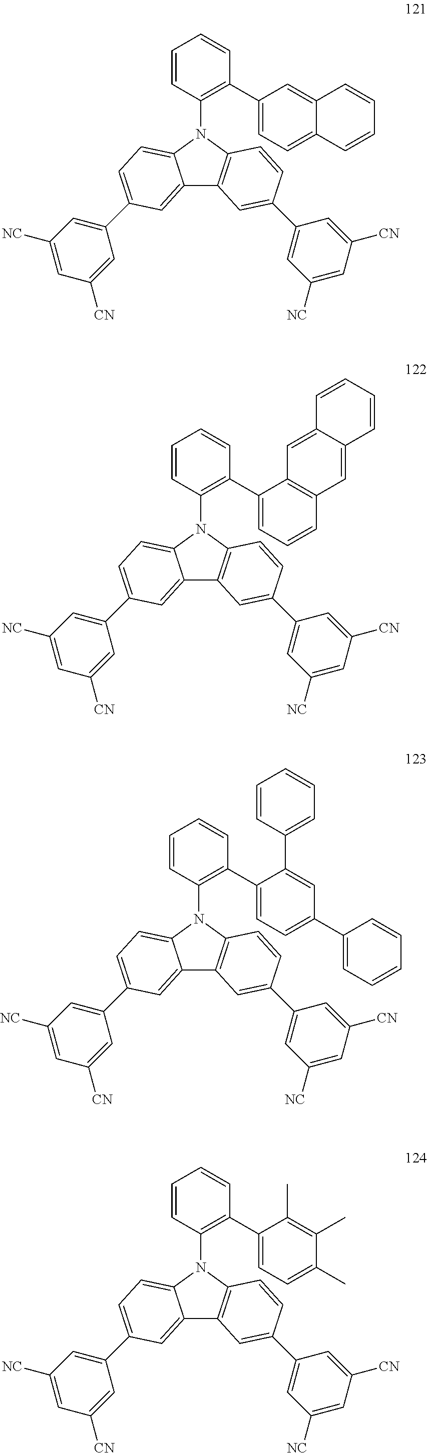 [SMILES: N#Cc1cc(C#N)cc(-c2ccc3c(c2)c2cc(-c4cc(C#N)cc(C#N)c4)ccc2n3-c2ccccc2-c2ccc3ccccc3c2)c1 ; N#Cc1cc(C#N)cc(-c2ccc3c(c2)c2cc(-c4cc(C#N)cc(C#N)c4)ccc2n3-c2ccccc2-c2cccc3cc4ccccc4cc23)c1 ; N#Cc1cc(C#N)cc(-c2ccc3c(c2)c2cc(-c4cc(C#N)cc(C#N)c4)ccc2n3-c2ccccc2-c2ccc(-c3ccccc3)cc2-c2ccccc2)c1 ; Cc1ccc(-c2ccccc2-n2c3ccc(-c4cc(C#N)cc(C#N)c4)cc3c3cc(-c4cc(C#N)cc(C#N)c4)ccc32)c(C)c1C]

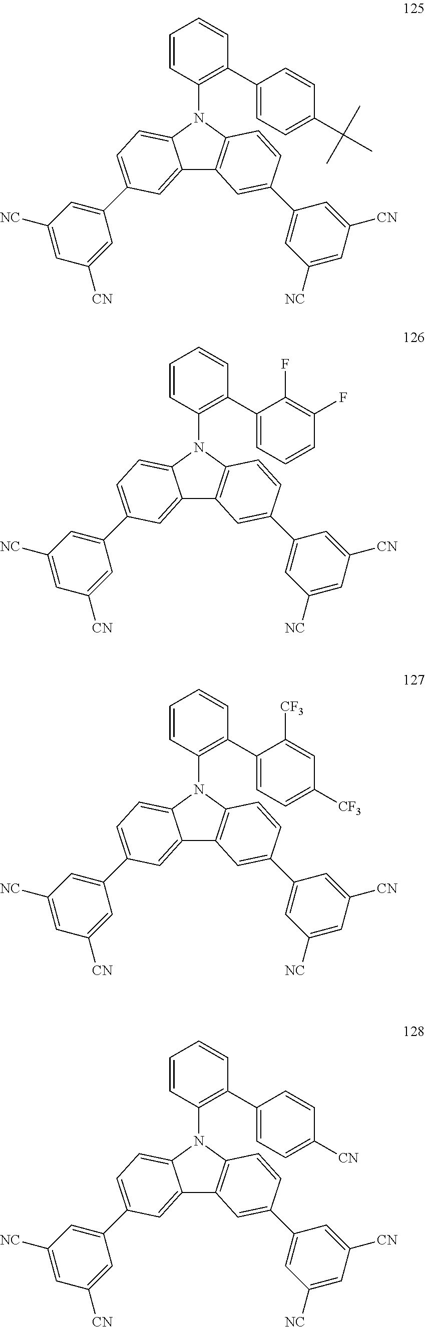 [SMILES: CC(C)(C)c1ccc(-c2ccccc2-n2c3ccc(-c4cc(C#N)cc(C#N)c4)cc3c3cc(-c4cc(C#N)cc(C#N)c4)ccc32)cc1 ; N#Cc1cc(C#N)cc(-c2ccc3c(c2)c2cc(-c4cc(C#N)cc(C#N)c4)ccc2n3-c2ccccc2-c2cccc(F)c2F)c1 ; N#Cc1cc(C#N)cc(-c2ccc3c(c2)c2cc(-c4cc(C#N)cc(C#N)c4)ccc2n3-c2ccccc2-c2ccc(C(F)(F)F)cc2C(F)(F)F)c1 ; N#Cc1ccc(-c2ccccc2-n2c3ccc(-c4cc(C#N)cc(C#N)c4)cc3c3cc(-c4cc(C#N)cc(C#N)c4)ccc32)cc1]

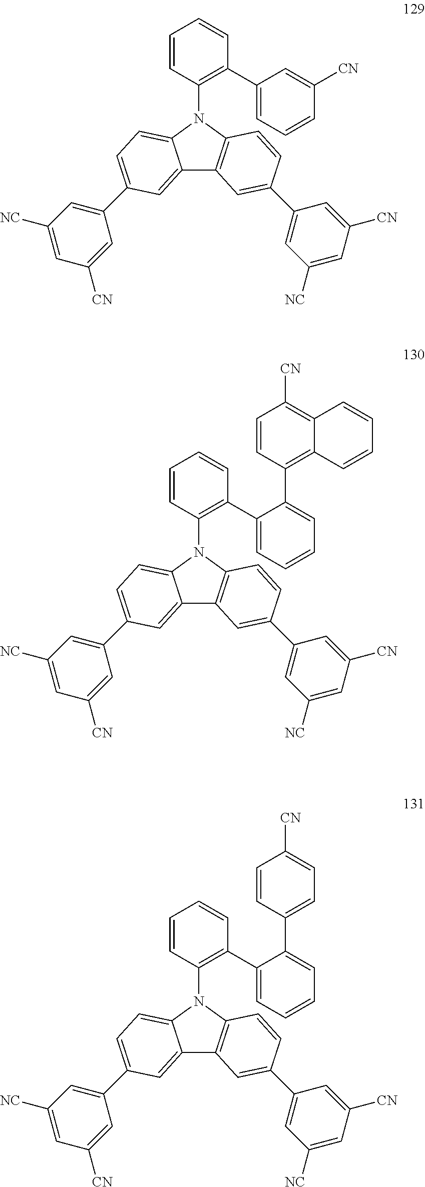 [SMILES: N#Cc1cc(C#N)cc(-c2ccc3c(c2)c2cc(-c4cc(C#N)cc(C#N)c4)ccc2n3-c2ccccc2-c2cccc(C#N)c2)c1 ; N#Cc1cc(C#N)cc(-c2ccc3c(c2)c2cc(-c4cc(C#N)cc(C#N)c4)ccc2n3-c2ccccc2-c2ccccc2-c2ccc(C#N)c3ccccc23)c1 ; N#Cc1ccc(-c2ccccc2-c2ccccc2-n2c3ccc(-c4cc(C#N)cc(C#N)c4)cc3c3cc(-c4cc(C#N)cc(C#N)c4)ccc32)cc1]

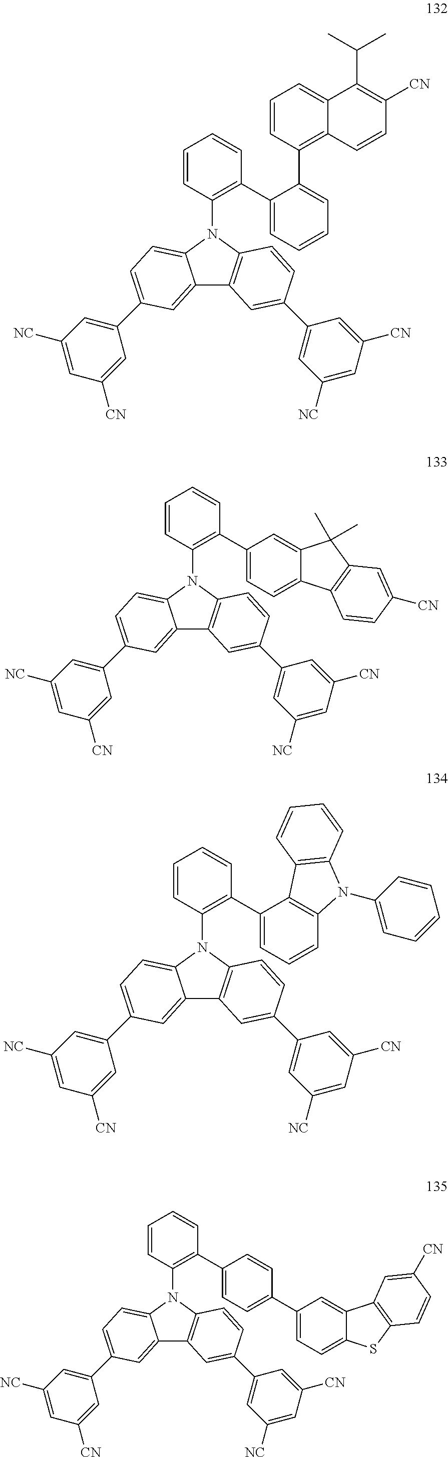 [SMILES: CC(C)c1c(C#N)ccc2c(-c3ccccc3-c3ccccc3-n3c4ccc(-c5cc(C#N)cc(C#N)c5)cc4c4cc(-c5cc(C#N)cc(C#N)c5)ccc43)cccc12 ; CC1(C)c2cc(C#N)ccc2-c2ccc(-c3ccccc3-n3c4ccc(-c5cc(C#N)cc(C#N)c5)cc4c4cc(-c5cc(C#N)cc(C#N)c5)ccc43)cc21 ; N#Cc1cc(C#N)cc(-c2ccc3c(c2)c2cc(-c4cc(C#N)cc(C#N)c4)ccc2n3-c2ccccc2-c2cccc3c2c2ccccc2n3-c2ccccc2)c1 ; N#Cc1cc(C#N)cc(-c2ccc3c(c2)c2cc(-c4cc(C#N)cc(C#N)c4)ccc2n3-c2ccccc2-c2ccc(-c3ccc4sc5ccc(C#N)cc5c4c3)cc2)c1]

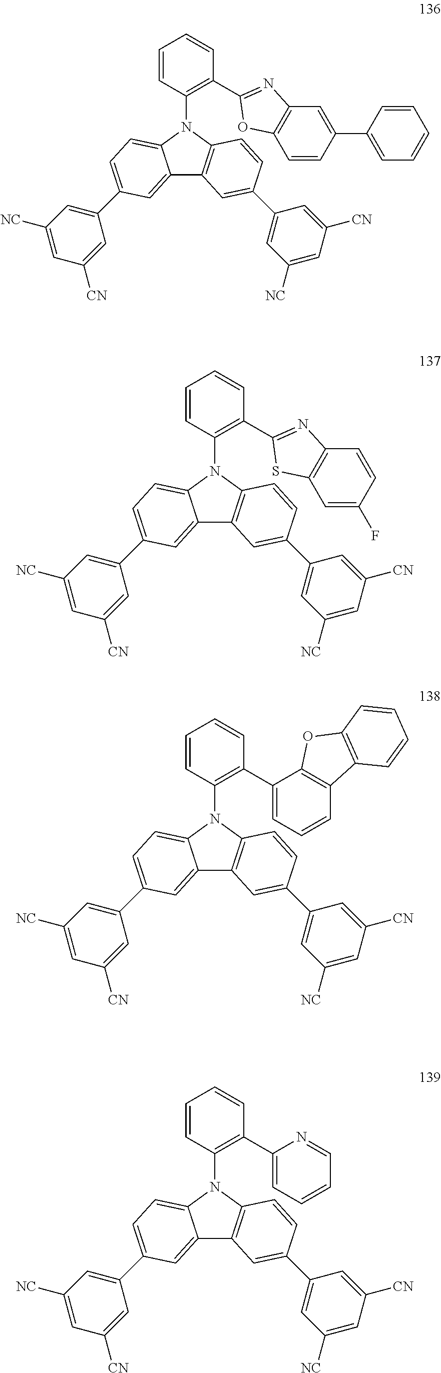 [SMILES: N#Cc1cc(C#N)cc(-c2ccc3c(c2)c2cc(-c4cc(C#N)cc(C#N)c4)ccc2n3-c2ccccc2-c2nc3cc(-c4ccccc4)ccc3o2)c1 ; N#Cc1cc(C#N)cc(-c2ccc3c(c2)c2cc(-c4cc(C#N)cc(C#N)c4)ccc2n3-c2ccccc2-c2nc3ccc(F)cc3s2)c1 ; N#Cc1cc(C#N)cc(-c2ccc3c(c2)c2cc(-c4cc(C#N)cc(C#N)c4)ccc2n3-c2ccccc2-c2cccc3c2oc2ccccc23)c1 ; N#Cc1cc(C#N)cc(-c2ccc3c(c2)c2cc(-c4cc(C#N)cc(C#N)c4)ccc2n3-c2ccccc2-c2ccccn2)c1]

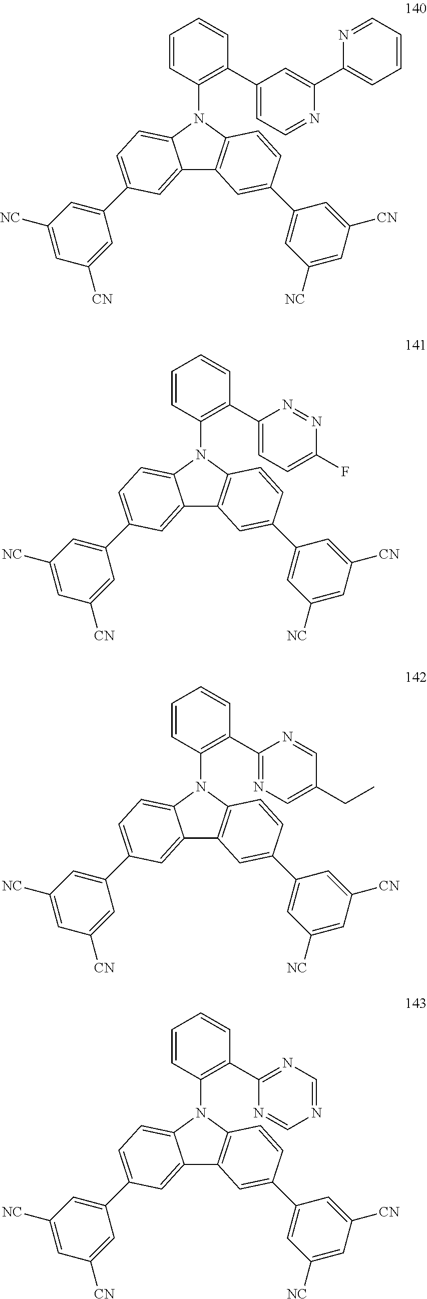 [SMILES: N#Cc1cc(C#N)cc(-c2ccc3c(c2)c2cc(-c4cc(C#N)cc(C#N)c4)ccc2n3-c2ccccc2-c2ccnc(-c3ccccn3)c2)c1 ; N#Cc1cc(C#N)cc(-c2ccc3c(c2)c2cc(-c4cc(C#N)cc(C#N)c4)ccc2n3-c2ccccc2-c2ccc(F)nn2)c1 ; CCc1cnc(-c2ccccc2-n2c3ccc(-c4cc(C#N)cc(C#N)c4)cc3c3cc(-c4cc(C#N)cc(C#N)c4)ccc32)nc1 ; N#Cc1cc(C#N)cc(-c2ccc3c(c2)c2cc(-c4cc(C#N)cc(C#N)c4)ccc2n3-c2ccccc2-c2ncncn2)c1]

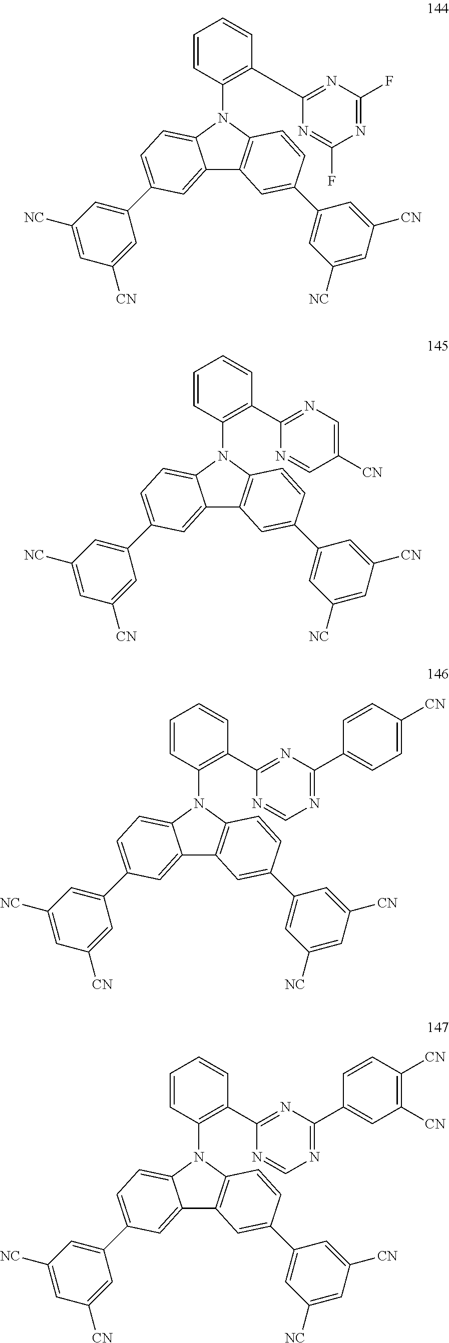 [SMILES: N#Cc1cc(C#N)cc(-c2ccc3c(c2)c2cc(-c4cc(C#N)cc(C#N)c4)ccc2n3-c2ccccc2-c2nc(F)nc(F)n2)c1 ; N#Cc1cnc(-c2ccccc2-n2c3ccc(-c4cc(C#N)cc(C#N)c4)cc3c3cc(-c4cc(C#N)cc(C#N)c4)ccc32)nc1 ; N#Cc1ccc(-c2ncnc(-c3ccccc3-n3c4ccc(-c5cc(C#N)cc(C#N)c5)cc4c4cc(-c5cc(C#N)cc(C#N)c5)ccc43)n2)cc1 ; N#Cc1cc(C#N)cc(-c2ccc3c(c2)c2cc(-c4cc(C#N)cc(C#N)c4)ccc2n3-c2ccccc2-c2ncnc(-c3ccc(C#N)c(C#N)c3)n2)c1]

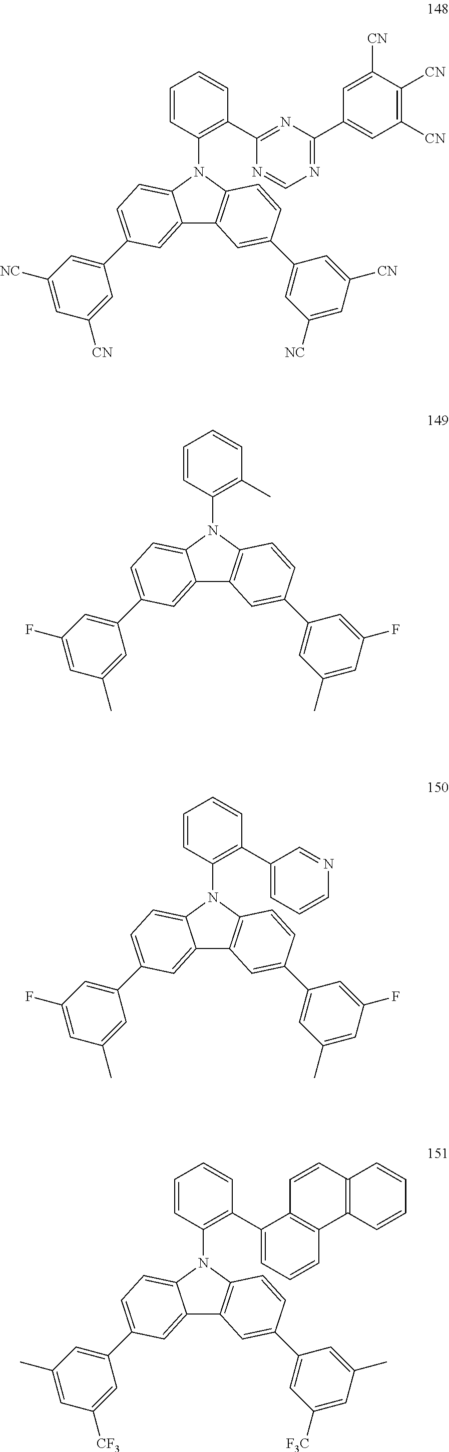 [SMILES: N#Cc1cc(C#N)cc(-c2ccc3c(c2)c2cc(-c4cc(C#N)cc(C#N)c4)ccc2n3-c2ccccc2-c2ncnc(-c3cc(C#N)c(C#N)c(C#N)c3)n2)c1 ; Cc1cc(F)cc(-c2ccc3c(c2)c2cc(-c4cc(C)cc(F)c4)ccc2n3-c2ccccc2C)c1 ; Cc1cc(F)cc(-c2ccc3c(c2)c2cc(-c4cc(C)cc(F)c4)ccc2n3-c2ccccc2-c2cccnc2)c1 ; Cc1cc(-c2ccc3c(c2)c2cc(-c4cc(C)cc(C(F)(F)F)c4)ccc2n3-c2ccccc2-c2cccc3c2ccc2ccccc23)cc(C(F)(F)F)c1]

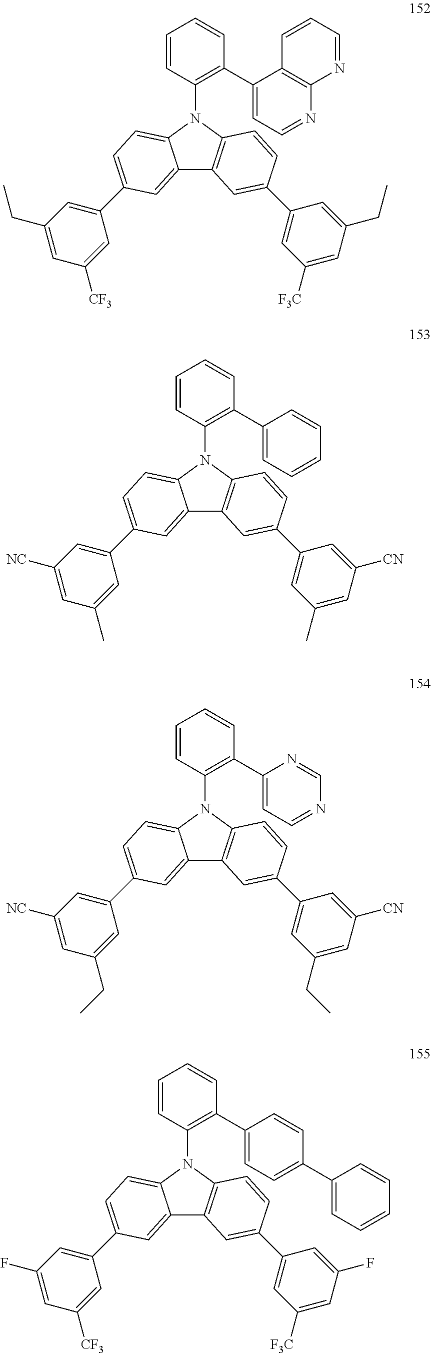 [SMILES: CCc1cc(-c2ccc3c(c2)c2cc(-c4cc(CC)cc(C(F)(F)F)c4)ccc2n3-c2ccccc2-c2ccnc3ncccc23)cc(C(F)(F)F)c1 ; Cc1cc(C#N)cc(-c2ccc3c(c2)c2cc(-c4cc(C)cc(C#N)c4)ccc2n3-c2ccccc2-c2ccccc2)c1 ; CCc1cc(C#N)cc(-c2ccc3c(c2)c2cc(-c4cc(C#N)cc(CC)c4)ccc2n3-c2ccccc2-c2ccncn2)c1 ; Fc1cc(-c2ccc3c(c2)c2cc(-c4cc(F)cc(C(F)(F)F)c4)ccc2n3-c2ccccc2-c2ccc(-c3ccccc3)cc2)cc(C(F)(F)F)c1]

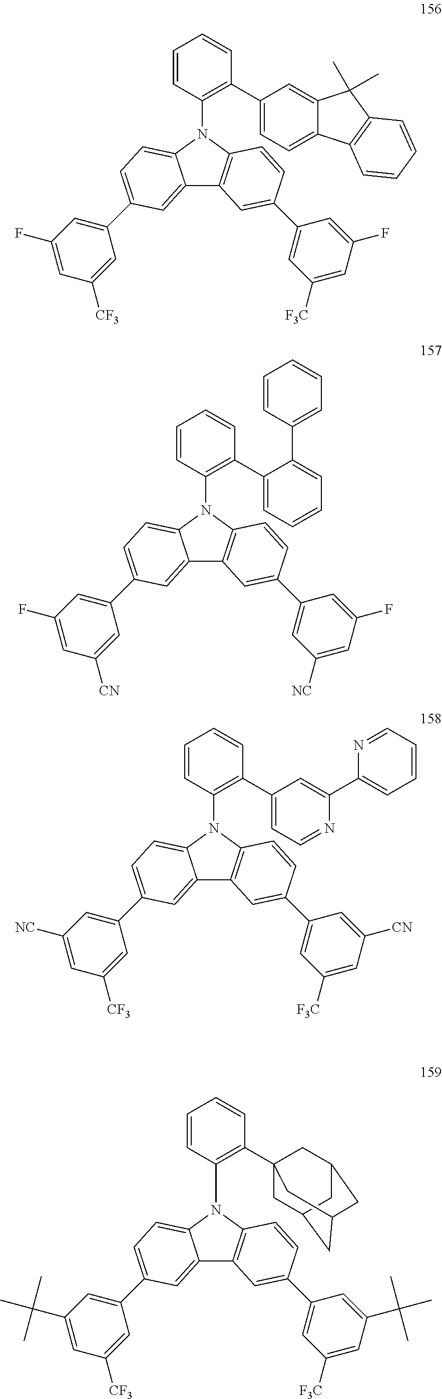

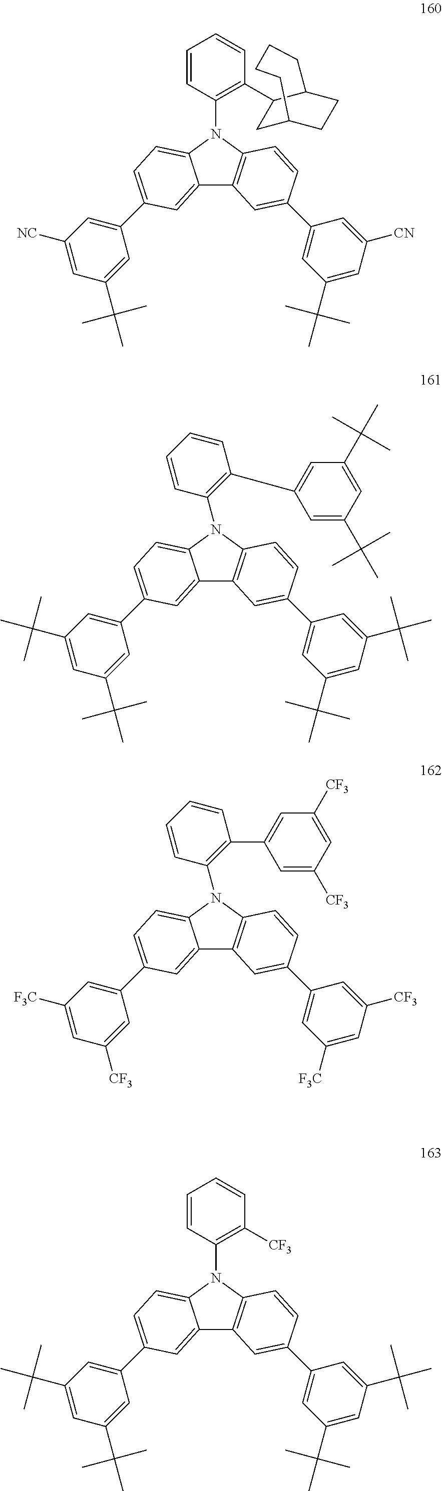

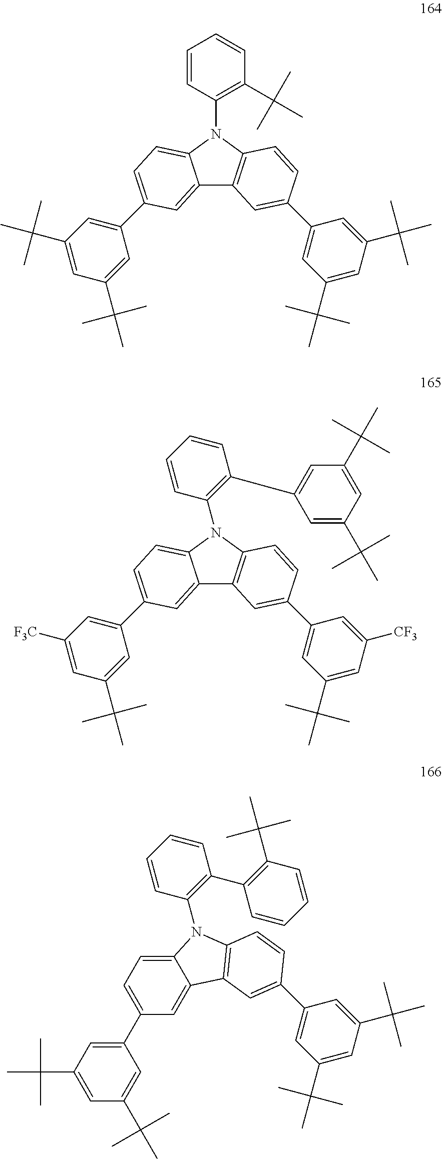

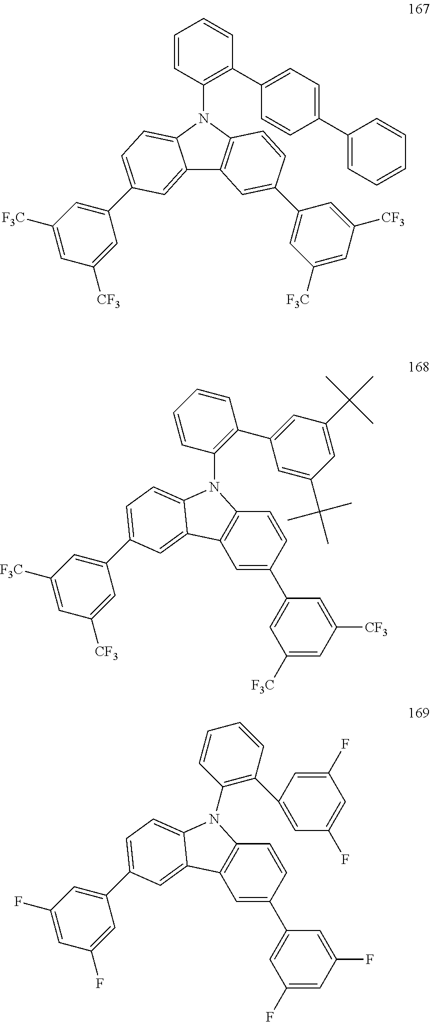 [SMILES: FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2)cc(C(F)(F)F)c1 ; CC(C)(C)c1cc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cc(C(C)(C)C)c1 ; Fc1cc(F)cc(-c2ccc3c(c2)c2cc(-c4cc(F)cc(F)c4)ccc2n3-c2ccccc2-c2cc(F)cc(F)c2)c1]

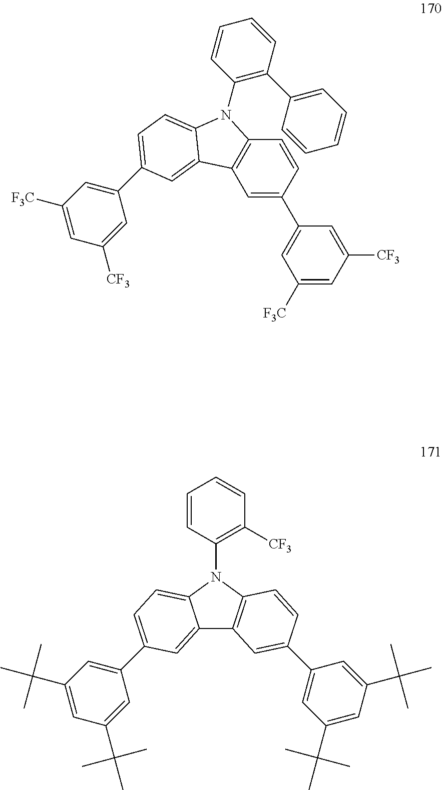 [SMILES: FC(F)(F)c1cc(-c2ccc3c(c2)c2cc(-c4cc(C(F)(F)F)cc(C(F)(F)F)c4)ccc2n3-c2ccccc2-c2ccccc2)cc(C(F)(F)F)c1 ; CC(C)(C)c1cc(-c2ccc3c(c2)c2cc(-c4cc(C(C)(C)C)cc(C(C)(C)C)c4)ccc2n3-c2ccccc2C(F)(F)F)cc(C(C)(C)C)c1]

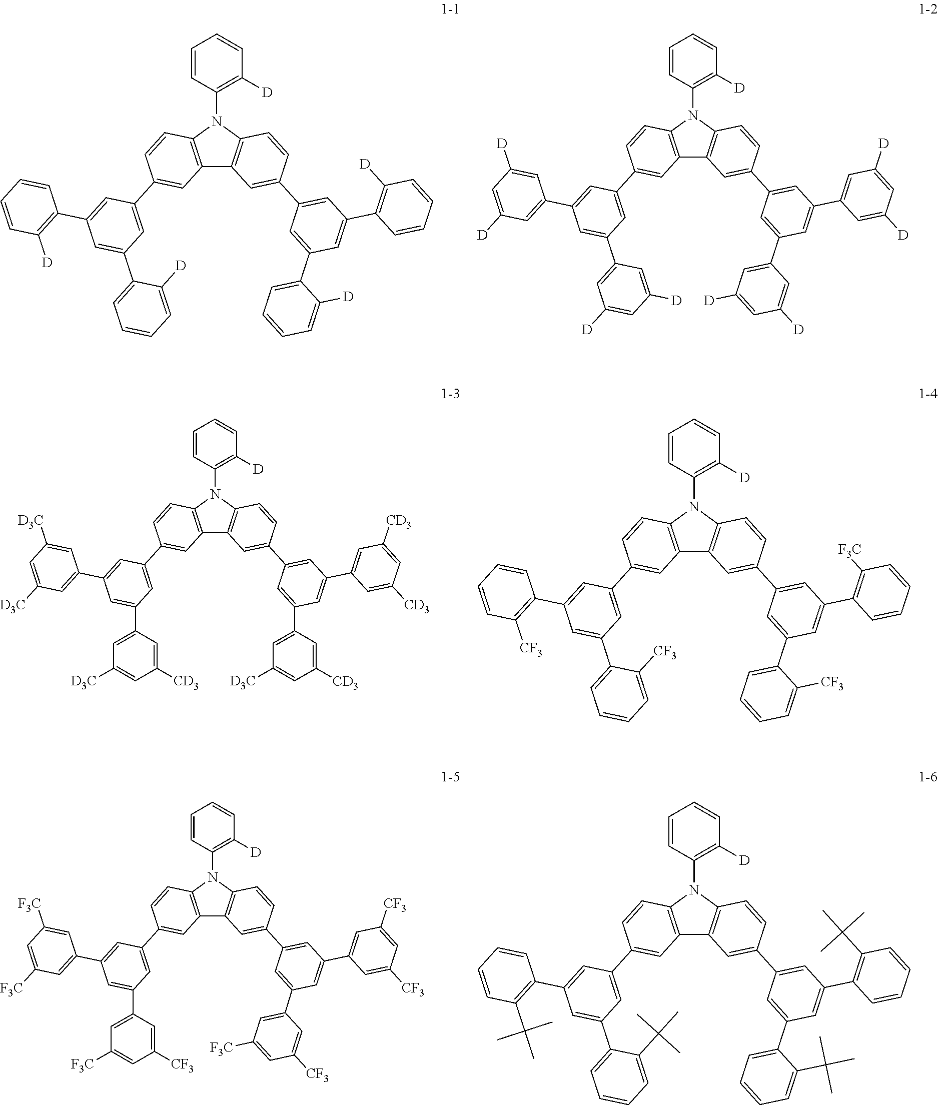

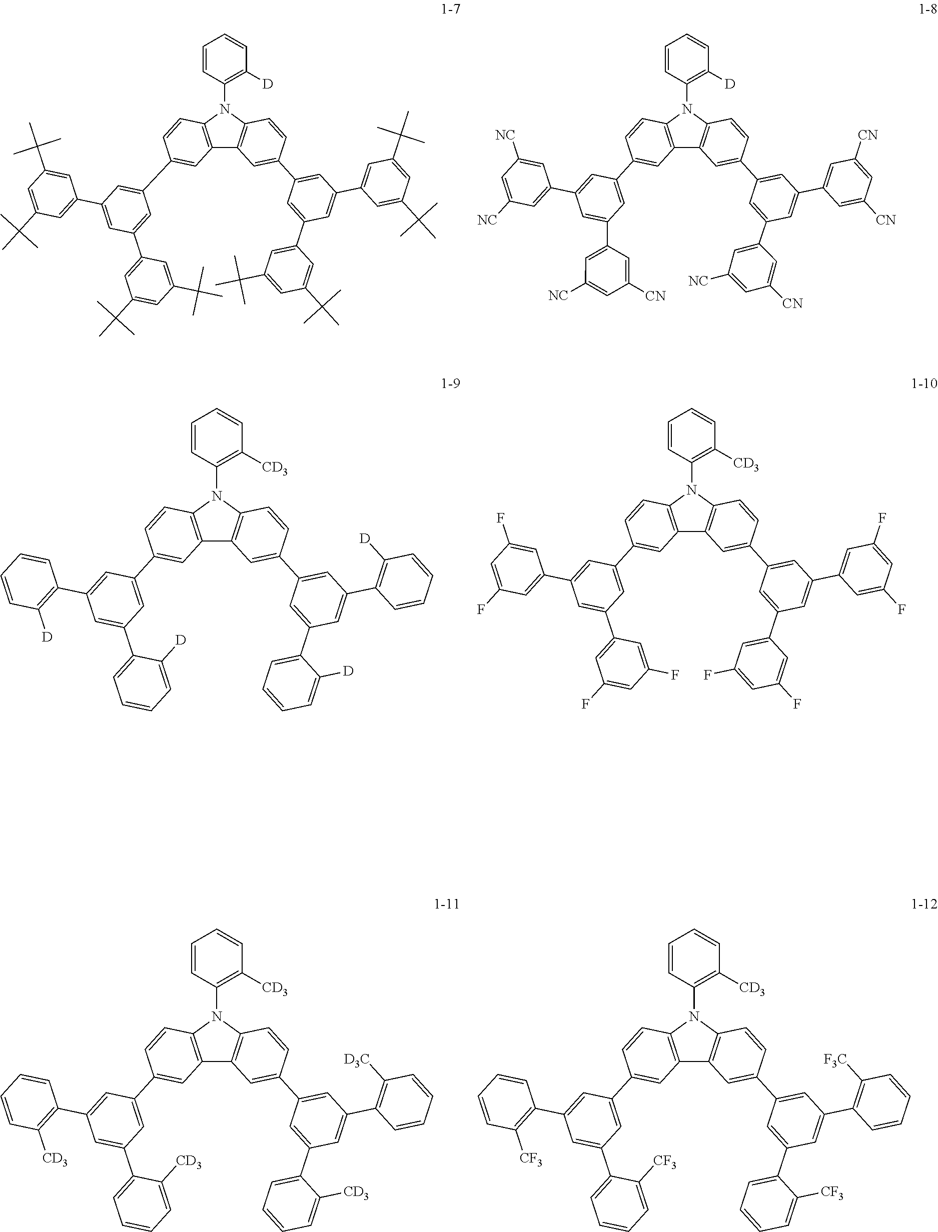

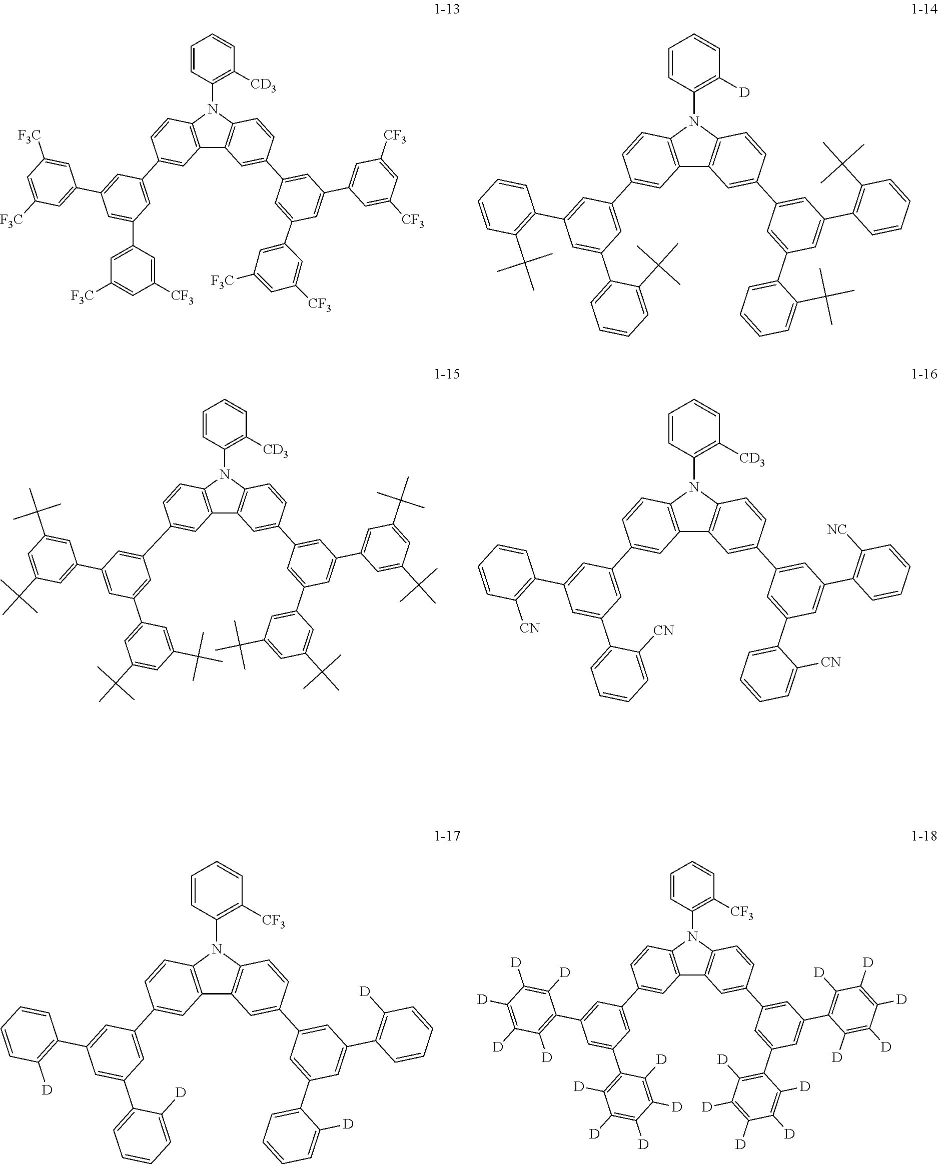

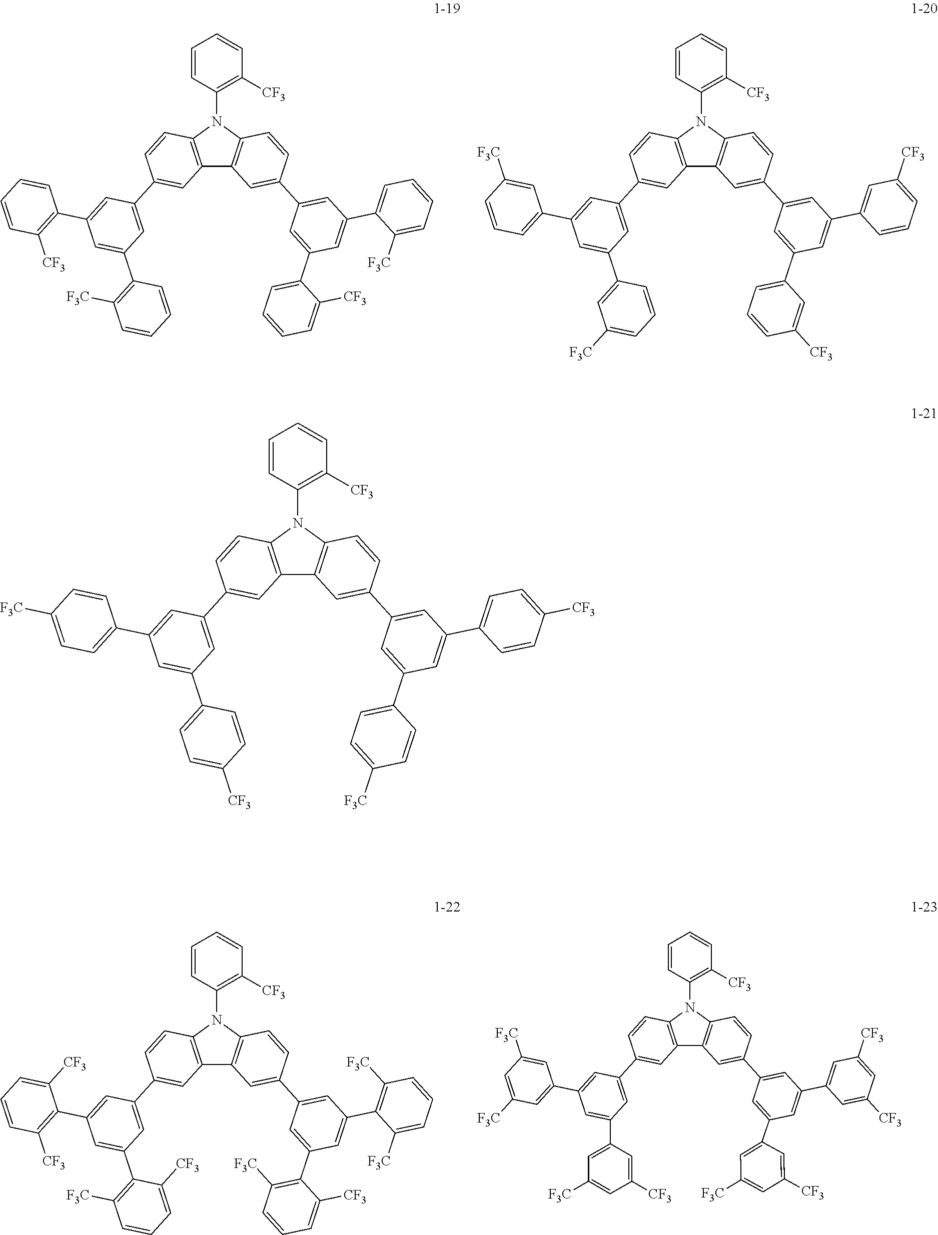

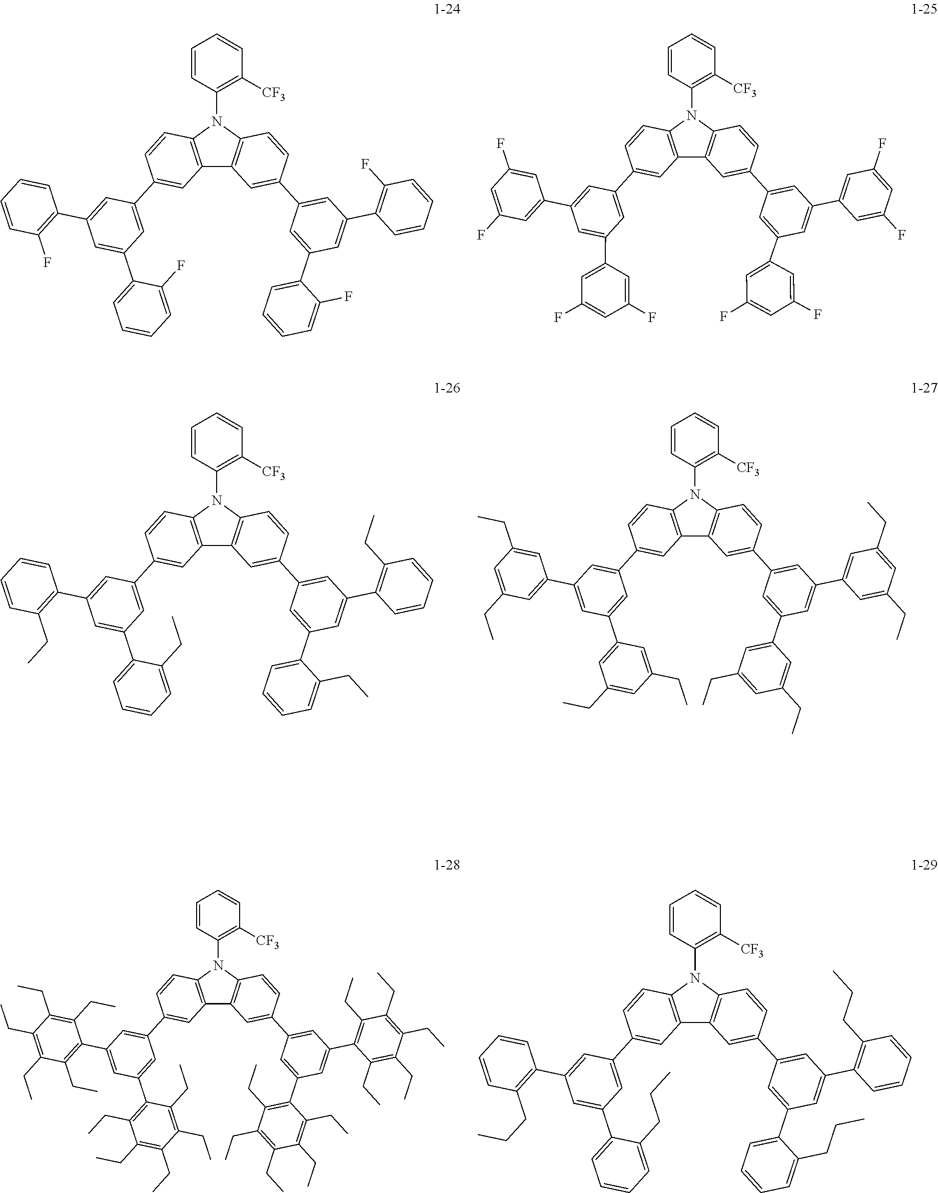

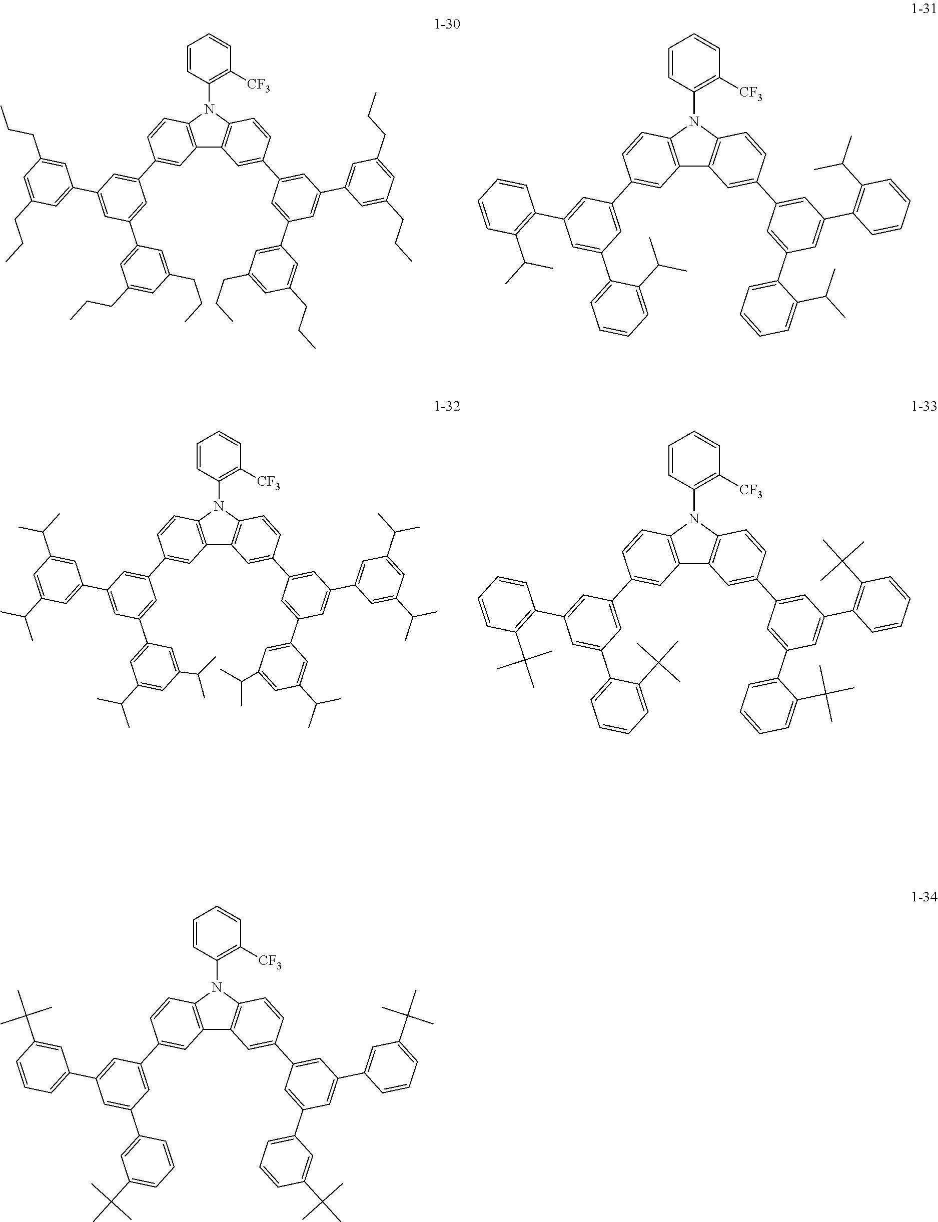

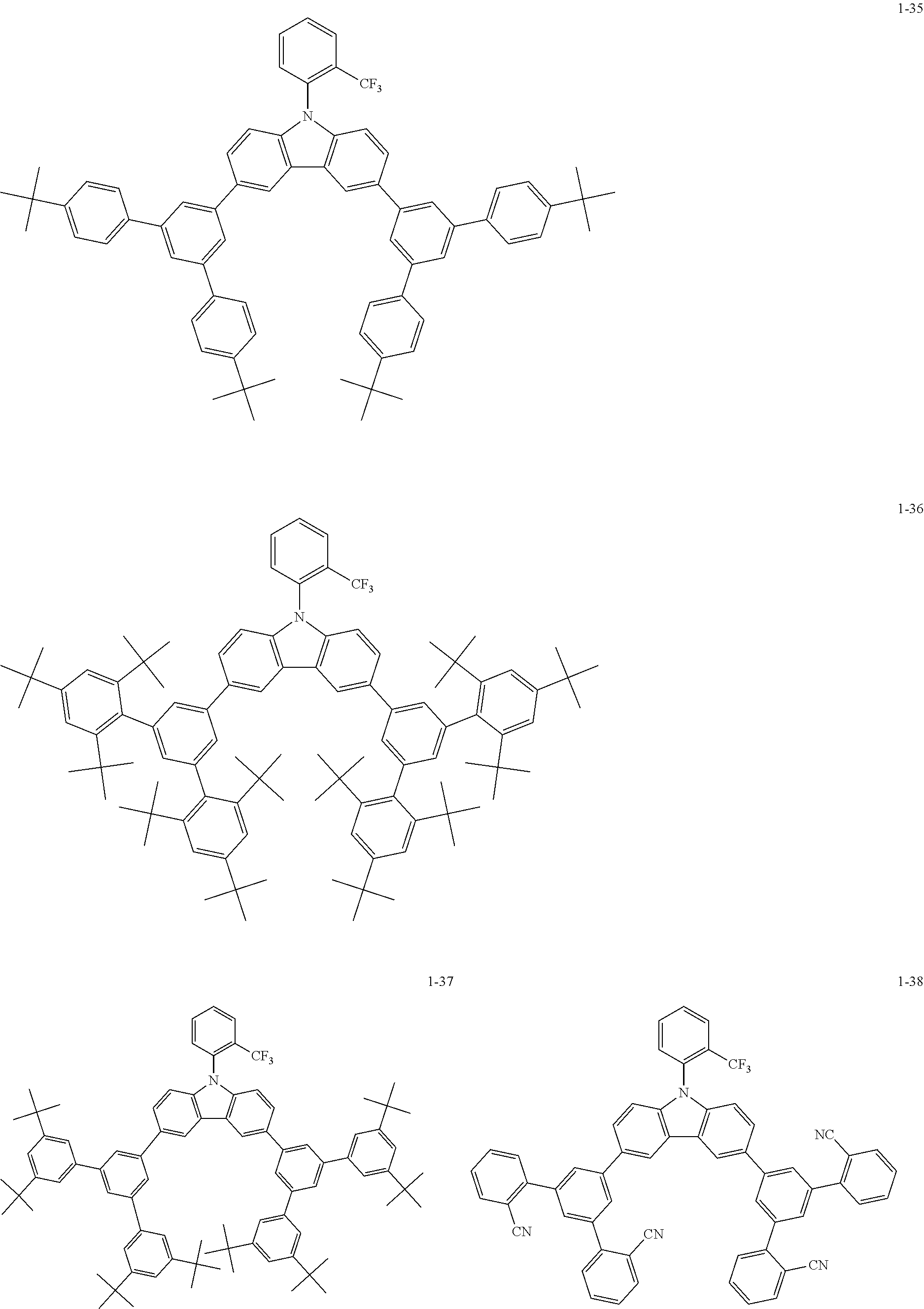

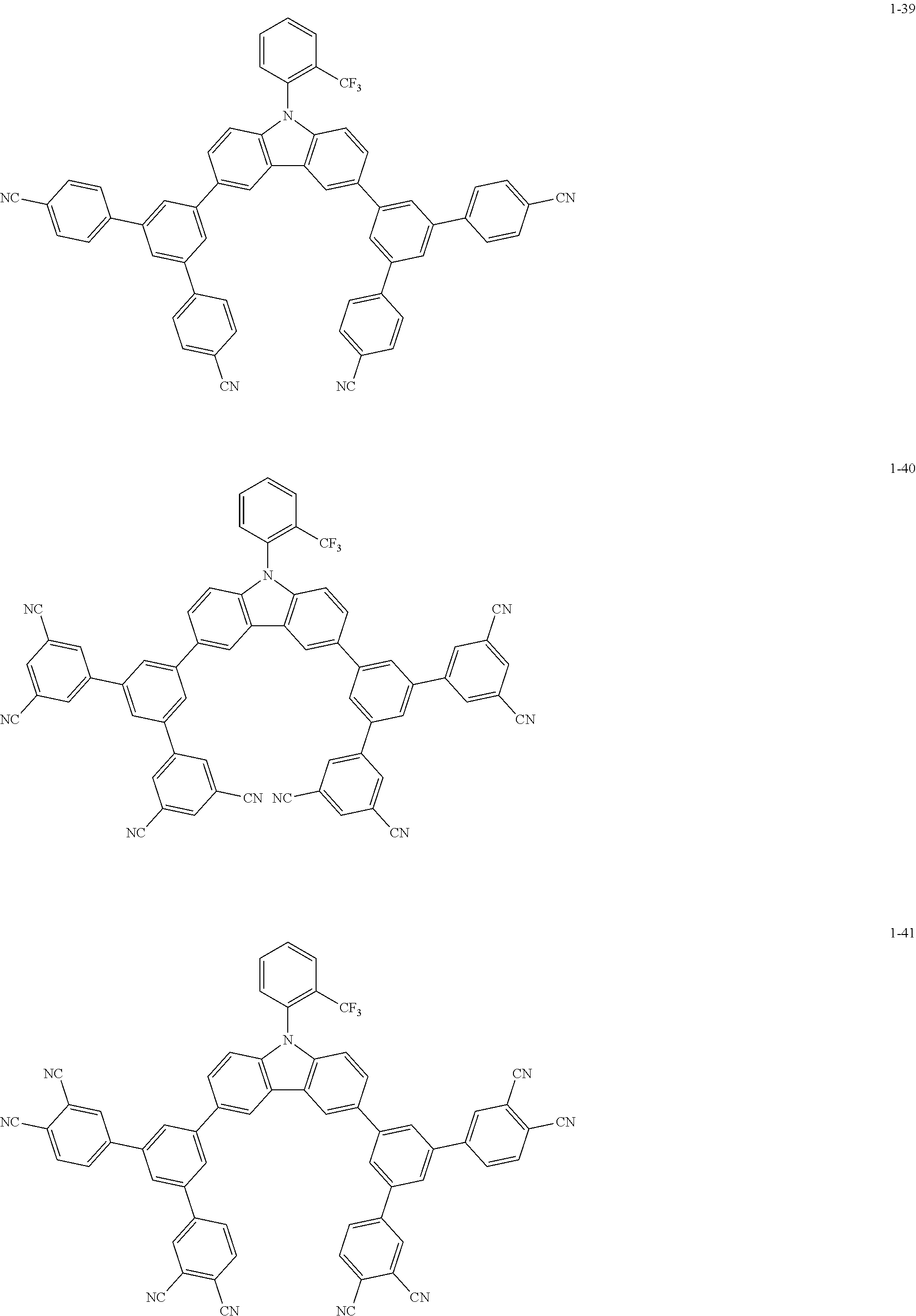 [SMILES: N#Cc1ccc(-c2cc(-c3ccc(C#N)cc3)cc(-c3ccc4c(c3)c3cc(-c5cc(-c6ccc(C#N)cc6)cc(-c6ccc(C#N)cc6)c5)ccc3n4-c3ccccc3C(F)(F)F)c2)cc1 ; N#Cc1cc(C#N)cc(-c2cc(-c3cc(C#N)cc(C#N)c3)cc(-c3ccc4c(c3)c3cc(-c5cc(-c6cc(C#N)cc(C#N)c6)cc(-c6cc(C#N)cc(C#N)c6)c5)ccc3n4-c3ccccc3C(F)(F)F)c2)c1 ; N#Cc1ccc(-c2cc(-c3ccc(C#N)c(C#N)c3)cc(-c3ccc4c(c3)c3cc(-c5cc(-c6ccc(C#N)c(C#N)c6)cc(-c6ccc(C#N)c(C#N)c6)c5)ccc3n4-c3ccccc3C(F)(F)F)c2)cc1C#N]

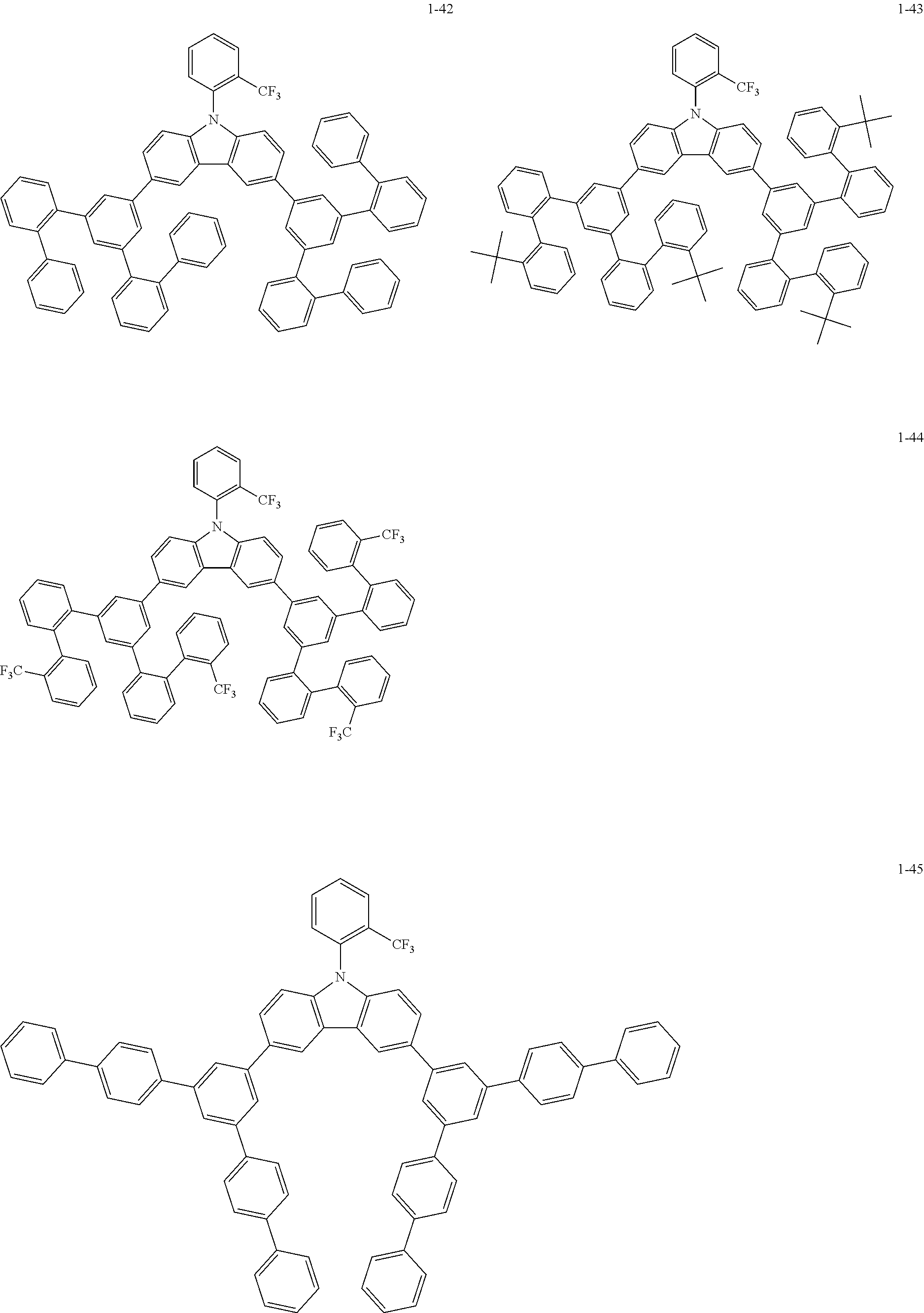

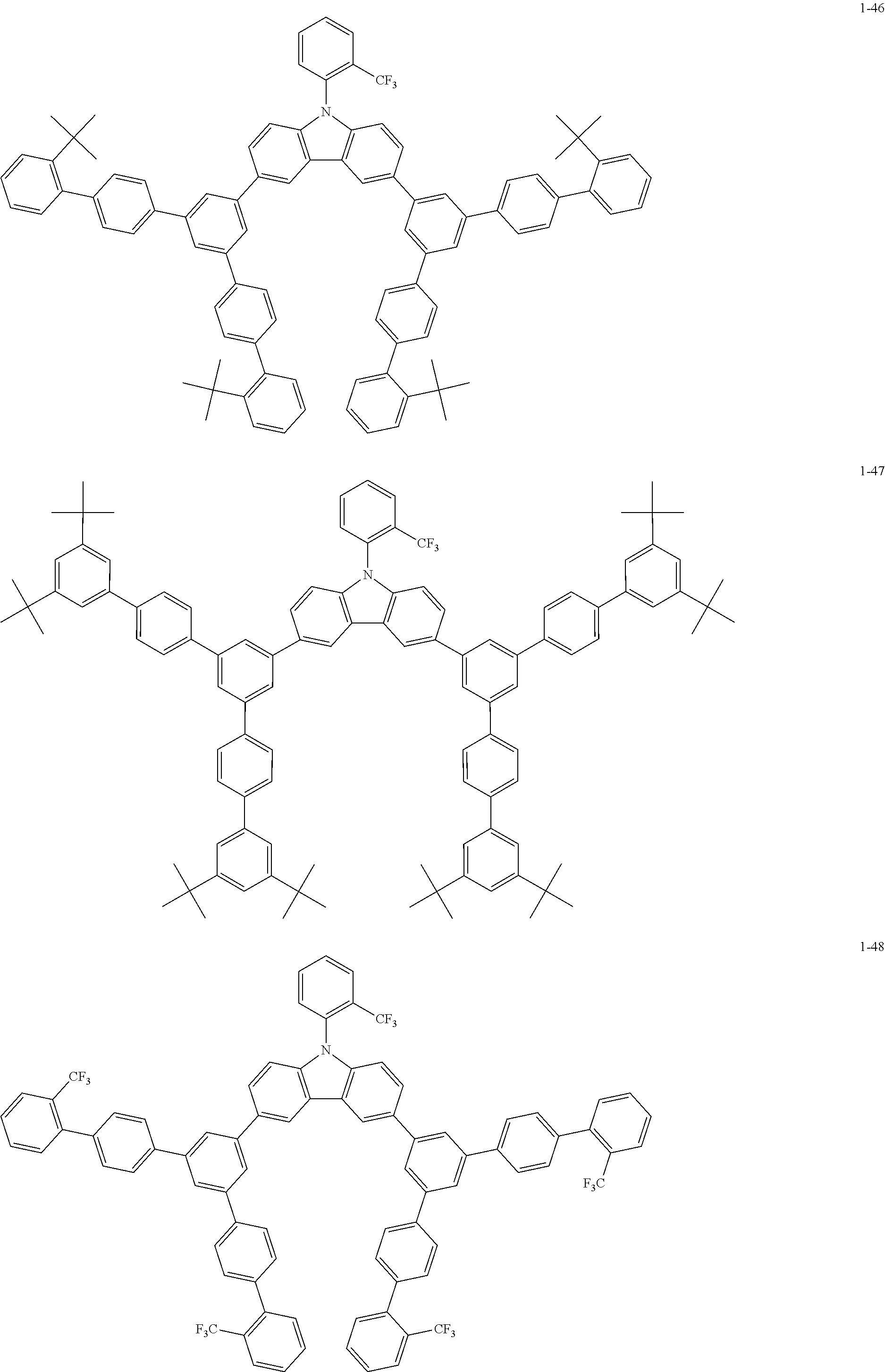

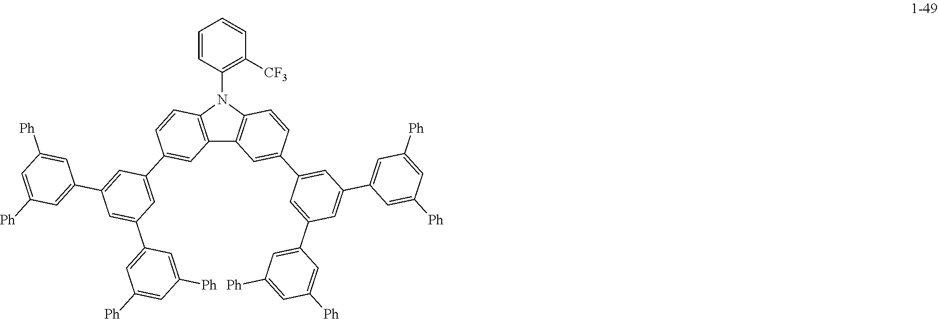 [SMILES: FC(F)(F)c1ccccc1-n1c2ccc(-c3cc(-c4cc(-c5ccccc5)cc(-c5ccccc5)c4)cc(-c4cc(-c5ccccc5)cc(-c5ccccc5)c4)c3)cc2c2cc(-c3cc(-c4cc(-c5ccccc5)cc(-c5ccccc5)c4)cc(-c4cc(-c5ccccc5)cc(-c5ccccc5)c4)c3)ccc21]

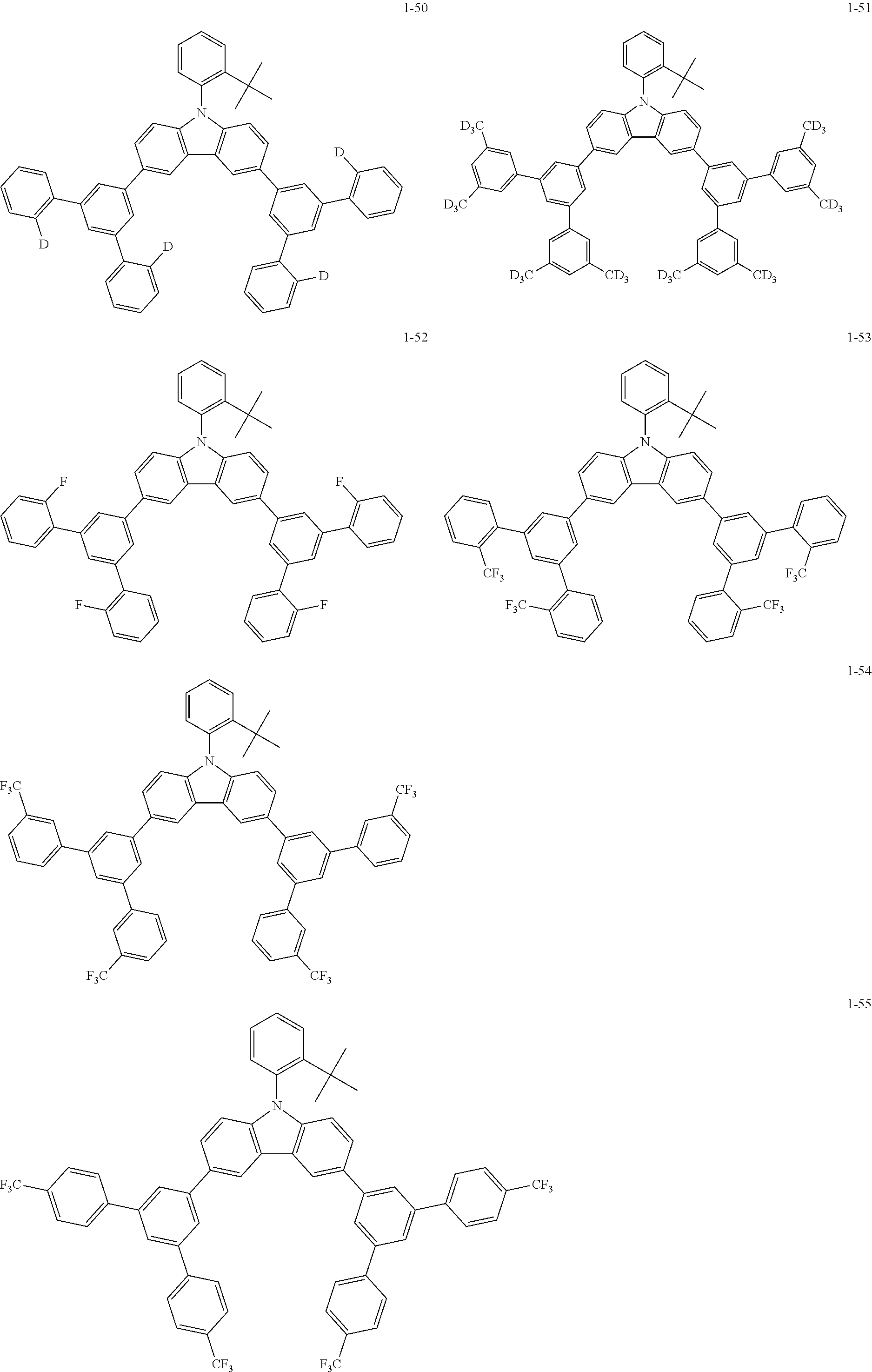

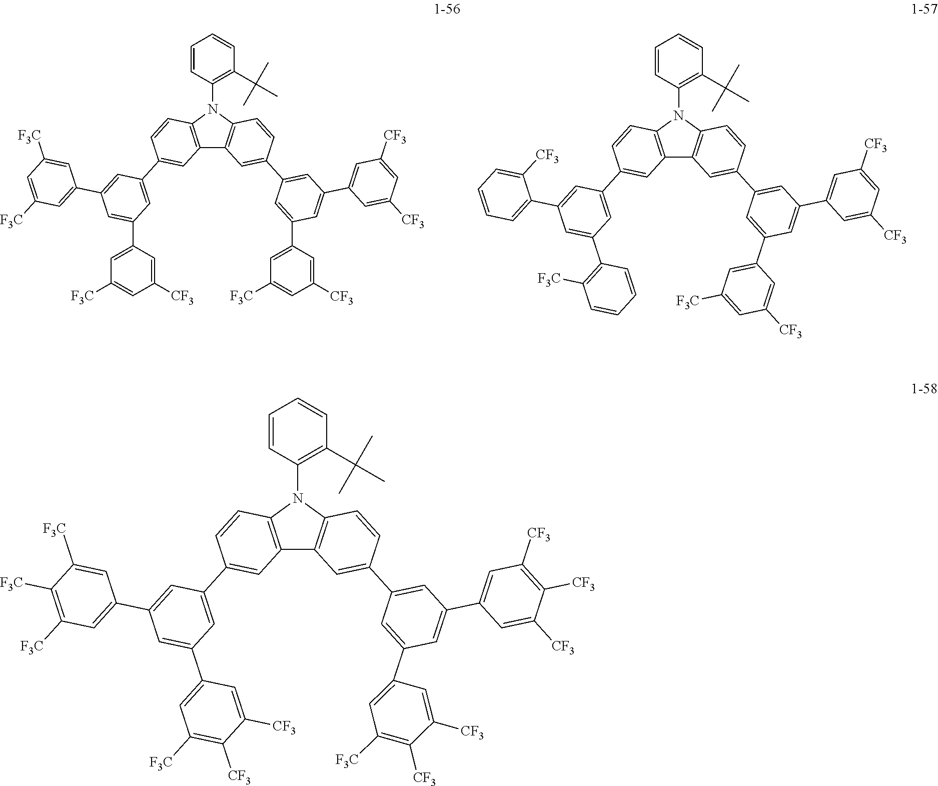

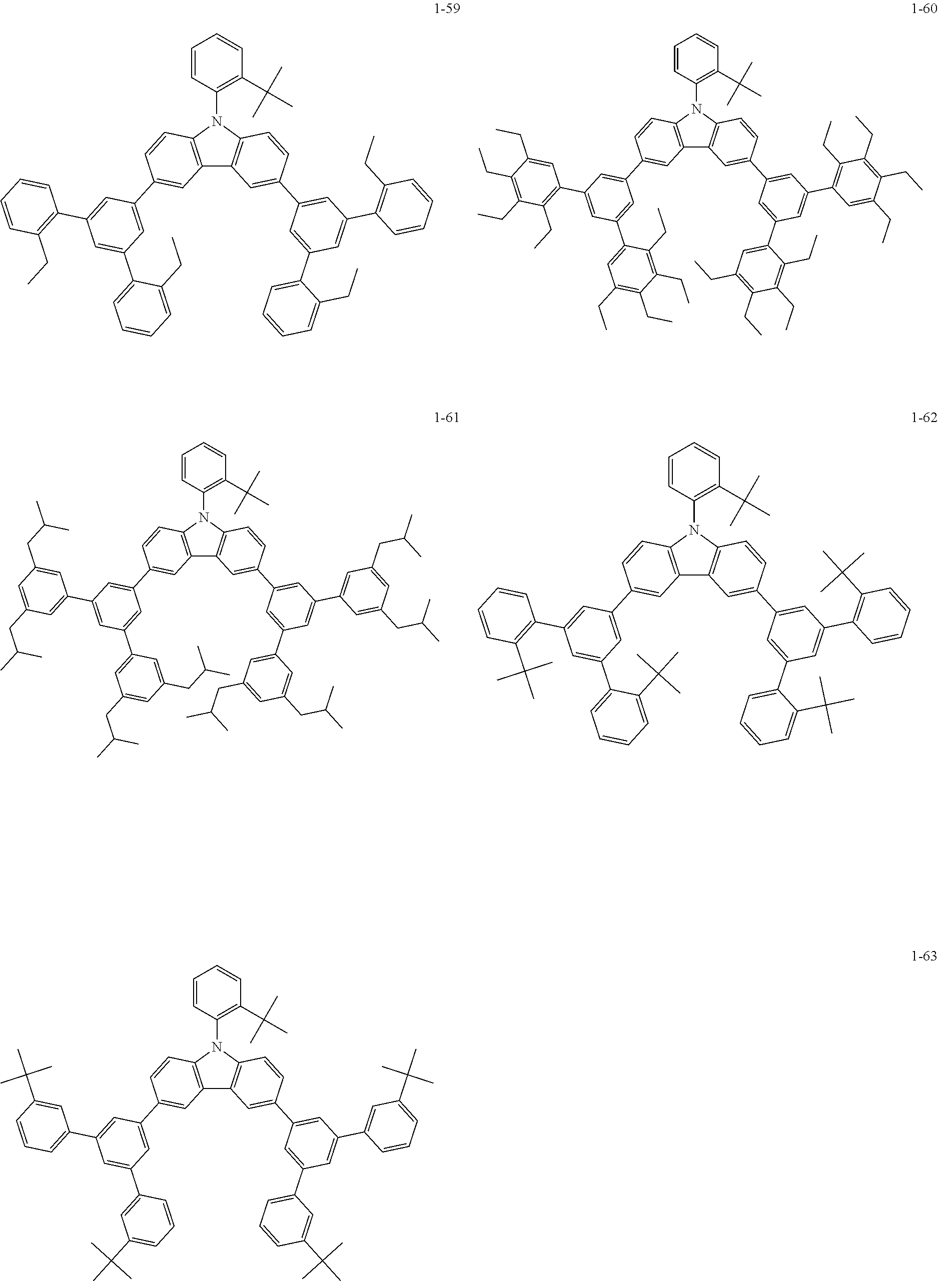

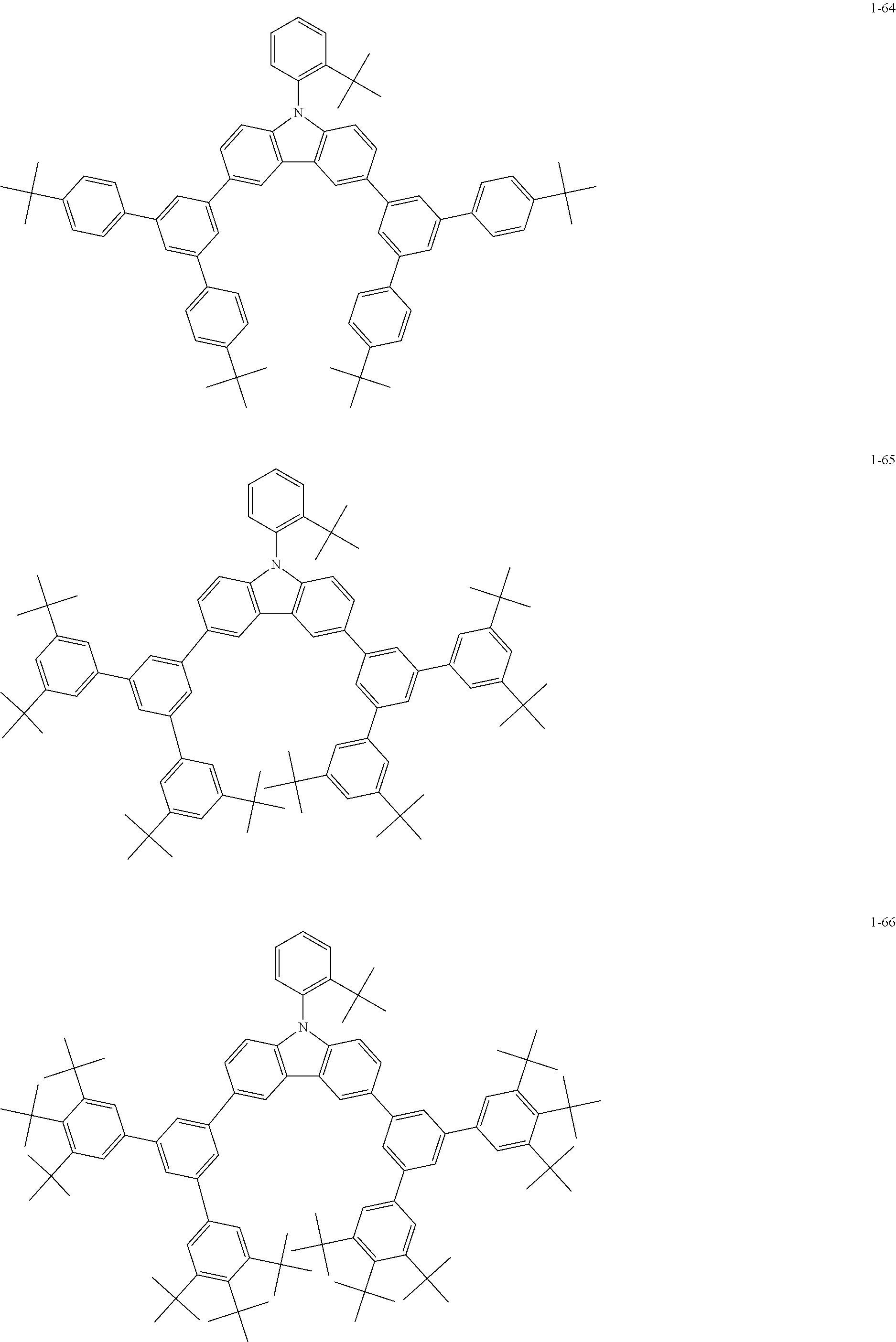

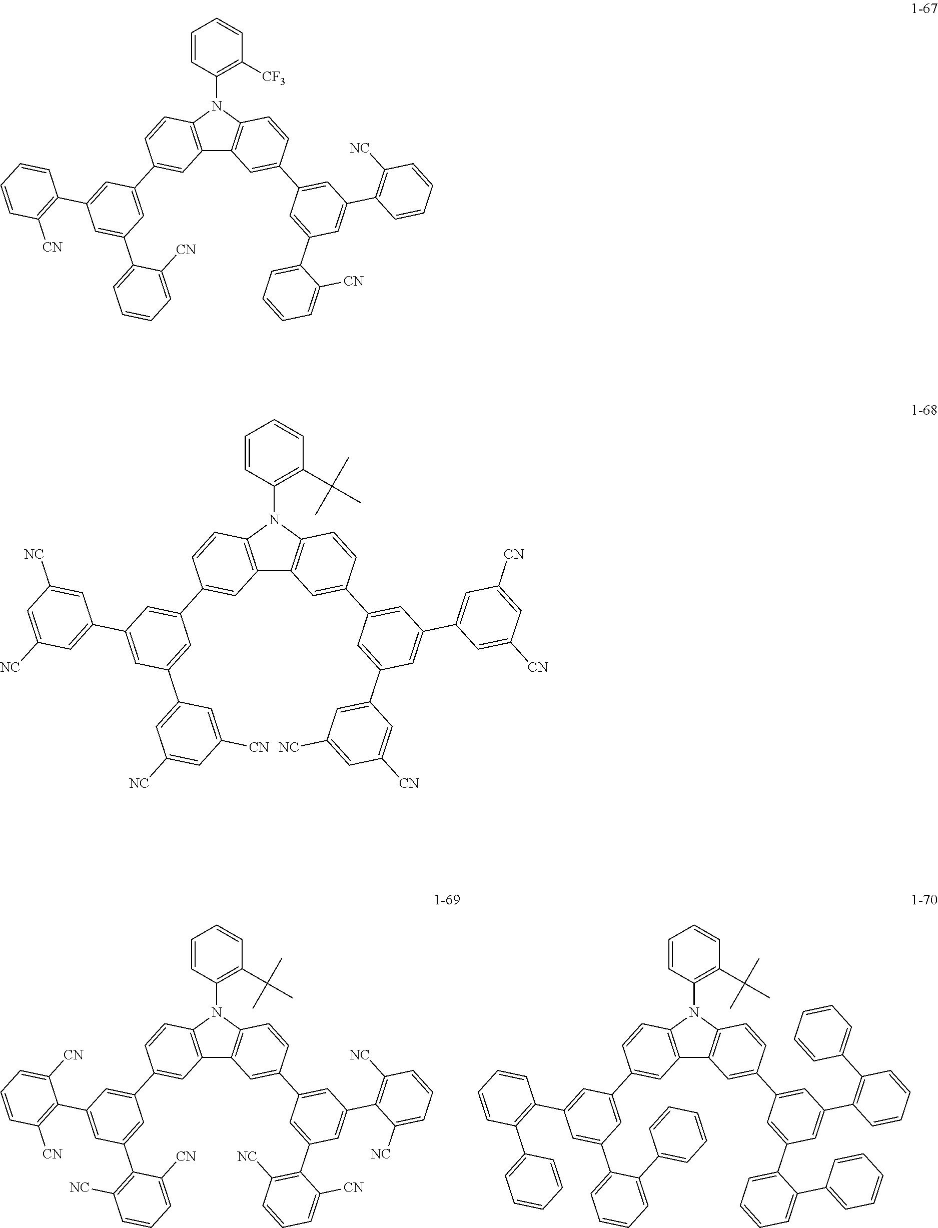

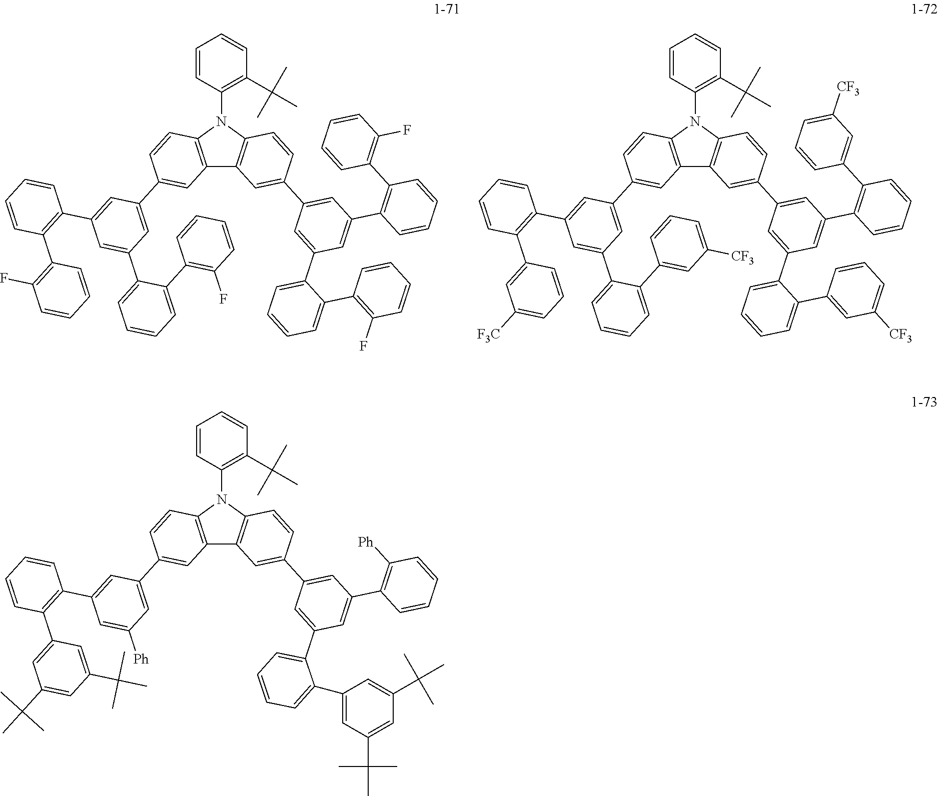 [SMILES: CC(C)(C)c1ccccc1-n1c2ccc(-c3cc(-c4ccccc4-c4ccccc4F)cc(-c4ccccc4-c4ccccc4F)c3)cc2c2cc(-c3cc(-c4ccccc4-c4ccccc4F)cc(-c4ccccc4-c4ccccc4F)c3)ccc21 ; CC(C)(C)c1ccccc1-n1c2ccc(-c3cc(-c4ccccc4-c4cccc(C(F)(F)F)c4)cc(-c4ccccc4-c4cccc(C(F)(F)F)c4)c3)cc2c2cc(-c3cc(-c4ccccc4-c4cccc(C(F)(F)F)c4)cc(-c4ccccc4-c4cccc(C(F)(F)F)c4)c3)ccc21 ; CC(C)(C)c1cc(-c2ccccc2-c2cc(-c3ccccc3)cc(-c3ccc4c(c3)c3cc(-c5cc(-c6ccccc6-c6ccccc6)cc(-c6ccccc6-c6cc(C(C)(C)C)cc(C(C)(C)C)c6)c5)ccc3n4-c3ccccc3C(C)(C)C)c2)cc(C(C)(C)C)c1]

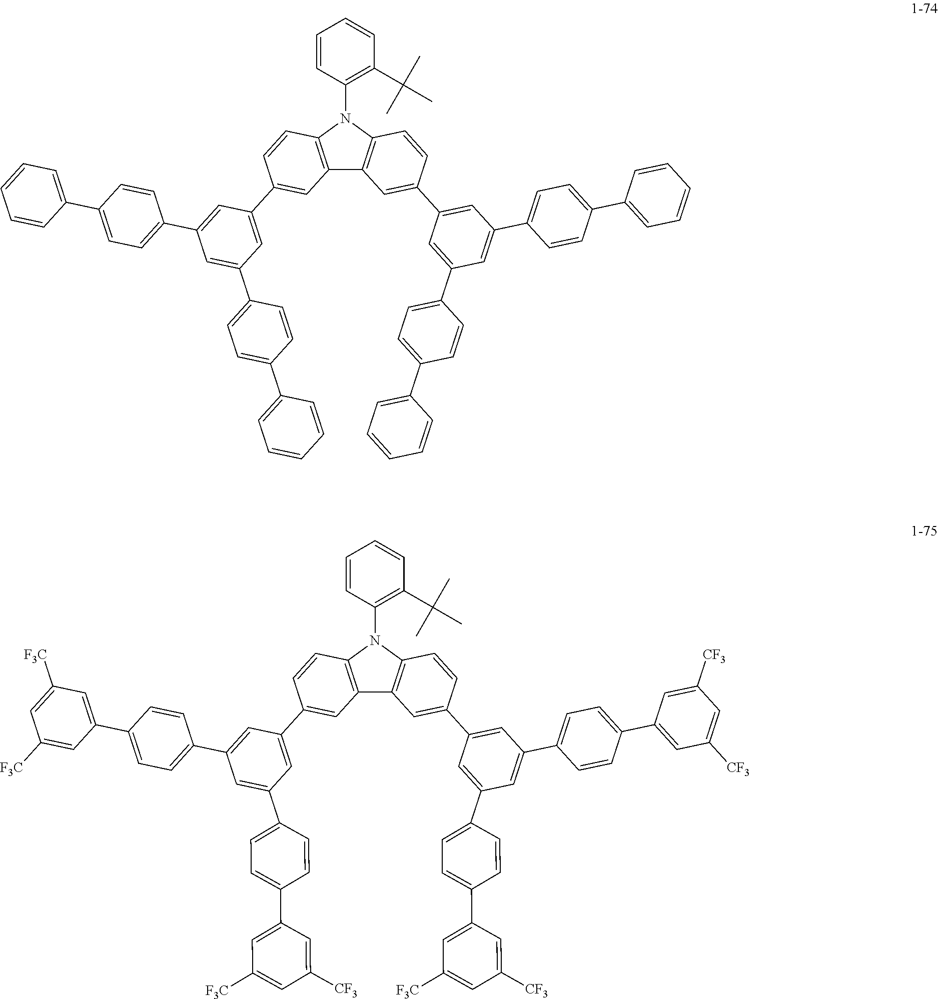 [SMILES: CC(C)(C)c1ccccc1-n1c2ccc(-c3cc(-c4ccc(-c5ccccc5)cc4)cc(-c4ccc(-c5ccccc5)cc4)c3)cc2c2cc(-c3cc(-c4ccc(-c5ccccc5)cc4)cc(-c4ccc(-c5ccccc5)cc4)c3)ccc21 ; CC(C)(C)c1ccccc1-n1c2ccc(-c3cc(-c4ccc(-c5cc(C(F)(F)F)cc(C(F)(F)F)c5)cc4)cc(-c4ccc(-c5cc(C(F)(F)F)cc(C(F)(F)F)c5)cc4)c3)cc2c2cc(-c3cc(-c4ccc(-c5cc(C(F)(F)F)cc(C(F)(F)F)c5)cc4)cc(-c4ccc(-c5cc(C(F)(F)F)cc(C(F)(F)F)c5)cc4)c3)ccc21]

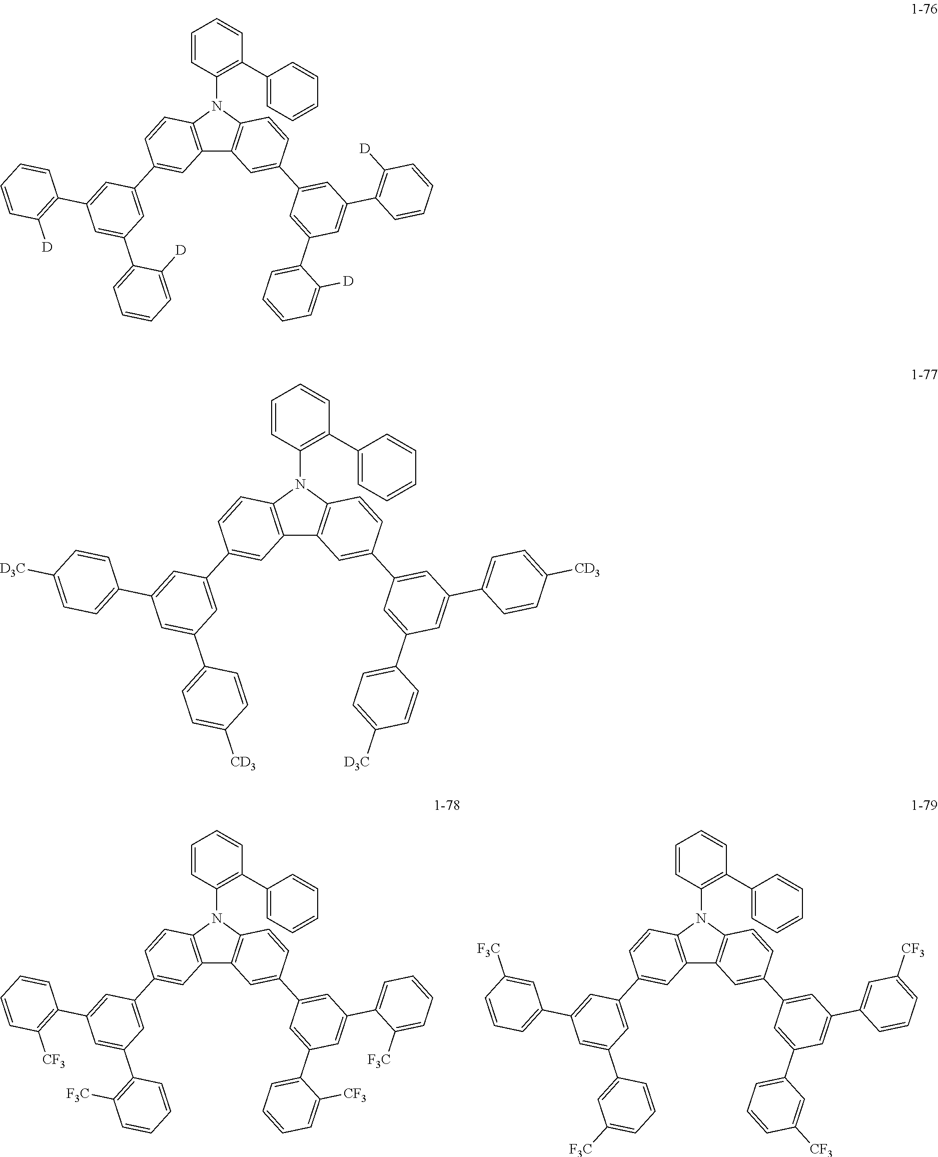

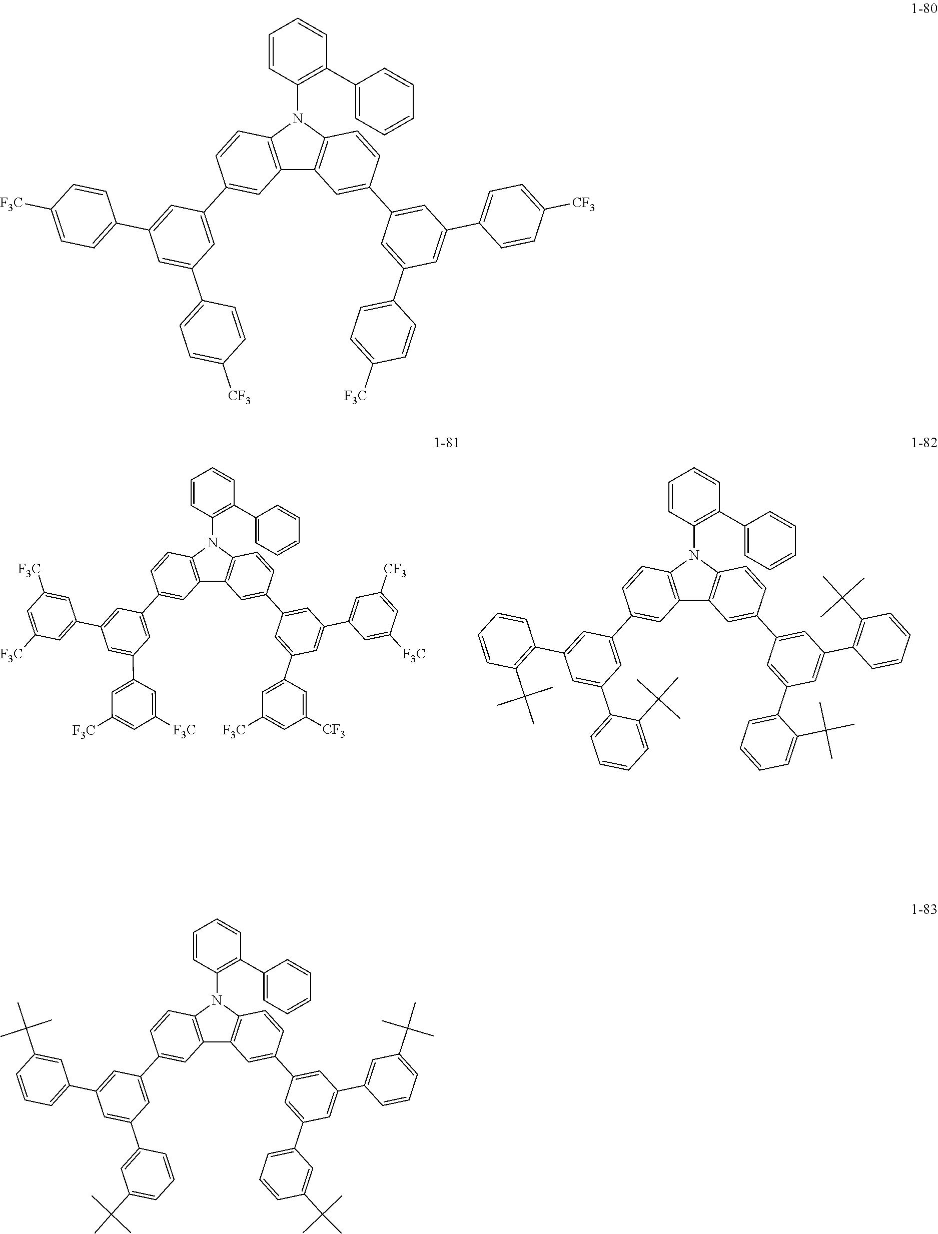

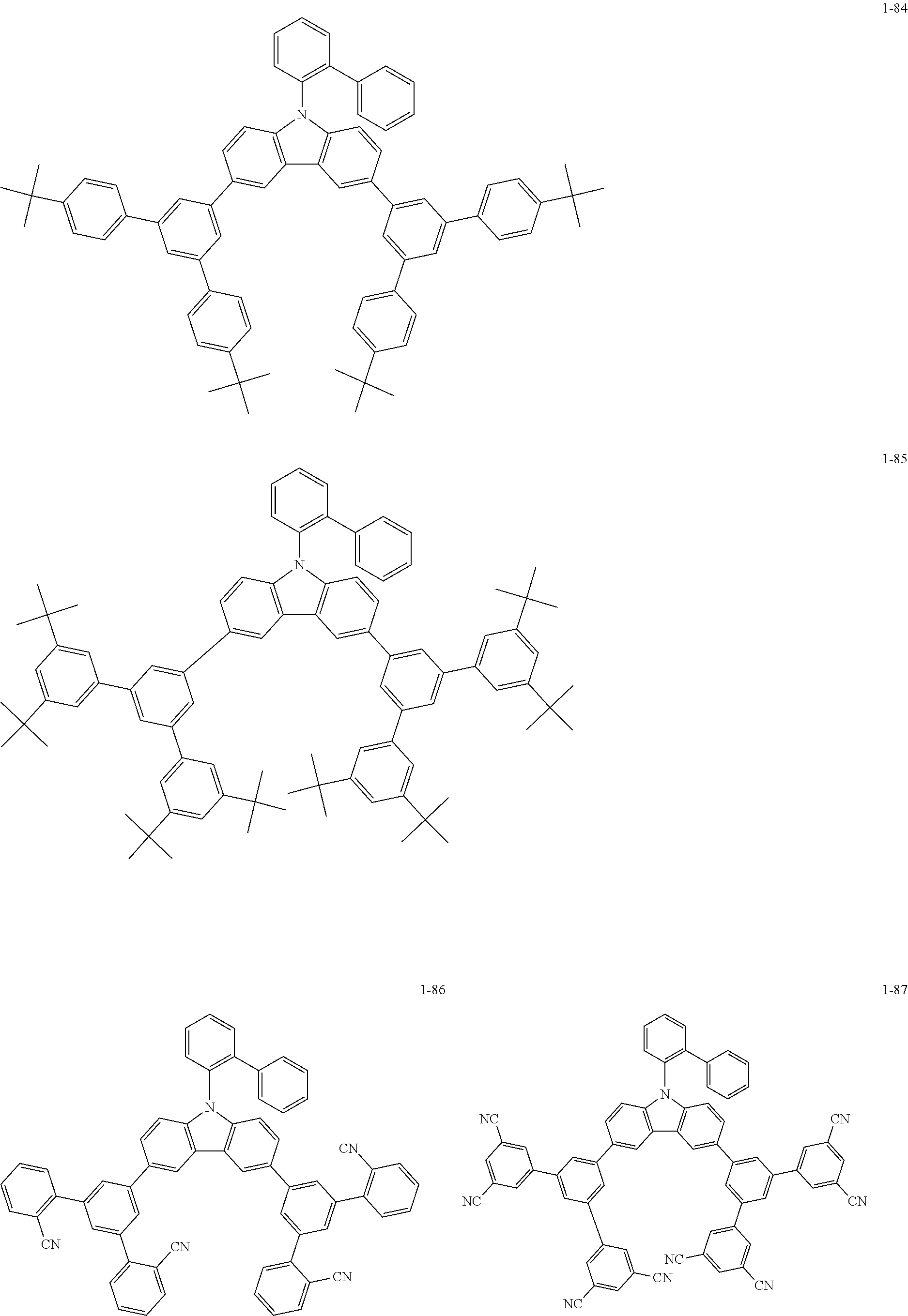

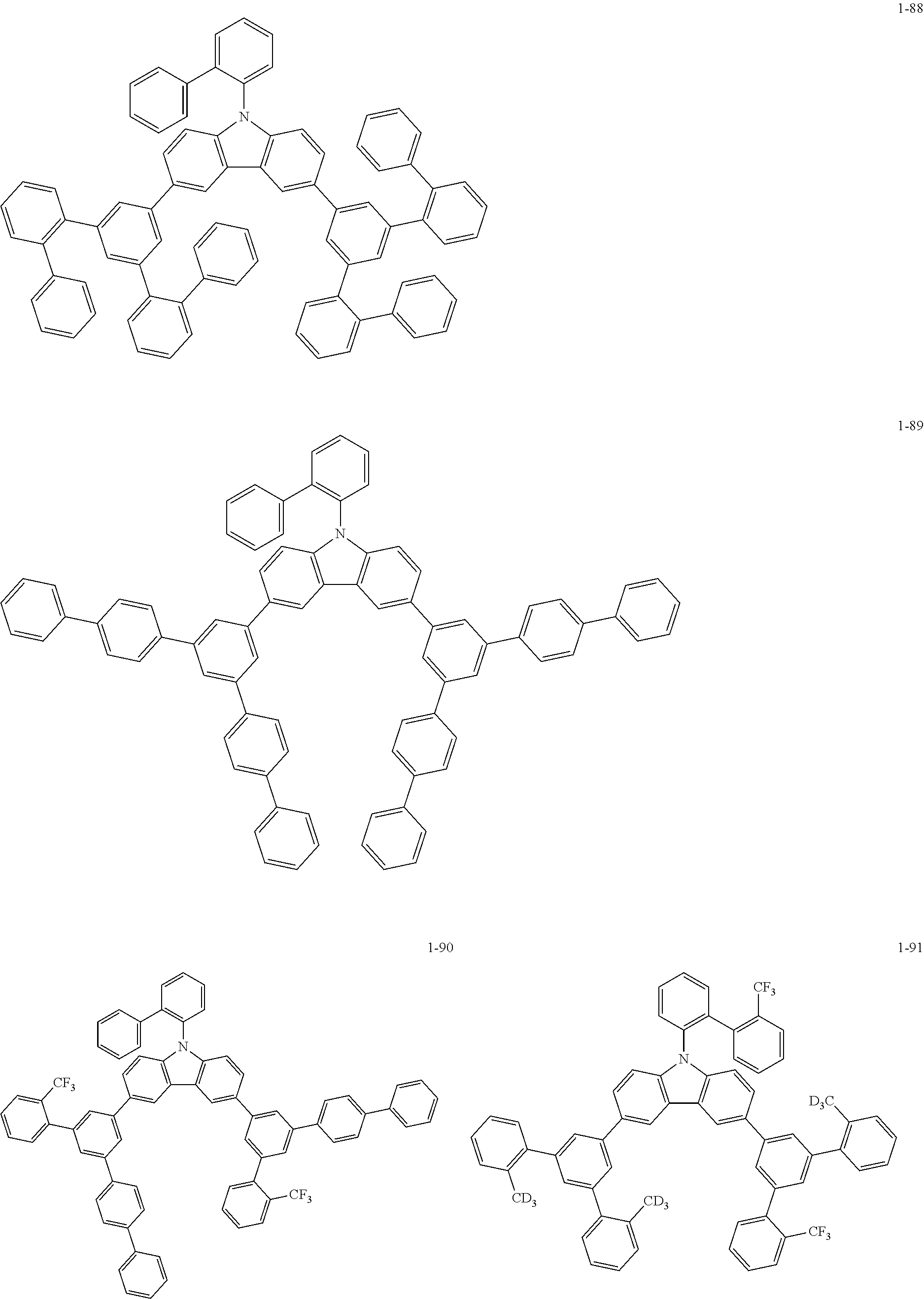

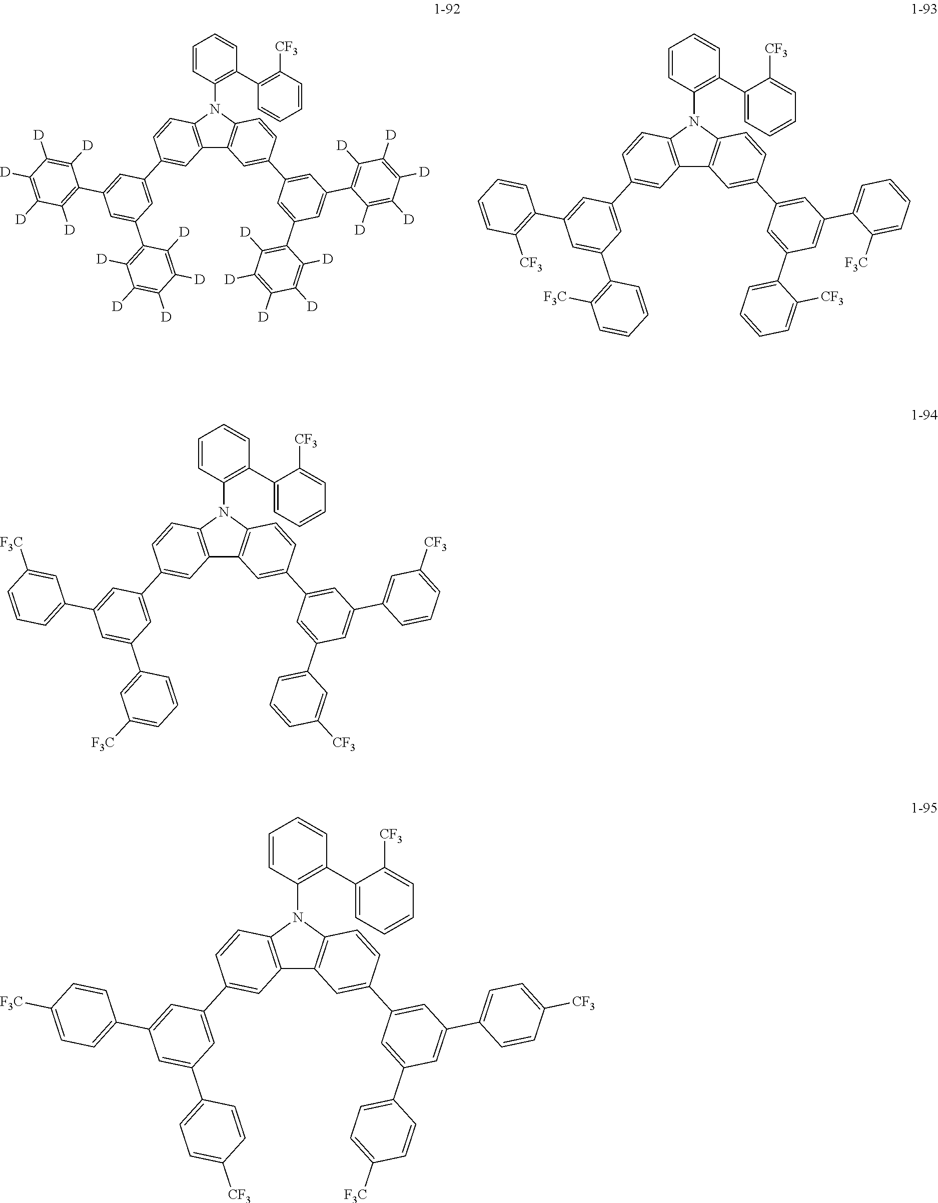

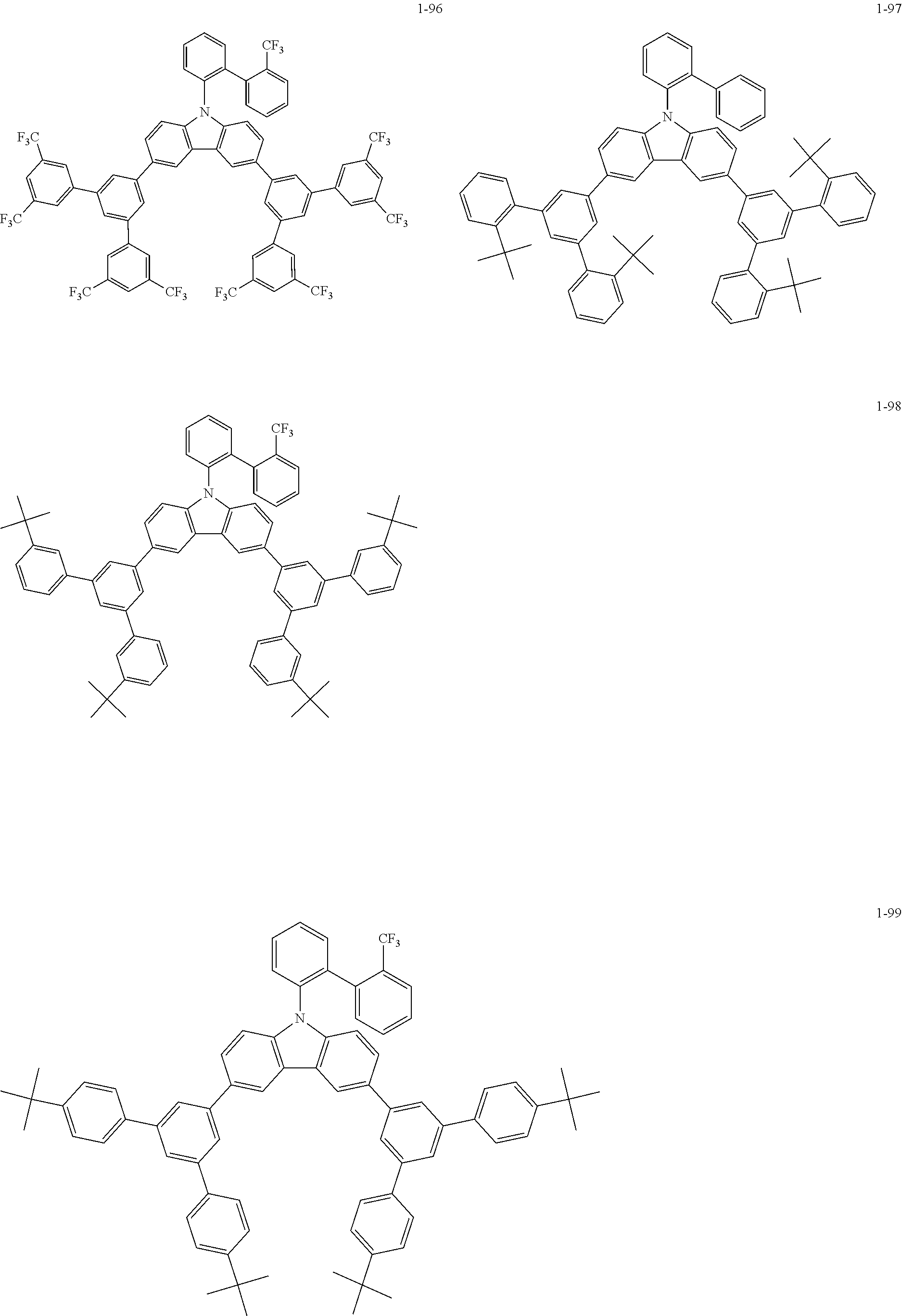

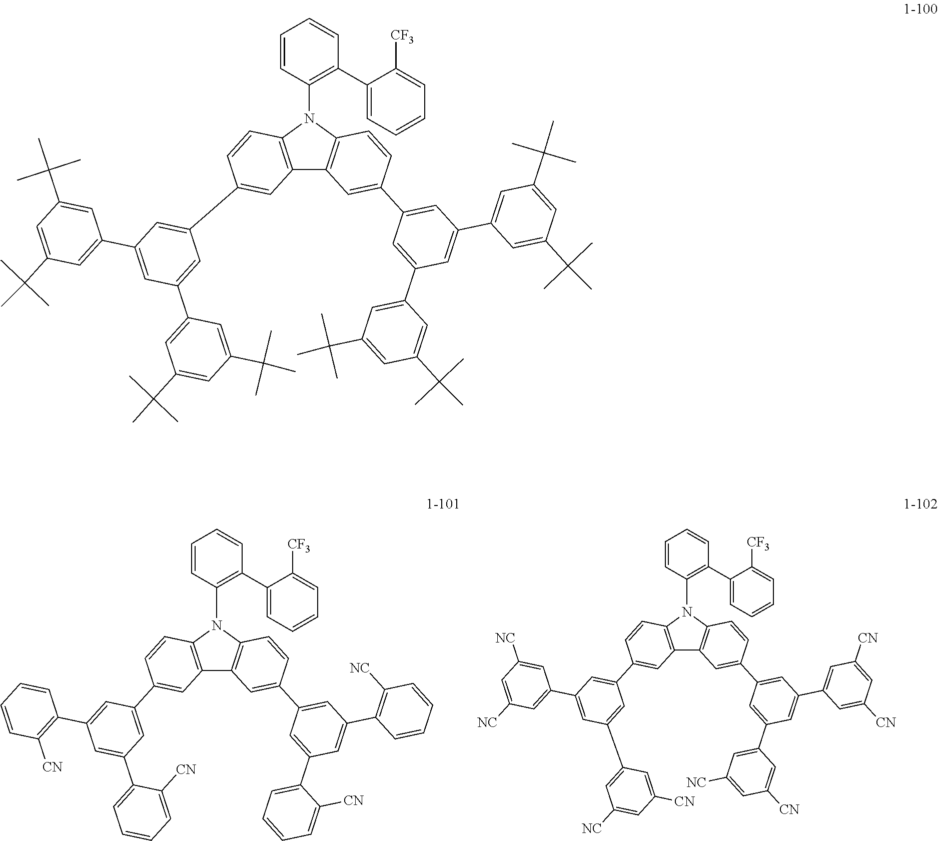 [SMILES: CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3-c3ccccc3C(F)(F)F)c2)cc(C(C)(C)C)c1 ; N#Cc1ccccc1-c1cc(-c2ccc3c(c2)c2cc(-c4cc(-c5ccccc5C#N)cc(-c5ccccc5C#N)c4)ccc2n3-c2ccccc2-c2ccccc2C(F)(F)F)cc(-c2ccccc2C#N)c1 ; N#Cc1cc(C#N)cc(-c2cc(-c3cc(C#N)cc(C#N)c3)cc(-c3ccc4c(c3)c3cc(-c5cc(-c6cc(C#N)cc(C#N)c6)cc(-c6cc(C#N)cc(C#N)c6)c5)ccc3n4-c3ccccc3-c3ccccc3C(F)(F)F)c2)c1]

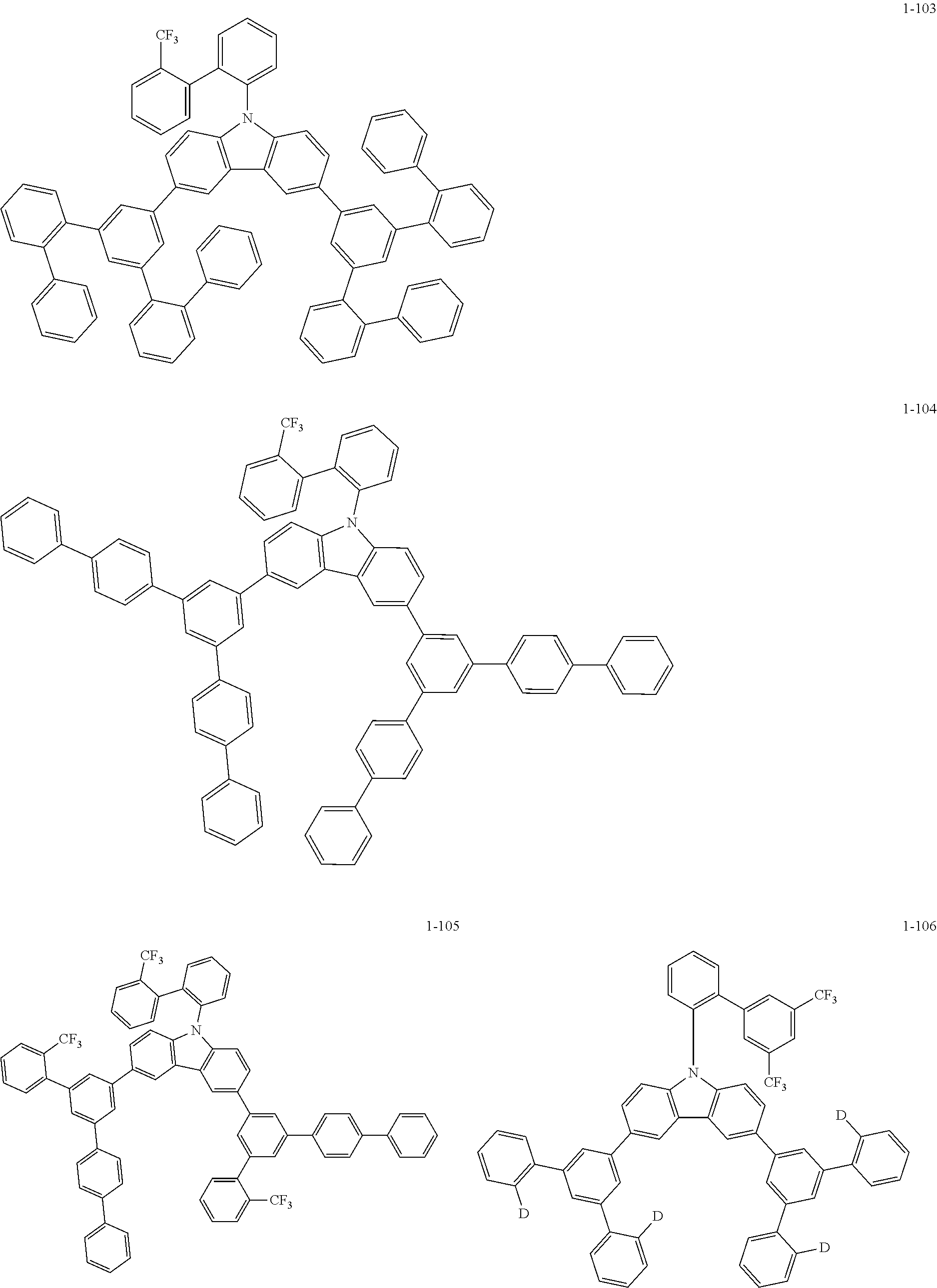

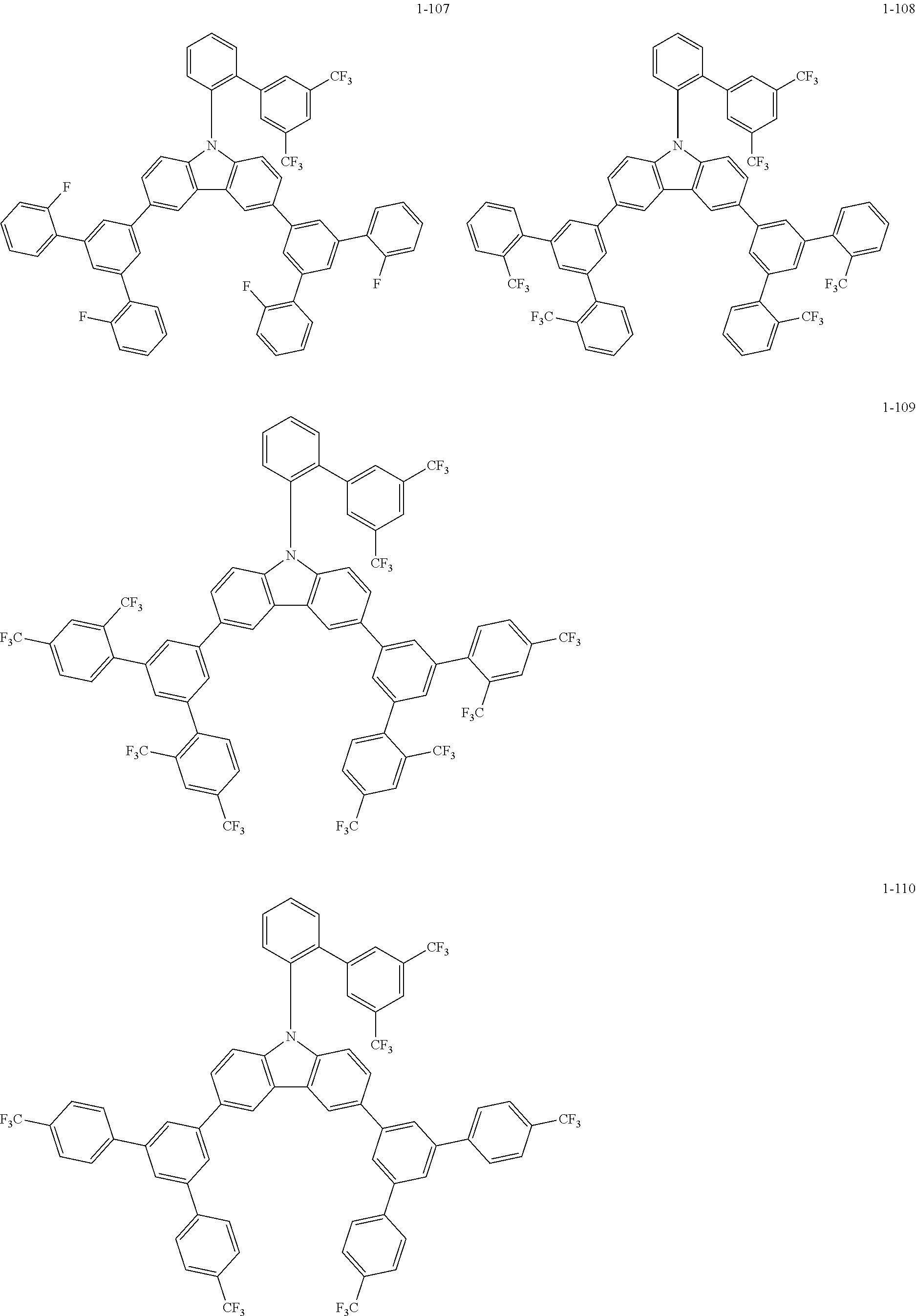

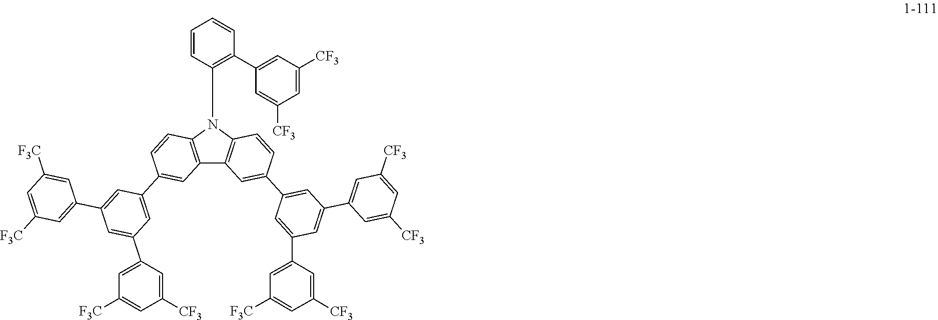 [SMILES: FC(F)(F)c1cc(-c2cc(-c3cc(C(F)(F)F)cc(C(F)(F)F)c3)cc(-c3ccc4c(c3)c3cc(-c5cc(-c6cc(C(F)(F)F)cc(C(F)(F)F)c6)cc(-c6cc(C(F)(F)F)cc(C(F)(F)F)c6)c5)ccc3n4-c3ccccc3-c3cc(C(F)(F)F)cc(C(F)(F)F)c3)c2)cc(C(F)(F)F)c1]

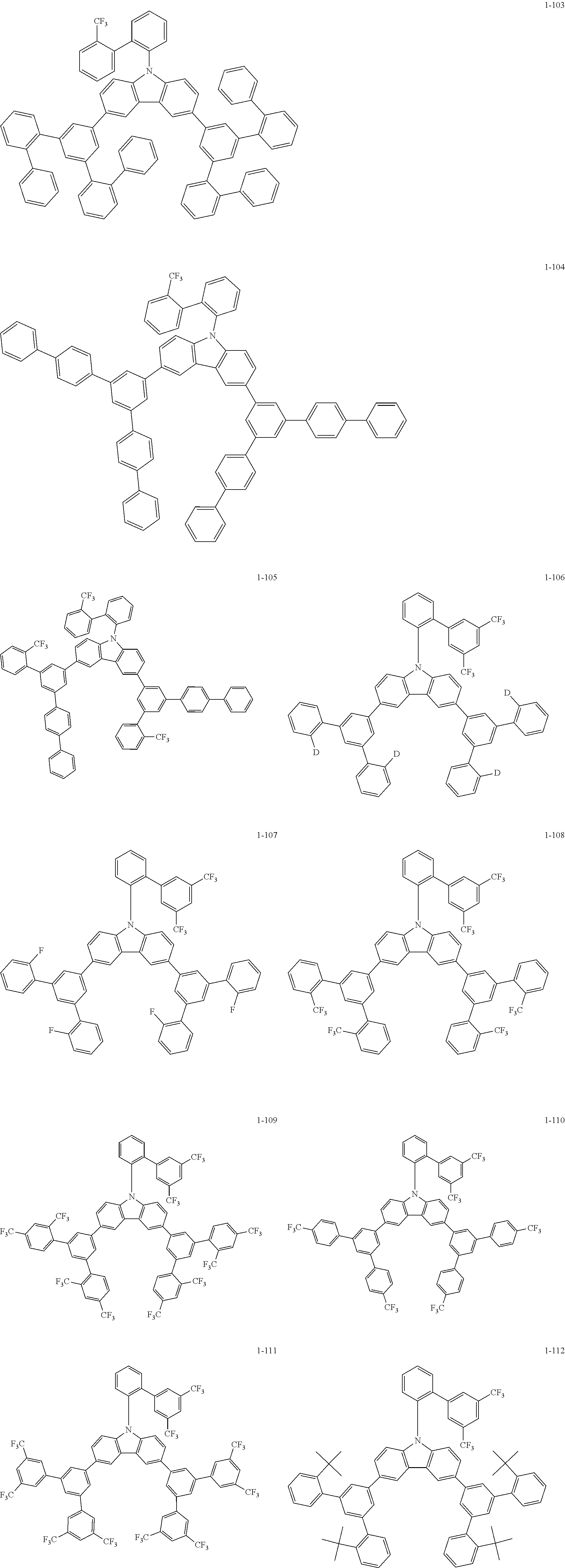

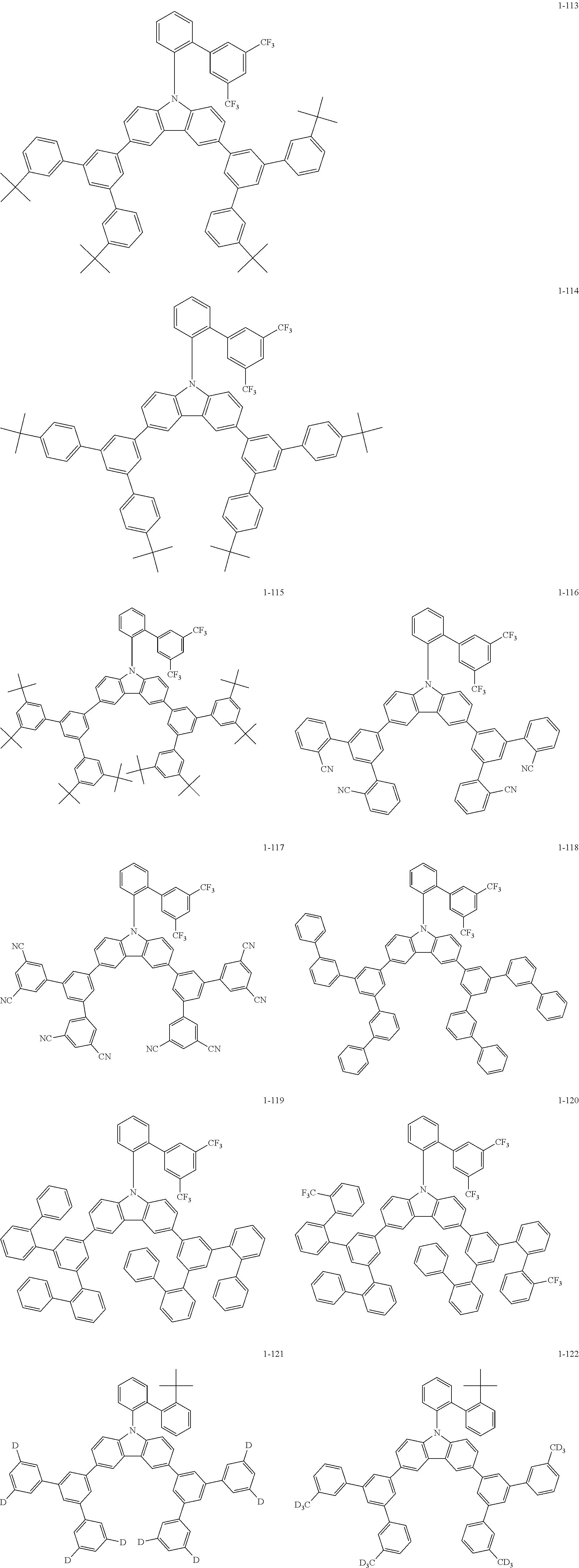

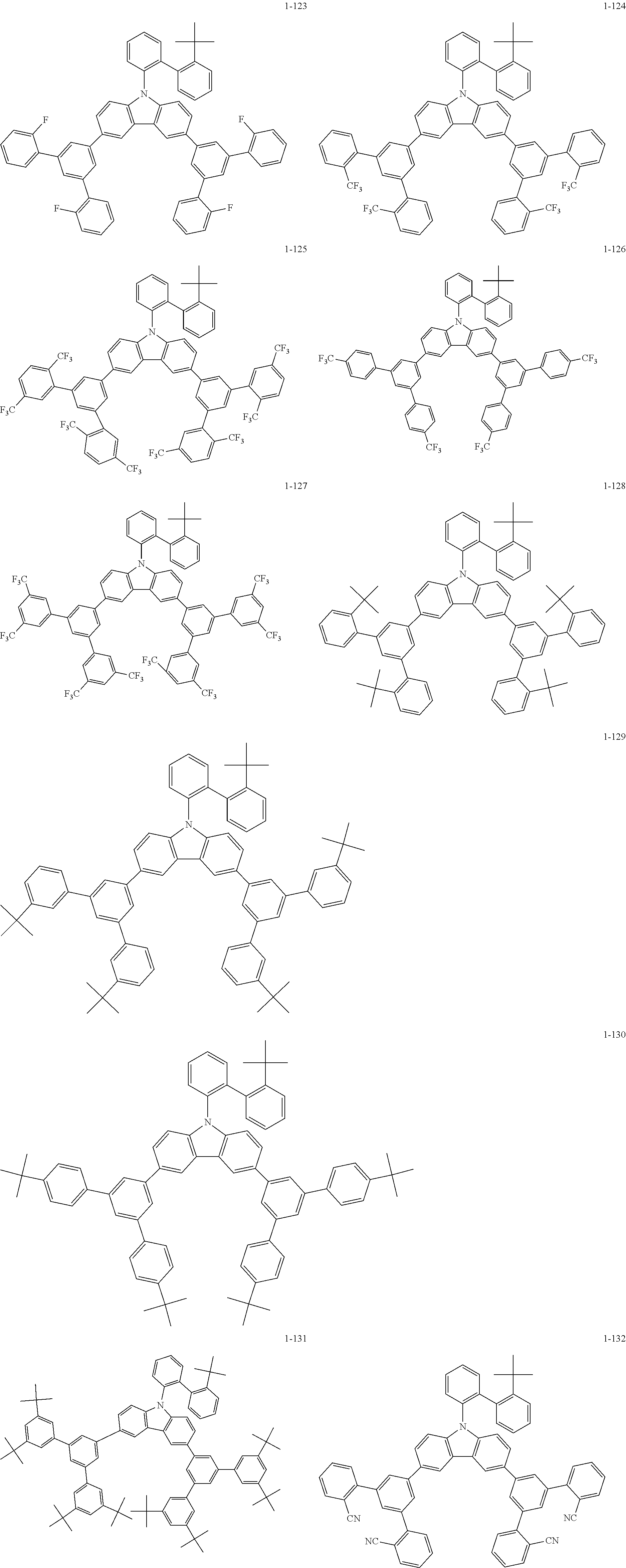

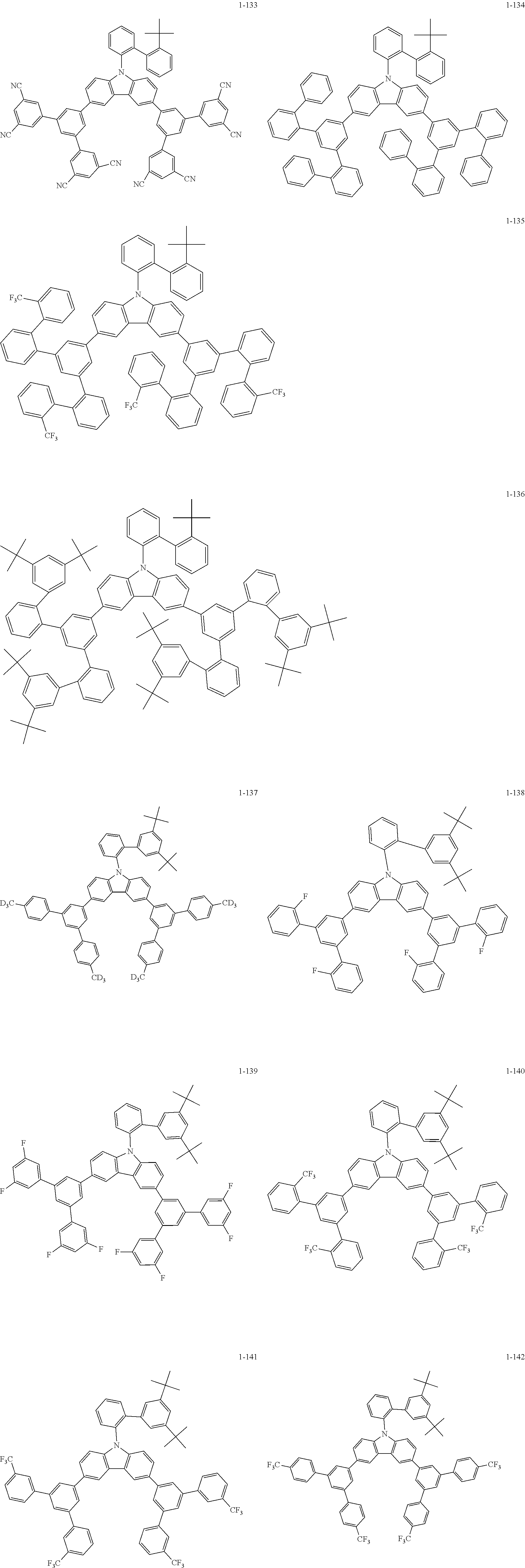

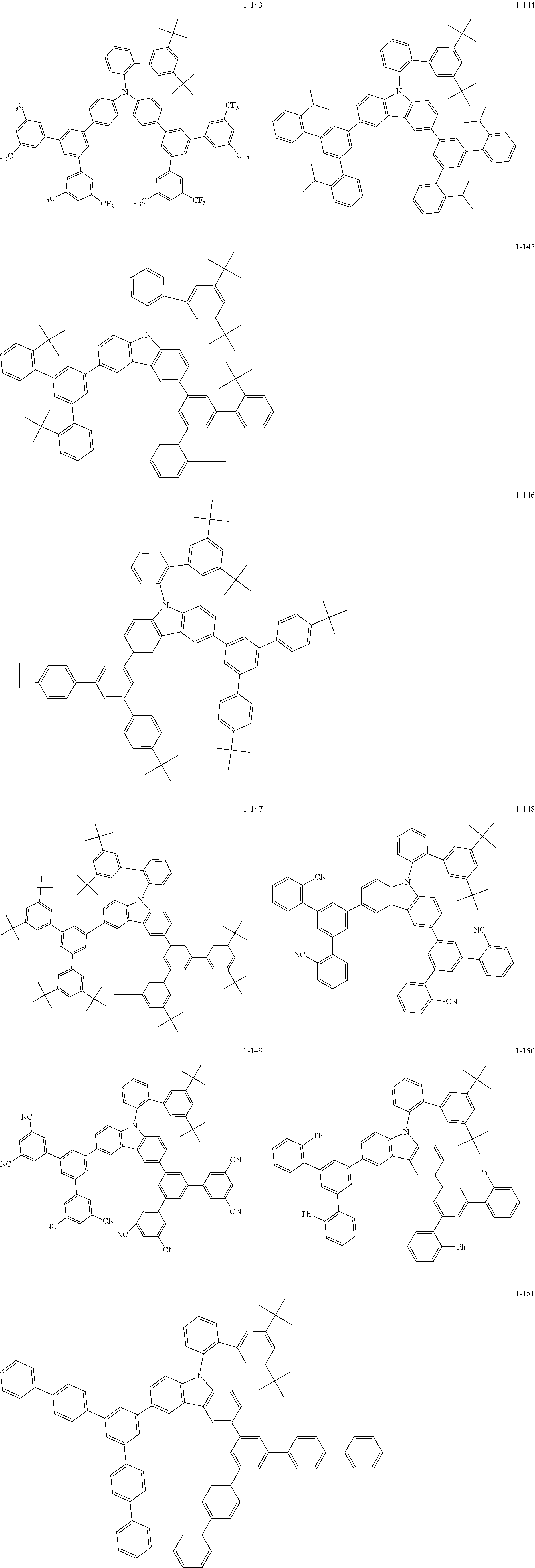

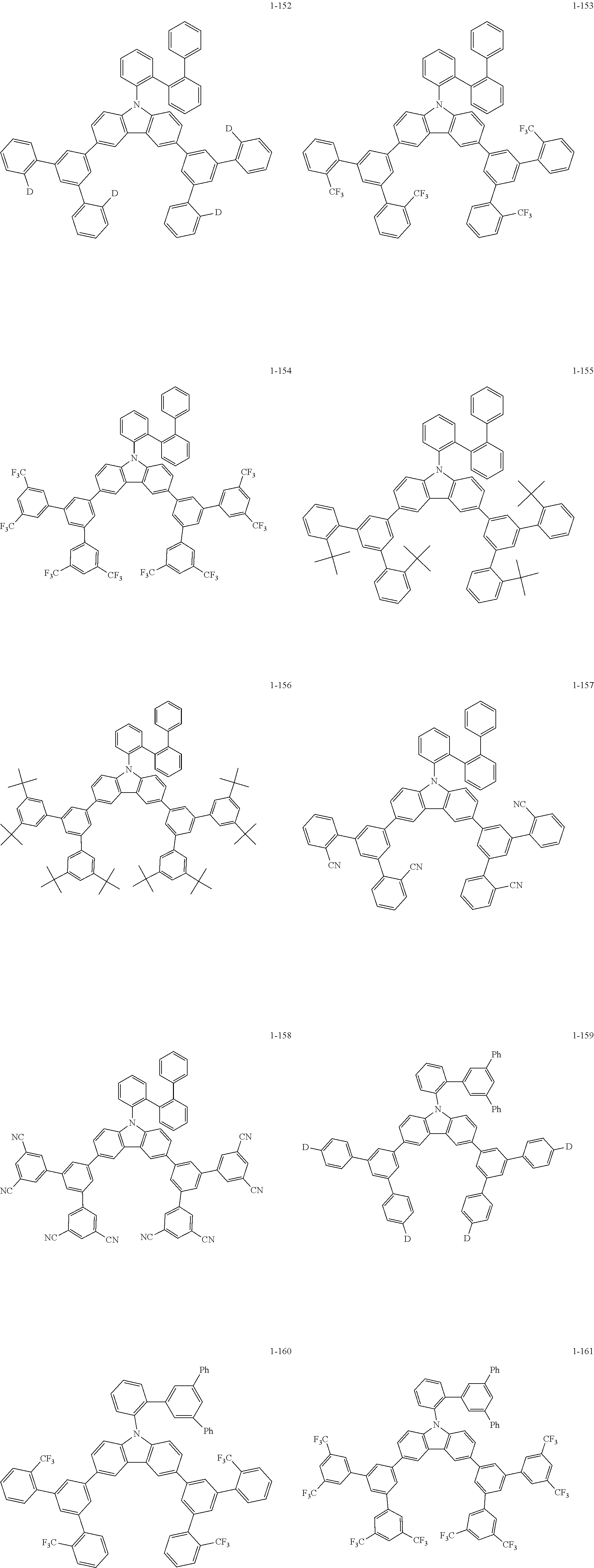

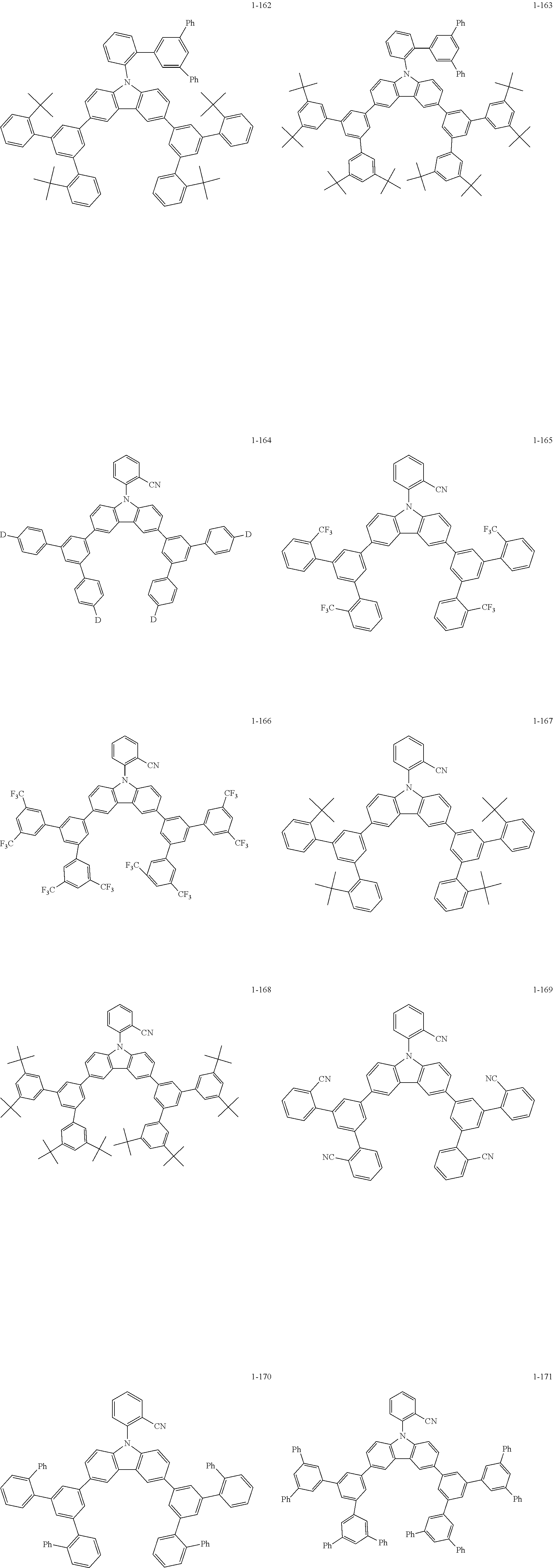

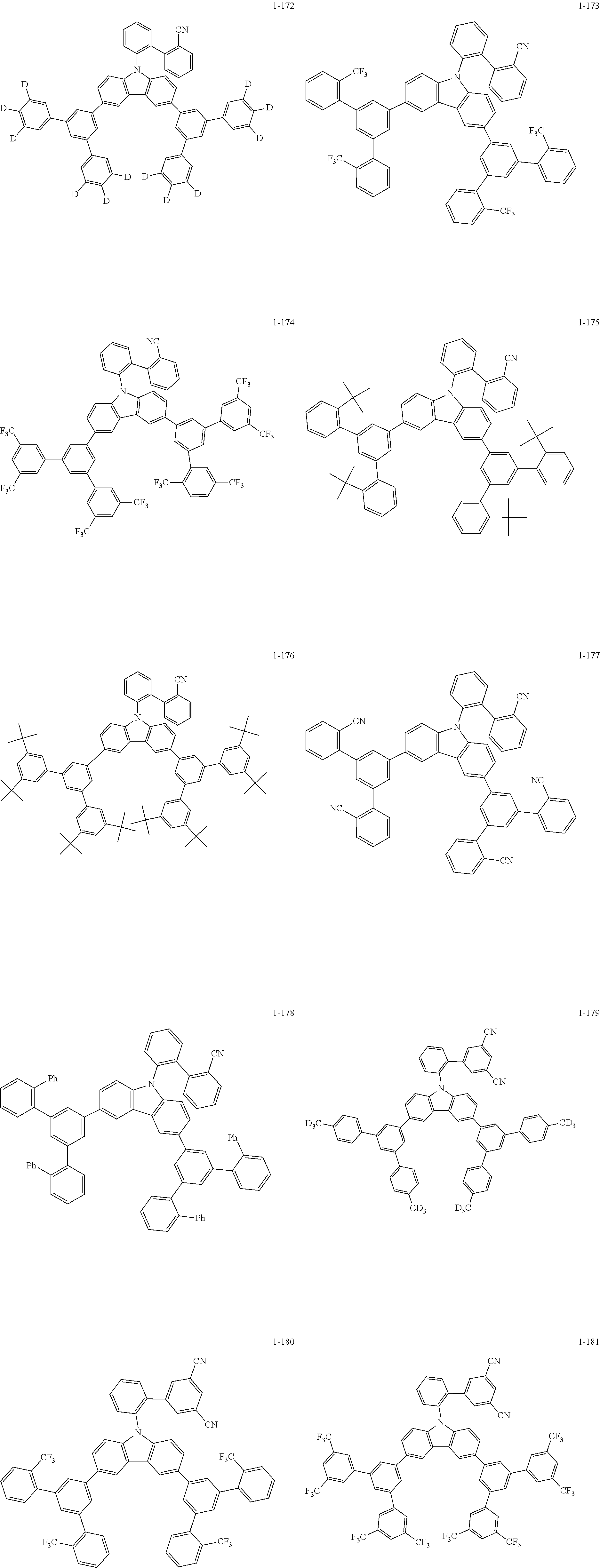

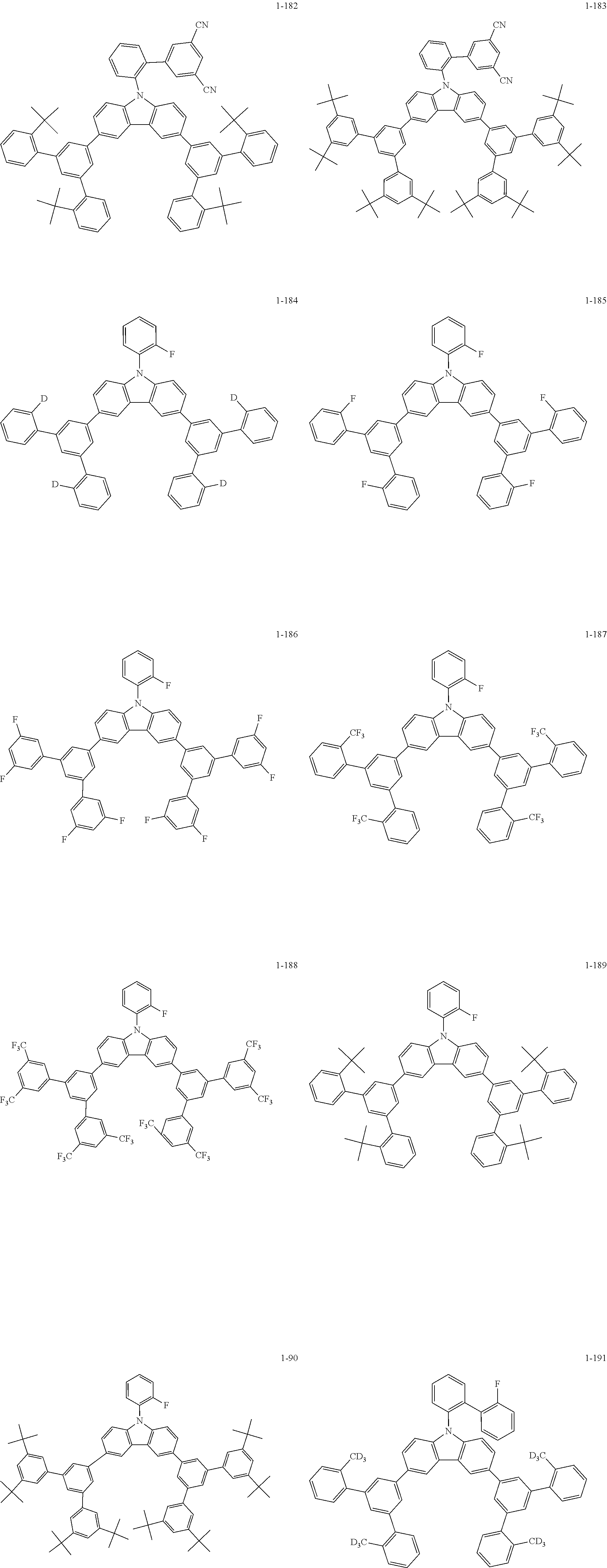

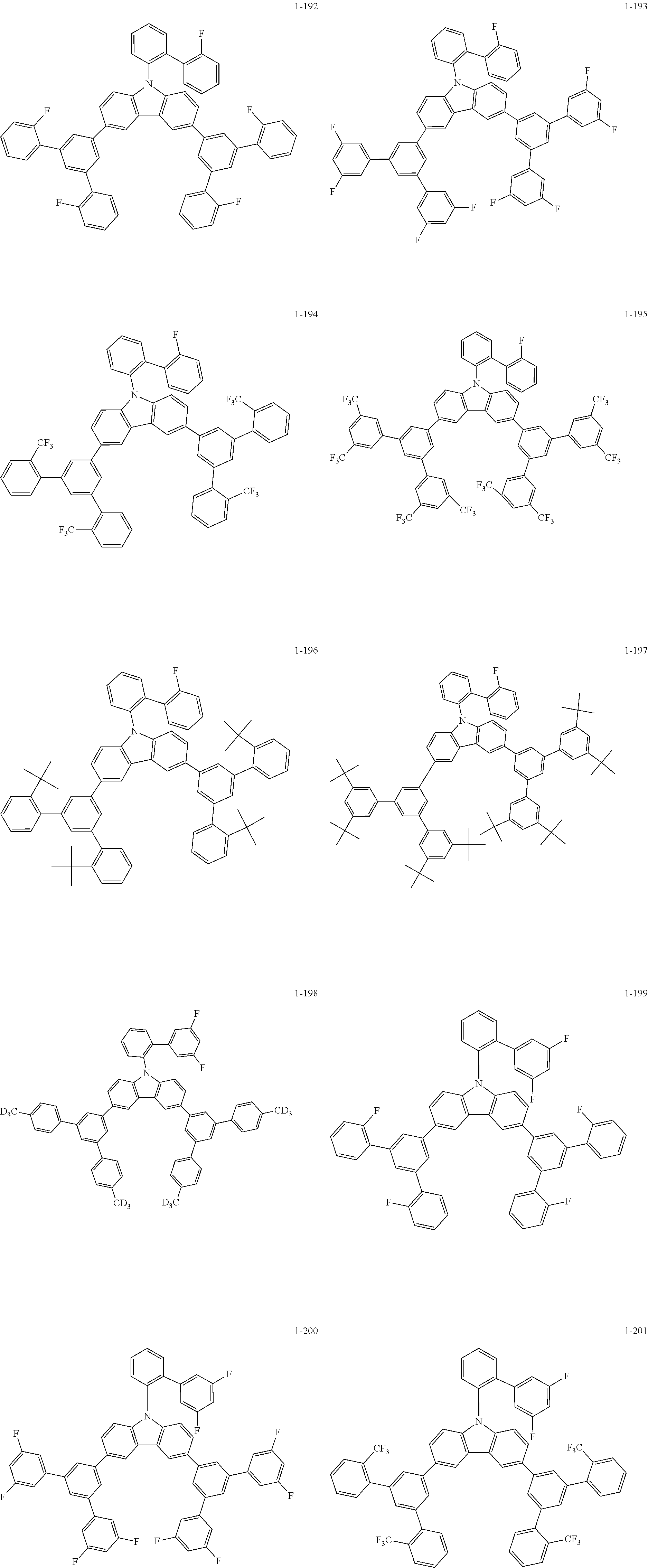

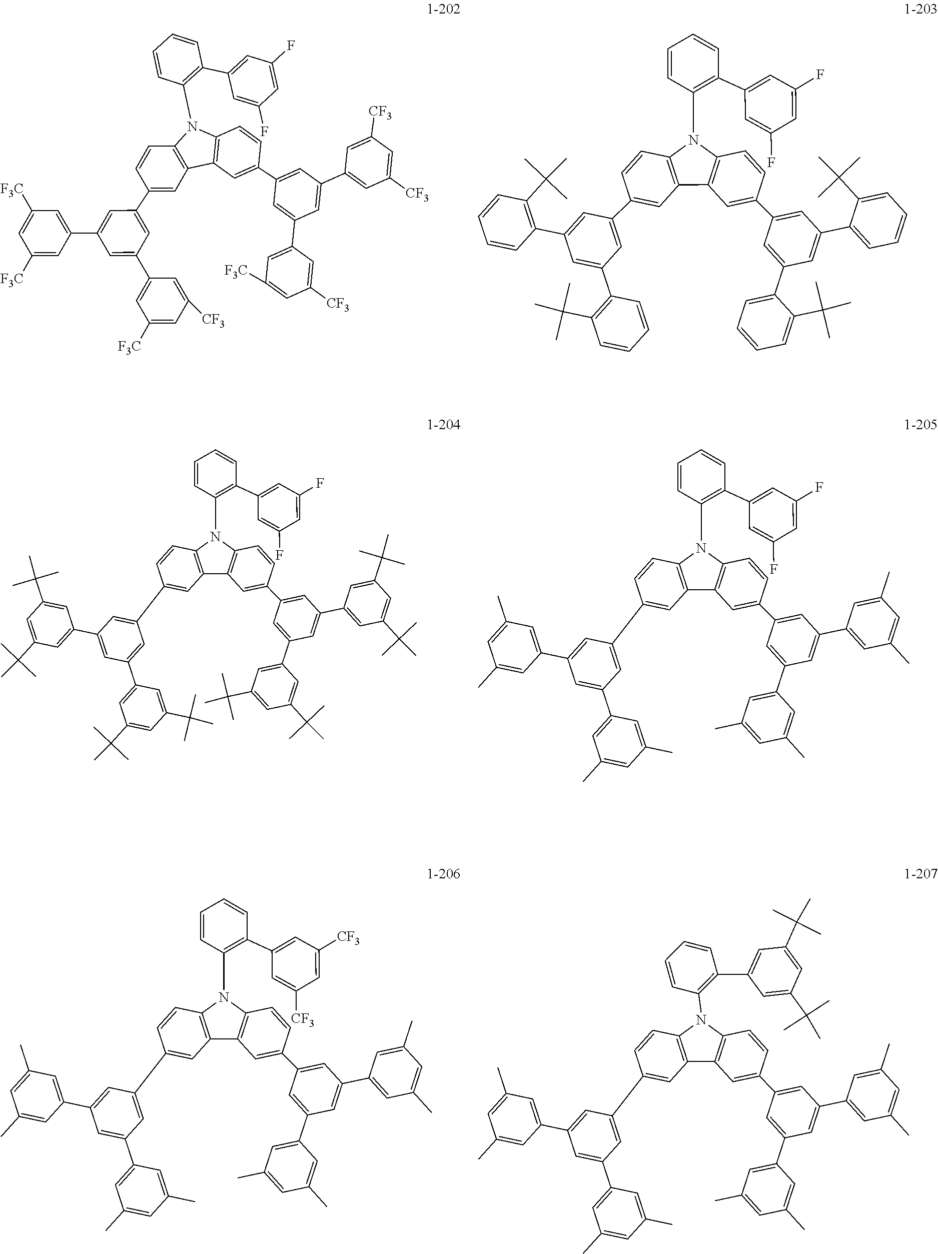

As described above, for the organic light emitting compound according to the present invention, an organic light emitting compound having various characteristics may be synthesized using a characteristic skeleton that exhibits unique properties and a moiety having unique properties introduced therein, and as a result, light emitting characteristics such as the light emitting efficiency of the device may be further improved by applying the organic light emitting compound according to the present invention to a light efficiency improving layer formed on the device.

In addition, the compound of the present invention may be applied to the device according to a general method for manufacturing an organic light emitting device. The organic light emitting device according to an exemplary embodiment of the present invention may be composed of a structure including a first electrode, a second electrode and an organic layer disposed therebetween, and may be manufactured using typical device manufacturing methods and materials, except that the organic light emitting compound according to the present invention is used in an organic layer of the device.

The organic layer of the organic light emitting device according to the present invention may be composed of a single-layered structure, but may also be composed of a multi-layered structure in which two or more organic layers are stacked. For example, the organic layer may have a structure including a hole injection layer, a hole transport layer, a light emitting layer, an electron transport layer, an electron injection layer, an electron blocking layer, a hole blocking layer, a light efficiency improving layer (capping layer), and the like. However, the structure of the organic layer is not limited thereto, and may include a fewer or greater number of organic layers.

Furthermore, an organic electroluminescent device according to an exemplary embodiment of the present invention includes a substrate, a first electrode (anode), an organic layer, a second electrode (cathode), and a light efficiency improving layer, and the light efficiency improving layer may be formed on the bottom of the first electrode (bottom emission) or on the top of the second electrode (top emission).

For the method of forming the light efficiency improving layer on the top of the second electrode (top emission), the light formed by the light emitting layer is emitted to the cathode side, but while the light emitted to the cathode side passes through the light efficiency improving layer (CPL) formed of the compound according to the present invention, the wavelength of light is amplified, and thus the light efficiency is increased. Further, for the method of forming the light efficiency improving layer on the bottom of the first electrode (bottom emission), the light efficiency of the organic electroluminescent device is also improved by adopting the compound according to the present invention in the light efficiency improving layer by the same principle.

The organic layer structure of a preferred organic light emitting device according to the present invention, and the like will be described in more detail in the Examples to be described below.

Further, the organic light emitting device according to the present invention may be manufactured by depositing a metal or a metal oxide having conductivity, or an alloy thereof on a substrate to form a positive electrode, forming an organic layer including a hole injection layer, a hole transport layer, a light emitting layer, and an electron transport layer thereon, and then depositing a material, which may be used as a negative electrode, thereon, by using a physical vapor deposition (PVD) method such as sputtering or e-beam evaporation.

In addition to the method as described above, an organic light emitting device may be made by sequentially depositing a negative electrode material, an organic layer, and a positive electrode material on a substrate. The organic layer may have a multi-layered structure including a hole injection layer, a hole transport layer, a light emitting layer, and an electron transport layer, and the like, but is not limited thereto and may have a single-layered structure. In addition, the organic layer may be manufactured to include a fewer number of layers by a method such as a solvent process, for example, spin coating, dip coating, doctor blading, screen printing, inkjet printing, or a thermal transfer method instead of a deposition method, using various polymer materials.

As the positive electrode material, materials having a high work function are usually preferred so as to facilitate the injection of holes into an organic layer. Specific examples of the positive electrode material which may be used in the present invention include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly [3,4-(ethylene-1,2-dioxy)thiophene] (PEDT), polypyrrole, and polyaniline; and the like, but are not limited thereto.

As the negative electrode material, materials having a low work function are usually preferred so as to facilitate the injection of electrons into an organic layer. Specific examples of the negative electrode material include: a metal such as magnesium, calcium, sodium, potassium, titanium, indium, yttrium, lithium, gadolinium, aluminum, silver, tin, and lead, or an alloy thereof, a multi-layer structured material, such as LiF/Al or LiO₂/Al, and the like, but are not limited thereto.

The hole injection material is a material which may proficiently accept holes from a positive electrode at low voltage, and the highest occupied molecular orbital (HOMO) of the hole injection material is preferably a value between the work function of the positive electrode material and the HOMO of the neighboring organic layer. Specific examples of the hole injection material include metal porphyrin, oligothiophene, arylamine-based organic materials, hexanitrile hexaazatriphenylene, quinacridone-based organic materials, perylene-based organic materials, anthraquinone, polyaniline-based and polythiophene-based conductive polymers, and the like, but are not limited thereto.

A hole transport material is suitably a material having high hole mobility which may accept holes from a positive electrode or a hole injection layer and transfer the holes to a light emitting layer. Specific examples thereof include arylamine-based organic materials, conductive polymers, block copolymers having both conjugated portions and non-conjugated portions, and the like.

The light emitting material is a material which may receive holes and electrons from a hole transport layer and an electron transport layer, and combine the holes and the electrons to emit light in a visible ray region, and is preferably a material having high quantum efficiency for fluorescence or phosphorescence. Specific examples thereof include 8-hydroxy-quinoline aluminum complexes (Alq₃), carbazole-based compounds, dimerized styryl compounds, BAlq, 10-hydroxybenzoquinoline-metal compounds, benzoxazole-based, benzothiazole-based and benzimidazole-based compounds, poly(p-phenylenevinylene) (PPV)-based polymers, spiro compounds, polyfluorene, lubrene, and the like, but are not limited thereto.

An electron transport material is suitably a material having high electron mobility which may proficiently accept electrons from a negative electrode and transfer the electrons to a light emitting layer. Specific examples thereof include Al complexes of 8-hydroxyquinoline, complexes including Alq₃, organic radical compounds, hydroxyflavone-metal complexes, and the like, but are not limited thereto.

The organic light emitting device according to the present invention may be a top emission type, a bottom emission type, or a dual emission type according to the material to be used.

Furthermore, the organic light emitting compound according to the present invention may be operated by a principle which is similar to the principle applied to an organic light emitting device, even in an organic electroluminescent device including an organic solar cell, an organic photoconductor, an organic transistor, and the like.

### MODE FOR INVENTION

Hereinafter, the present invention will be exemplified in more detail through preferred examples. However, these examples are for more specifically describing the present invention, the scope of the present invention is not limited thereto, and it will be obvious to a person with ordinary skill in the art that various changes and modifications can be made within the scope of the present invention and the scope of the technical spirit.

### Synthesis Example 1: Synthesis of Compound 4

### (1) Preparation Example 1: Synthesis of Intermediate 4-1

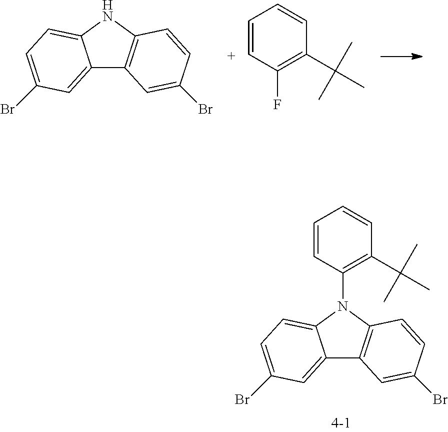 [SMILES: Brc1ccc2[nH]c3ccc(Br)cc3c2c1 ; CC(C)(C)c1ccccc1F ; CC(C)(C)c1ccccc1-n1c2ccc(Br)cc2c2cc(Br)ccc21]

500 mL of DMF was added to 3,6-dibromocarbazole (10.0 g, 0.031 mol), 1-(tert-butyl)-2-fluorobenzene (5.6 g, 0.037 mol), and cesium carbonate (6.4 g, 0.046 mol), and the resulting mixture was stirred under reflux at 150° C. for 12 hours and reacted. After completion of the reaction, the resulting product was extracted and concentrated, and then subjected to column chromatography and recrystallization to obtain 9.7 g (yield 68.9%) of <Intermediate 4-1>.

### (2) Preparation Example 2: Synthesis of Compound 4

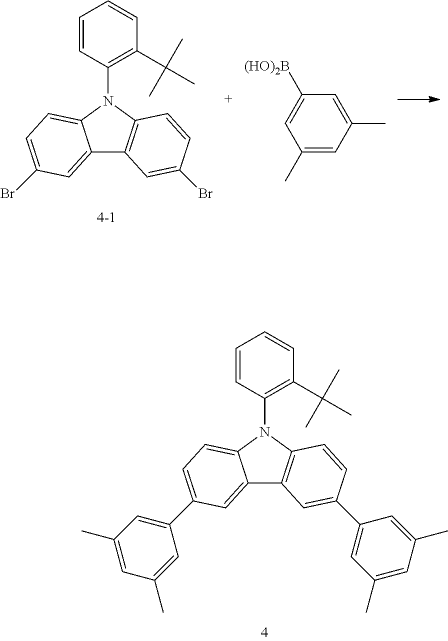

Intermediate 4-1 (10.0 g, 0.022 mol), 3,5-dimethylphenylboronic acid (7.87 g, 0.052 mol), potassium carbonate (15.1 g, 0.109 mol), Pd(PPh₃)₄ (1.26 g, 0.001 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 7.8 g (yield 70.2%) of <Compound 4>.

LC/MS: m/z=507[(M)⁺]

### Synthesis Example 2: Synthesis of Compound 42

### (1) Preparation Example 1: Synthesis of Intermediate 42-1

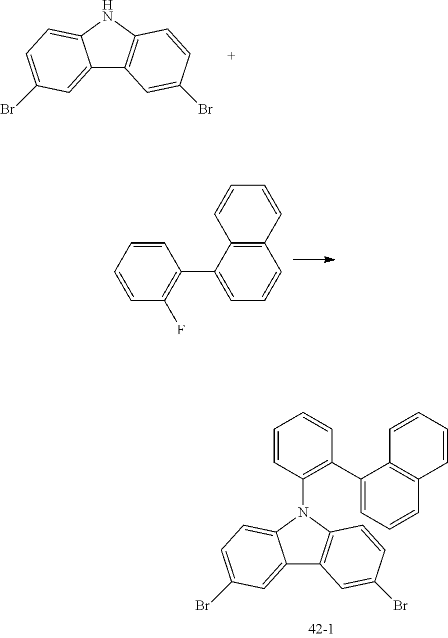 [SMILES: Brc1ccc2[nH]c3ccc(Br)cc3c2c1 ; Fc1ccccc1-c1cccc2ccccc12 ; Brc1ccc2c(c1)c1cc(Br)ccc1n2-c1ccccc1-c1cccc2ccccc12]

500 mL of DMF was added to 3,6-dibromocarbazole (10.0 g, 0.031 mol), 1-(2-fluorophenyl)naphthalene (8.2 g, 0.037 mol), and cesium carbonate (6.4 g, 0.046 mol), and the resulting mixture was stirred under reflux at 150° C. for 15 hours and reacted. After completion of the reaction, the resulting product was extracted and concentrated, and then subjected to column chromatography and recrystallization to obtain 11.2 g (yield 69.0%) of <Intermediate 42-1>.

### (2) Preparation Example 2: Synthesis of Compound 42

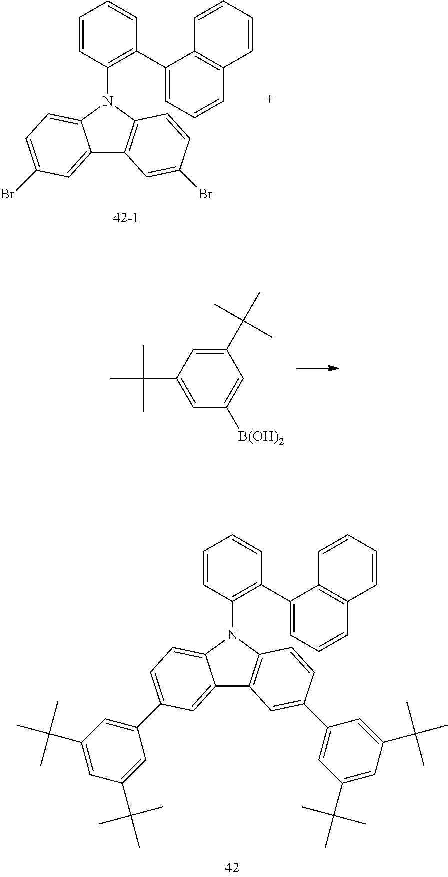 [SMILES: Brc1ccc2c(c1)c1cc(Br)ccc1n2-c1ccccc1-c1cccc2ccccc12 ; CC(C)(C)c1cc(B(O)O)cc(C(C)(C)C)c1 ; CC(C)(C)c1cc(-c2ccc3c(c2)c2cc(-c4cc(C(C)(C)C)cc(C(C)(C)C)c4)ccc2n3-c2ccccc2-c2cccc3ccccc23)cc(C(C)(C)C)c1]

Intermediate 4-1 (10.0 g, 0.019 mol), 3,5-di-tert-butylphenylboronic acid (10.7 g, 0.046 mol), potassium carbonate (13.1 g, 0.095 mol), Pd(PPh₃)₄ (1.10 g, 0.001 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 9.6 g (yield 67.8%) of <Compound 42>.

LC/MS: m/z=745[(M)⁺]

### Synthesis Example 3: Synthesis of Compound 50

### (1) Preparation Example 1: Synthesis of Intermediate 50-1

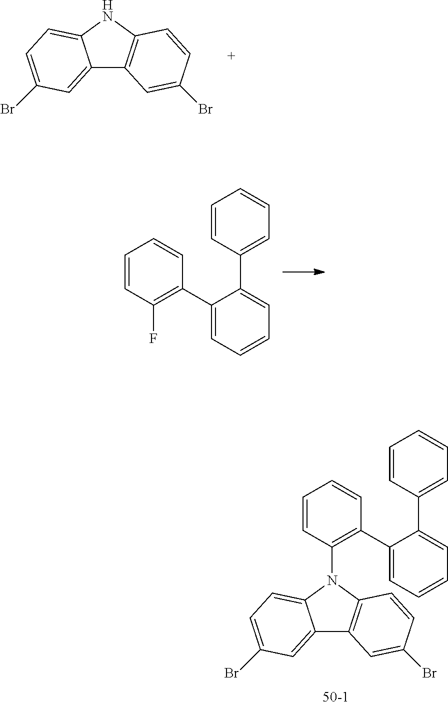

500 mL of DMF was added to 3,6-dibromocarbazole (10.0 g, 0.031 mol), 1-(2-fluorophenyl)-2-phenylbenzene (9.2 g, 0.037 mol), and cesium carbonate (6.4 g, 0.046 mol), and the resulting mixture was stirred under reflux at 150° C. for 15 hours and reacted. After completion of the reaction, the resulting product was extracted and concentrated, and then subjected to column chromatography and recrystallization to obtain 11.7 g (yield 68.7%) of <Intermediate 50-1>.

### (2) Preparation Example 2: Synthesis of Compound 50

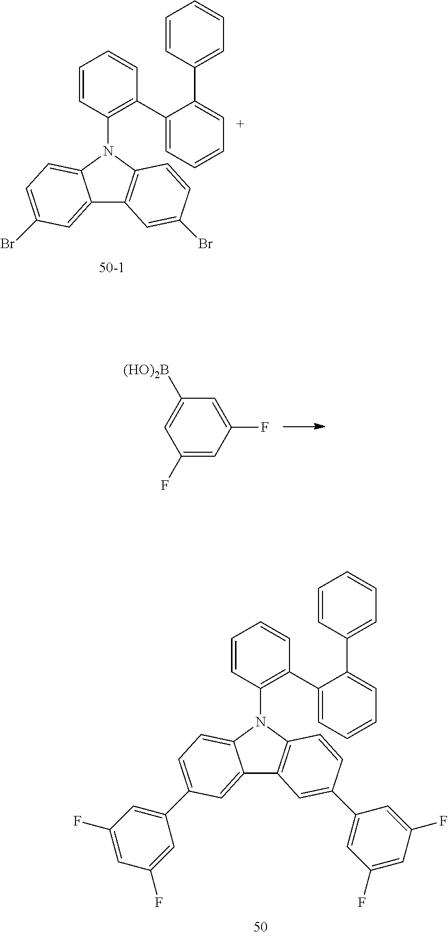 [SMILES: Brc1ccc2c(c1)c1cc(Br)ccc1n2-c1ccccc1-c1ccccc1-c1ccccc1 ; OB(O)c1cc(F)cc(F)c1 ; Fc1cc(F)cc(-c2ccc3c(c2)c2cc(-c4cc(F)cc(F)c4)ccc2n3-c2ccccc2-c2ccccc2-c2ccccc2)c1]

Intermediate 50-1 (10.0 g, 0.018 mol), 3,5-difluorophenylboronic acid (6.8 g, 0.043 mol), potassium carbonate (12.5 g, 0.090 mol), Pd(PPh₃)₄ (1.04 g, 0.001 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 7.5 g (yield 66.9%) of <Compound 50>.

LC/MS: m/z=619[(M)⁺]

### Synthesis Example 4: Synthesis of Compound 100

### (1) Preparation Example 1: Synthesis of Intermediate 100-1

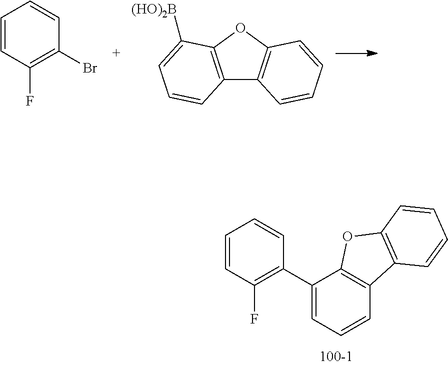 [SMILES: Fc1ccccc1Br ; OB(O)c1cccc2c1oc1ccccc12 ; Fc1ccccc1-c1cccc2c1oc1ccccc12]

1-Bromo-2-fluorobenzene (10.0 g, 0.057 mol), dibenzofuran-4-boronic acid (14.5 g, 0.069 mol), potassium carbonate (23.7 g, 0.171 mol), Pd(PPh₃)₄ (3.3 g, 0.003 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 9.5 g (yield 63.4%) of <Compound 100-1>.

### (2) Preparation Example 2: Synthesis of Intermediate 100-2

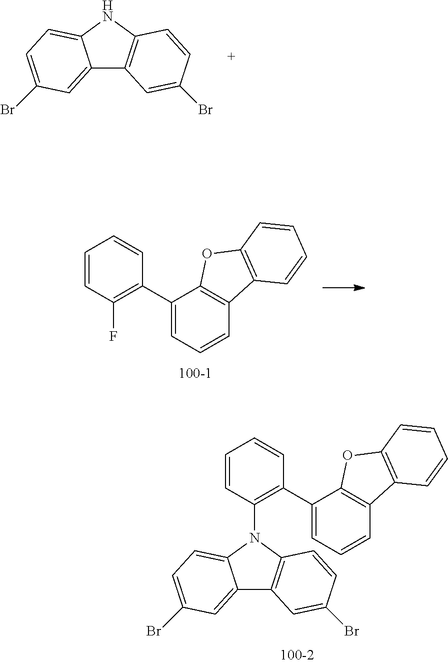 [SMILES: Brc1ccc2[nH]c3ccc(Br)cc3c2c1 ; Fc1ccccc1-c1cccc2c1oc1ccccc12 ; Brc1ccc2c(c1)c1cc(Br)ccc1n2-c1ccccc1-c1cccc2c1oc1ccccc12]

500 mL of DMF was added to 3,6-dibromocarbazole (10.0 g, 0.031 mol), Intermediate 100-1 (9.7 g, 0.037 mol), and cesium carbonate (6.4 g, 0.046 mol), and the resulting mixture was stirred under reflux at 150° C. for 15 hours and reacted. After completion of the reaction, the resulting product was extracted and concentrated, and then subjected to column chromatography and recrystallization to obtain 11.9 g (yield 68.2%) of <Intermediate 100-2>.

### (3) Preparation Example 3: Synthesis of Compound 100

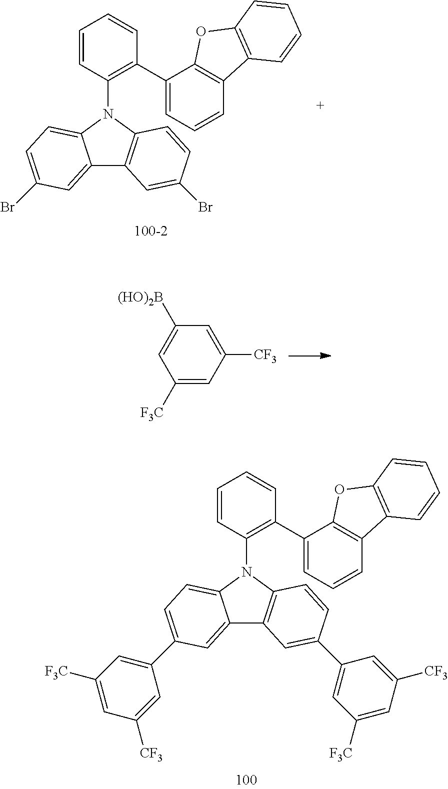 [SMILES: Brc1ccc2c(c1)c1cc(Br)ccc1n2-c1ccccc1-c1cccc2c1oc1ccccc12 ; OB(O)c1cc(C(F)(F)F)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc3c2oc2ccccc23)cc(C(F)(F)F)c1]

Intermediate 100-2 (10.0 g, 0.018 mol), 3,5-bis(trifluoromethyl)phenylboronic acid (10.9 g, 0.042 mol), potassium carbonate (12.2 g, 0.088 mol), Pd(PPh₃)₄ (1.10 g, 0.001 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 9.6 g (yield 65.3%) of <Compound 100>.

LC/MS: m/z=833[(M)⁺]

### Synthesis Example 5: Synthesis of Compound 127

### (1) Preparation Example 1: Synthesis of Intermediate 127-1

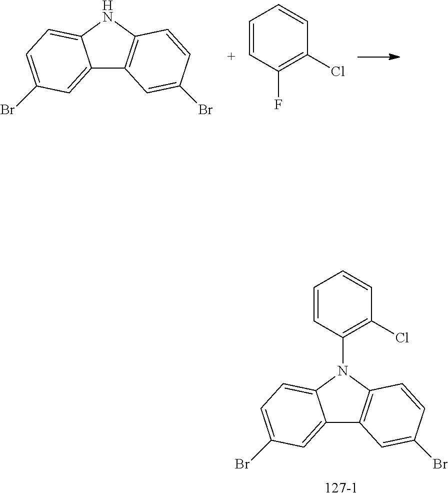 [SMILES: Brc1ccc2[nH]c3ccc(Br)cc3c2c1 ; Fc1ccccc1Cl ; Clc1ccccc1-n1c2ccc(Br)cc2c2cc(Br)ccc21]

500 mL of DMF was added to 3,6-dibromocarbazole (10.0 g, 0.031 mol), 1-chloro-2-fluorobenzene (4.8 g, 0.037 mol), and cesium carbonate (6.4 g, 0.046 mol), and the resulting mixture was stirred under reflux at 150° C. for 15 hours and reacted. After completion of the reaction, the resulting product was extracted and concentrated, and then subjected to column chromatography to obtain 9.1 g (yield 67.9%) of <Intermediate 127-1>.

### (2) Preparation Example 2: Synthesis of Intermediate 127-2

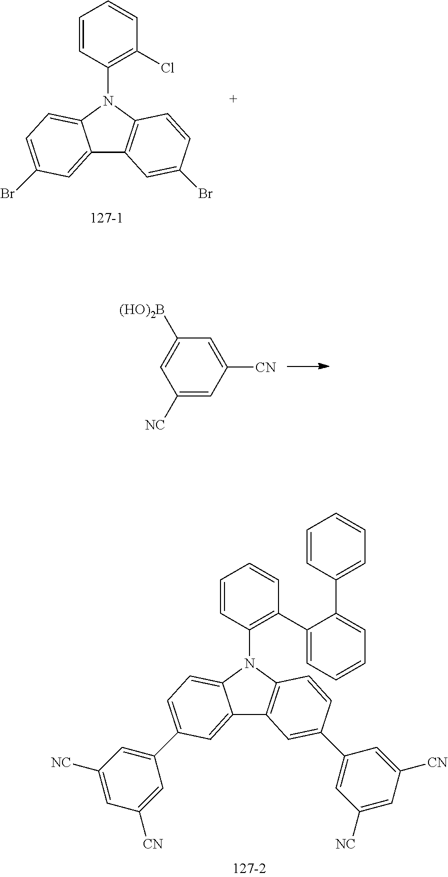 [SMILES: Clc1ccccc1-n1c2ccc(Br)cc2c2cc(Br)ccc21 ; N#Cc1cc(C#N)cc(B(O)O)c1 ; N#Cc1cc(C#N)cc(-c2ccc3c(c2)c2cc(-c4cc(C#N)cc(C#N)c4)ccc2n3-c2ccccc2-c2ccccc2-c2ccccc2)c1]

Intermediate 127-1 (10.0 g, 0.023 mol), 3,5-dicyanophenylboronic acid (9.5 g, 0.055 mol), potassium carbonate (15.9 g, 0.115 mol), Pd(PPh₃)₄ (1.33 g, 0.001 mol), 200 mL of toluene, 50 mL of ethanol, and 50 mL of H₂O were put into a container, and the resulting mixture was stirred at 100° C. for 6 hours and reacted. After completion of the reaction, the resulting product was extracted, concentrated, and subjected to column chromatography and recrystallization to obtain 7.9 g (yield 64.9%) of <Intermediate 127-2>.

### (3) Preparation Example 3: Synthesis of Compound 127

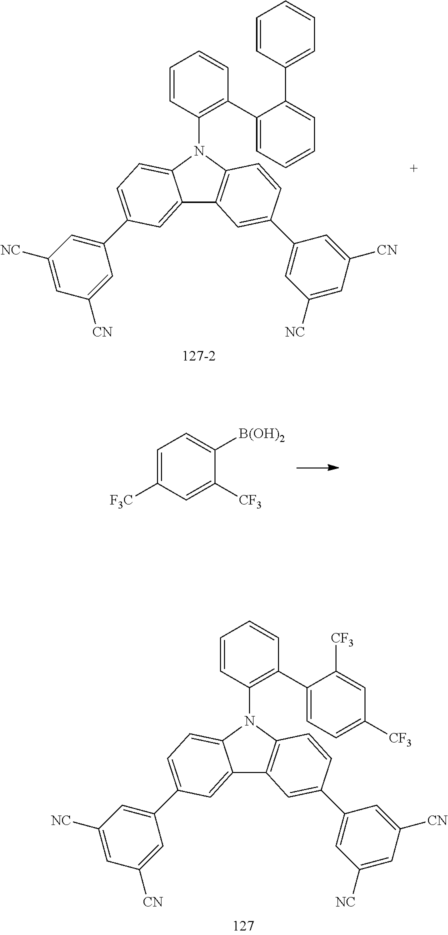 [SMILES: N#Cc1cc(C#N)cc(-c2ccc3c(c2)c2cc(-c4cc(C#N)cc(C#N)c4)ccc2n3-c2ccccc2-c2ccccc2-c2ccccc2)c1 ; OB(O)c1ccc(C(F)(F)F)cc1C(F)(F)F ; N#Cc1cc(C#N)cc(-c2ccc3c(c2)c2cc(-c4cc(C#N)cc(C#N)c4)ccc2n3-c2ccccc2-c2ccc(C(F)(F)F)cc2C(F)(F)F)c1]

Intermediate 127-2 (10.0 g, 0.019 mol), 2,4-bis(trifluoromethyl)phenylboronic acid (5.84 g, 0.023 mol), potassium carbonate (7.8 g, 0.057 mol), a catalyst Pd(OAc)₂ (1.09 g, 0.001 mol), a ligand X-Phos (0.99 g, 0.002 mol), 200 mL of THF, 50 mL of H₂O, and 50 mL of ethanol were put into a container, and the resulting mixture was stirred at 90° C. for 6 hours and reacted. After completion of the reaction, the resulting product was extracted and concentrated, and then subjected to column chromatography and recrystallization to obtain 8.1 g (yield 60.7%) of <Compound 127>.

LC/MS: m/z=707[(M)⁺]

### Synthesis Example 6: Synthesis of Compound 152

### (1) Preparation Example 1: Synthesis of Intermediate 152-1

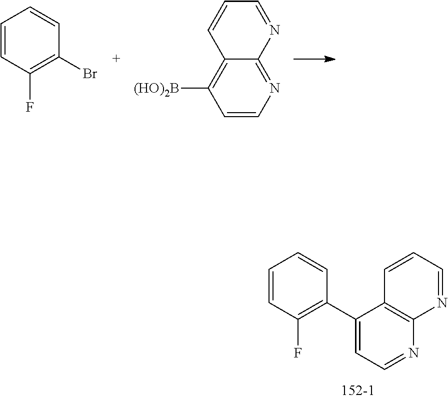 [SMILES: Fc1ccccc1Br ; OB(O)c1ccnc2ncccc12 ; Fc1ccccc1-c1ccnc2ncccc12]

1-Bromo-2-fluorobenzene (10.0 g, 0.057 mol), 1,8-naphthyridin-4-ylboronic acid (11.9 g, 0.069 mol), potassium carbonate (23.7 g, 0.171 mol), Pd(PPh₃)₄ (3.3 g, 0.003 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography to obtain 8.5 g (yield 66.3%) of <Compound 152-1>.

### (2) Preparation Example 2: Synthesis of Intermediate 152-2

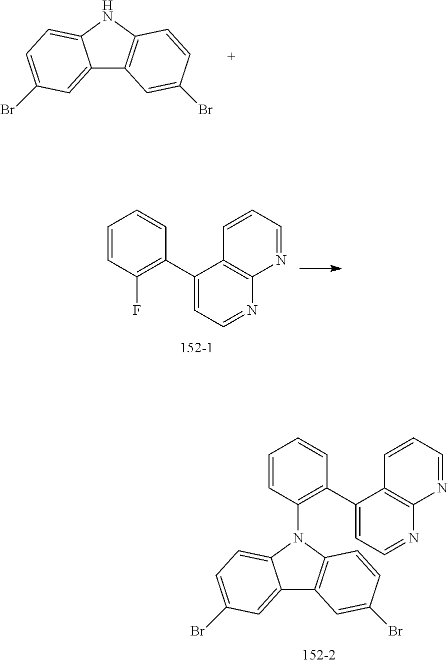 [SMILES: Brc1ccc2[nH]c3ccc(Br)cc3c2c1 ; Fc1ccccc1-c1ccnc2ncccc12 ; Brc1ccc2c(c1)c1cc(Br)ccc1n2-c1ccccc1-c1ccnc2ncccc12]

500 mL of DMF was put into 3,6-dibromocarbazole (10.0 g, 0.031 mol), Intermediate 152-1 (8.3 g, 0.037 mol), and cesium carbonate (6.4 g, 0.046 mol), and the resulting mixture was stirred under reflux at 150° C. for 15 hours and reacted. After completion of the reaction, the resulting product was extracted and concentrated, and then subjected to column chromatography and recrystallization to obtain 10.2 g (yield 62.6%) of <Intermediate 152-2>.

### (3) Preparation Example 3: Synthesis of Compound 152

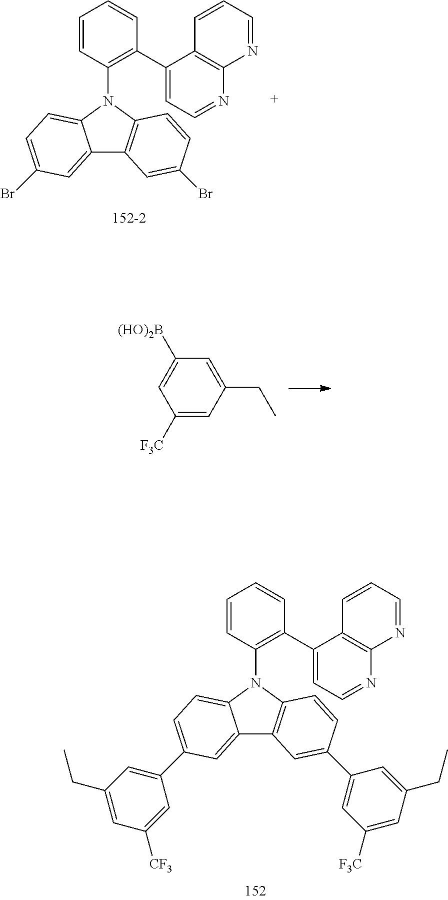 [SMILES: Brc1ccc2c(c1)c1cc(Br)ccc1n2-c1ccccc1-c1ccnc2ncccc12 ; CCc1cc(B(O)O)cc(C(F)(F)F)c1 ; CCc1cc(-c2ccc3c(c2)c2cc(-c4cc(CC)cc(C(F)(F)F)c4)ccc2n3-c2ccccc2-c2ccnc3ncccc23)cc(C(F)(F)F)c1]

Intermediate 152-2 (10.0 g, 0.019 mol), 3-ethyl-5-(trifluoromethyl)phenylboronic acid (4.9 g, 0.023 mol), potassium carbonate (7.8 g, 0.057 mol), Pd(PPh₃)₄ (1.09 g, 0.001 mol), 100 mL of toluene, 30 mL of H₂O, and 30 mL of ethanol were put into a container, and the resulting mixture was stirred at 95° C. for 6 hours and reacted. After completion of the reaction, the resulting product was extracted and concentrated, and then subjected to column chromatography and recrystallization to obtain 8.9 g (yield 65.8%) of <Compound 152>.

LC/MS: m/z=715[(M)⁺]

### Synthesis Example 7: Synthesis of Compound 1-19

### (1) Preparation Example 1: Synthesis of Intermediate 1-19-1

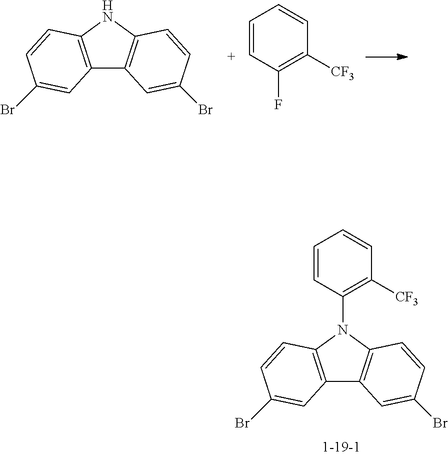 [SMILES: Brc1ccc2[nH]c3ccc(Br)cc3c2c1 ; Fc1ccccc1C(F)(F)F ; FC(F)(F)c1ccccc1-n1c2ccc(Br)cc2c2cc(Br)ccc21]

500 mL of DMF was added to 3,6-dibromocarbazole (10.0 g, 0.031 mol), 2-fluorobenzotrifluoride (6.1 g, 0.037 mol), and Cs₂CO₃ (6.4 g, 0.046 mol), and the resulting mixture was stirred under reflux at 150° C. for 12 hours and reacted. After completion of the reaction, the resulting product was extracted and concentrated, and then subjected to column chromatography and recrystallization to obtain 9.8 g (yield 67.9%) of <Intermediate 1-19-1>.

### (2) Preparation Example 2: Synthesis of Intermediate 1-19-2

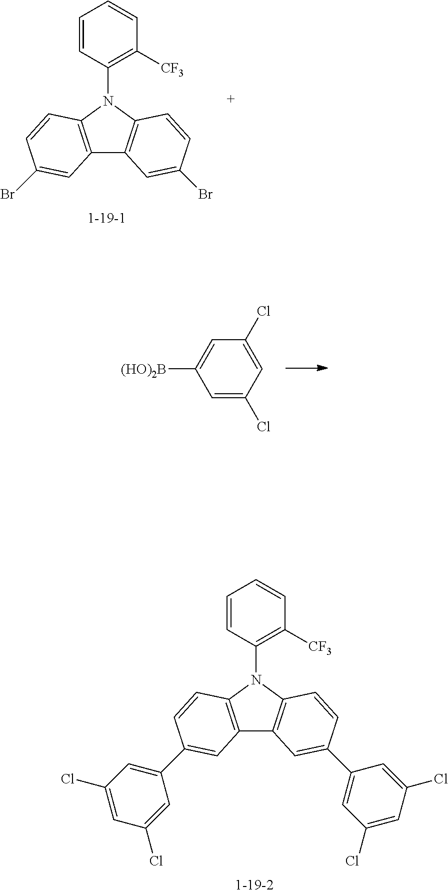 [SMILES: FC(F)(F)c1ccccc1-n1c2ccc(Br)cc2c2cc(Br)ccc21 ; OB(O)c1cc(Cl)cc(Cl)c1 ; FC(F)(F)c1ccccc1-n1c2ccc(-c3cc(Cl)cc(Cl)c3)cc2c2cc(-c3cc(Cl)cc(Cl)c3)ccc21]

Intermediate 1-19-1 (10.0 g, 0.021 mol), 3,5-dichlorophenylboronic acid (9.8 g, 0.051 mol), K₂CO₃ (17.7 g, 0.128 mol), Pd(PPh₃)₄ (0.5 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 8.2 g (yield 63.9%) of <Intermediate 1-19-2>.

### (3) Preparation Example 3: Synthesis of Compound 1-19

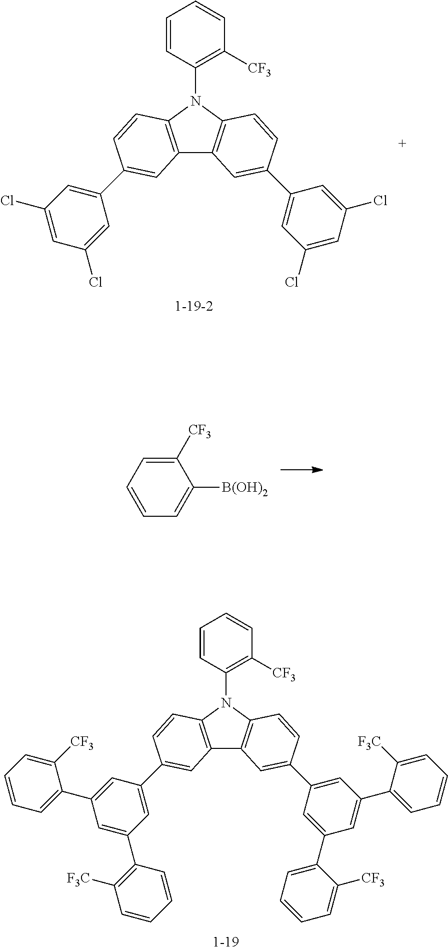 [SMILES: FC(F)(F)c1ccccc1-n1c2ccc(-c3cc(Cl)cc(Cl)c3)cc2c2cc(-c3cc(Cl)cc(Cl)c3)ccc21 ; OB(O)c1ccccc1C(F)(F)F ; FC(F)(F)c1ccccc1-c1cc(-c2ccc3c(c2)c2cc(-c4cc(-c5ccccc5C(F)(F)F)cc(-c5ccccc5C(F)(F)F)c4)ccc2n3-c2ccccc2C(F)(F)F)cc(-c2ccccc2C(F)(F)F)c1]

Intermediate 1-19-2 (10.0 g, 0.017 mol), 2-trifluoromethylbenzeneboronic acid (15.2 g, 0.080 mol), K₂CO₃ (27.6 g, 0.200 mol), a catalyst Pd(OAc)₂ (1.9 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 12.5 g (yield 72.3%) of <Compound 1-19>.

LC/MS: m/z=1039[(M)⁺]

### Synthesis Example 2: Synthesis of Compound 1-23

### (1) Preparation Example 1: Synthesis of Compound 1-23

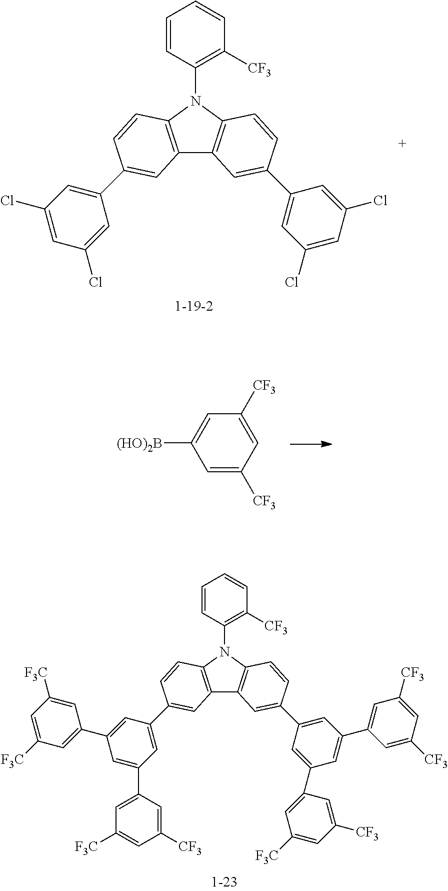 [SMILES: FC(F)(F)c1ccccc1-n1c2ccc(-c3cc(Cl)cc(Cl)c3)cc2c2cc(-c3cc(Cl)cc(Cl)c3)ccc21 ; OB(O)c1cc(C(F)(F)F)cc(C(F)(F)F)c1 ; FC(F)(F)c1cc(-c2cc(-c3cc(C(F)(F)F)cc(C(F)(F)F)c3)cc(-c3ccc4c(c3)c3cc(-c5cc(-c6cc(C(F)(F)F)cc(C(F)(F)F)c6)cc(-c6cc(C(F)(F)F)cc(C(F)(F)F)c6)c5)ccc3n4-c3ccccc3C(F)(F)F)c2)cc(C(F)(F)F)c1]

Intermediate 1-19-2 (10.0 g, 0.017 mol), 3,5-bis(trifluoromethyl)phenylboronic acid (20.6 g, 0.080 mol), K₂CO₃ (27.6 g, 0.200 mol), a catalyst Pd(OAc)₂ (1.9 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 15.5 g (yield 71.0%) of <Compound 1-23>.

LC/MS: m/z=1311[(M)⁺]

### Synthesis Example 3: Synthesis of Compound 1-27

(1) Preparation Example 1: Synthesis of Compound 1-27

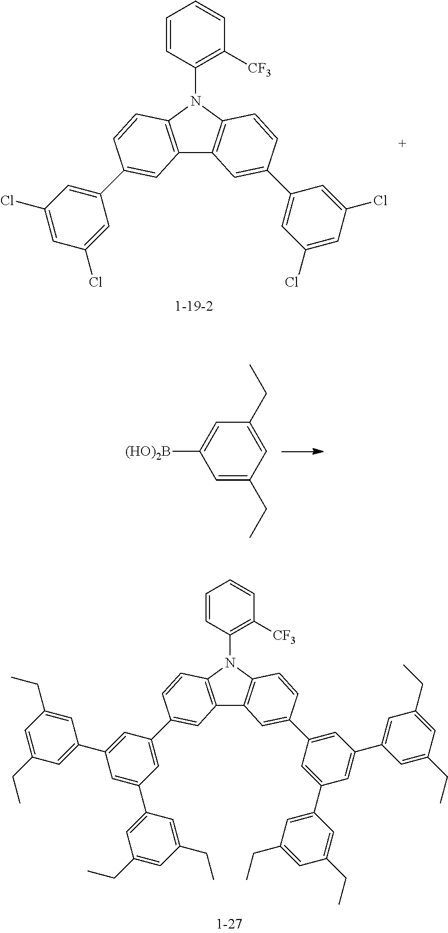 [SMILES: FC(F)(F)c1ccccc1-n1c2ccc(-c3cc(Cl)cc(Cl)c3)cc2c2cc(-c3cc(Cl)cc(Cl)c3)ccc21 ; CCc1cc(CC)cc(B(O)O)c1 ; CCc1cc(CC)cc(-c2cc(-c3cc(CC)cc(CC)c3)cc(-c3ccc4c(c3)c3cc(-c5cc(-c6cc(CC)cc(CC)c6)cc(-c6cc(CC)cc(CC)c6)c5)ccc3n4-c3ccccc3C(F)(F)F)c2)c1]

Intermediate 1-19-2 (10.0 g, 0.017 mol), (3,5-diethylphenyl)boronic acid (14.2 g, 0.080 mol), K₂CO₃ (27.6 g, 0.200 mol), a catalyst Pd(OAc)₂ (1.9 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 10.1 g (yield 73.1%) of <Compound 1-27>.

LC/MS: m/z=991[(M)⁺]

### Synthesis Example 4: Synthesis of Compound 1-37

### (1) Preparation Example 1: Synthesis of Compound 1-37

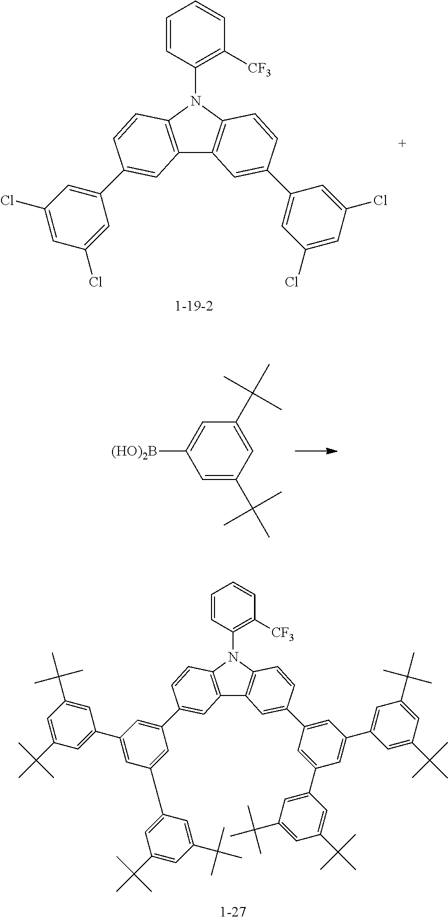 [SMILES: FC(F)(F)c1ccccc1-n1c2ccc(-c3cc(Cl)cc(Cl)c3)cc2c2cc(-c3cc(Cl)cc(Cl)c3)ccc21 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3C(F)(F)F)c2)cc(C(C)(C)C)c1]

Intermediate 1-19-2 (10.0 g, 0.017 mol), 3,5-di-tert-butylphenylboronic acid (18.7 g, 0.080 mol), K₂CO₃ (27.6 g, 0.200 mol), a catalyst Pd(OAc)₂ (1.9 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 15.5 g (yield 76.6%) of <Compound 1-37>.

LC/MS: m/z=1215[(M)⁺]

### Synthesis Example 5: Synthesis of Compound 1-56

### (1) Preparation Example 1: Synthesis of Intermediate 1-56-1

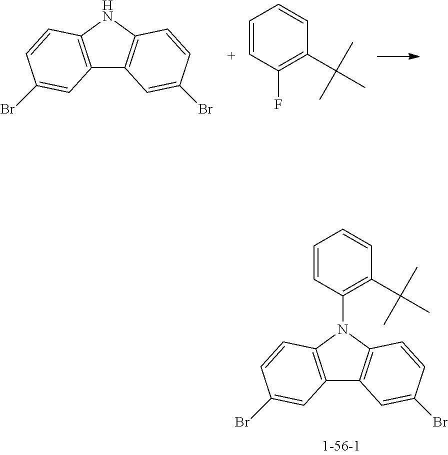 [SMILES: Brc1ccc2[nH]c3ccc(Br)cc3c2c1 ; CC(C)(C)c1ccccc1F ; CC(C)(C)c1ccccc1-n1c2ccc(Br)cc2c2cc(Br)ccc21]

500 mL of DMF was added to 3,6-dibromocarbazole (10 g, 0.031 mol), 1-(tert-butyl)-2-fluorobenzene (5.6 g, 0.037 mol), and Cs₂CO₃ (6.4 g, 0.046 mol), and the resulting mixture was stirred under reflux at 150° C. for 12 hours and reacted. After completion of the reaction, the resulting product was extracted and concentrated, and then subjected to column chromatography to obtain 9.5 g (yield 67.5%) of <Intermediate 1-56-1>.

### (2) Preparation Example 2: Synthesis of Intermediate 1-56-2

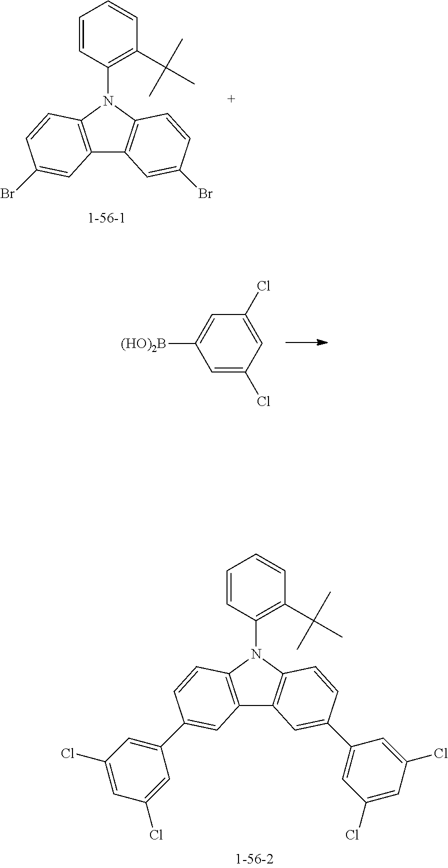 [SMILES: CC(C)(C)c1ccccc1-n1c2ccc(Br)cc2c2cc(Br)ccc21 ; OB(O)c1cc(Cl)cc(Cl)c1 ; CC(C)(C)c1ccccc1-n1c2ccc(-c3cc(Cl)cc(Cl)c3)cc2c2cc(-c3cc(Cl)cc(Cl)c3)ccc21]

Intermediate 1-56-1 (10.0 g, 0.022 mol), 3,5-dichlorophenylboronic acid (10.0 g, 0.053 mol), K₂CO₃ (18.1 g, 0.131 mol), Pd(PPh₃)₄ (0.5 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 9.5 g (yield 73.7%) of <Intermediate 1-56-2>.

### (3) Preparation Example 3: Synthesis of Compound 1-56

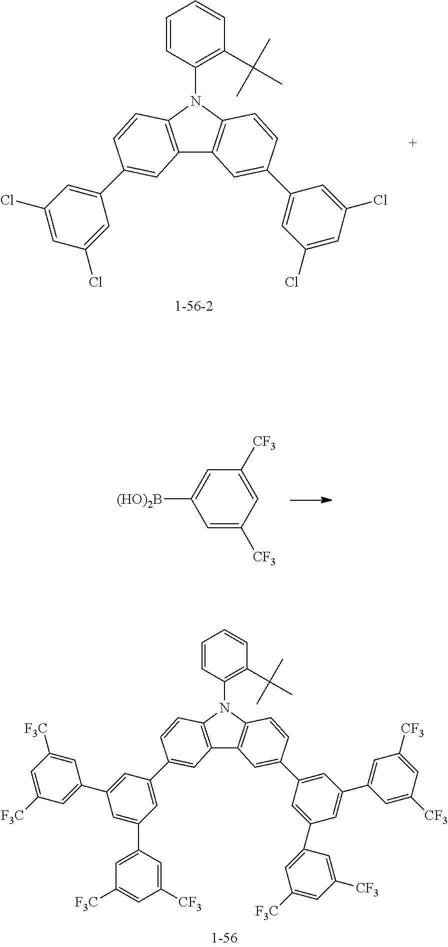 [SMILES: CC(C)(C)c1ccccc1-n1c2ccc(-c3cc(Cl)cc(Cl)c3)cc2c2cc(-c3cc(Cl)cc(Cl)c3)ccc21 ; OB(O)c1cc(C(F)(F)F)cc(C(F)(F)F)c1 ; CC(C)(C)c1ccccc1-n1c2ccc(-c3cc(-c4cc(C(F)(F)F)cc(C(F)(F)F)c4)cc(-c4cc(C(F)(F)F)cc(C(F)(F)F)c4)c3)cc2c2cc(-c3cc(-c4cc(C(F)(F)F)cc(C(F)(F)F)c4)cc(-c4cc(C(F)(F)F)cc(C(F)(F)F)c4)c3)ccc21]

Intermediate 1-56-2 (10.0 g, 0.017 mol), 3,5-bis(trifluoromethyl)phenylboronic acid (21.0 g, 0.081 mol), K₂CO₃ (28.1 g, 0.204 mol), a catalyst Pd(OAc)₂ (2.0 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 15.3 g (yield 69.4%) of <Compound 1-56>.

LC/MS: m/z=1299[(M)⁺]

### Synthesis Example 6: Synthesis of Compound 1-65

### (1) Preparation Example 1: Synthesis of Compound 1-65

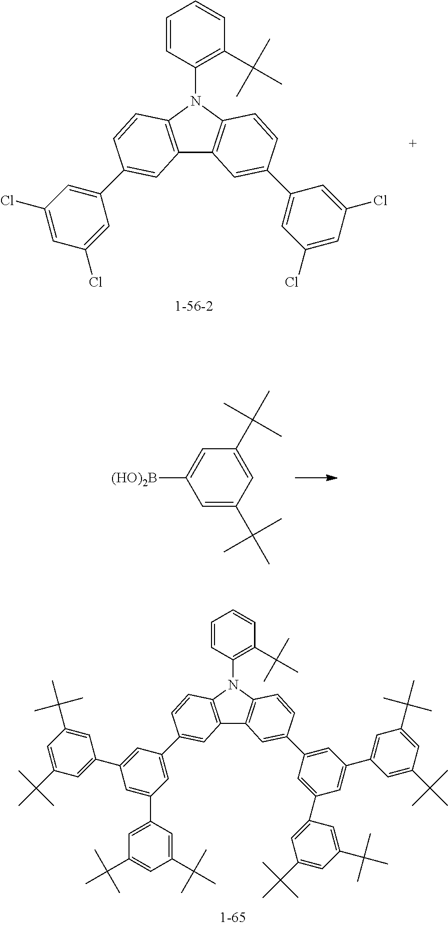 [SMILES: CC(C)(C)c1ccccc1-n1c2ccc(-c3cc(Cl)cc(Cl)c3)cc2c2cc(-c3cc(Cl)cc(Cl)c3)ccc21 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3C(C)(C)C)c2)cc(C(C)(C)C)c1]

Intermediate 1-56-2 (10.0 g, 0.017 mol), 3,5-di-tert-butylphenylboronic acid (19.1 g, 0.081 mol), K₂CO₃ (28.1 g, 0.204 mol), a catalyst Pd(OAc)₂ (2.0 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 15.7 g (yield 76.8%) of <Compound 1-65>.

LC/MS: m/z=1203[(M)⁺]

### Synthesis Example 7: Synthesis of Compound 1-67

### (1) Preparation Example 1: Synthesis of Compound 1-67

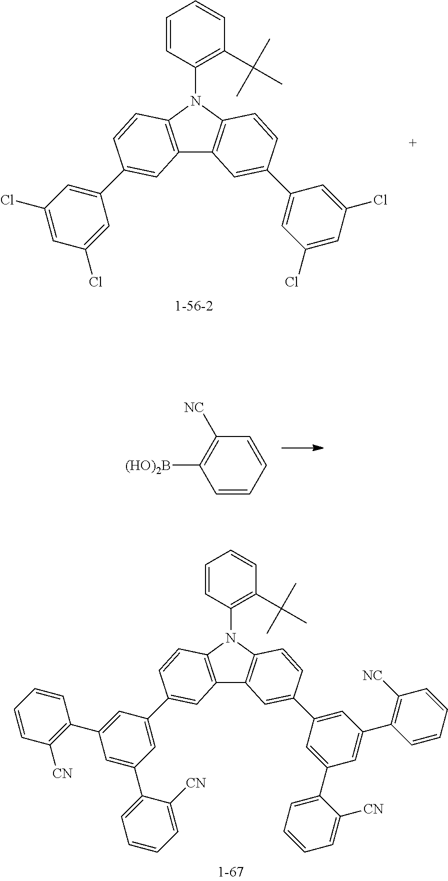 [SMILES: CC(C)(C)c1ccccc1-n1c2ccc(-c3cc(Cl)cc(Cl)c3)cc2c2cc(-c3cc(Cl)cc(Cl)c3)ccc21 ; N#Cc1ccccc1B(O)O ; CC(C)(C)c1ccccc1-n1c2ccc(-c3cc(-c4ccccc4C#N)cc(-c4ccccc4C#N)c3)cc2c2cc(-c3cc(-c4ccccc4C#N)cc(-c4ccccc4C#N)c3)ccc21]

Intermediate 1-56-2 (10.0 g, 0.017 mol), 2-cyanophenylboronic acid (12.0 g, 0.081 mol), K₂CO₃ (28.1 g, 0.204 mol), a catalyst Pd(OAc)₂ (2.0 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 11.2 g (yield 77.1%) of <Compound 1-67>.

LC/MS: m/z=855[(M)⁺]

### Synthesis Example 8: Synthesis of Compound 1-78

### (1) Preparation Example 1: Synthesis of Intermediate 1-78-1

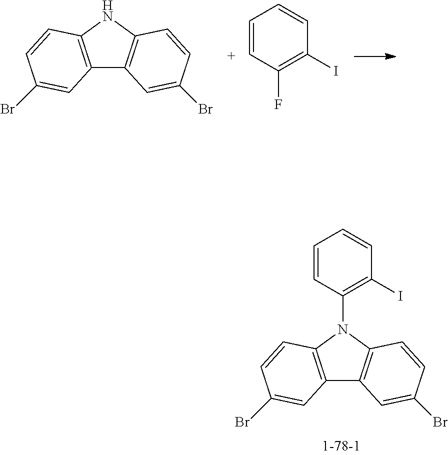 [SMILES: Brc1ccc2[nH]c3ccc(Br)cc3c2c1 ; Fc1ccccc1I ; Brc1ccc2c(c1)c1cc(Br)ccc1n2-c1ccccc1I]

500 mL of DMF was added to 3,6-dibromocarbazole (10 g, 0.019 mol), 2-fluoro-1-iodobenzene (3.2 g, 0.023 mol), and Cs₂CO₃ (3.9 g, 0.029 mol), and the resulting mixture was stirred under reflux at 150° C. for 12 hours and reacted. After completion of the reaction, the resulting product was extracted and concentrated, and then subjected to column chromatography to obtain 5.8 g (yield 61.7%) of <Intermediate 1-78-1>.

### (2) Preparation Example 2: Synthesis of Intermediate 1-78-2

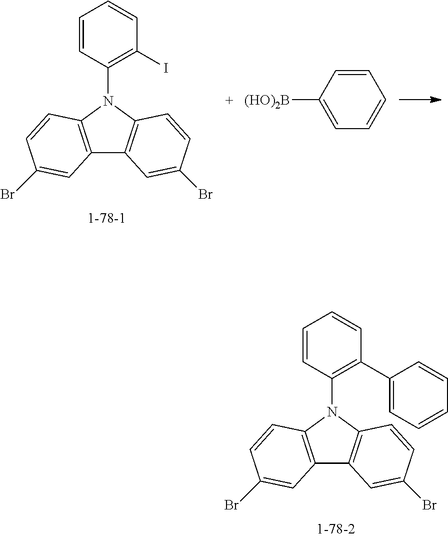 [SMILES: Brc1ccc2c(c1)c1cc(Br)ccc1n2-c1ccccc1I ; OB(O)c1ccccc1 ; Brc1ccc2c(c1)c1cc(Br)ccc1n2-c1ccccc1-c1ccccc1]

Intermediate 1-78-1 (10.0 g, 0.019 mol), phenylboronic acid (2.8 g, 0.023 mol), K₂CO₃ (7.9 g, 0.057 mol), Pd(PPh₃)₄ (0.4 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography to obtain 5.7 g (yield 63.0%) of <Intermediate 1-78-2>.

### (3) Preparation Example 3: Synthesis of Intermediate 1-78-3

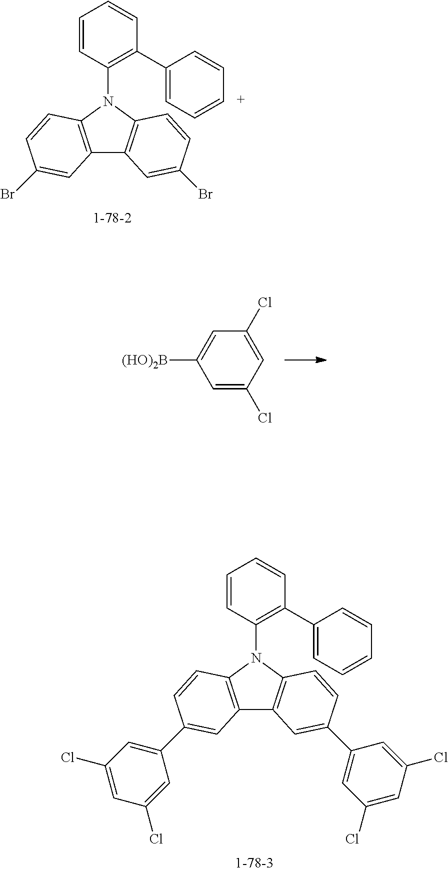 [SMILES: Brc1ccc2c(c1)c1cc(Br)ccc1n2-c1ccccc1-c1ccccc1 ; OB(O)c1cc(Cl)cc(Cl)c1 ; Clc1cc(Cl)cc(-c2ccc3c(c2)c2cc(-c4cc(Cl)cc(Cl)c4)ccc2n3-c2ccccc2-c2ccccc2)c1]

Intermediate 1-78-2 (10.0 g, 0.021 mol), 3,5-dichlorophenylboronic acid (9.6 g, 0.050 mol), K₂CO₃ (17.4 g, 0.126 mol), Pd(PPh₃)₄ (0.5 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 9.7 g (yield 76.0%) of <Intermediate 1-78-3>.

### (3) Preparation Example 3: Synthesis of Compound 1-78

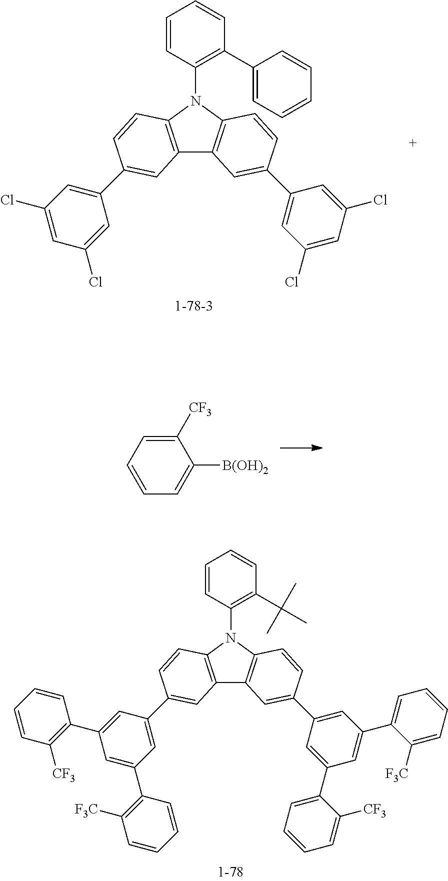 [SMILES: Clc1cc(Cl)cc(-c2ccc3c(c2)c2cc(-c4cc(Cl)cc(Cl)c4)ccc2n3-c2ccccc2-c2ccccc2)c1 ; OB(O)c1ccccc1C(F)(F)F ; CC(C)(C)c1ccccc1-n1c2ccc(-c3cc(-c4ccccc4C(F)(F)F)cc(-c4ccccc4C(F)(F)F)c3)cc2c2cc(-c3cc(-c4ccccc4C(F)(F)F)cc(-c4ccccc4C(F)(F)F)c3)ccc21]

Intermediate 1-78-3 (10.0 g, 0.016 mol), 2-(trifluoromethyl)phenylboronic acid (15.0 g, 0.079 mol), K₂CO₃ (27.2 g, 0.197 mol), a catalyst Pd(OAc)₂ (1.9 g, 0.002 mol), a ligand X-Phos (2.4 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 10.9 g (yield 63.4%) of <Compound 1-78>.

LC/MS: m/z=1047[(M)⁺]

### Synthesis Example 9: Synthesis of Compound 1-85

### (1) Preparation Example 1: Synthesis of Compound 1-85

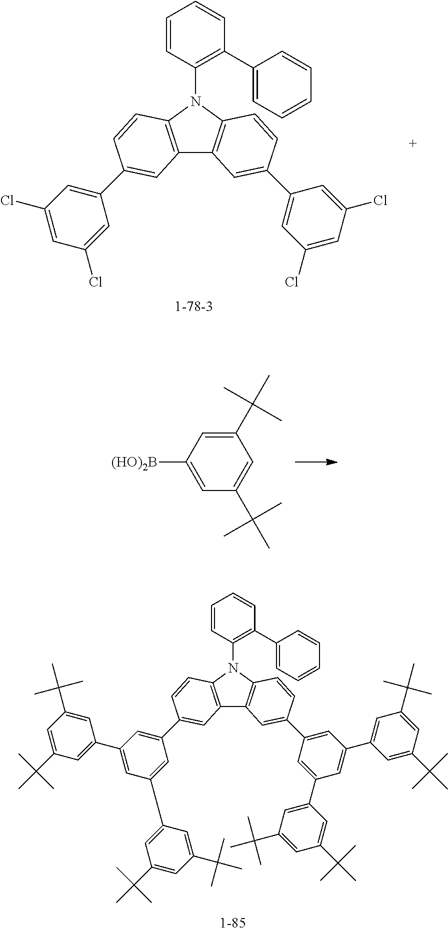 [SMILES: Clc1cc(Cl)cc(-c2ccc3c(c2)c2cc(-c4cc(Cl)cc(Cl)c4)ccc2n3-c2ccccc2-c2ccccc2)c1 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3-c3ccccc3)c2)cc(C(C)(C)C)c1]

Intermediate 1-78-3 (10.0 g, 0.016 mol), 3,5-di-tert-butylphenylboronic acid (18.4 g, 0.079 mol), K₂CO₃ (27.2 g, 0.197 mol), a catalyst Pd(OAc)₂ (1.90 g, 0.002 mol), a ligand X-Phos (2.3 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 15.2 g (yield 75.6%) of <Compound 1-85>.

LC/MS: m/z=1223[(M)⁺]

### Synthesis Example 10: Synthesis of Compound 1-108

### (1) Preparation Example 1: Synthesis of Intermediate 1-108-1

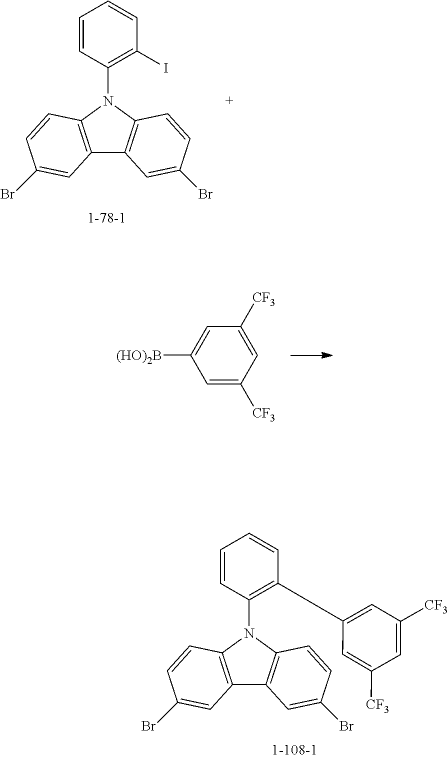 [SMILES: Brc1ccc2c(c1)c1cc(Br)ccc1n2-c1ccccc1I ; OB(O)c1cc(C(F)(F)F)cc(C(F)(F)F)c1 ; FC(F)(F)c1cc(-c2ccccc2-n2c3ccc(Br)cc3c3cc(Br)ccc32)cc(C(F)(F)F)c1]

500 mL of DMF was added to Intermediate 1-78-1 (10 g, 0.019 mol), 3,5-bis(trifluoromethyl)phenylboronic acid (3.2 g, 0.023 mol), and Cs₂CO₃ (3.9 g, 0.029 mol), and the resulting mixture was stirred under reflux at 150° C. for 12 hours and reacted. After completion of the reaction, the resulting product was extracted and concentrated, and then subjected to column chromatography to obtain 5.8 g (yield 61.7%) of <Intermediate 1-108-1>.

### (2) Preparation Example 2: Synthesis of Intermediate 1-108-2

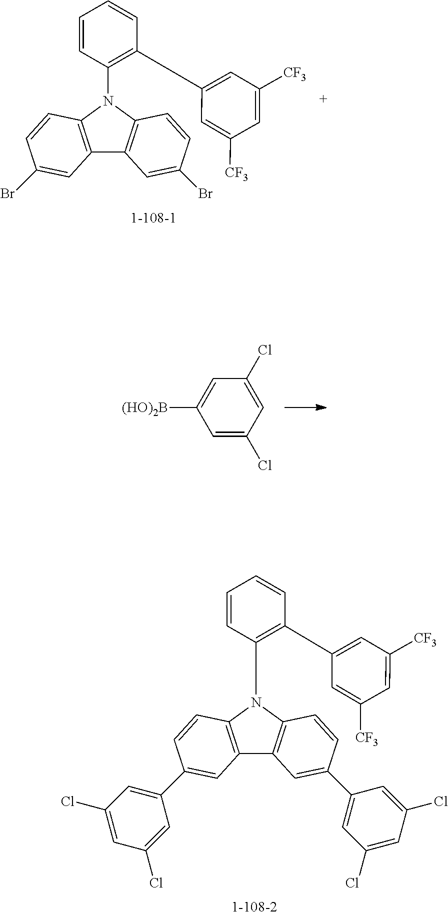 [SMILES: FC(F)(F)c1cc(-c2ccccc2-n2c3ccc(Br)cc3c3cc(Br)ccc32)cc(C(F)(F)F)c1 ; OB(O)c1cc(Cl)cc(Cl)c1 ; FC(F)(F)c1cc(-c2ccccc2-n2c3ccc(-c4cc(Cl)cc(Cl)c4)cc3c3cc(-c4cc(Cl)cc(Cl)c4)ccc32)cc(C(F)(F)F)c1]

Intermediate 1-108-1 (10.0 g, 0.016 mol), 3,5-dichlorophenylboronic acid (5.3 g, 0.038 mol), K₂CO₃ (11.0 g, 0.080 mol), a catalyst Pd(OAc)₂ (1.8 g, 0.002 mol), a ligand X-Phos (1.5 g, 0.003 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 8.5 g (yield 61.9%) of <Intermediate 1-108-2>.

### (3) Preparation Example 3: Synthesis of Compound 1-108

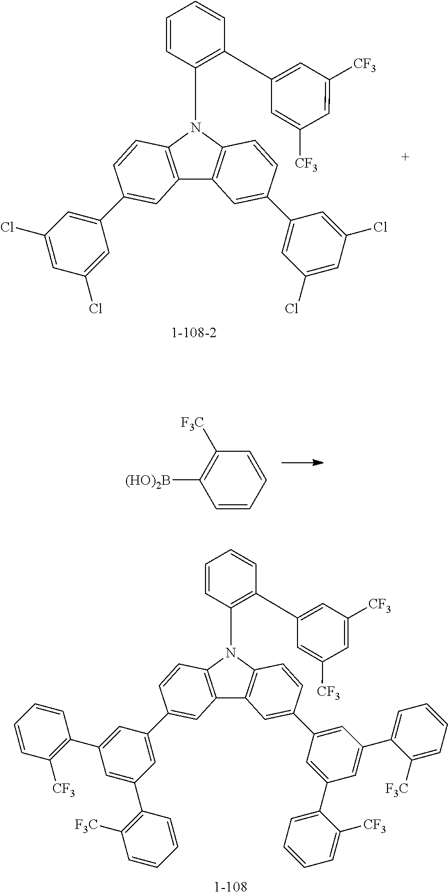 [SMILES: FC(F)(F)c1cc(-c2ccccc2-n2c3ccc(-c4cc(Cl)cc(Cl)c4)cc3c3cc(-c4cc(Cl)cc(Cl)c4)ccc32)cc(C(F)(F)F)c1 ; OB(O)c1ccccc1C(F)(F)F ; FC(F)(F)c1cc(-c2ccccc2-n2c3ccc(-c4cc(-c5ccccc5C(F)(F)F)cc(-c5ccccc5C(F)(F)F)c4)cc3c3cc(-c4cc(-c5ccccc5C(F)(F)F)cc(-c5ccccc5C(F)(F)F)c4)ccc32)cc(C(F)(F)F)c1]

Intermediate 1-108-2 (10.0 g, 0.013 mol), 2-(trifluoromethyl)phenylboronic acid (12.2 g, 0.064 mol), K₂CO₃ (22.3 g, 0.161 mol), a catalyst Pd(OAc)₂ (1.6 g, 0.001 mol), a ligand X-Phos (2.0 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 10.2 g (yield 64.2%) of <Compound 1-108>.

LC/MS: m/z=1183[(M)⁺]

### Synthesis Example 11: Synthesis of Compound 1-115

### (1) Preparation Example 1: Synthesis of Compound 1-115

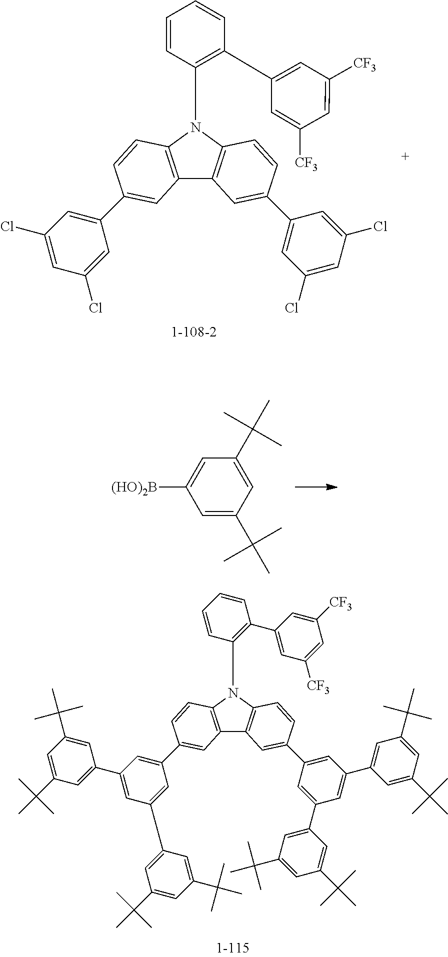

Intermediate 1-108-2 (10.0 g, 0.013 mol), 3,5-di-tert-butylphenylboronic acid (15.1 g, 0.064 mol), K₂CO₃ (22.3 g, 0.161 mol), a catalyst Pd(OAc)₂ (1.6 g, 0.001 mol), a ligand X-Phos (1.9 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 11.2 g (yield 61.3%) of <Compound 1-115>.

LC/MS: m/z=1360[(M)⁺]

### Synthesis Example 12: Synthesis of Compound 1-140

### (1) Preparation Example 1: Synthesis of Intermediate 1-140-1

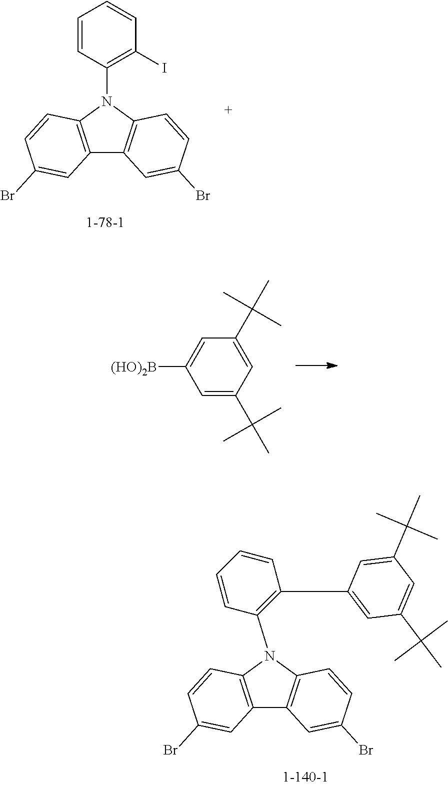 [SMILES: Brc1ccc2c(c1)c1cc(Br)ccc1n2-c1ccccc1I ; CC(C)(C)c1cc(B(O)O)cc(C(C)(C)C)c1 ; CC(C)(C)c1cc(-c2ccccc2-n2c3ccc(Br)cc3c3cc(Br)ccc32)cc(C(C)(C)C)c1]

Intermediate 1-78-1 (10.0 g, 0.019 mol), 3,5-di-tert-butylphenylboronic acid (5.3 g, 0.023 mol), K₂CO₃ (7.9 g, 0.057 mol), Pd(PPh₃)₄ (0.4 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography to obtain 6.3 g (yield 56.3%) of <Intermediate 1-140-1>.

### (2) Preparation Example 2: Synthesis of Intermediate 1-140-2

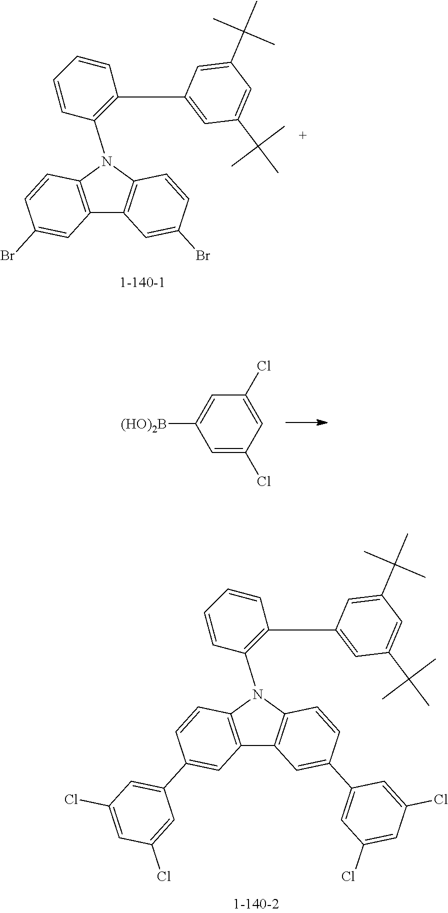 [SMILES: CC(C)(C)c1cc(-c2ccccc2-n2c3ccc(Br)cc3c3cc(Br)ccc32)cc(C(C)(C)C)c1 ; OB(O)c1cc(Cl)cc(Cl)c1 ; CC(C)(C)c1cc(-c2ccccc2-n2c3ccc(-c4cc(Cl)cc(Cl)c4)cc3c3cc(-c4cc(Cl)cc(Cl)c4)ccc32)cc(C(C)(C)C)c1]

Intermediate 1-140-1 (10.0 g, 0.017 mol), 3,5-dichlorophenylboronic acid (7.8 g, 0.041 mol), K₂CO₃ (14.1 g, 0.102 mol), Pd(PPh₃)₄ (0.4 g, 0.0003 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 9.1 g (yield 74.3%) of <Intermediate 1-140-2>.

### (3) Preparation Example 3: Synthesis of Compound 1-140

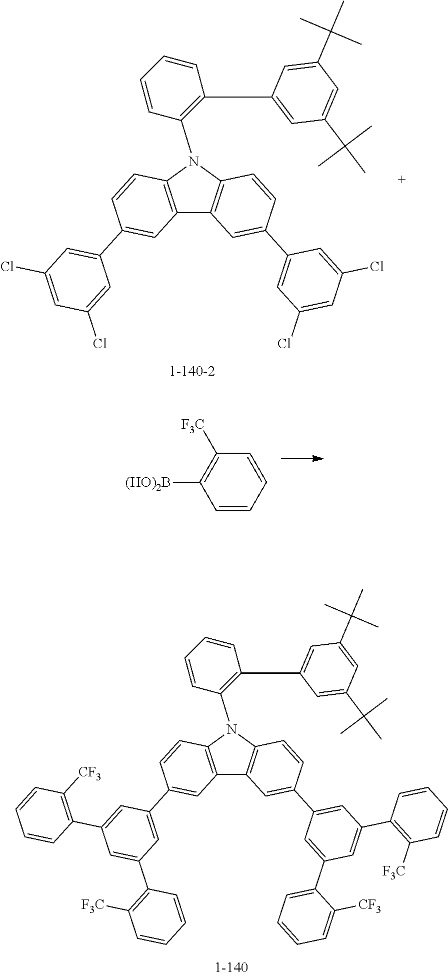 [SMILES: CC(C)(C)c1cc(-c2ccccc2-n2c3ccc(-c4cc(Cl)cc(Cl)c4)cc3c3cc(-c4cc(Cl)cc(Cl)c4)ccc32)cc(C(C)(C)C)c1 ; OB(O)c1ccccc1C(F)(F)F ; CC(C)(C)c1cc(-c2ccccc2-n2c3ccc(-c4cc(-c5ccccc5C(F)(F)F)cc(-c5ccccc5C(F)(F)F)c4)cc3c3cc(-c4cc(-c5ccccc5C(F)(F)F)cc(-c5ccccc5C(F)(F)F)c4)ccc32)cc(C(C)(C)C)c1]

Intermediate 1-140-2 (10.0 g, 0.014 mol), 2-(trifluoromethyl)phenylboronic acid (12.6 g, 0.067 mol), K₂CO₃ (23.0 g, 0.166 mol), a catalyst Pd(OAc)₂ (1.6 g, 0.001 mol), a ligand X-Phos (1.9 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 12.3 g (yield 76.5%) of <Compound 1-140>.

LC/MS: m/z=1159[(M)⁺]

### Synthesis Example 13: Synthesis of Compound 1-147

### (1) Preparation Example 1: Synthesis of Compound 1-147

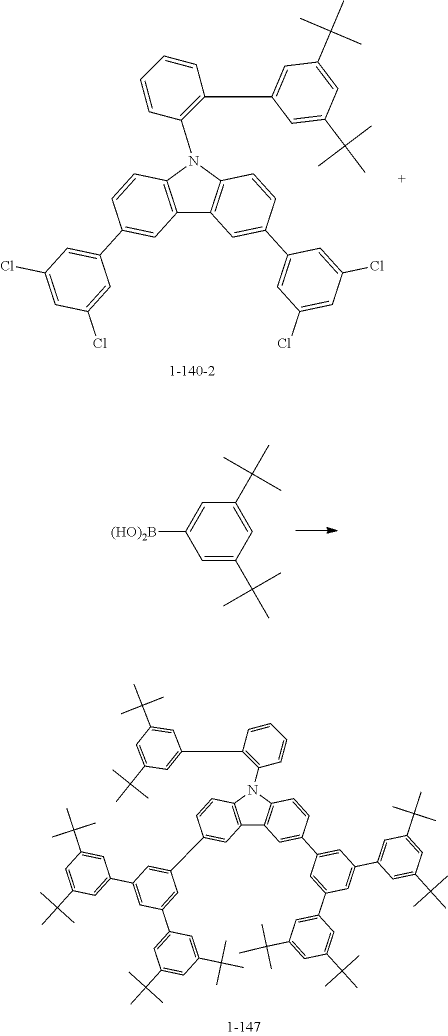 [SMILES: CC(C)(C)c1cc(-c2ccccc2-n2c3ccc(-c4cc(Cl)cc(Cl)c4)cc3c3cc(-c4cc(Cl)cc(Cl)c4)ccc32)cc(C(C)(C)C)c1 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3-c3cc(C(C)(C)C)cc(C(C)(C)C)c3)c2)cc(C(C)(C)C)c1]

Intermediate 1-140-2 (10.0 g, 0.014 mol), 3,5-di-tert-butylphenylboronic acid (15.6 g, 0.067 mol), K₂CO₃ (23.0 g, 0.166 mol), a catalyst Pd(OAc)₂ (1.60 g, 0.001 mol), a ligand X-Phos (2.0 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 12.8 g (yield 69.1%) of <Compound 1-147>.

LC/MS: m/z=1336[(M)⁺]

### Synthesis Example 14: Synthesis of Compound 1-149

### (1) Preparation Example 1: Synthesis of Compound 1-149

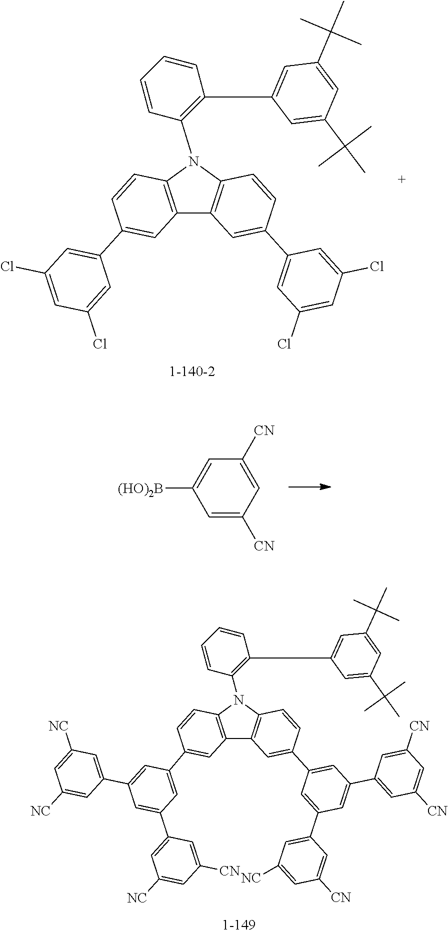 [SMILES: CC(C)(C)c1cc(-c2ccccc2-n2c3ccc(-c4cc(Cl)cc(Cl)c4)cc3c3cc(-c4cc(Cl)cc(Cl)c4)ccc32)cc(C(C)(C)C)c1 ; N#Cc1cc(C#N)cc(B(O)O)c1 ; CC(C)(C)c1cc(-c2ccccc2-n2c3ccc(-c4cc(-c5cc(C#N)cc(C#N)c5)cc(-c5cc(C#N)cc(C#N)c5)c4)cc3c3cc(-c4cc(-c5cc(C#N)cc(C#N)c5)cc(-c5cc(C#N)cc(C#N)c5)c4)ccc32)cc(C(C)(C)C)c1]

Intermediate 1-140-2 (10.0 g, 0.014 mol), 3,5-dicyanophenylboronic acid (11.4 g, 0.067 mol), K₂CO₃ (23.0 g, 0.166 mol), a catalyst Pd(OAc)₂ (1.6 g, 0.001 mol), a ligand X-Phos (2.0 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 10.1 g (yield 73.1%) of <Compound 1-149>.

LC/MS: m/z=1087[(M)⁺]

### Synthesis Example 15: Synthesis of Compound 1-154

### (1) Preparation Example 1: Synthesis of Intermediate 1-154-1

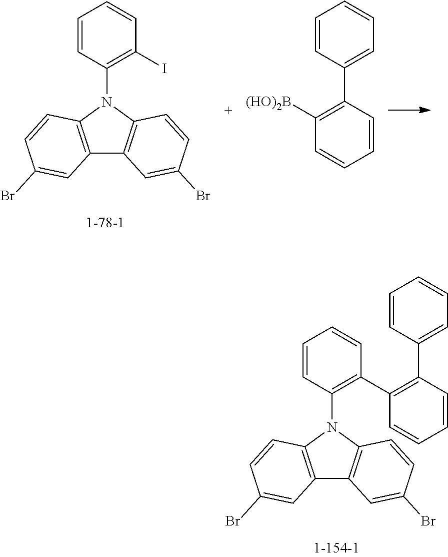 [SMILES: Brc1ccc2c(c1)c1cc(Br)ccc1n2-c1ccccc1I ; OB(O)c1ccccc1-c1ccccc1 ; Brc1ccc2c(c1)c1cc(Br)ccc1n2-c1ccccc1-c1ccccc1-c1ccccc1]

500 mL of DMF was added to Intermediate 1-78-1 (10 g, 0.019 mol), 2-biphenylboronic acid (4.5 g, 0.023 mol), and Cs₂CO₃ (7.9 g, 0.057 mol), and the resulting mixture was stirred under reflux at 150° C. for 12 hours and reacted. After completion of the reaction, the resulting product was extracted and concentrated, and then subjected to column chromatography to obtain 7.2 g (yield 68.6%) of <Intermediate 1-154-1>.

### (2) Preparation Example 2: Synthesis of Intermediate 1-154-2

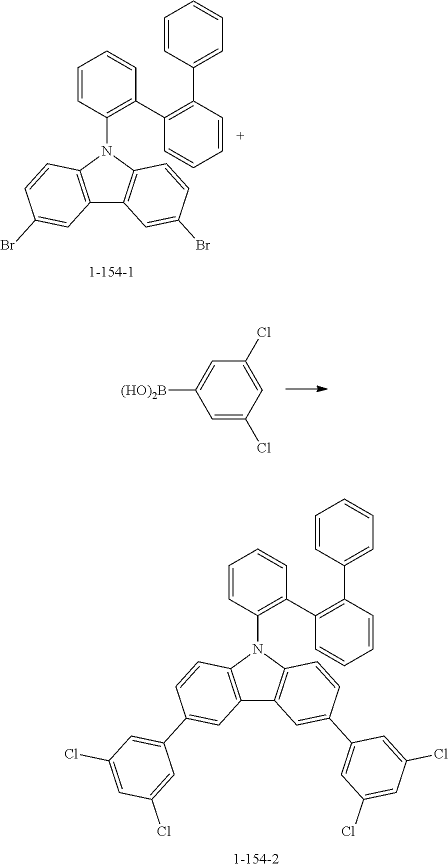 [SMILES: Brc1ccc2c(c1)c1cc(Br)ccc1n2-c1ccccc1-c1ccccc1-c1ccccc1 ; OB(O)c1cc(Cl)cc(Cl)c1 ; Clc1cc(Cl)cc(-c2ccc3c(c2)c2cc(-c4cc(Cl)cc(Cl)c4)ccc2n3-c2ccccc2-c2ccccc2-c2ccccc2)c1]

Intermediate 1-154-1 (10.0 g, 0.018 mol), 3,5-dichlorophenylboronic acid (8.3 g, 0.043 mol), K₂CO₃ (15.0 g, 0.108 mol), Pd(PPh₃)₄ (0.4 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 9.1 g (yield 73.4%) of <Intermediate 1-154-2>.

### (2) Preparation Example 3: Synthesis of Compound 1-154

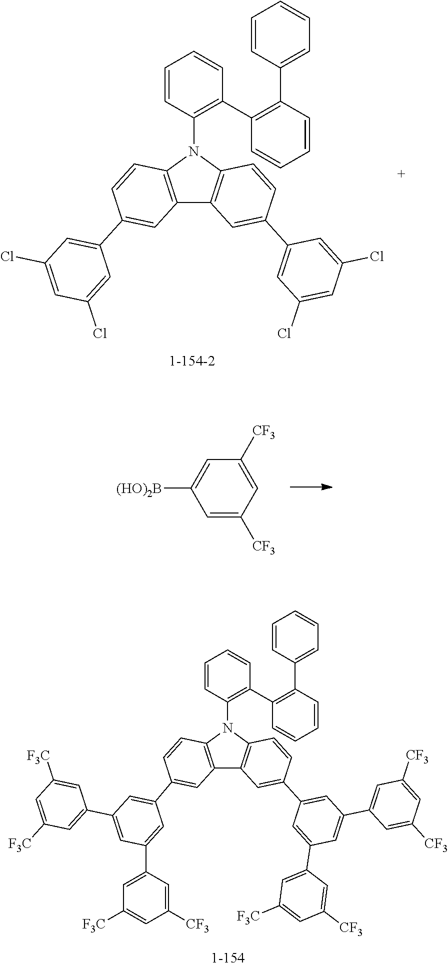 [SMILES: Clc1cc(Cl)cc(-c2ccc3c(c2)c2cc(-c4cc(Cl)cc(Cl)c4)ccc2n3-c2ccccc2-c2ccccc2-c2ccccc2)c1 ; OB(O)c1cc(C(F)(F)F)cc(C(F)(F)F)c1 ; FC(F)(F)c1cc(-c2cc(-c3cc(C(F)(F)F)cc(C(F)(F)F)c3)cc(-c3ccc4c(c3)c3cc(-c5cc(-c6cc(C(F)(F)F)cc(C(F)(F)F)c6)cc(-c6cc(C(F)(F)F)cc(C(F)(F)F)c6)c5)ccc3n4-c3ccccc3-c3ccccc3-c3ccccc3)c2)cc(C(F)(F)F)c1]

Intermediate 1-154-2 (10.0 g, 0.015 mol), 3,5-bis(trifluoromethyl)benzeneboronic acid (18.1 g, 0.070 mol), K₂CO₃ (24.2 g, 0.175 mol), a catalyst Pd(OAc)₂ (2.41 g, 0.002 mol), a ligand X-Phos (1.99 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 13.3 g (yield 65.3%) of <Compound 1-154>.

LC/MS: m/z=1395[(M)⁺]

### Synthesis Example 16: Synthesis of Compound 1-167

### (1) Preparation Example 1: Synthesis of Intermediate 1-167-1

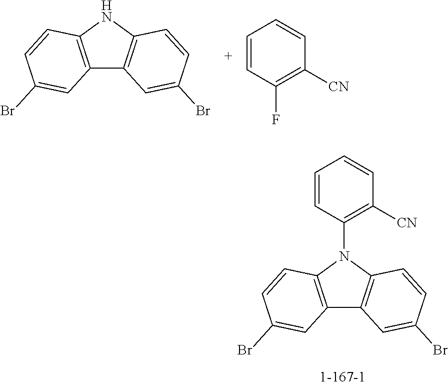 [SMILES: Brc1ccc2[nH]c3ccc(Br)cc3c2c1 ; N#Cc1ccccc1F ; N#Cc1ccccc1-n1c2ccc(Br)cc2c2cc(Br)ccc21]

500 mL of DMF was added to 3,6-dibromocarbazole (10 g, 0.031 mol), 2-fluorobenzenenitrile (4.5 g, 0.037 mol), and Cs₂CO₃ (6.4 g, 0.046 mol), and the resulting mixture was stirred under reflux at 150° C. for 12 hours and reacted. After completion of the reaction, the resulting product was extracted and concentrated, and then subjected to column chromatography to obtain 8.8 g (yield 67.1%) of <Intermediate 1-167-1>.

### (2) Preparation Example 2: Synthesis of Intermediate 1-167-2

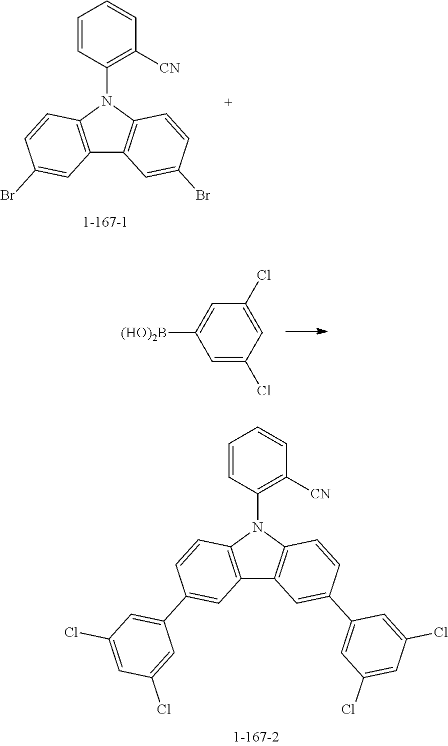

Intermediate 1-167-1 (10.0 g, 0.024 mol), 3,5-dichlorophenylboronic acid (10.8 g, 0.056 mol), K₂CO₃ (19.5 g, 0.141 mol), Pd(PPh₃)₄ (0.5 g, 0.0005 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography and recrystallization to obtain 9.8 g (yield 74.8%) of <Intermediate 1-167-2>.

### (3) Preparation Example 3: Synthesis of Compound 1-167

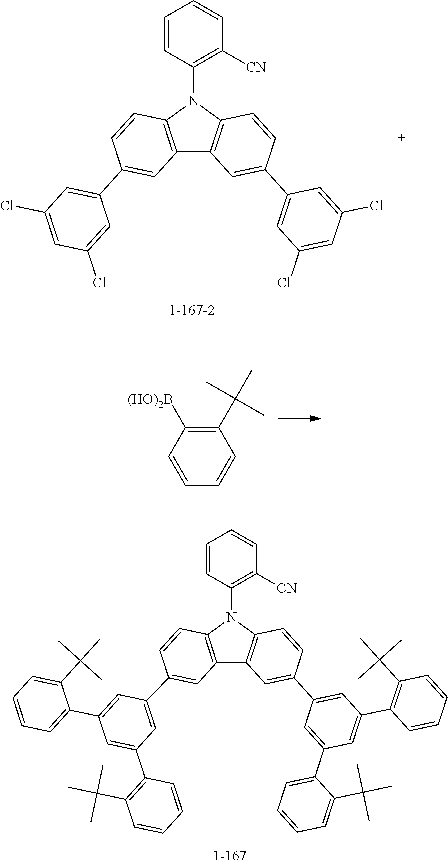 [SMILES: N#Cc1ccccc1-n1c2ccc(-c3cc(Cl)cc(Cl)c3)cc2c2cc(-c3cc(Cl)cc(Cl)c3)ccc21 ; CC(C)(C)c1ccccc1B(O)O ; CC(C)(C)c1ccccc1-c1cc(-c2ccc3c(c2)c2cc(-c4cc(-c5ccccc5C(C)(C)C)cc(-c5ccccc5C(C)(C)C)c4)ccc2n3-c2ccccc2C#N)cc(-c2ccccc2C(C)(C)C)c1]

Intermediate 1-167-2 (10.0 g, 0.017 mol), (2-tert-butylphenyl)boronic acid (8.0 g, 0.025 mol), K₂CO₃ (17.3 g, 0.125 mol), a catalyst Pd(OAc)₂ (2.41 g, 0.002 mol), a ligand X-Phos (1.99 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 12.8 g (yield 75.3%) of <Compound 1-167>.

LC/MS: m/z=948[(M)⁺]

### Synthesis Example 17: Synthesis of Compound 1-191

### (1) Preparation Example 1: Synthesis of Intermediate 1-191-1

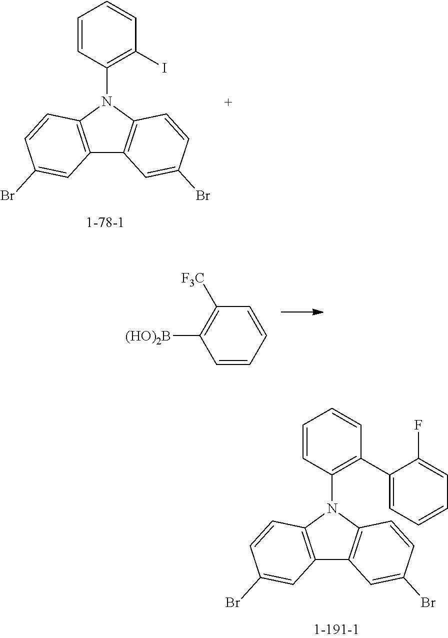 [SMILES: Brc1ccc2c(c1)c1cc(Br)ccc1n2-c1ccccc1I ; OB(O)c1ccccc1C(F)(F)F ; Fc1ccccc1-c1ccccc1-n1c2ccc(Br)cc2c2cc(Br)ccc21]

Intermediate 1-78-1 (10.0 g, 0.019 mol), 2-fluorophenylboronic acid (3.2 g, 0.023 mol), K₂CO₃ (7.9 g, 0.057 mol), Pd(PPh₃)₄ (0.4 g, 0.0004 mol), 200 mL of toluene, 50 mL of ethanol, and 50 mL of H₂O were put into a container, and the resulting mixture was stirred at 100° C. for 6 hours and reacted. After completion of the reaction, the resulting product was extracted and concentrated, and then subjected to column chromatography to obtain 5.9 g (yield 62.8%) of <Intermediate 1-191-1>.

### (2) Preparation Example 2: Synthesis of Intermediate 1-191-2

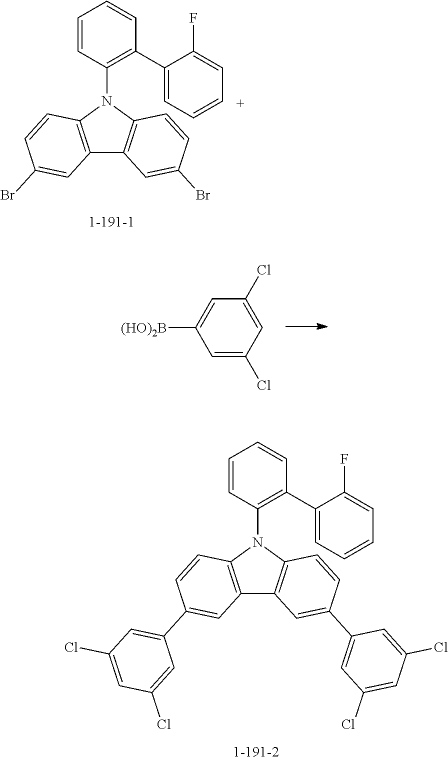 [SMILES: Fc1ccccc1-c1ccccc1-n1c2ccc(Br)cc2c2cc(Br)ccc21 ; OB(O)c1cc(Cl)cc(Cl)c1 ; Fc1ccccc1-c1ccccc1-n1c2ccc(-c3cc(Cl)cc(Cl)c3)cc2c2cc(-c3cc(Cl)cc(Cl)c3)ccc21]

Intermediate 1-191-1 (10.0 g, 0.020 mol), 3,5-dichlorophenylboronic acid (9.3 g, 0.049 mol), K₂CO₃ (14.0 g, 0.101 mol), a catalyst Pd(OAc)₂ (2.3 g, 0.002 mol), a ligand X-Phos (1.9 g, 0.004 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 8.5 g (yield 61.9%) of <Intermediate 1-191-2>.

### (3) Preparation Example 3: Synthesis of Compound 1-191

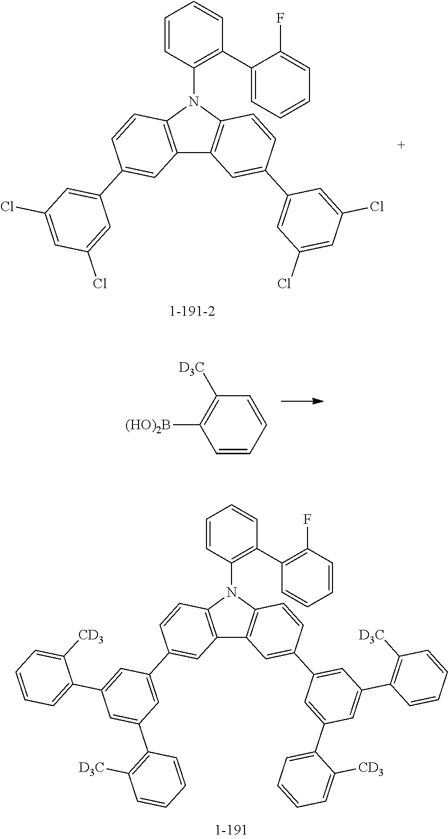 [SMILES: Fc1ccccc1-c1ccccc1-n1c2ccc(-c3cc(Cl)cc(Cl)c3)cc2c2cc(-c3cc(Cl)cc(Cl)c3)ccc21 ; [2H]C([2H])([2H])c1ccccc1B(O)O ; [2H]C([2H])([2H])c1ccccc1-c1cc(-c2ccc3c(c2)c2cc(-c4cc(-c5ccccc5C([2H])([2H])[2H])cc(-c5ccccc5C([2H])([2H])[2H])c4)ccc2n3-c2ccccc2-c2ccccc2F)cc(-c2ccccc2C([2H])([2H])[2H])c1]

Intermediate 1-191-2 (10.0 g, 0.016 mol), 2-(methyl-d3)-phenylboronic acid (10.6 g, 0.077 mol), K₂CO₃ (26.4 g, 0.191 mol), a catalyst Pd(OAc)₂ (1.8 g, 0.002 mol), a ligand X-Phos (2.3 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 8.5 g (yield 61.9%) of <Compound 1-191>.

LC/MS: m/z=861[(M)⁺]

### Synthesis Example 18: Synthesis of Compound 1-197

### (1) Preparation Example 1: Synthesis of Compound 1-197

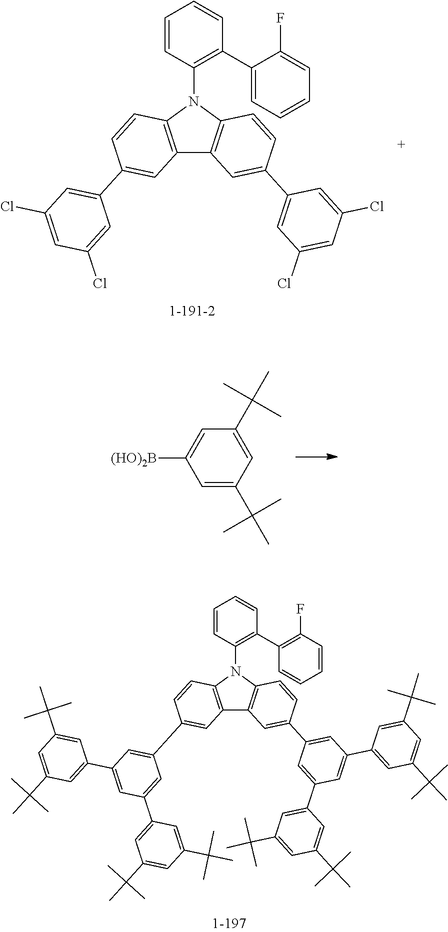 [SMILES: Fc1ccccc1-c1ccccc1-n1c2ccc(-c3cc(Cl)cc(Cl)c3)cc2c2cc(-c3cc(Cl)cc(Cl)c3)ccc21 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(-c3ccc4c(c3)c3cc(-c5cc(-c6cc(C(C)(C)C)cc(C(C)(C)C)c6)cc(-c6cc(C(C)(C)C)cc(C(C)(C)C)c6)c5)ccc3n4-c3ccccc3-c3ccccc3F)c2)cc(C(C)(C)C)c1]

Intermediate 1-191-2 (10.0 g, 0.016 mol), 3,5-di-tert-butylphenylboronic acid (17.9 g, 0.077 mol), K₂CO₃ (26.4 g, 0.191 mol), a catalyst Pd(OAc)₂ (1.8 g, 0.002 mol), a ligand X-Phos (2.3 g, 0.005 mol), 200 mL of THF, and 50 mL of H₂O were put into a container, and the resulting mixture was stirred at 90° C. for 6 hours and reacted. After completion of the reaction, the resulting product was extracted and concentrated, and then subjected to column chromatography and recrystallization to obtain 10.2 g (yield 51.5%) of <Compound 1-197>.

LC/MS: m/z=1227[(M)⁺]

### Device Examples (Capping Layer)

In exemplary embodiments according to the present invention, an ITO transparent electrode was patterned using an ITO glass substrate including Ag of 25 mm×25 mm×0.7 mm such that a light emitting area had a size of 2 mm×2 mm, and then washed. After the substrate was mounted in a vacuum chamber, a base pressure was set to 1×10⁻⁶ torr or more, and organic substances and a metal were vapor-deposited to have the following structure on the ITO glass substrate including Ag.

### Device Examples 1 to 90

A blue organic light emitting device having the following device structure was manufactured by adopting a compound implemented by the present invention for a light efficiency improving layer, and light emitting characteristics including a light emitting efficiency were measured.

Ag/ITO/hole injection layer (HAT-CN, 5 nm)/hole transport layer (α-NPB, 100 nm)/electron blocking layer (TCTA, 10 nm)/light emitting layer (20 nm)/electron transport layer (ET1:Liq, 30 nm)/LiF (1 nm)/Mg:Ag (15 nm)/light efficiency improving layer (70 nm)

In order to form a hole injection layer on an ITO transparent electrode including Ag on a glass substrate, HAT-CN was film-formed to a thickness of 5 nm, and then, the hole transport layer was film-formed to 100 nm with α-NPB. An electron blocking layer was film-formed to a thickness of 10 nm using TCTA. Further, BH1 was used as a host compound and BD1 was used as a dopant compound in the light emitting layer, and co-deposited to 20 nm. Additionally, an electron transport layer (doped with the following [ET1] compound Liq 50%) and LiF were film-formed to a thickness of 30 nm and 1 nm, respectively. Subsequently, Mg and Ag were film-formed to 15 nm at a ratio of 1:9.

Hereinafter, an organic light emitting device was manufactured by using the compound implemented in the present invention shown in the following [Table 1] to film-form a light efficiency improving layer to a thickness of 70 nm as a light efficiency improving layer (capping layer) compound.

### Device Comparative Example 1

An organic light emitting device for Device Comparative Example 1 was manufactured in the same manner as in the device structure in the Examples, except that the light efficiency improving layer was not used.

### Device Comparative Example 2

An organic light emitting device for Device Comparative Example 2 was manufactured in the same manner as in the device structure in the Examples, except that as the light efficiency improving layer compound, Alq₃ was used instead of the compound of the present invention.

### Device Comparative Example 3

An organic light emitting device for Device Comparative Example 3 was manufactured in the same manner as in the device structure in the Examples, except that as the light efficiency improving layer compound, the following [CP 1] was used instead of the compound according to the present invention.

### Device Comparative Example 4

An organic light emitting device for Device Comparative Example 4 was manufactured in the same manner as in the device structure in the Examples, except that as the light efficiency improving layer compound, the following [CP 2] was used instead of the compound according to the present invention.

### Device Comparative Example 5

An organic light emitting device for Device Comparative Example 5 was manufactured in the same manner as in the device structure in the Examples, except that as the light efficiency improving layer compound, the following [CP 3] was used instead of the compound according to the present invention.

### Experimental Example 1: Light Emitting Characteristics of Device Examples 1 to 90

For the organic light emitting devices manufactured by the Examples, driving voltage, current efficiency and color coordinate were measured using a Source meter (Model237, Keithley) and a luminance meter (PR-650, Photo Research), and the result values based on 1,000 nits are shown in the following [Table 1].

TABLE 1

| | Light efficiency | | | | |
|---|---|---|---|---|---|
| Example | improving layer | V | cd/A | CIEx | CIEy |
| 1 | Chemical | 3.6 | 8.7 | 0.140 | 0.050 |
| | Formula 4 | | | | |
| 2 | Chemical | 3.9 | 8.4 | 0.141 | 0.052 |
| | Formula 15 | | | | |
| 3 | Chemical | 3.7 | 8.6 | 0.142 | 0.053 |
| | Formula 42 | | | | |
| 4 | Chemical | 3.8 | 8.8 | 0.142 | 0.052 |
| | Formula 50 | | | | |
| 5 | Chemical | 3.6 | 8.9 | 0.140 | 0.050 |
| | Formula 60 | | | | |
| 6 | Chemical | 3.7 | 9.0 | 0.142 | 0.051 |
| | Formula 100 | | | | |
| 7 | Chemical | 3.7 | 8.8 | 0.141 | 0.051 |
| | Formula 120 | | | | |
| 8 | Chemical | 3.6 | 8.7 | 0.143 | 0.052 |
| | Formula 152 | | | | |
| 9 | Chemical | 3.5 | 8.9 | 0.142 | 0.055 |
| | Formula 161 | | | | |
| 10 | Chemical | 3.4 | 8.4 | 0.147 | 0.050 |
| | Formula 162 | | | | |
| 11 | Chemical | 3.6 | 8.8 | 0.138 | 0.057 |
| | Formula 166 | | | | |
| 12 | Chemical | 3.8 | 8.6 | 0.145 | 0.053 |
| | Formula 167 | | | | |
| 13 | Chemical | 3.6 | 8.7 | 0.143 | 0.052 |
| | Formula 168 | | | | |
| 14 | Chemical | 3.5 | 8.9 | 0.138 | 0.062 |
| | Formula 169 | | | | |
| 15 | Chemical | 3.9 | 8.3 | 0.144 | 0.048 |
| | Formula 170 | | | | |
| 16 | Chemical | 3.7 | 8.5 | 0.141 | 0.052 |
| | Formula 171 | | | | |
| 17 | Chemical | 3.6 | 8.6 | 0.143 | 0.053 |
| | Formula 1-1 | | | | |
| 18 | Chemical | 3.5 | 8.8 | 0.139 | 0.061 |
| | Formula 1-2 | | | | |
| 19 | Chemical | 3.7 | 8.7 | 0.141 | 0.056 |
| | Formula 1-4 | | | | |
| 20 | Chemical | 3.5 | 8.8 | 0.139 | 0.061 |
| | Formula 1-5 | | | | |
| 21 | Chemical | 3.8 | 8.4 | 0.144 | 0.050 |
| | Formula 1-6 | | | | |
| 22 | Chemical | 3.6 | 8.7 | 0.141 | 0.054 |
| | Formula 1-7 | | | | |
| 23 | Chemical | 3.4 | 8.4 | 0.146 | 0.051 |
| | Formula 1-12 | | | | |
| 24 | Chemical | 3.5 | 9.0 | 0.139 | 0.060 |
| | Formula 1-13 | | | | |
| 25 | Chemical | 3.6 | 8.6 | 0.141 | 0.054 |
| | Formula 1-14 | | | | |
| 26 | Chemical | 3.7 | 8.8 | 0.138 | 0.061 |
| | Formula 1-15 | | | | |
| 27 | Chemical | 3.4 | 9.0 | 0.137 | 0.059 |
| | Formula 1-19 | | | | |
| 28 | Chemical | 3.5 | 8.8 | 0.139 | 0.061 |
| | Formula 1-23 | | | | |
| 29 | Chemical | 3.8 | 8.3 | 0.146 | 0.048 |
| | Formula 1-27 | | | | |
| 30 | Chemical | 3.6 | 8.7 | 0.140 | 0.054 |
| | Formula 1-33 | | | | |
| 31 | Chemical | 3.4 | 9.0 | 0.138 | 0.059 |
| | Formula 1-42 | | | | |
| 32 | Chemical | 3.5 | 8.8 | 0.137 | 0.061 |
| | Formula 1-47 | | | | |
| 33 | Chemical | 3.6 | 8.6 | 0.142 | 0.055 |
| | Formula 1-50 | | | | |
| 34 | Chemical | 3.8 | 8.4 | 0.146 | 0.045 |
| | Formula 1-53 | | | | |
| 35 | Chemical | 3.4 | 9.0 | 0.135 | 0.06 |
| | Formula 1-56 | | | | |
| 36 | Chemical | 3.5 | 8.9 | 0.139 | 0.063 |
| | Formula 1-62 | | | | |
| 37 | Chemical | 3.6 | 8.6 | 0.141 | 0.055 |
| | Formula 1-65 | | | | |
| 38 | Chemical | 3.8 | 8.9 | 0.139 | 0.059 |
| | Formula 1-67 | | | | |
| 39 | Chemical | 3.6 | 8.7 | 0.141 | 0.054 |
| | Formula 1-78 | | | | |
| 40 | Chemical | 3.5 | 8.8 | 0.140 | 0.062 |
| | Formula 1-81 | | | | |
| 41 | Chemical | 3.4 | 9.0 | 0.137 | 0.057 |
| | Formula 1-82 | | | | |
| 42 | Chemical | 3.8 | 8.4 | 0.144 | 0.049 |
| | Formula 1-85 | | | | |
| 43 | Chemical | 3.5 | 8.9 | 0.139 | 0.060 |
| | Formula 1-93 | | | | |
| 44 | Chemical | 3.8 | 8.4 | 0.145 | 0.048 |
| | Formula 1-96 | | | | |
| 45 | Chemical | 3.6 | 8.9 | 0.137 | 0.059 |
| | Formula 1-98 | | | | |
| 46 | Chemical | 3.4 | 9.0 | 0.138 | 0.060 |
| | Formula 1-99 | | | | |
| 47 | Chemical | 3.5 | 8.8 | 0.137 | 0.064 |
| | Formula 1-100 | | | | |
| 48 | Chemical | 3.6 | 8.7 | 0.141 | 0.055 |
| | Formula 1-101 | | | | |
| 49 | Chemical | 3.8 | 8.4 | 0.143 | 0.052 |
| | Formula 1-102 | | | | |
| 50 | Chemical | 3.6 | 8.7 | 0.140 | 0.053 |
| | Formula 1-106 | | | | |
| 51 | Chemical | 3.5 | 8.8 | 0.139 | 0.062 |
| | Formula 1-108 | | | | |
| 52 | Chemical | 3.7 | 8.5 | 0.143 | 0.051 |
| | Formula 1-109 | | | | |
| 53 | Chemical | 3.6 | 8.7 | 0.141 | 0.057 |
| | Formula 1-115 | | | | |
| 54 | Chemical | 3.8 | 8.3 | 0.146 | 0.052 |
| | Formula 1-123 | | | | |
| 55 | Chemical | 3.7 | 8.5 | 0.143 | 0.049 |
| | Formula 1-124 | | | | |
| 56 | Chemical | 3.4 | 9.1 | 0.135 | 0.061 |
| | Formula 1-127 | | | | |
| 57 | Chemical | 3.5 | 8.9 | 0.138 | 0.059 |
| | Formula 1-132 | | | | |
| 58 | Chemical | 3.5 | 8.9 | 0.139 | 0.060 |
| | Formula 1-138 | | | | |
| 59 | Chemical | 3.8 | 8.4 | 0.144 | 0.047 |
| | Formula 1-139 | | | | |
| 60 | Chemical | 3.6 | 8.7 | 0.141 | 0.056 |
| | Formula 1-140 | | | | |
| 61 | Chemical | 3.8 | 8.4 | 0.145 | 0.049 |
| | Formula 1-143 | | | | |
| 62 | Chemical | 3.5 | 8.6 | 0.142 | 0.052 |
| | Formula 1-147 | | | | |
| 63 | Chemical | 3.8 | 8.3 | 0.146 | 0.047 |
| | Formula 1-149 | | | | |
| 64 | Chemical | 3.7 | 8.7 | 0.141 | 0.054 |
| | Formula 1-154 | | | | |
| 65 | Chemical | 3.6 | 8.6 | 0.146 | 0.058 |
| | Formula 1-156 | | | | |
| 66 | Chemical | 3.7 | 8.9 | 0.139 | 0.061 |
| | Formula 1-161 | | | | |
| 67 | Chemical | 3.6 | 8.8 | 0.141 | 0.054 |
| | Formula 1-162 | | | | |
| 68 | Chemical | 3.5 | 8.9 | 0.143 | 0.062 |
| | Formula 1-166 | | | | |
| 69 | Chemical | 3.8 | 8.8 | 0.146 | 0.049 |
| | Formula 1-167 | | | | |
| 70 | Chemical | 3.4 | 9.1 | 0.138 | 0.061 |
| | Formula 1-168 | | | | |
| 71 | Chemical | 3.5 | 8.9 | 0.139 | 0.061 |
| | Formula 1-174 | | | | |
| 72 | Chemical | 3.8 | 8.4 | 0.147 | 0.049 |
| | Formula 1-180 | | | | |
| 73 | Chemical | 3.6 | 8.8 | 0.141 | 0.055 |
| | Formula 1-183 | | | | |
| 74 | Chemical | 3.5 | 8.9 | 0.140 | 0.062 |
| | Formula 1-185 | | | | |
| 75 | Chemical | 3.6 | 8.8 | 0.145 | 0.053 |
| | Formula 1-186 | | | | |
| 76 | Chemical | 3.4 | 9.1 | 0.137 | 0.056 |
| | Formula 1-187 | | | | |
| 77 | Chemical | 3.8 | 8.5 | 0.145 | 0.053 |
| | Formula 1-188 | | | | |
| 78 | Chemical | 3.5 | 9.1 | 0.138 | 0.061 |
| | Formula 1-189 | | | | |
| 79 | Chemical | 3.8 | 8.4 | 0.144 | 0.049 |
| | Formula 1-190 | | | | |
| 80 | Chemical | 3.5 | 8.9 | 0.140 | 0.062 |
| | Formula 1-191 | | | | |
| 81 | Chemical | 3.6 | 8.7 | 0.143 | 0.056 |
| | Formula 1-192 | | | | |
| 82 | Chemical | 3.8 | 8.4 | 0.147 | 0.049 |
| | Formula 1-193 | | | | |
| 83 | Chemical | 3.4 | 9.1 | 0.135 | 0.060 |
| | Formula 1-194 | | | | |
| 84 | Chemical | 3.6 | 8.8 | 0.144 | 0.055 |
| | Formula 1-195 | | | | |
| 85 | Chemical | 3.8 | 8.5 | 0.146 | 0.049 |
| | Formula 1-196 | | | | |
| 86 | Chemical | 3.5 | 8.9 | 0.139 | 0.062 |
| | Formula 1-197 | | | | |
| 87 | Chemical | 3.6 | 8.7 | 0.143 | 0.056 |
| | Formula 1-201 | | | | |
| 88 | Chemical | 3.8 | 8.5 | 0.147 | 0.047 |
| | Formula 1-202 | | | | |
| 89 | Chemical | 3.6 | 8.8 | 0.142 | 0.053 |
| | Formula 1-203 | | | | |
| 90 | Chemical | 3.5 | 8.9 | 0.140 | 0.061 |
| | Formula 1-204 | | | | |
| Comparative | Not used | 4.6 | 7.0 | 0.150 | 0.141 |
| Example 1 | | | | | |
| Comparative | Alq₃ | 4.3 | 7.8 | 0.147 | 0.058 |
| Example 2 | | | | | |
| Comparative | CP1 | 4.5 | 7.1 | 0.149 | 0.052 |
| Example 3 | | | | | |
| Comparative | CP2 | 4.4 | 7.5 | 0.148 | 0.061 |
| Example 4 | | | | | |
| Comparative | CP3 | 4.5 | 7.3 | 0.145 | 0.069 |
| Example 5 | | | | | |

Referring to the results shown in [Table 1], it can be confirmed that the organic light emitting device in which the compound according to the present invention is applied to a light efficiency improving layer has excellent light emitting characteristics because the driving voltage thereof is reduced and the current efficiency thereof is improved compared to a device which does not include a light efficiency improving layer in the related art and devices (Comparative Examples 1 to 5) in which compounds used as a material for a light efficiency improving layer in the related art are adopted.

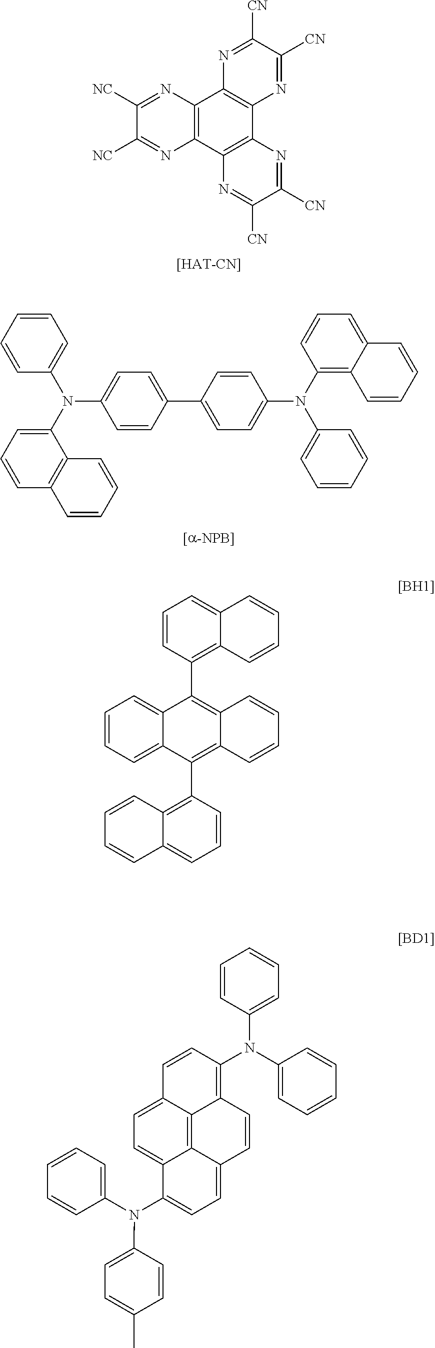

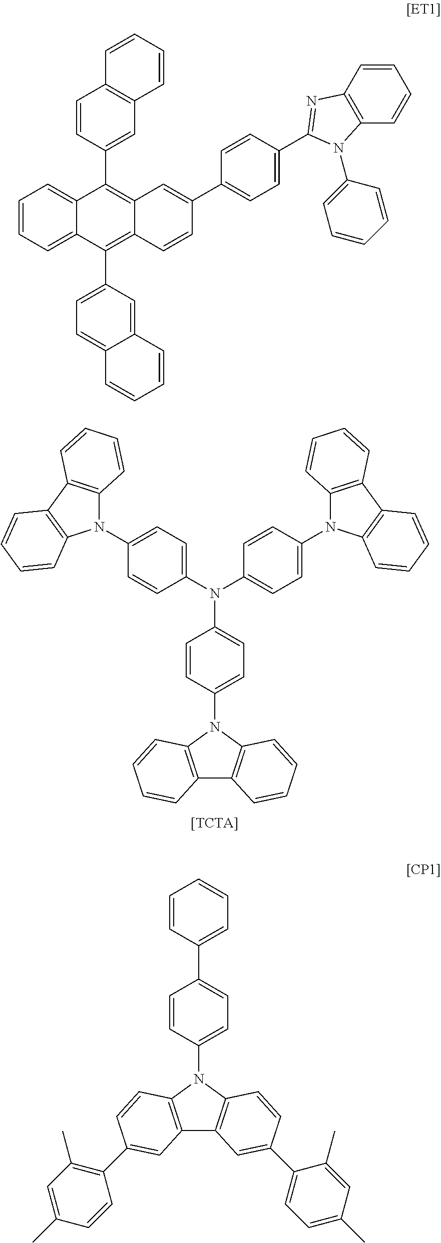

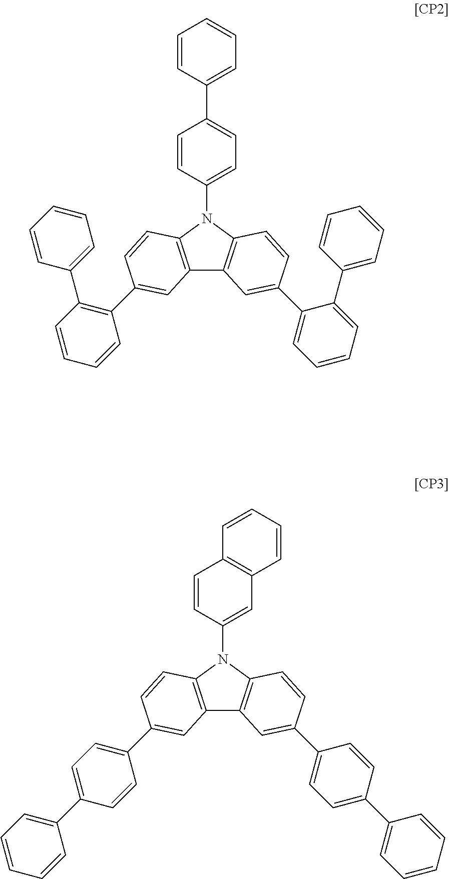 [SMILES: c1ccc(-c2ccc(-n3c4ccc(-c5ccccc5-c5ccccc5)cc4c4cc(-c5ccccc5-c5ccccc5)ccc43)cc2)cc1 ; c1ccc(-c2ccc(-c3ccc4c(c3)c3cc(-c5ccc(-c6ccccc6)cc5)ccc3n4-c3ccc4ccccc4c3)cc2)cc1]

### INDUSTRIAL APPLICABILITY

When the organic light emitting compound according to the present invention is used as a material for a light efficiency improving layer provided in an organic light emitting device, the organic light emitting compound can be industrially usefully used for various display devices, lighting devices, and the like because it is possible to implement various light emitting characteristics such as low voltage driving, excellent color purity and excellent light emitting efficiency of the device.

1. An organic light emitting compound represented by the following [Chemical Formula I]: 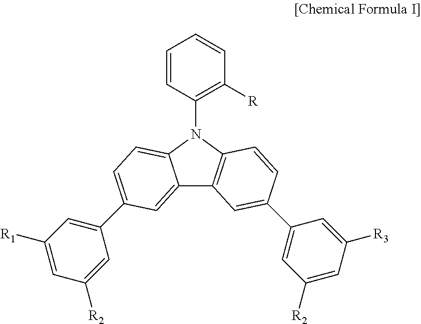
in [Chemical Formula I],
R is any one selected among deuterium, a cyano group, a halogen group, a substituted or unsubstituted alkyl group having 1 to 20 carbon atoms, a substituted or unsubstituted deuterated alkyl group having 1 to 20 carbon atoms, a substituted or unsubstituted halogenated alkyl group having 1 to 20 carbon atoms, a substituted or unsubstituted cycloalkyl group having 3 to 20 carbon atoms, a substituted or unsubstituted aryl group having 6 to 30 carbon atoms and a substituted or unsubstituted heteroaryl group having 3 to 30 carbon atoms, and
R₁ to R₄ are the same as or different from each other, and are each independently any one selected among deuterium, a halogen group, a cyano group, a substituted or unsubstituted alkyl group having 1 to 20 carbon atoms, a substituted or unsubstituted cycloalkyl group having 3 to 20 carbon atoms, a substituted or unsubstituted deuterated alkyl group having 1 to 20 carbon atoms, a substituted or unsubstituted halogenated alkyl group having 1 to 20 carbon atoms and a substituted or unsubstituted aryl group having 6 to 30 carbon atoms.

2. The organic light emitting compound of claim 1, wherein R₁ to R₄ are the same as or different from each other, and are each independently a substituted or unsubstituted aryl group having 6 to 30 carbon atoms.

3. The organic light emitting compound of claim 1, wherein [Chemical Formula I] is represented by the following [Chemical Formula I-1]: 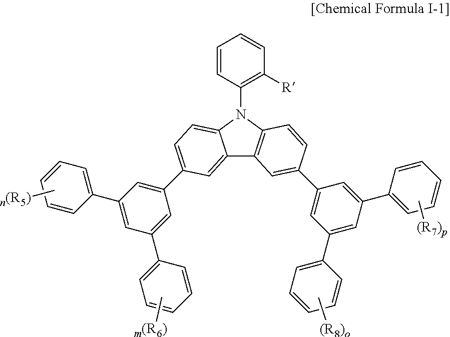
in [Chemical Formula I-1],
R′ is the same as the definition of R of [Chemical Formula I] of claim 1, and R₅ to R₈ are the same as the definitions of R₁ to R₄ of [Chemical Formula I] of claim 1, and n, m, o and p are each an integer from 1 to 5, and when n, m, o and p are each 2 or higher, a plurality of R₅ to R₈ are each the same as or different from each other.

4. The organic light emitting compound of claim 3, wherein R′ and R₅ to R₈ are the same as or different from each other, and are each independently any one selected among deuterium, a halogen group, a cyano group, a substituted or unsubstituted halogenated alkyl group having 1 to 20 carbon atoms, a substituted or unsubstituted deuterated alkyl group having 1 to 20 carbon atoms, a substituted or unsubstituted alkyl group having 1 to 20 carbon atoms and a substituted or unsubstituted aryl group having 6 to 30 carbon atoms.

5. The organic light emitting compound of claim 1, wherein the ‘substituted or unsubstituted’ means being substituted with one or two or more substituents selected from the group consisting of deuterium, a halogen group, a cyano group, a silyl group, an alkyl group, a halogenated alkyl group, a deuterated alkyl group, a cycloalkyl group, an alkoxy group, an aryl group and a heterocyclic group, being substituted with a substituent to which two or more substituents among the aforementioned substituents are linked, or having no substituent.

6. The organic light emitting compound of claim 1, wherein [Chemical Formula I] is any one selected among the following Compounds [1] to [171] and [1-1] to [1-207]:

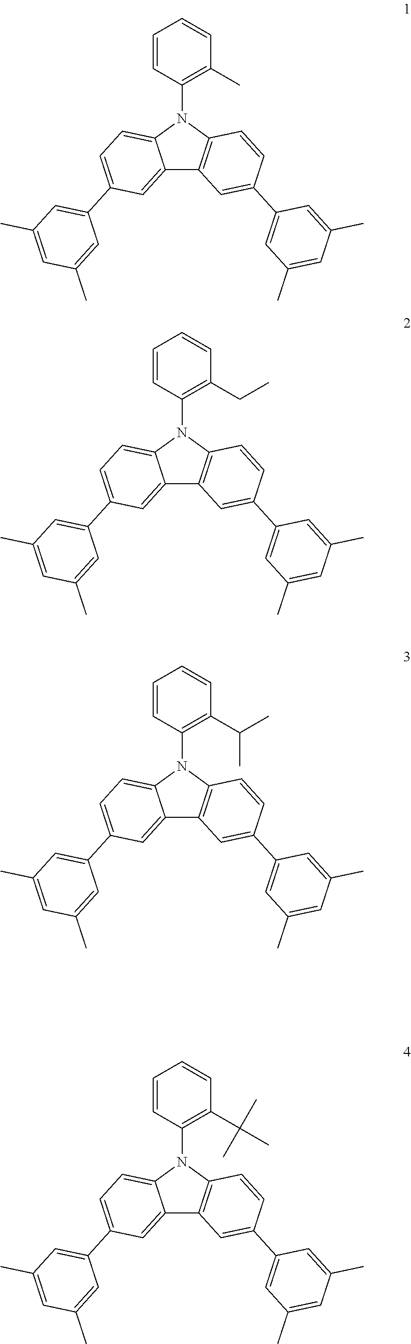

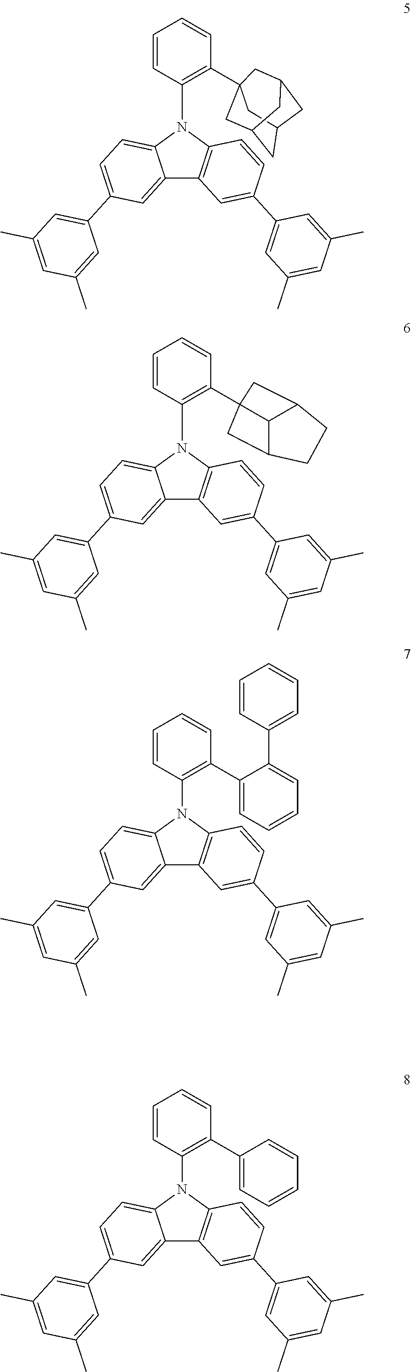

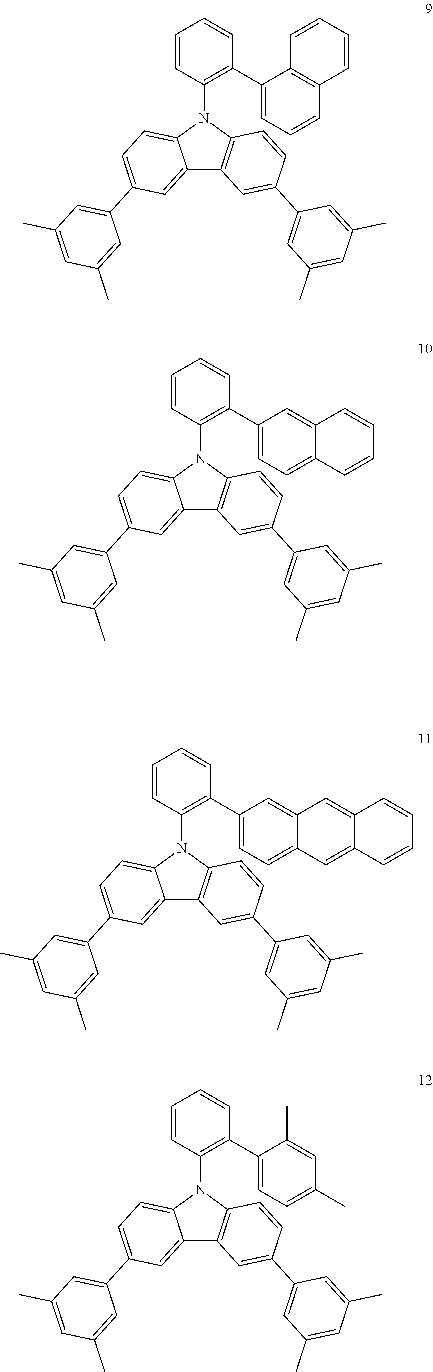

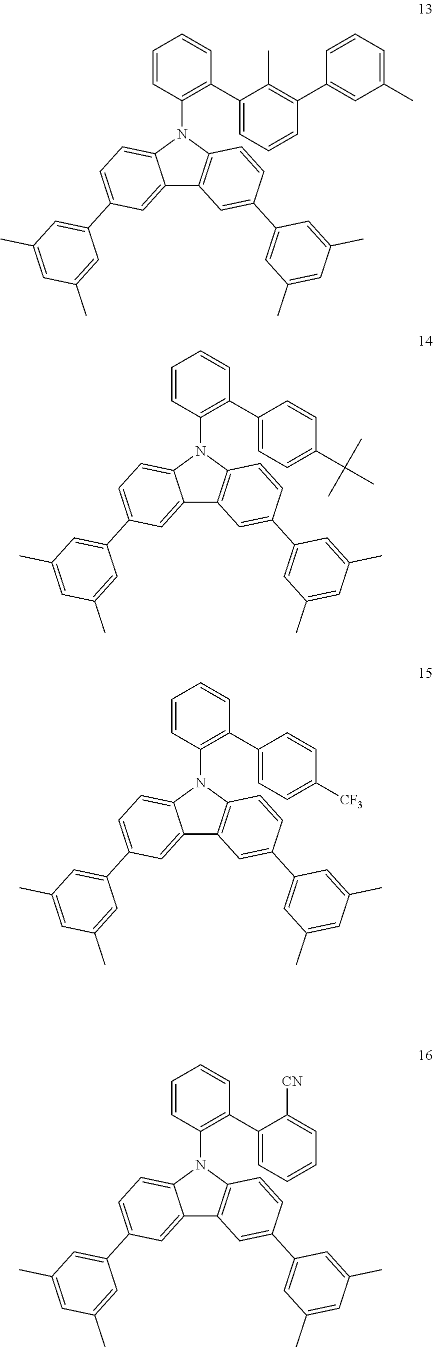

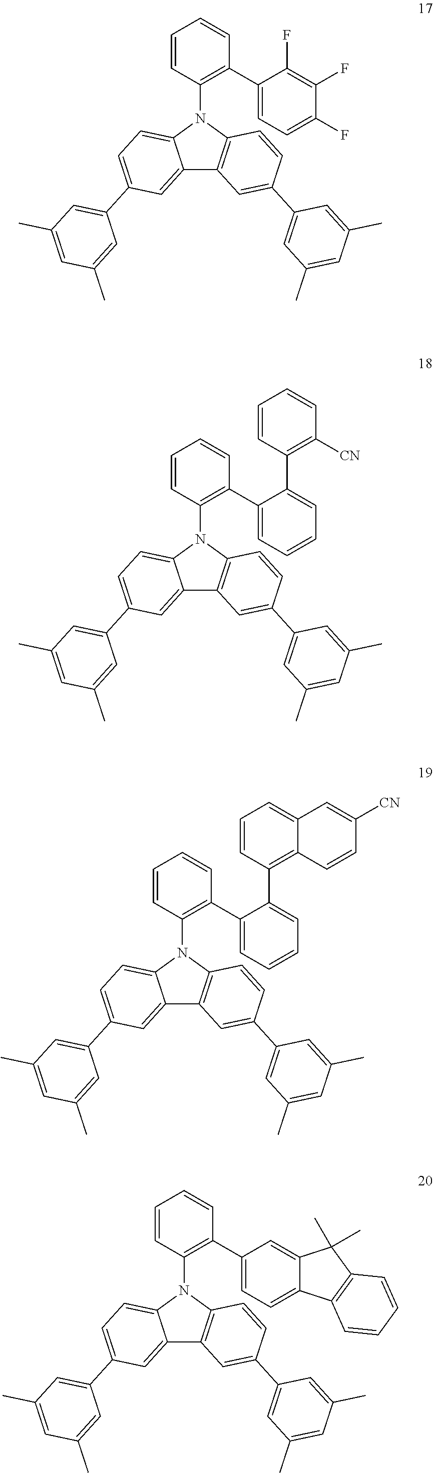

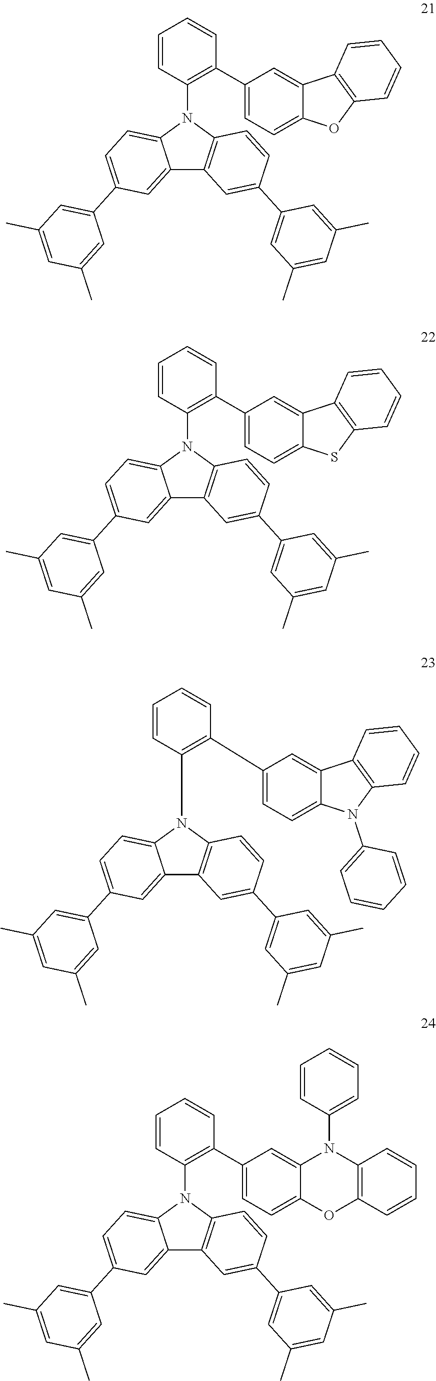 [SMILES: Cc1cc(C)cc(-c2ccc3c(c2)c2cc(-c4cc(C)cc(C)c4)ccc2n3-c2ccccc2-c2ccc3oc4ccccc4c3c2)c1 ; Cc1cc(C)cc(-c2ccc3c(c2)c2cc(-c4cc(C)cc(C)c4)ccc2n3-c2ccccc2-c2ccc3sc4ccccc4c3c2)c1 ; Cc1cc(C)cc(-c2ccc3c(c2)c2cc(-c4cc(C)cc(C)c4)ccc2n3-c2ccccc2-c2ccc3c(c2)c2ccccc2n3-c2ccccc2)c1 ; Cc1cc(C)cc(-c2ccc3c(c2)c2cc(-c4cc(C)cc(C)c4)ccc2n3-c2ccccc2-c2ccc3c(c2)N(c2ccccc2)c2ccccc2O3)c1]

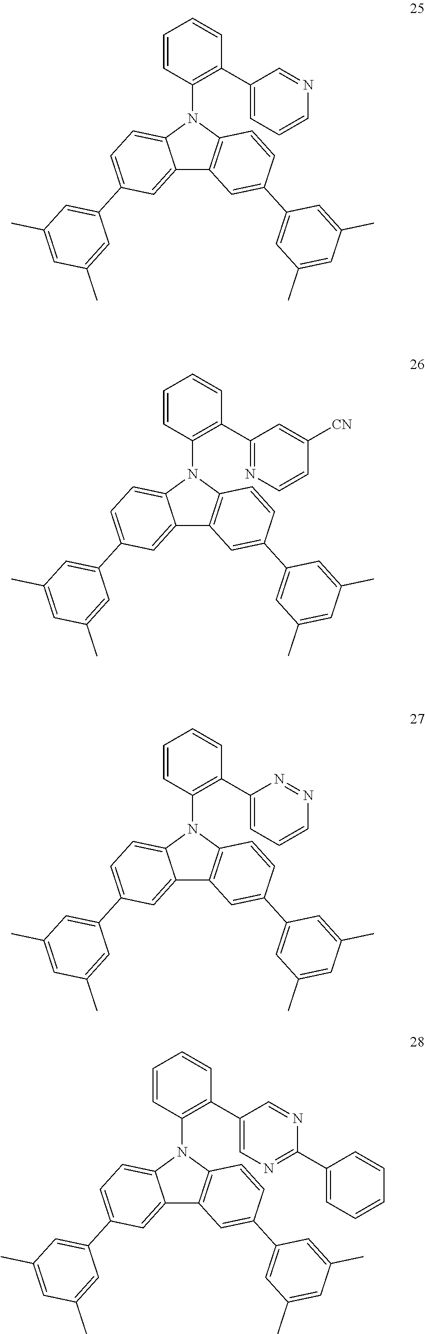

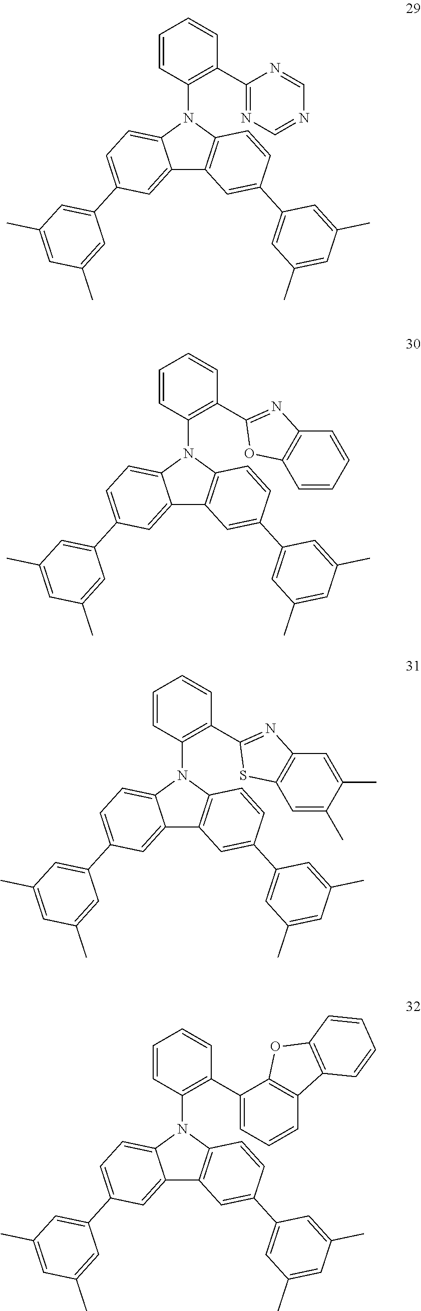

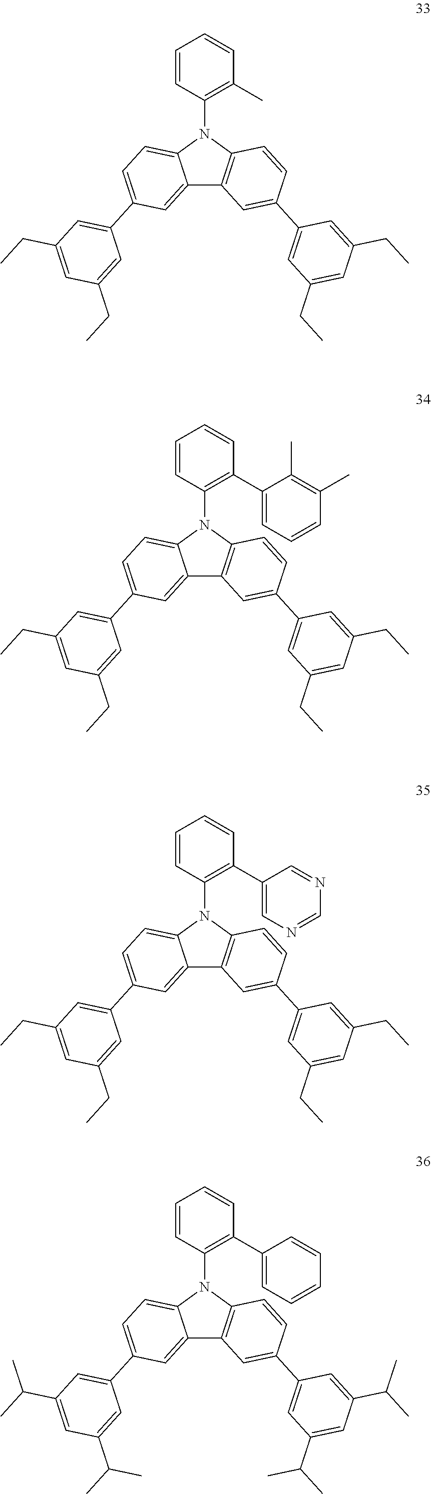

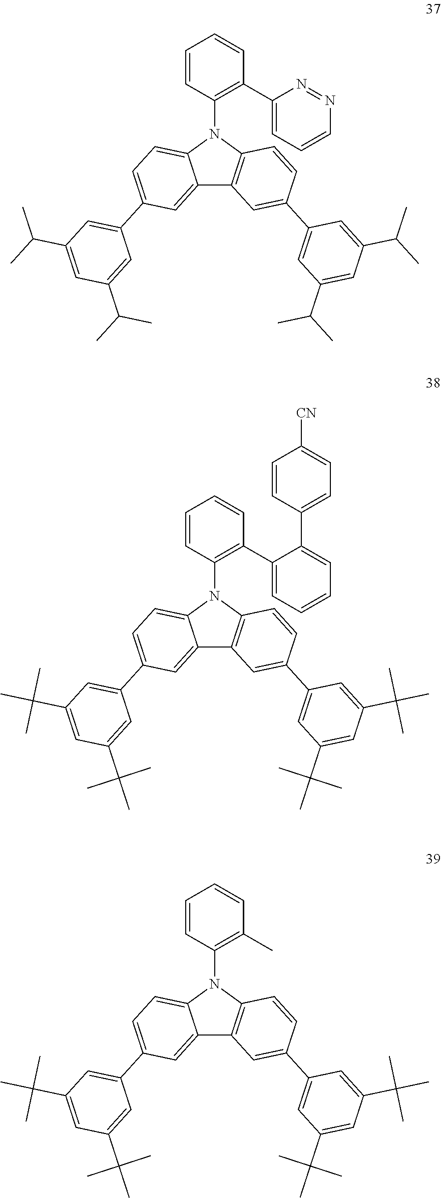

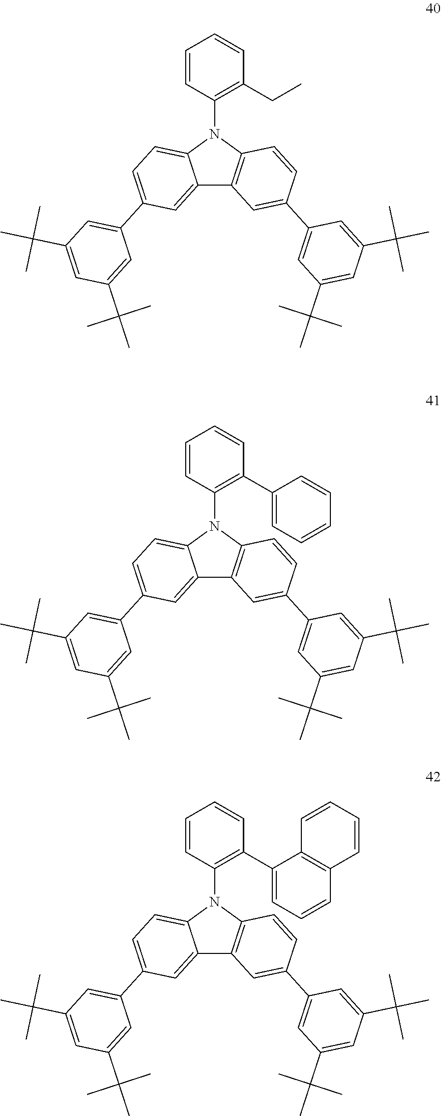

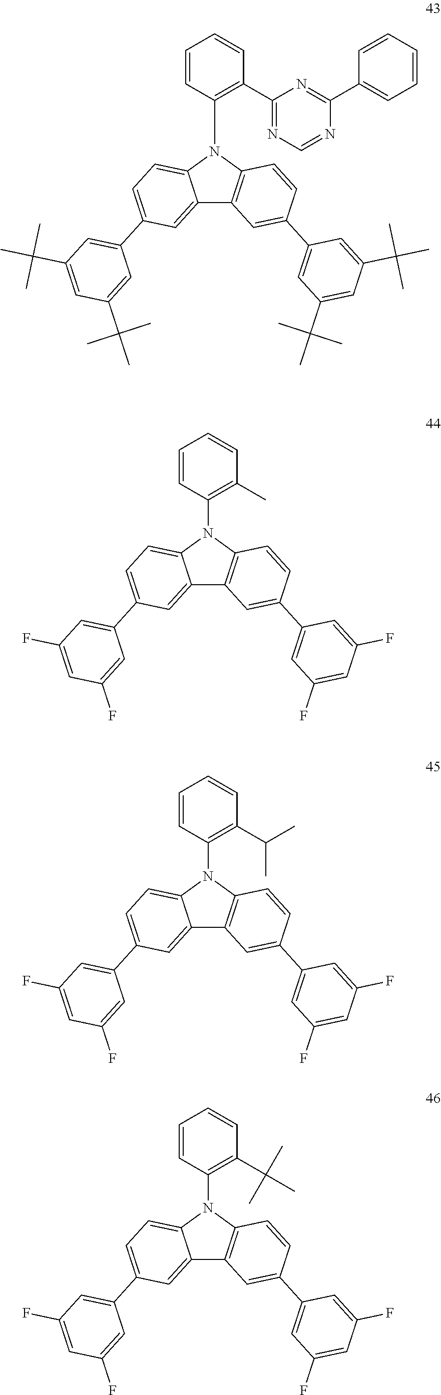 [SMILES: CC(C)(C)c1cc(-c2ccc3c(c2)c2cc(-c4cc(C(C)(C)C)cc(C(C)(C)C)c4)ccc2n3-c2ccccc2-c2ncnc(-c3ccccc3)n2)cc(C(C)(C)C)c1 ; Cc1ccccc1-n1c2ccc(-c3cc(F)cc(F)c3)cc2c2cc(-c3cc(F)cc(F)c3)ccc21 ; CC(C)c1ccccc1-n1c2ccc(-c3cc(F)cc(F)c3)cc2c2cc(-c3cc(F)cc(F)c3)ccc21 ; CC(C)(C)c1ccccc1-n1c2ccc(-c3cc(F)cc(F)c3)cc2c2cc(-c3cc(F)cc(F)c3)ccc21]

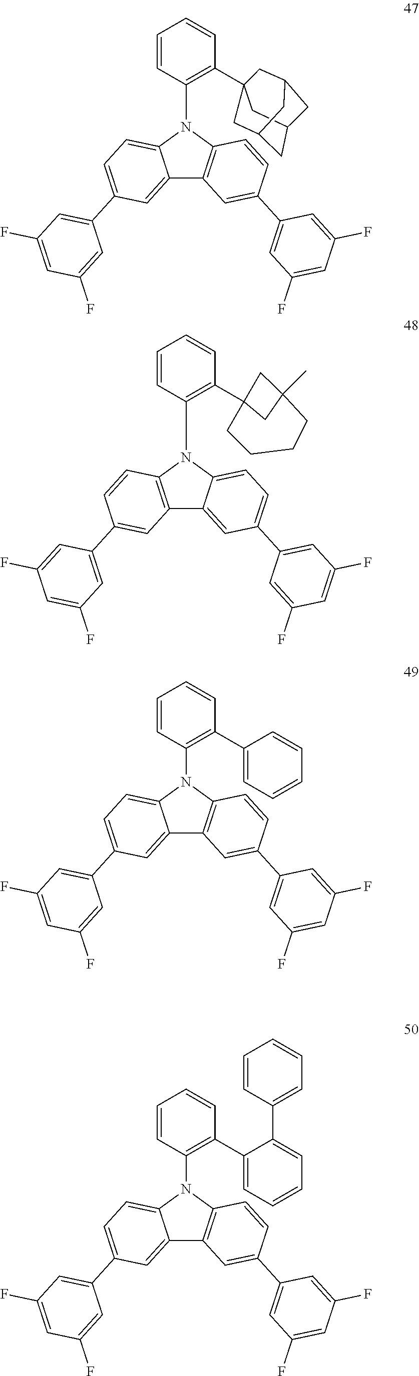 [SMILES: Fc1cc(F)cc(-c2ccc3c(c2)c2cc(-c4cc(F)cc(F)c4)ccc2n3-c2ccccc2C23CC4CC(CC(C4)C2)C3)c1 ; CC12CCCCC(c3ccccc3-n3c4ccc(-c5cc(F)cc(F)c5)cc4c4cc(-c5cc(F)cc(F)c5)ccc43)(C1)C2 ; Fc1cc(F)cc(-c2ccc3c(c2)c2cc(-c4cc(F)cc(F)c4)ccc2n3-c2ccccc2-c2ccccc2)c1 ; Fc1cc(F)cc(-c2ccc3c(c2)c2cc(-c4cc(F)cc(F)c4)ccc2n3-c2ccccc2-c2ccccc2-c2ccccc2)c1]

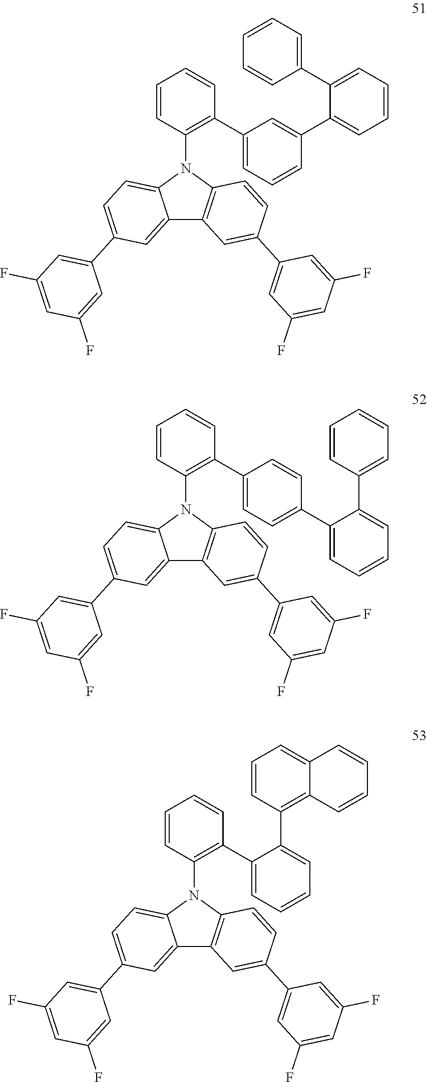 [SMILES: Fc1cc(F)cc(-c2ccc3c(c2)c2cc(-c4cc(F)cc(F)c4)ccc2n3-c2ccccc2-c2cccc(-c3ccccc3-c3ccccc3)c2)c1 ; Fc1cc(F)cc(-c2ccc3c(c2)c2cc(-c4cc(F)cc(F)c4)ccc2n3-c2ccccc2-c2ccc(-c3ccccc3-c3ccccc3)cc2)c1 ; Fc1cc(F)cc(-c2ccc3c(c2)c2cc(-c4cc(F)cc(F)c4)ccc2n3-c2ccccc2-c2ccccc2-c2cccc3ccccc23)c1]

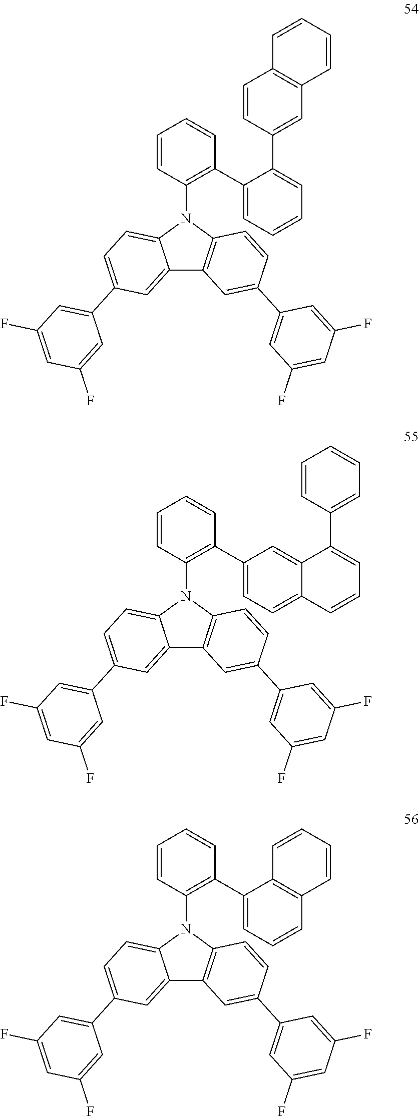 [SMILES: Fc1cc(F)cc(-c2ccc3c(c2)c2cc(-c4cc(F)cc(F)c4)ccc2n3-c2ccccc2-c2ccccc2-c2ccc3ccccc3c2)c1 ; Fc1cc(F)cc(-c2ccc3c(c2)c2cc(-c4cc(F)cc(F)c4)ccc2n3-c2ccccc2-c2ccc3cccc(-c4ccccc4)c3c2)c1 ; Fc1cc(F)cc(-c2ccc3c(c2)c2cc(-c4cc(F)cc(F)c4)ccc2n3-c2ccccc2-c2cccc3ccccc23)c1]

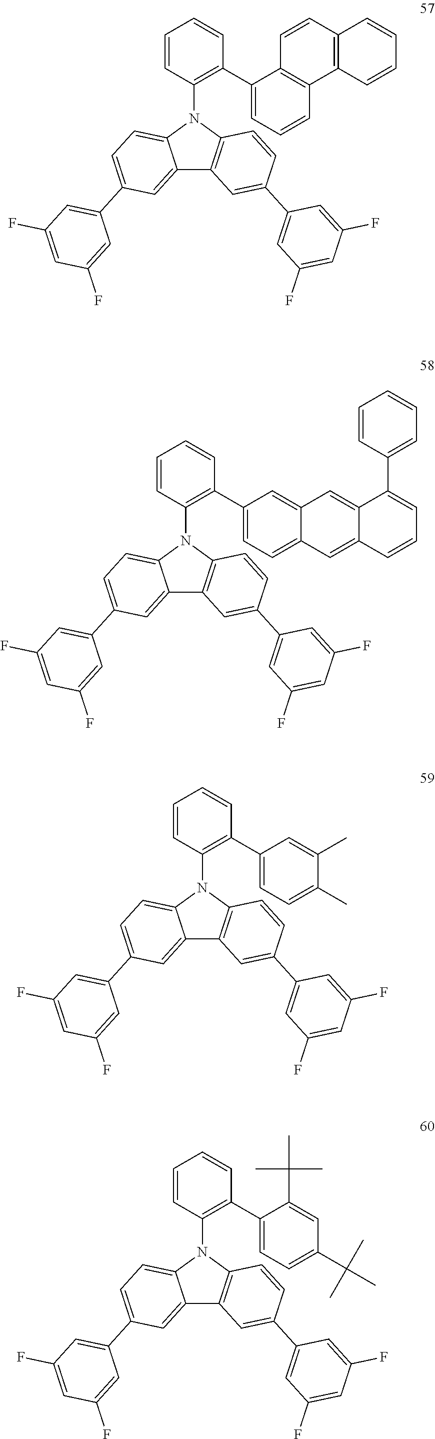 [SMILES: Fc1cc(F)cc(-c2ccc3c(c2)c2cc(-c4cc(F)cc(F)c4)ccc2n3-c2ccccc2-c2cccc3c2ccc2ccccc23)c1 ; Fc1cc(F)cc(-c2ccc3c(c2)c2cc(-c4cc(F)cc(F)c4)ccc2n3-c2ccccc2-c2ccc3cc4cccc(-c5ccccc5)c4cc3c2)c1 ; Cc1ccc(-c2ccccc2-n2c3ccc(-c4cc(F)cc(F)c4)cc3c3cc(-c4cc(F)cc(F)c4)ccc32)cc1C ; CC(C)(C)c1ccc(-c2ccccc2-n2c3ccc(-c4cc(F)cc(F)c4)cc3c3cc(-c4cc(F)cc(F)c4)ccc32)c(C(C)(C)C)c1]

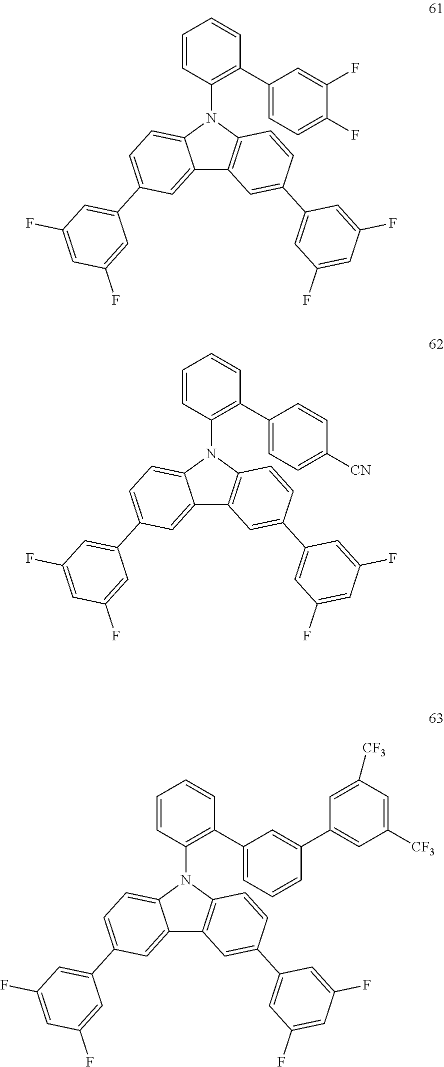 [SMILES: Fc1cc(F)cc(-c2ccc3c(c2)c2cc(-c4cc(F)cc(F)c4)ccc2n3-c2ccccc2-c2ccc(F)c(F)c2)c1 ; N#Cc1ccc(-c2ccccc2-n2c3ccc(-c4cc(F)cc(F)c4)cc3c3cc(-c4cc(F)cc(F)c4)ccc32)cc1 ; Fc1cc(F)cc(-c2ccc3c(c2)c2cc(-c4cc(F)cc(F)c4)ccc2n3-c2ccccc2-c2cccc(-c3cc(C(F)(F)F)cc(C(F)(F)F)c3)c2)c1]

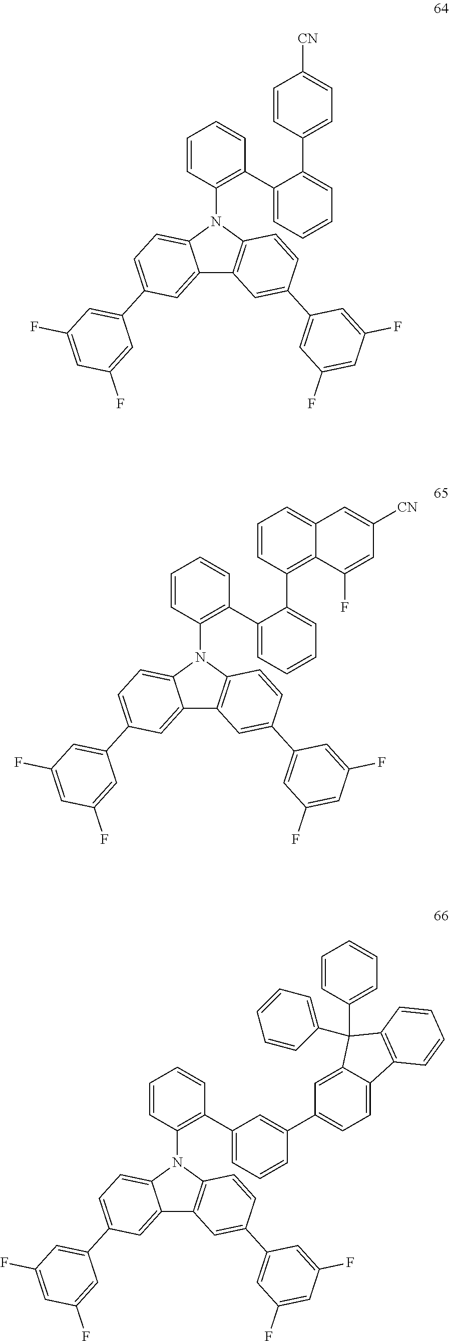 [SMILES: N#Cc1ccc(-c2ccccc2-c2ccccc2-n2c3ccc(-c4cc(F)cc(F)c4)cc3c3cc(-c4cc(F)cc(F)c4)ccc32)cc1 ; N#Cc1cc(F)c2c(-c3ccccc3-c3ccccc3-n3c4ccc(-c5cc(F)cc(F)c5)cc4c4cc(-c5cc(F)cc(F)c5)ccc43)cccc2c1 ; Fc1cc(F)cc(-c2ccc3c(c2)c2cc(-c4cc(F)cc(F)c4)ccc2n3-c2ccccc2-c2cccc(-c3ccc4c(c3)C(c3ccccc3)(c3ccccc3)c3ccccc3-4)c2)c1]

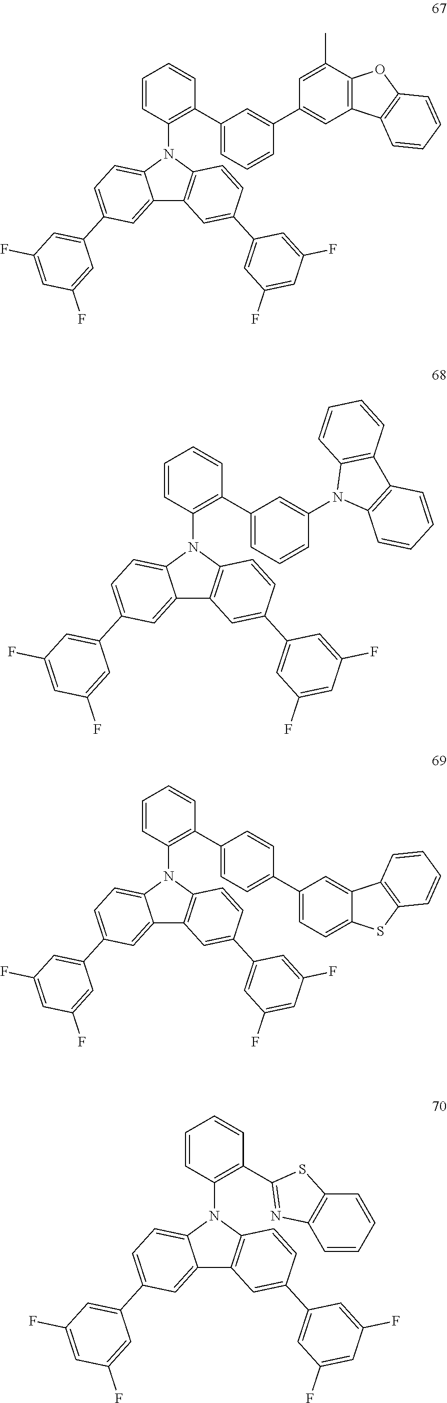 [SMILES: Cc1cc(-c2cccc(-c3ccccc3-n3c4ccc(-c5cc(F)cc(F)c5)cc4c4cc(-c5cc(F)cc(F)c5)ccc43)c2)cc2c1oc1ccccc12 ; Fc1cc(F)cc(-c2ccc3c(c2)c2cc(-c4cc(F)cc(F)c4)ccc2n3-c2ccccc2-c2cccc(-n3c4ccccc4c4ccccc43)c2)c1 ; Fc1cc(F)cc(-c2ccc3c(c2)c2cc(-c4cc(F)cc(F)c4)ccc2n3-c2ccccc2-c2ccc(-c3ccc4sc5ccccc5c4c3)cc2)c1 ; Fc1cc(F)cc(-c2ccc3c(c2)c2cc(-c4cc(F)cc(F)c4)ccc2n3-c2ccccc2-c2nc3ccccc3s2)c1]

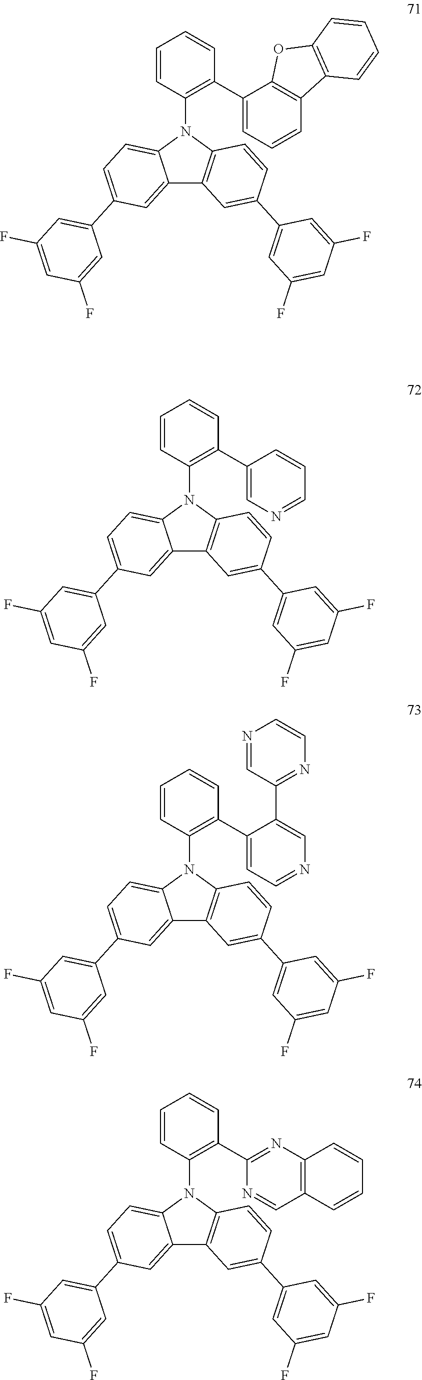

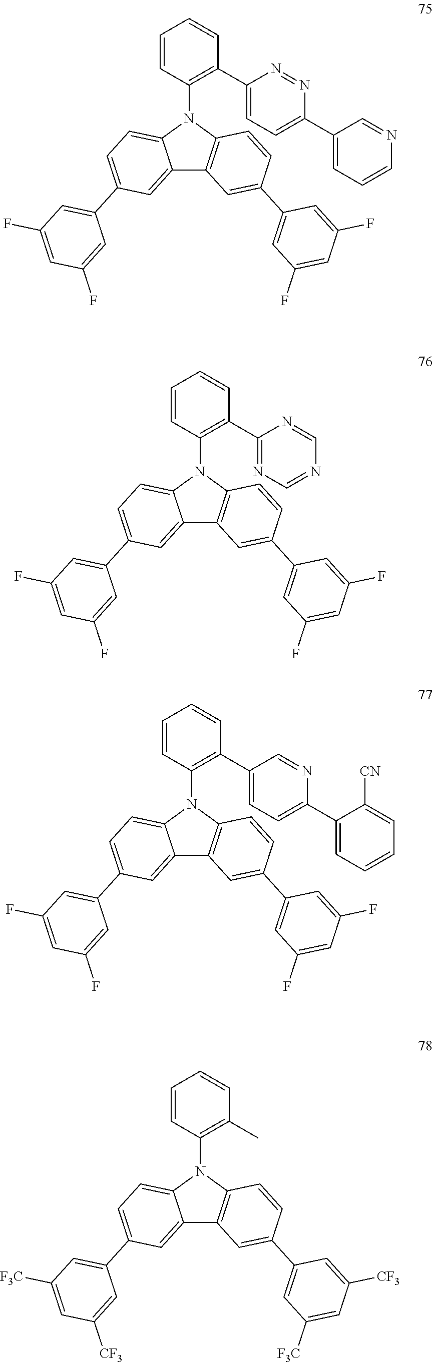 [SMILES: Fc1cc(F)cc(-c2ccc3c(c2)c2cc(-c4cc(F)cc(F)c4)ccc2n3-c2ccccc2-c2ccc(-c3cccnc3)nn2)c1 ; Fc1cc(F)cc(-c2ccc3c(c2)c2cc(-c4cc(F)cc(F)c4)ccc2n3-c2ccccc2-c2ncncn2)c1 ; N#Cc1ccccc1-c1ccc(-c2ccccc2-n2c3ccc(-c4cc(F)cc(F)c4)cc3c3cc(-c4cc(F)cc(F)c4)ccc32)cn1 ; Cc1ccccc1-n1c2ccc(-c3cc(C(F)(F)F)cc(C(F)(F)F)c3)cc2c2cc(-c3cc(C(F)(F)F)cc(C(F)(F)F)c3)ccc21]

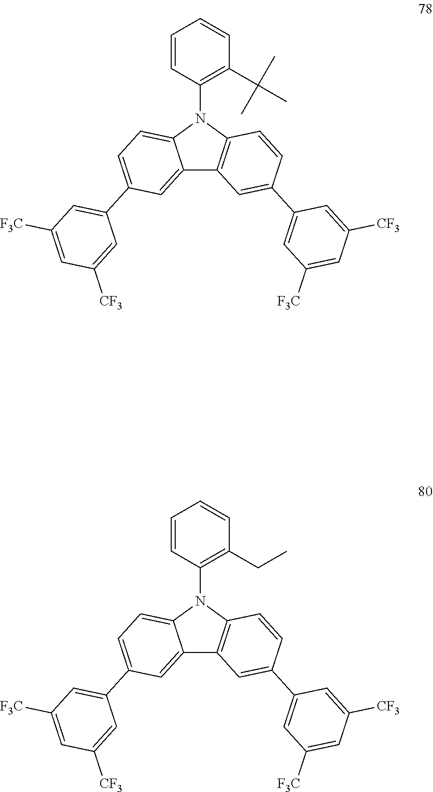 [SMILES: CC(C)(C)c1ccccc1-n1c2ccc(-c3cc(C(F)(F)F)cc(C(F)(F)F)c3)cc2c2cc(-c3cc(C(F)(F)F)cc(C(F)(F)F)c3)ccc21 ; CCc1ccccc1-n1c2ccc(-c3cc(C(F)(F)F)cc(C(F)(F)F)c3)cc2c2cc(-c3cc(C(F)(F)F)cc(C(F)(F)F)c3)ccc21]

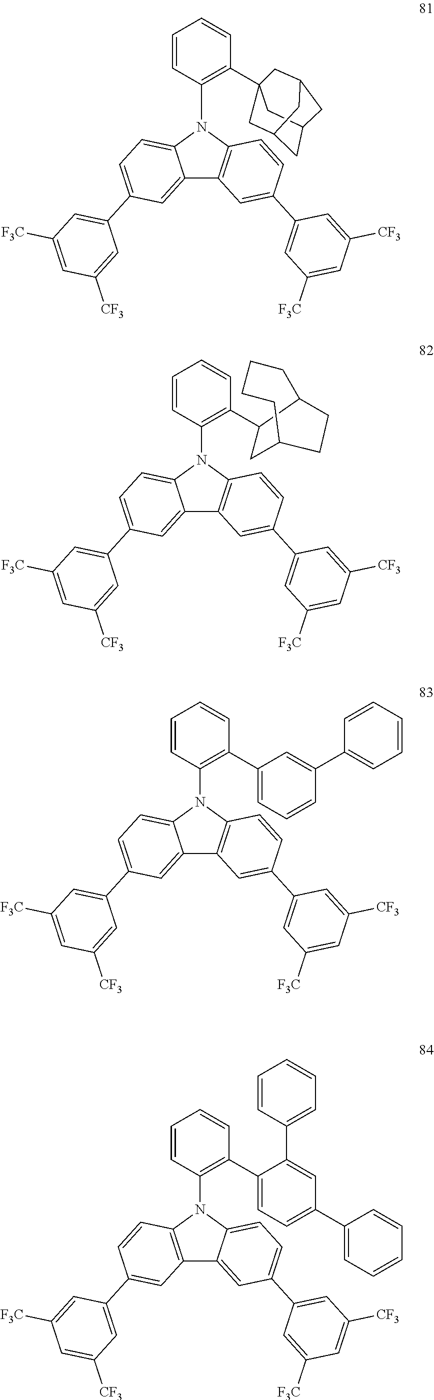 [SMILES: FC(F)(F)c1cc(-c2ccc3c(c2)c2cc(-c4cc(C(F)(F)F)cc(C(F)(F)F)c4)ccc2n3-c2ccccc2C23CC4CC(CC(C4)C2)C3)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2C2CC3CCCCC2CC3)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc(-c3ccccc3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2-c2ccccc2)cc(C(F)(F)F)c1]

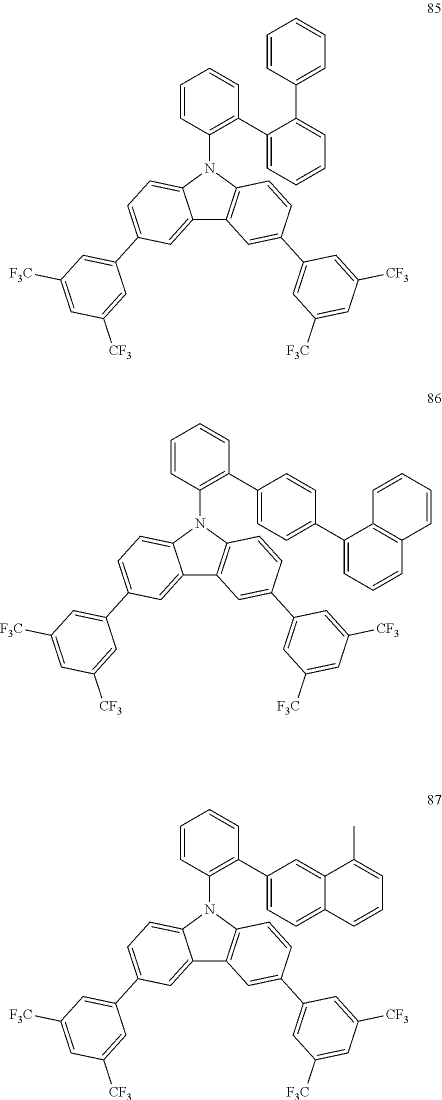 [SMILES: FC(F)(F)c1cc(-c2ccc3c(c2)c2cc(-c4cc(C(F)(F)F)cc(C(F)(F)F)c4)ccc2n3-c2ccccc2-c2ccccc2-c2ccccc2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3cccc4ccccc34)cc2)cc(C(F)(F)F)c1 ; Cc1cccc2ccc(-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)cc12]

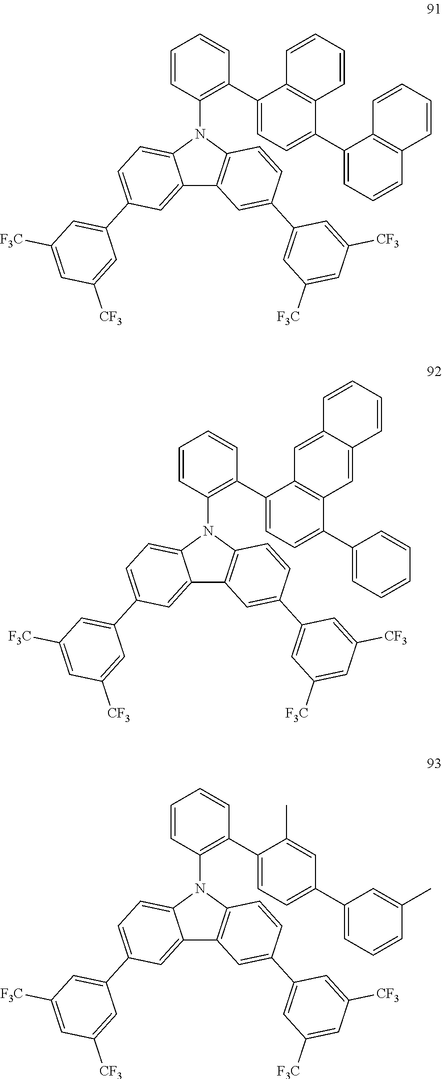 [SMILES: FC(F)(F)c1cc(-c2ccc3c(c2)c2cc(-c4cc(C(F)(F)F)cc(C(F)(F)F)c4)ccc2n3-c2ccccc2-c2ccc(-c3cccc4ccccc34)c3ccccc23)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)c3cc4ccccc4cc23)cc(C(F)(F)F)c1 ; Cc1cccc(-c2ccc(-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)c(C)c2)c1]

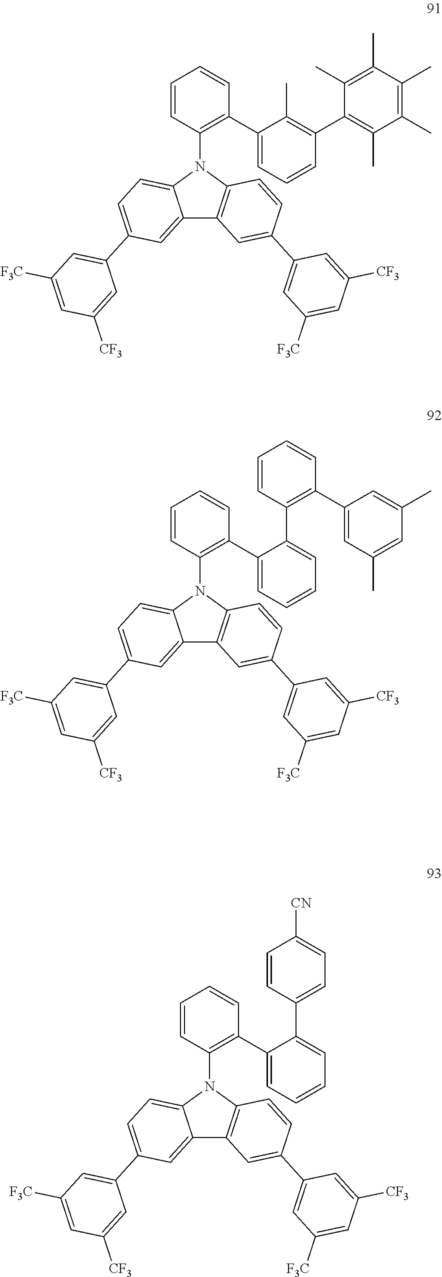 [SMILES: Cc1c(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cccc1-c1c(C)c(C)c(C)c(C)c1C ; Cc1cc(C)cc(-c2ccccc2-c2ccccc2-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c1 ; N#Cc1ccc(-c2ccccc2-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cc1]

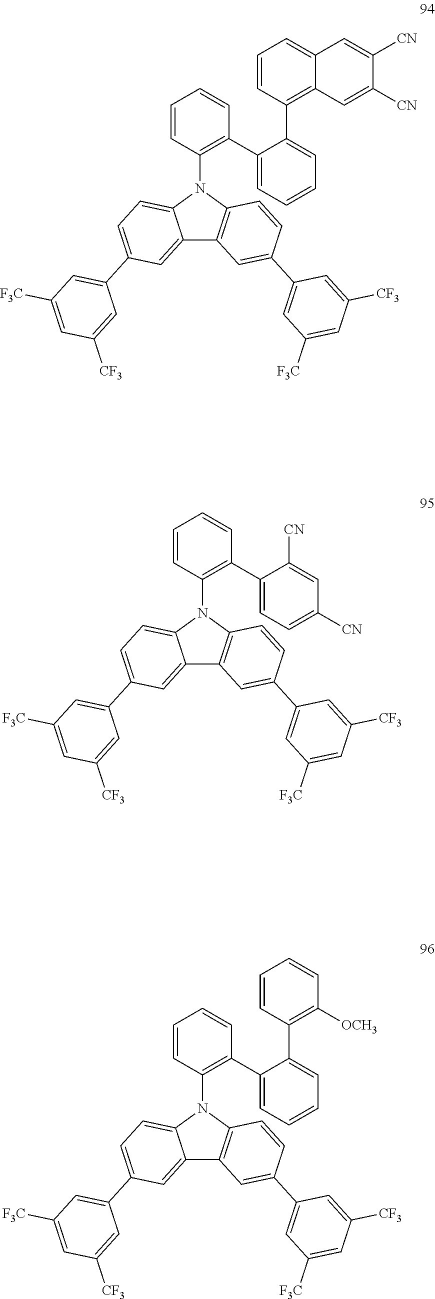 [SMILES: N#Cc1cc2cccc(-c3ccccc3-c3ccccc3-n3c4ccc(-c5cc(C(F)(F)F)cc(C(F)(F)F)c5)cc4c4cc(-c5cc(C(F)(F)F)cc(C(F)(F)F)c5)ccc43)c2cc1C#N ; N#Cc1ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c(C#N)c1 ; COc1ccccc1-c1ccccc1-c1ccccc1-n1c2ccc(-c3cc(C(F)(F)F)cc(C(F)(F)F)c3)cc2c2cc(-c3cc(C(F)(F)F)cc(C(F)(F)F)c3)ccc21]

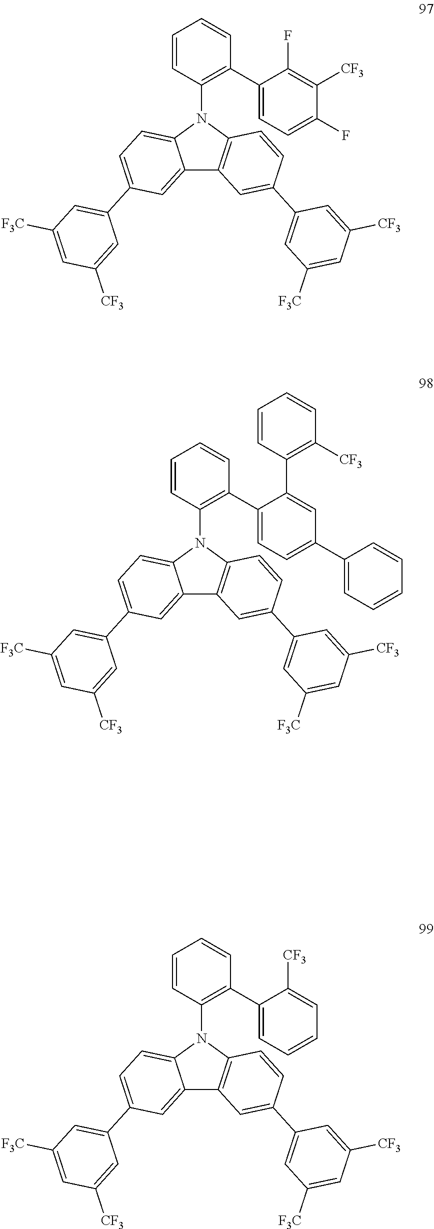 [SMILES: Fc1ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)c(F)c1C(F)(F)F ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2-c2ccccc2C(F)(F)F)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccccc2C(F)(F)F)cc(C(F)(F)F)c1]

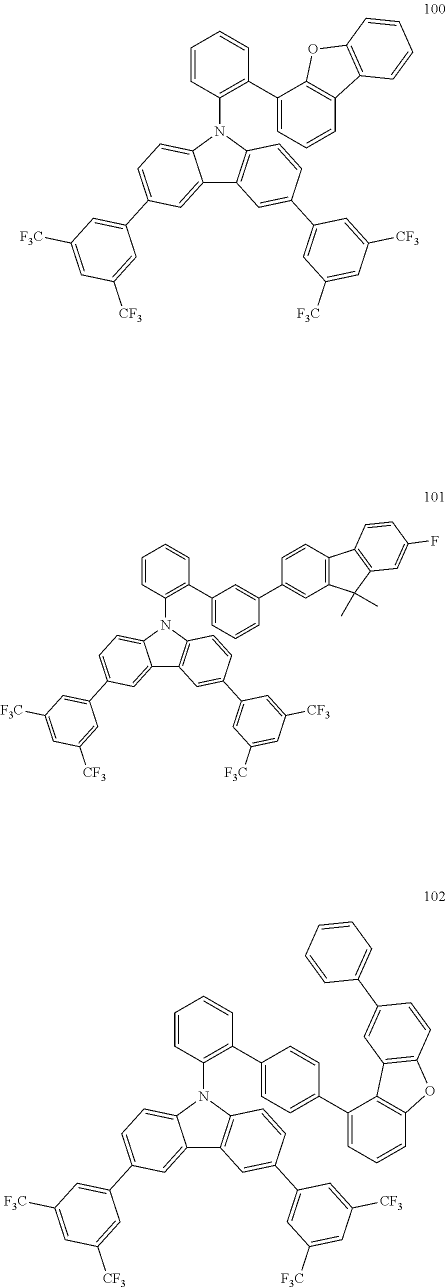 [SMILES: FC(F)(F)c1cc(-c2ccc3c(c2)c2cc(-c4cc(C(F)(F)F)cc(C(F)(F)F)c4)ccc2n3-c2ccccc2-c2cccc3c2oc2ccccc23)cc(C(F)(F)F)c1 ; CC1(C)c2cc(F)ccc2-c2ccc(-c3cccc(-c4ccccc4-n4c5ccc(-c6cc(C(F)(F)F)cc(C(F)(F)F)c6)cc5c5cc(-c6cc(C(F)(F)F)cc(C(F)(F)F)c6)ccc54)c3)cc21 ; FC(F)(F)c1cc(-c2ccc3c(c2)c2cc(-c4cc(C(F)(F)F)cc(C(F)(F)F)c4)ccc2n3-c2ccccc2-c2ccc(-c3cccc4oc5ccc(-c6ccccc6)cc5c34)cc2)cc(C(F)(F)F)c1]

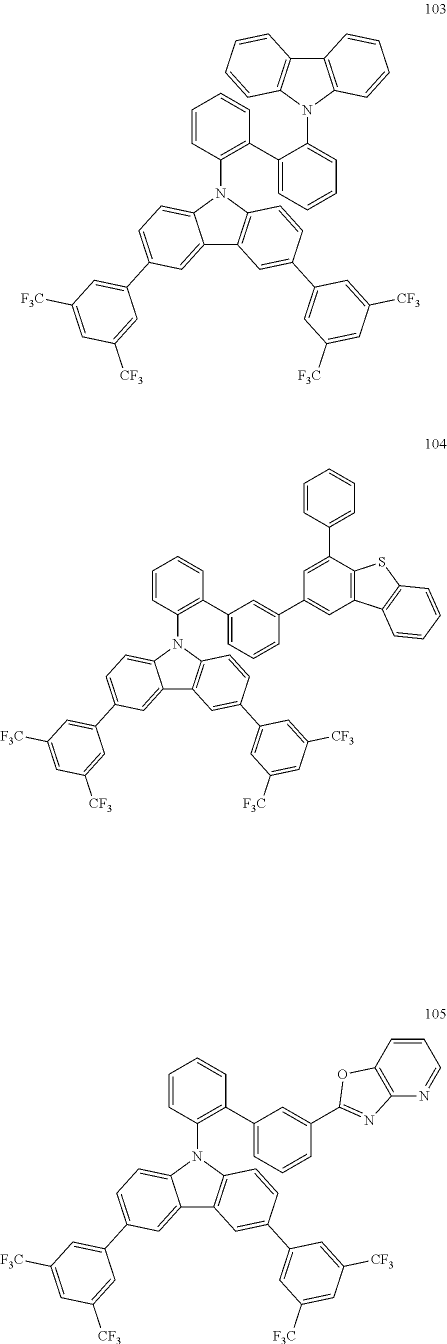 [SMILES: FC(F)(F)c1cc(-c2ccc3c(c2)c2cc(-c4cc(C(F)(F)F)cc(C(F)(F)F)c4)ccc2n3-c2ccccc2-c2ccccc2-n2c3ccccc3c3ccccc32)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc(-c3cc(-c4ccccc4)c4sc5ccccc5c4c3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cccc(-c3nc4ncccc4o3)c2)cc(C(F)(F)F)c1]

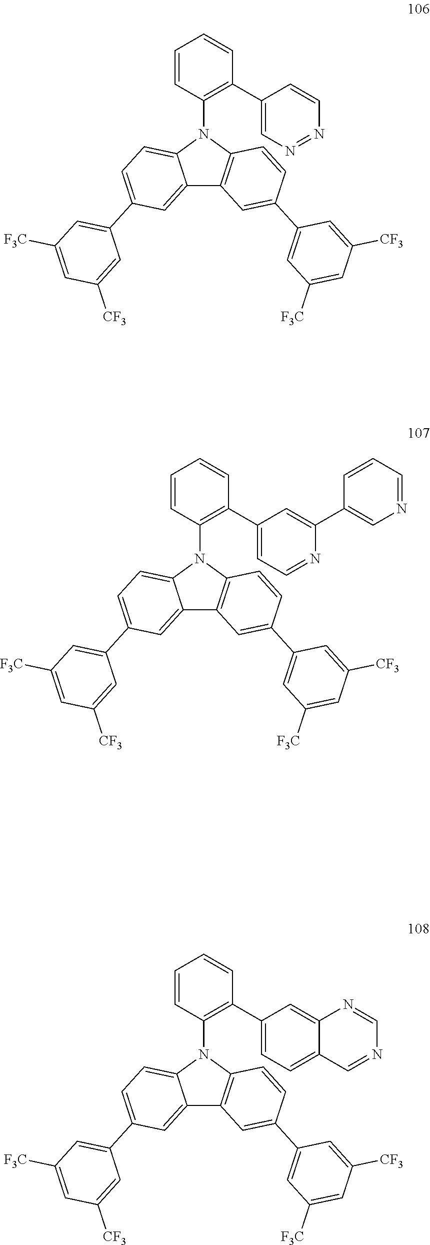 [SMILES: FC(F)(F)c1cc(-c2ccc3c(c2)c2cc(-c4cc(C(F)(F)F)cc(C(F)(F)F)c4)ccc2n3-c2ccccc2-c2ccnnc2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccnc(-c3cccnc3)c2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ccc3cncnc3c2)cc(C(F)(F)F)c1]

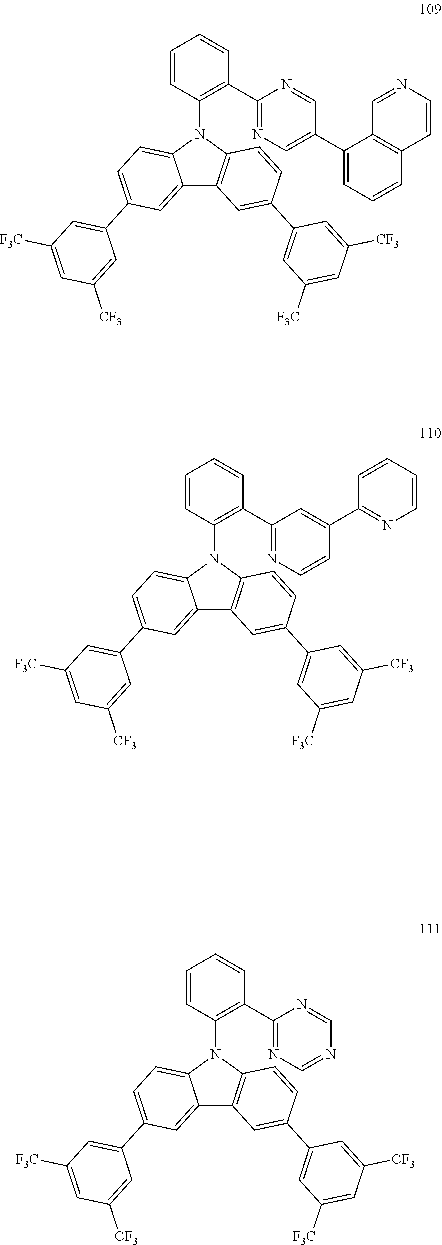 [SMILES: FC(F)(F)c1cc(-c2ccc3c(c2)c2cc(-c4cc(C(F)(F)F)cc(C(F)(F)F)c4)ccc2n3-c2ccccc2-c2ncc(-c3cccc4ccncc34)cn2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2cc(-c3ccccn3)ccn2)cc(C(F)(F)F)c1 ; FC(F)(F)c1cc(-c2ccc3c(c2)c2cc(-c4cc(C(F)(F)F)cc(C(F)(F)F)c4)ccc2n3-c2ccccc2-c2ncncn2)cc(C(F)(F)F)c1]

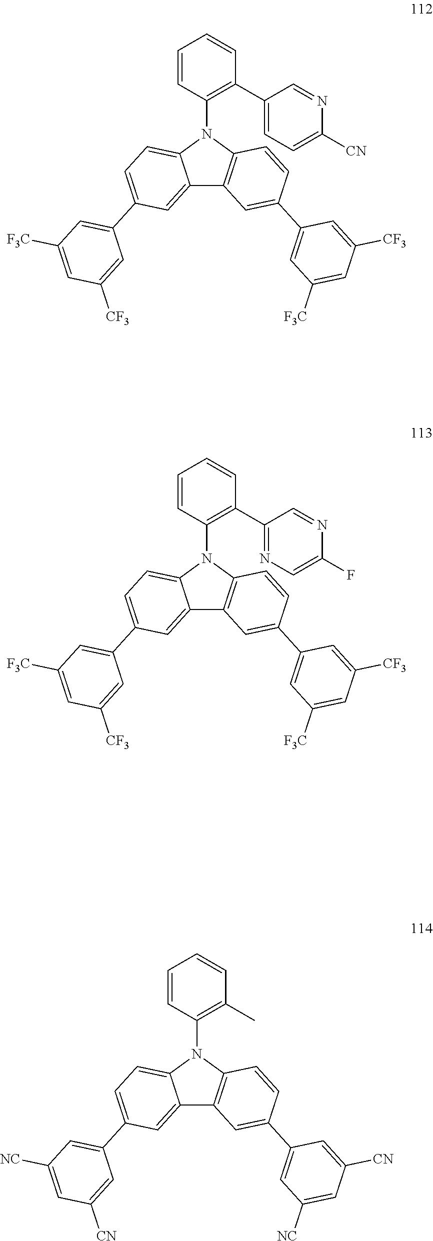 [SMILES: N#Cc1ccc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cn1 ; Fc1cnc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cn1 ; Cc1ccccc1-n1c2ccc(-c3cc(C#N)cc(C#N)c3)cc2c2cc(-c3cc(C#N)cc(C#N)c3)ccc21]

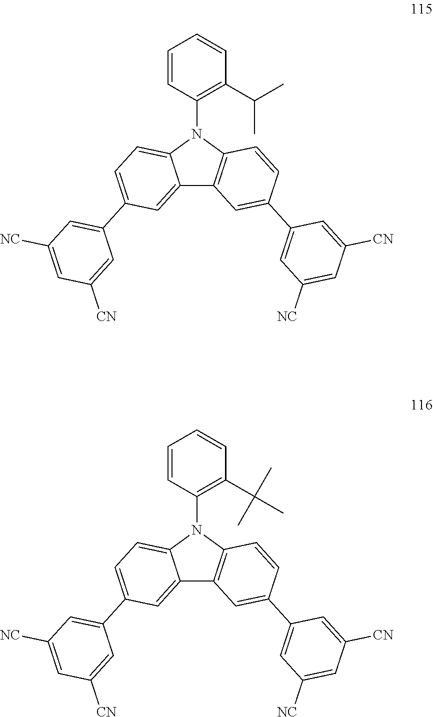 [SMILES: CC(C)c1ccccc1-n1c2ccc(-c3cc(C#N)cc(C#N)c3)cc2c2cc(-c3cc(C#N)cc(C#N)c3)ccc21 ; CC(C)(C)c1ccccc1-n1c2ccc(-c3cc(C#N)cc(C#N)c3)cc2c2cc(-c3cc(C#N)cc(C#N)c3)ccc21]

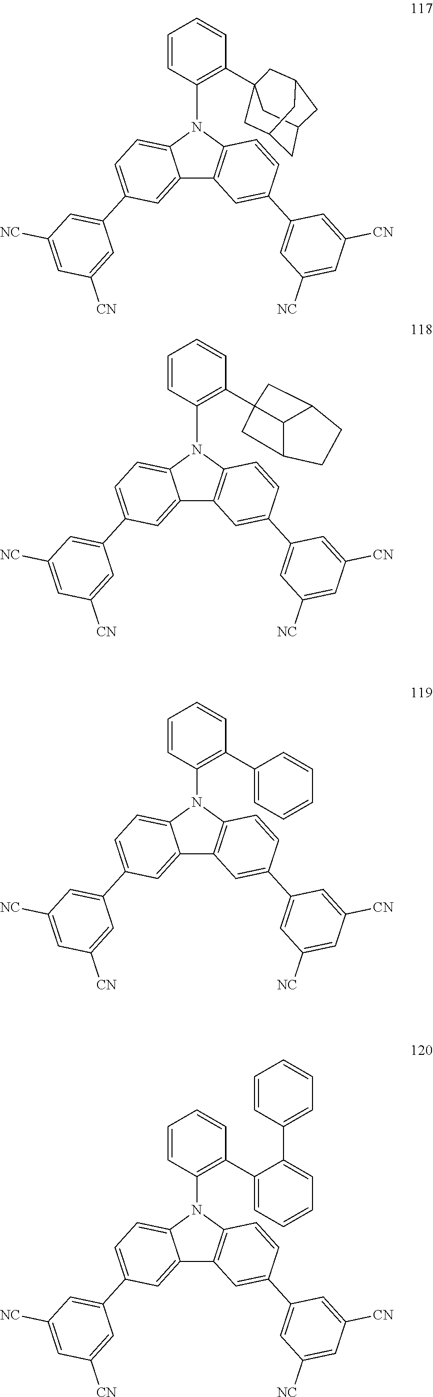 [SMILES: N#Cc1cc(C#N)cc(-c2ccc3c(c2)c2cc(-c4cc(C#N)cc(C#N)c4)ccc2n3-c2ccccc2C23CC4CC(CC(C4)C2)C3)c1 ; N#Cc1cc(C#N)cc(-c2ccc3c(c2)c2cc(-c4cc(C#N)cc(C#N)c4)ccc2n3-c2ccccc2C23CC4CCC(C2)C43)c1 ; N#Cc1cc(C#N)cc(-c2ccc3c(c2)c2cc(-c4cc(C#N)cc(C#N)c4)ccc2n3-c2ccccc2-c2ccccc2)c1 ; N#Cc1cc(C#N)cc(-c2ccc3c(c2)c2cc(-c4cc(C#N)cc(C#N)c4)ccc2n3-c2ccccc2-c2ccccc2-c2ccccc2)c1]

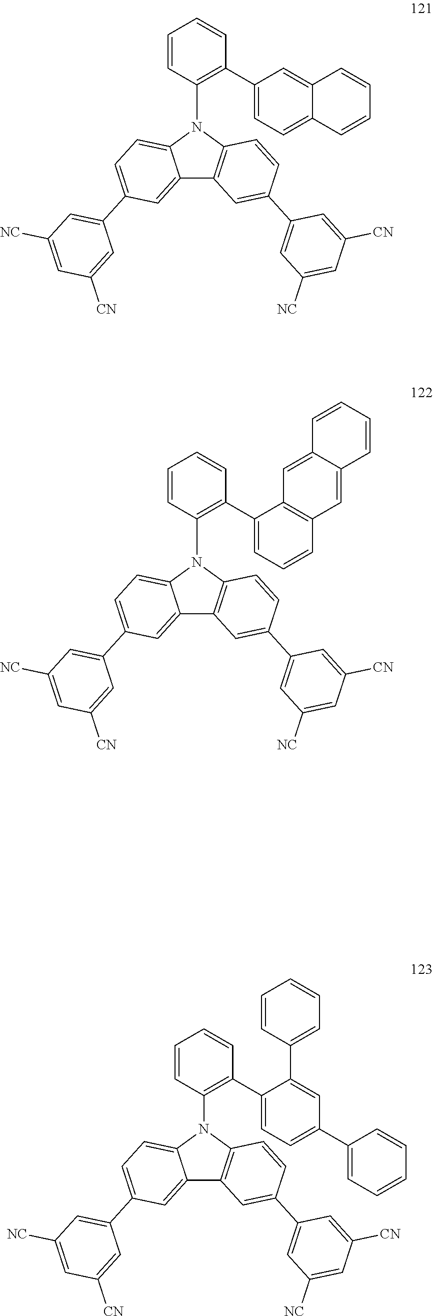 [SMILES: N#Cc1cc(C#N)cc(-c2ccc3c(c2)c2cc(-c4cc(C#N)cc(C#N)c4)ccc2n3-c2ccccc2-c2ccc3ccccc3c2)c1 ; N#Cc1cc(C#N)cc(-c2ccc3c(c2)c2cc(-c4cc(C#N)cc(C#N)c4)ccc2n3-c2ccccc2-c2cccc3cc4ccccc4cc23)c1 ; N#Cc1cc(C#N)cc(-c2ccc3c(c2)c2cc(-c4cc(C#N)cc(C#N)c4)ccc2n3-c2ccccc2-c2ccc(-c3ccccc3)cc2-c2ccccc2)c1]

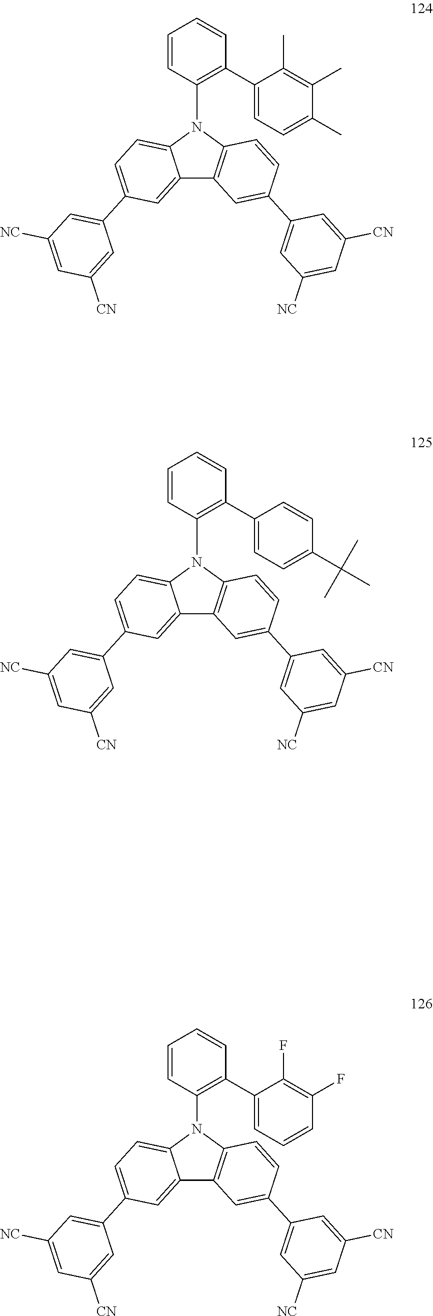 [SMILES: Cc1ccc(-c2ccccc2-n2c3ccc(-c4cc(C#N)cc(C#N)c4)cc3c3cc(-c4cc(C#N)cc(C#N)c4)ccc32)c(C)c1C ; CC(C)(C)c1ccc(-c2ccccc2-n2c3ccc(-c4cc(C#N)cc(C#N)c4)cc3c3cc(-c4cc(C#N)cc(C#N)c4)ccc32)cc1 ; N#Cc1cc(C#N)cc(-c2ccc3c(c2)c2cc(-c4cc(C#N)cc(C#N)c4)ccc2n3-c2ccccc2-c2cccc(F)c2F)c1]

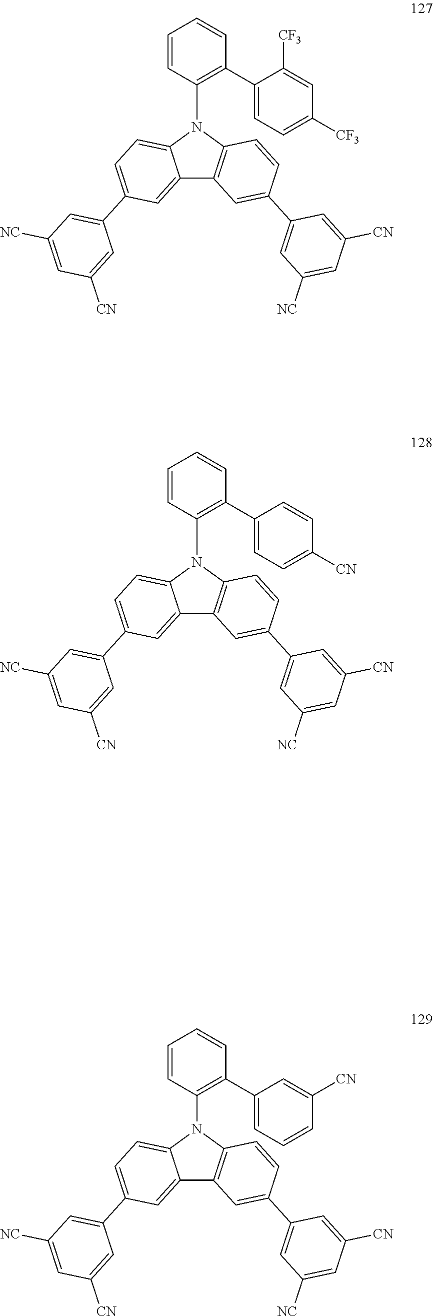 [SMILES: N#Cc1cc(C#N)cc(-c2ccc3c(c2)c2cc(-c4cc(C#N)cc(C#N)c4)ccc2n3-c2ccccc2-c2ccc(C(F)(F)F)cc2C(F)(F)F)c1 ; N#Cc1ccc(-c2ccccc2-n2c3ccc(-c4cc(C#N)cc(C#N)c4)cc3c3cc(-c4cc(C#N)cc(C#N)c4)ccc32)cc1 ; N#Cc1cc(C#N)cc(-c2ccc3c(c2)c2cc(-c4cc(C#N)cc(C#N)c4)ccc2n3-c2ccccc2-c2cccc(C#N)c2)c1]

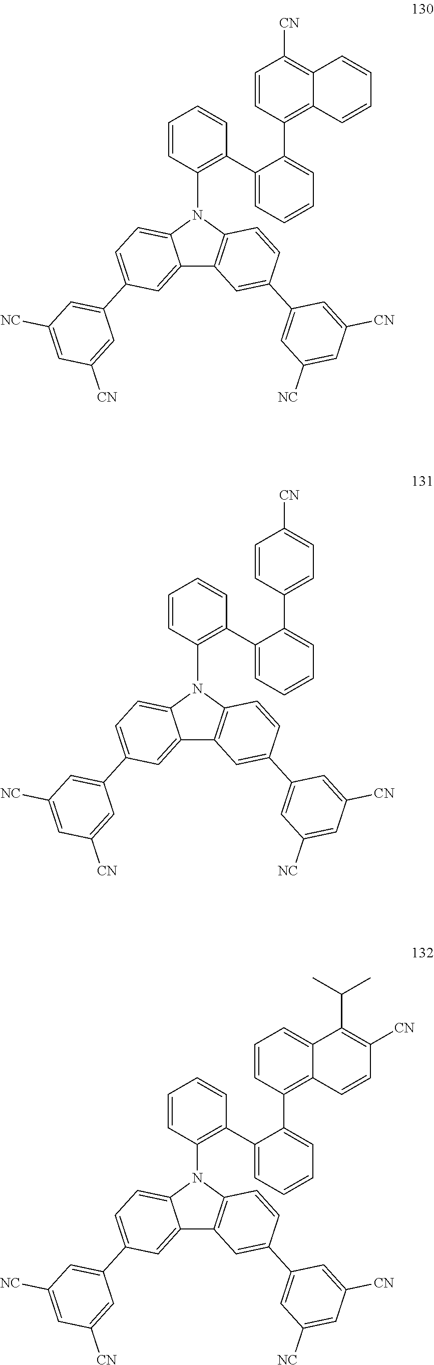 [SMILES: N#Cc1cc(C#N)cc(-c2ccc3c(c2)c2cc(-c4cc(C#N)cc(C#N)c4)ccc2n3-c2ccccc2-c2ccccc2-c2ccc(C#N)c3ccccc23)c1 ; N#Cc1ccc(-c2ccccc2-c2ccccc2-n2c3ccc(-c4cc(C#N)cc(C#N)c4)cc3c3cc(-c4cc(C#N)cc(C#N)c4)ccc32)cc1 ; CC(C)c1c(C#N)ccc2c(-c3ccccc3-c3ccccc3-n3c4ccc(-c5cc(C#N)cc(C#N)c5)cc4c4cc(-c5cc(C#N)cc(C#N)c5)ccc43)cccc12]

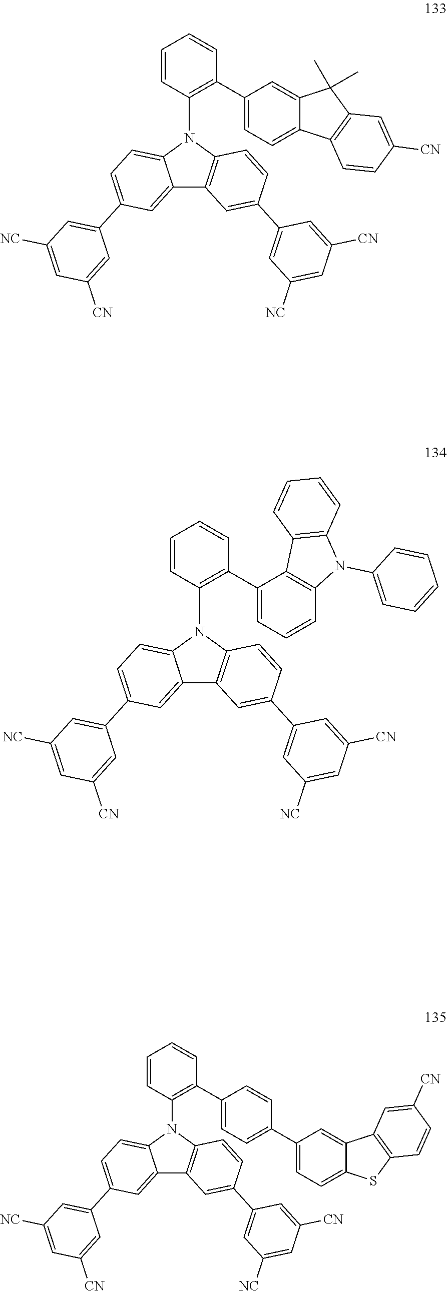 [SMILES: CC1(C)c2cc(C#N)ccc2-c2ccc(-c3ccccc3-n3c4ccc(-c5cc(C#N)cc(C#N)c5)cc4c4cc(-c5cc(C#N)cc(C#N)c5)ccc43)cc21 ; N#Cc1cc(C#N)cc(-c2ccc3c(c2)c2cc(-c4cc(C#N)cc(C#N)c4)ccc2n3-c2ccccc2-c2cccc3c2c2ccccc2n3-c2ccccc2)c1 ; N#Cc1cc(C#N)cc(-c2ccc3c(c2)c2cc(-c4cc(C#N)cc(C#N)c4)ccc2n3-c2ccccc2-c2ccc(-c3ccc4sc5ccc(C#N)cc5c4c3)cc2)c1]

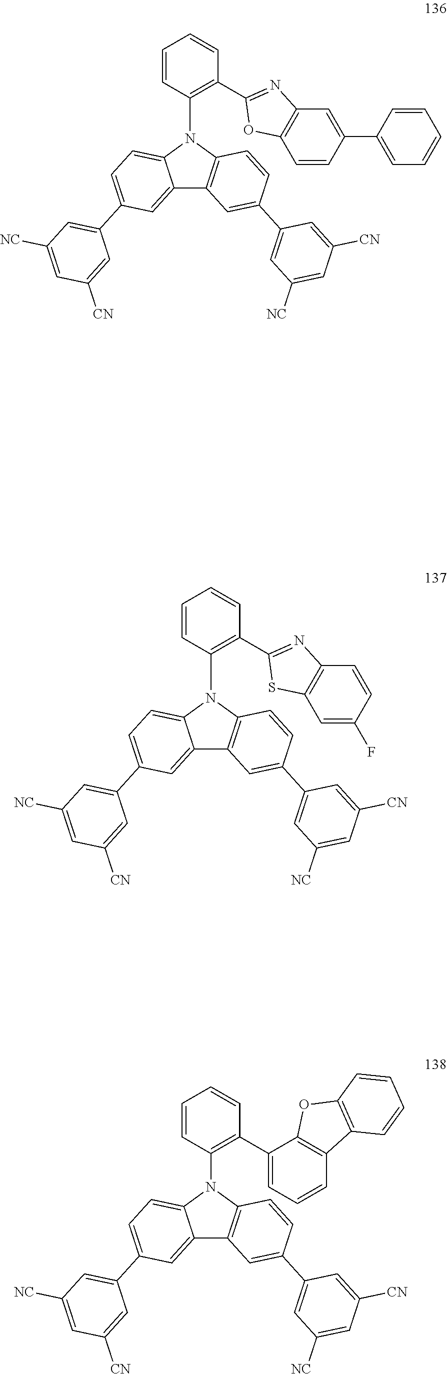 [SMILES: N#Cc1cc(C#N)cc(-c2ccc3c(c2)c2cc(-c4cc(C#N)cc(C#N)c4)ccc2n3-c2ccccc2-c2nc3cc(-c4ccccc4)ccc3o2)c1 ; N#Cc1cc(C#N)cc(-c2ccc3c(c2)c2cc(-c4cc(C#N)cc(C#N)c4)ccc2n3-c2ccccc2-c2nc3ccc(F)cc3s2)c1 ; N#Cc1cc(C#N)cc(-c2ccc3c(c2)c2cc(-c4cc(C#N)cc(C#N)c4)ccc2n3-c2ccccc2-c2cccc3c2oc2ccccc23)c1]

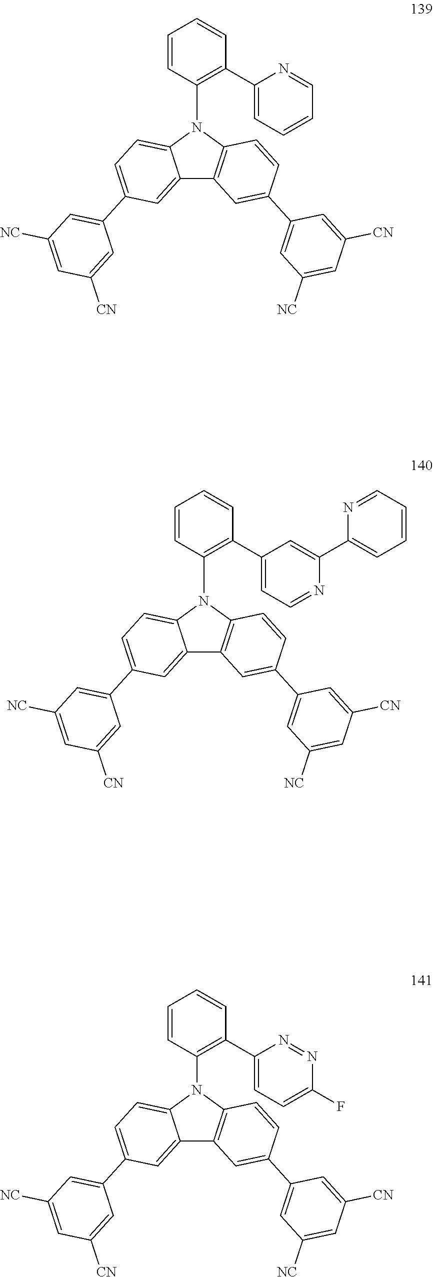 [SMILES: N#Cc1cc(C#N)cc(-c2ccc3c(c2)c2cc(-c4cc(C#N)cc(C#N)c4)ccc2n3-c2ccccc2-c2ccccn2)c1 ; N#Cc1cc(C#N)cc(-c2ccc3c(c2)c2cc(-c4cc(C#N)cc(C#N)c4)ccc2n3-c2ccccc2-c2ccnc(-c3ccccn3)c2)c1 ; N#Cc1cc(C#N)cc(-c2ccc3c(c2)c2cc(-c4cc(C#N)cc(C#N)c4)ccc2n3-c2ccccc2-c2ccc(F)nn2)c1]

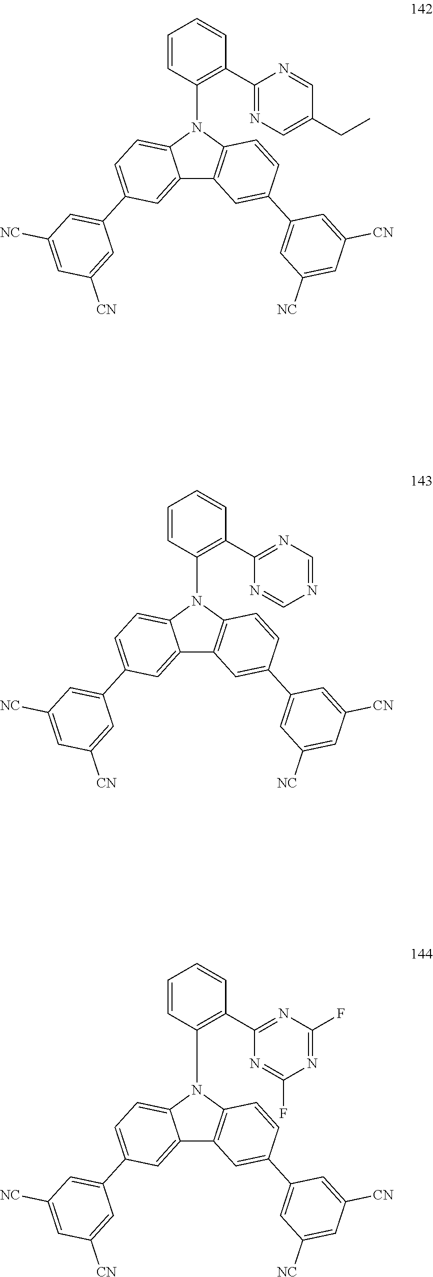 [SMILES: CCc1cnc(-c2ccccc2-n2c3ccc(-c4cc(C#N)cc(C#N)c4)cc3c3cc(-c4cc(C#N)cc(C#N)c4)ccc32)nc1 ; N#Cc1cc(C#N)cc(-c2ccc3c(c2)c2cc(-c4cc(C#N)cc(C#N)c4)ccc2n3-c2ccccc2-c2ncncn2)c1 ; N#Cc1cc(C#N)cc(-c2ccc3c(c2)c2cc(-c4cc(C#N)cc(C#N)c4)ccc2n3-c2ccccc2-c2nc(F)nc(F)n2)c1]

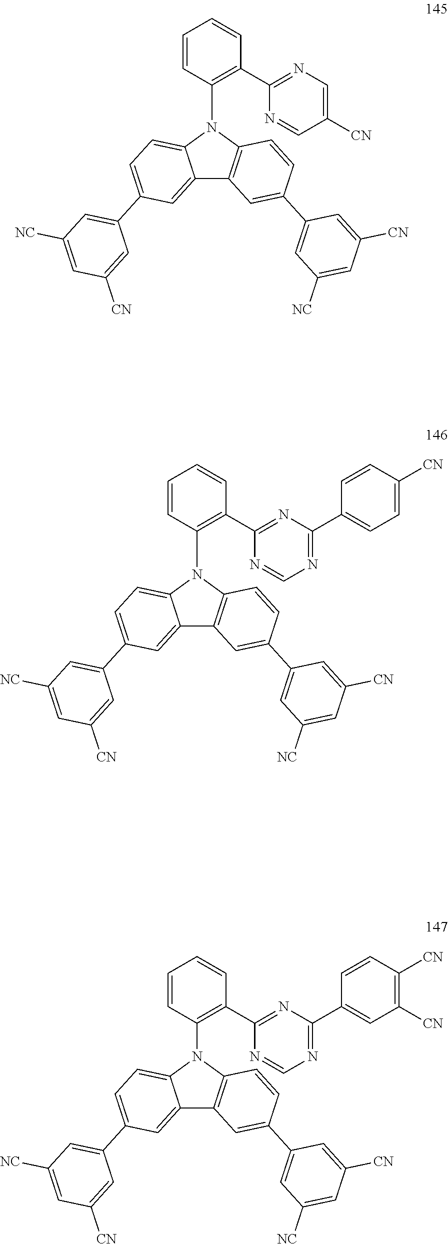 [SMILES: N#Cc1cnc(-c2ccccc2-n2c3ccc(-c4cc(C#N)cc(C#N)c4)cc3c3cc(-c4cc(C#N)cc(C#N)c4)ccc32)nc1 ; N#Cc1ccc(-c2ncnc(-c3ccccc3-n3c4ccc(-c5cc(C#N)cc(C#N)c5)cc4c4cc(-c5cc(C#N)cc(C#N)c5)ccc43)n2)cc1 ; N#Cc1cc(C#N)cc(-c2ccc3c(c2)c2cc(-c4cc(C#N)cc(C#N)c4)ccc2n3-c2ccccc2-c2ncnc(-c3ccc(C#N)c(C#N)c3)n2)c1]

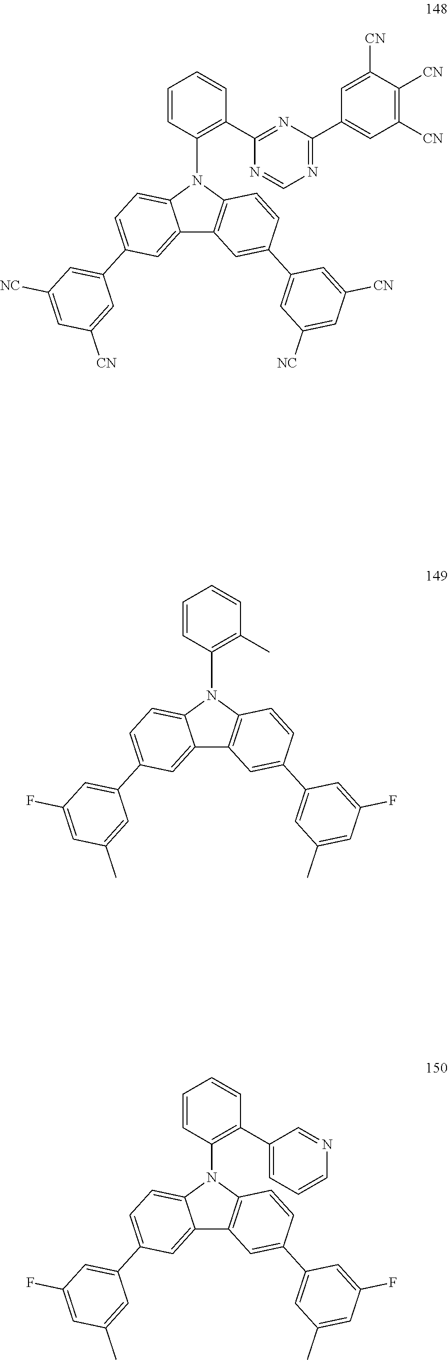 [SMILES: N#Cc1cc(C#N)cc(-c2ccc3c(c2)c2cc(-c4cc(C#N)cc(C#N)c4)ccc2n3-c2ccccc2-c2ncnc(-c3cc(C#N)c(C#N)c(C#N)c3)n2)c1 ; Cc1cc(F)cc(-c2ccc3c(c2)c2cc(-c4cc(C)cc(F)c4)ccc2n3-c2ccccc2C)c1 ; Cc1cc(F)cc(-c2ccc3c(c2)c2cc(-c4cc(C)cc(F)c4)ccc2n3-c2ccccc2-c2cccnc2)c1]

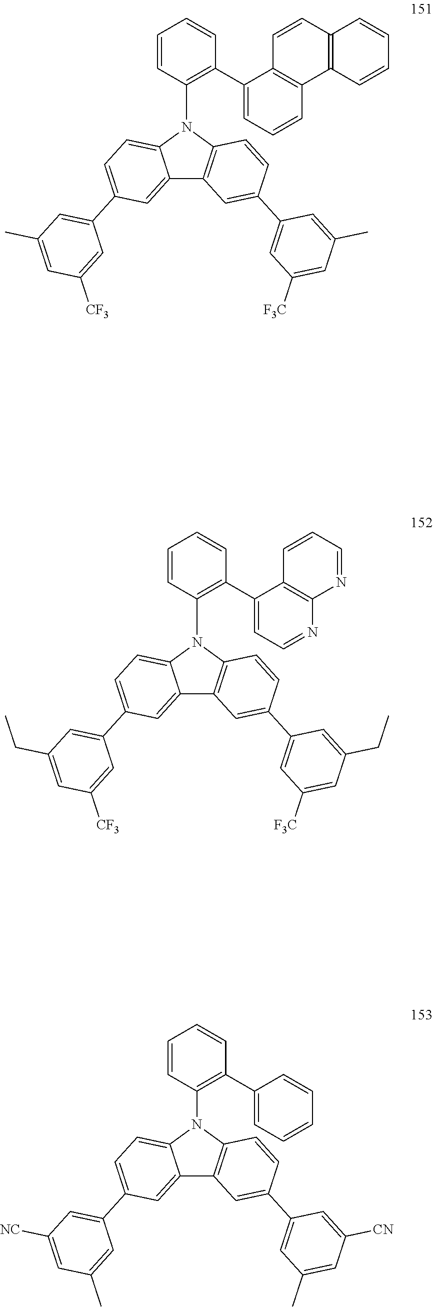 [SMILES: Cc1cc(-c2ccc3c(c2)c2cc(-c4cc(C)cc(C(F)(F)F)c4)ccc2n3-c2ccccc2-c2cccc3c2ccc2ccccc23)cc(C(F)(F)F)c1 ; CCc1cc(-c2ccc3c(c2)c2cc(-c4cc(CC)cc(C(F)(F)F)c4)ccc2n3-c2ccccc2-c2ccnc3ncccc23)cc(C(F)(F)F)c1 ; Cc1cc(C#N)cc(-c2ccc3c(c2)c2cc(-c4cc(C)cc(C#N)c4)ccc2n3-c2ccccc2-c2ccccc2)c1]

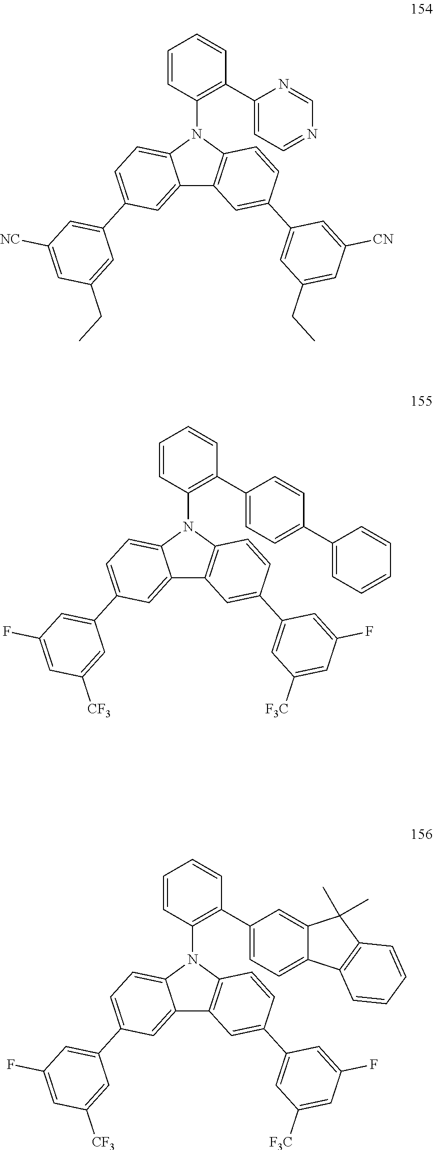 [SMILES: CCc1cc(C#N)cc(-c2ccc3c(c2)c2cc(-c4cc(C#N)cc(CC)c4)ccc2n3-c2ccccc2-c2ccncn2)c1 ; Fc1cc(-c2ccc3c(c2)c2cc(-c4cc(F)cc(C(F)(F)F)c4)ccc2n3-c2ccccc2-c2ccc(-c3ccccc3)cc2)cc(C(F)(F)F)c1 ; CC1(C)c2ccccc2-c2ccc(-c3ccccc3-n3c4ccc(-c5cc(F)cc(C(F)(F)F)c5)cc4c4cc(-c5cc(F)cc(C(F)(F)F)c5)ccc43)cc21]

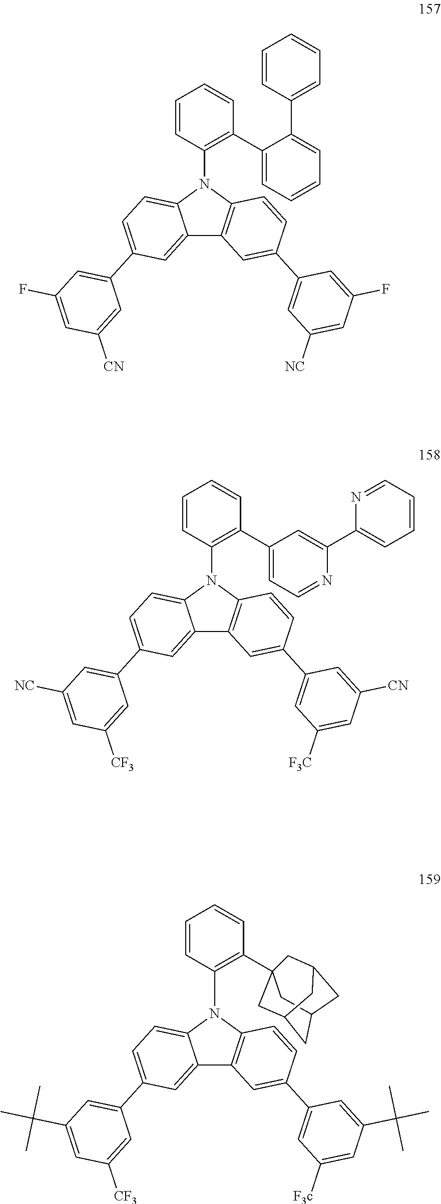

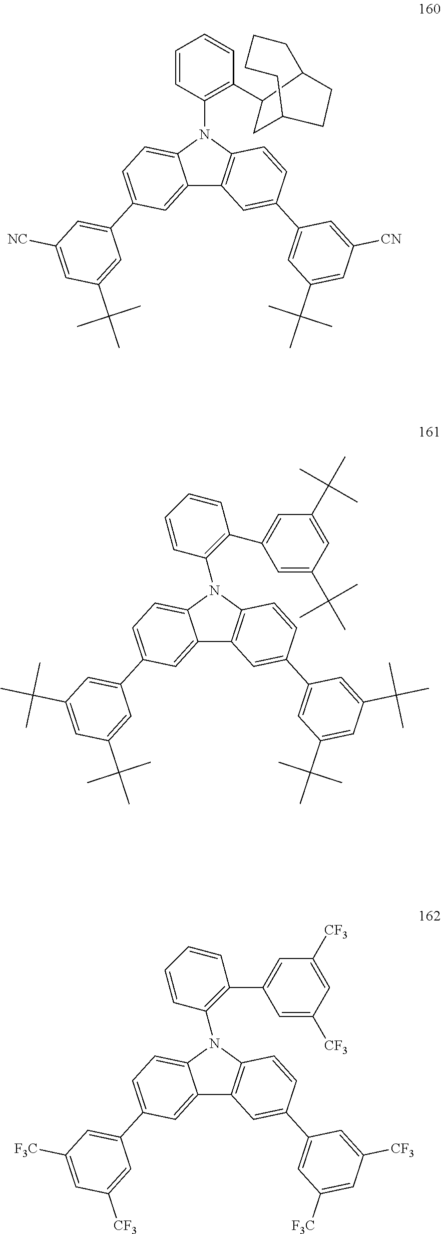 [SMILES: CC(C)(C)c1cc(C#N)cc(-c2ccc3c(c2)c2cc(-c4cc(C#N)cc(C(C)(C)C)c4)ccc2n3-c2ccccc2C2CC3CCCCC2CC3)c1 ; CC(C)(C)c1cc(-c2ccc3c(c2)c2cc(-c4cc(C(C)(C)C)cc(C(C)(C)C)c4)ccc2n3-c2ccccc2-c2cc(C(C)(C)C)cc(C(C)(C)C)c2)cc(C(C)(C)C)c1 ; FC(F)(F)c1cc(-c2ccc3c(c2)c2cc(-c4cc(C(F)(F)F)cc(C(F)(F)F)c4)ccc2n3-c2ccccc2-c2cc(C(F)(F)F)cc(C(F)(F)F)c2)cc(C(F)(F)F)c1]

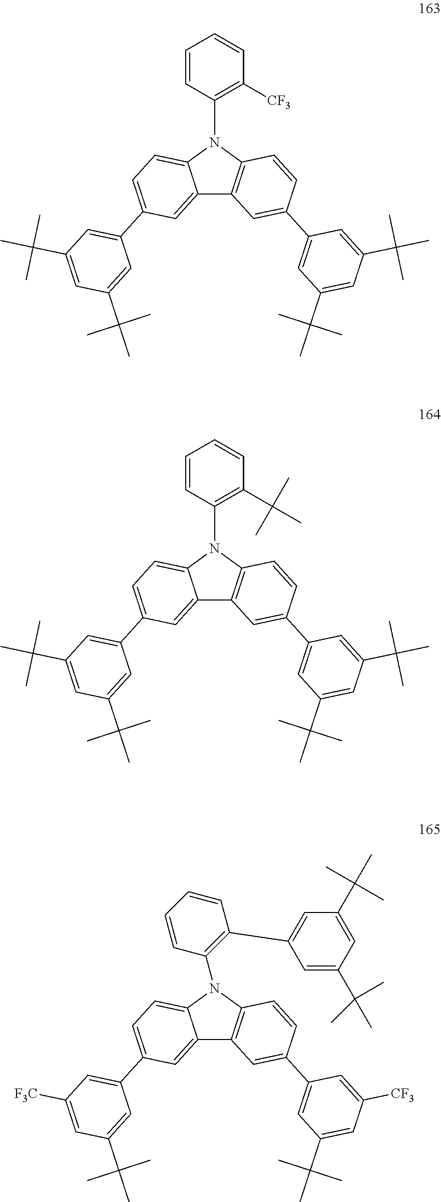

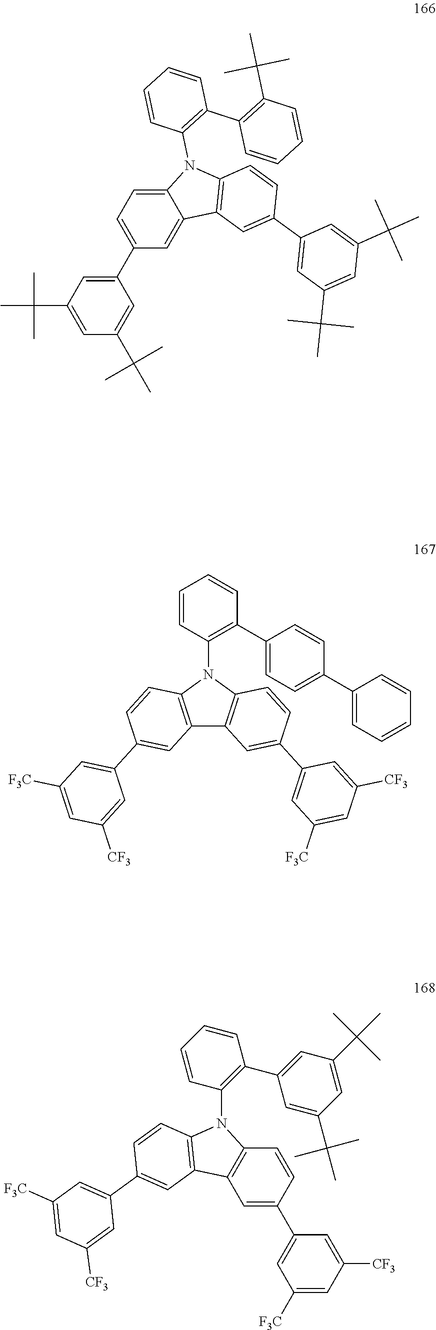 [SMILES: CC(C)(C)c1cc(-c2ccc3c(c2)c2cc(-c4cc(C(C)(C)C)cc(C(C)(C)C)c4)ccc2n3-c2ccccc2-c2ccccc2C(C)(C)C)cc(C(C)(C)C)c1 ; FC(F)(F)c1cc(-c2ccc3c(c2)c2cc(-c4cc(C(F)(F)F)cc(C(F)(F)F)c4)ccc2n3-c2ccccc2-c2ccc(-c3ccccc3)cc2)cc(C(F)(F)F)c1 ; CC(C)(C)c1cc(-c2ccccc2-n2c3ccc(-c4cc(C(F)(F)F)cc(C(F)(F)F)c4)cc3c3cc(-c4cc(C(F)(F)F)cc(C(F)(F)F)c4)ccc32)cc(C(C)(C)C)c1]

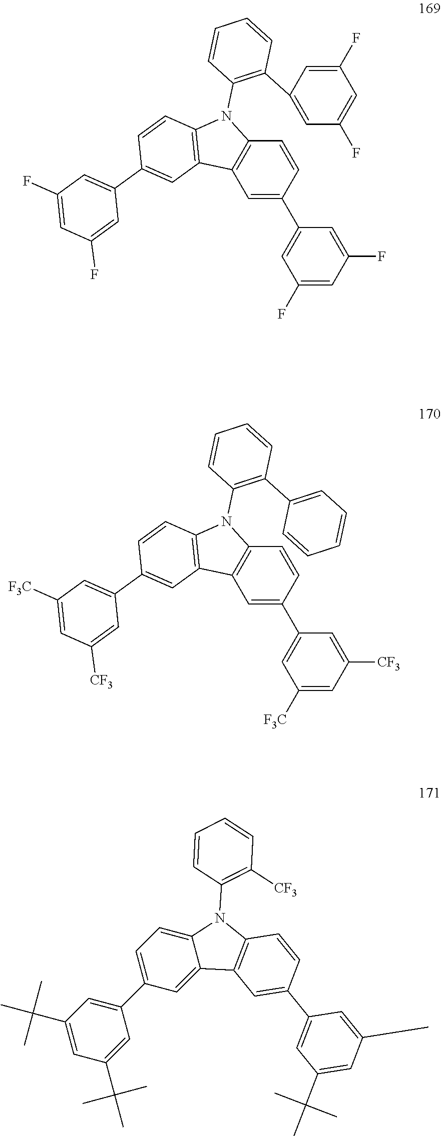 [SMILES: Fc1cc(F)cc(-c2ccc3c(c2)c2cc(-c4cc(F)cc(F)c4)ccc2n3-c2ccccc2-c2cc(F)cc(F)c2)c1 ; FC(F)(F)c1cc(-c2ccc3c(c2)c2cc(-c4cc(C(F)(F)F)cc(C(F)(F)F)c4)ccc2n3-c2ccccc2-c2ccccc2)cc(C(F)(F)F)c1 ; CCc1cc(-c2ccc3c(c2)c2cc(-c4cc(C(C)(C)C)cc(C(C)(C)C)c4)ccc2n3-c2ccccc2C(F)(F)F)cc(C(C)(C)C)c1]

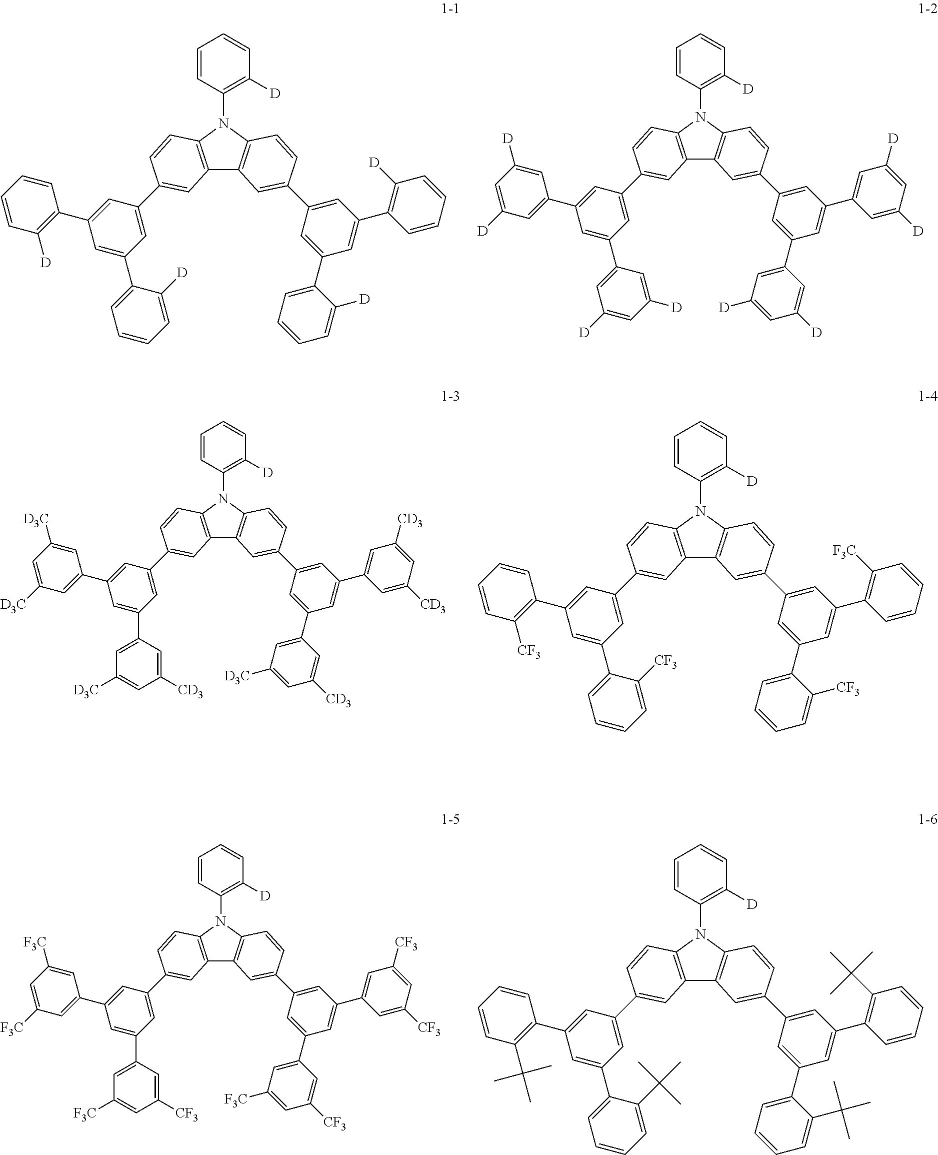

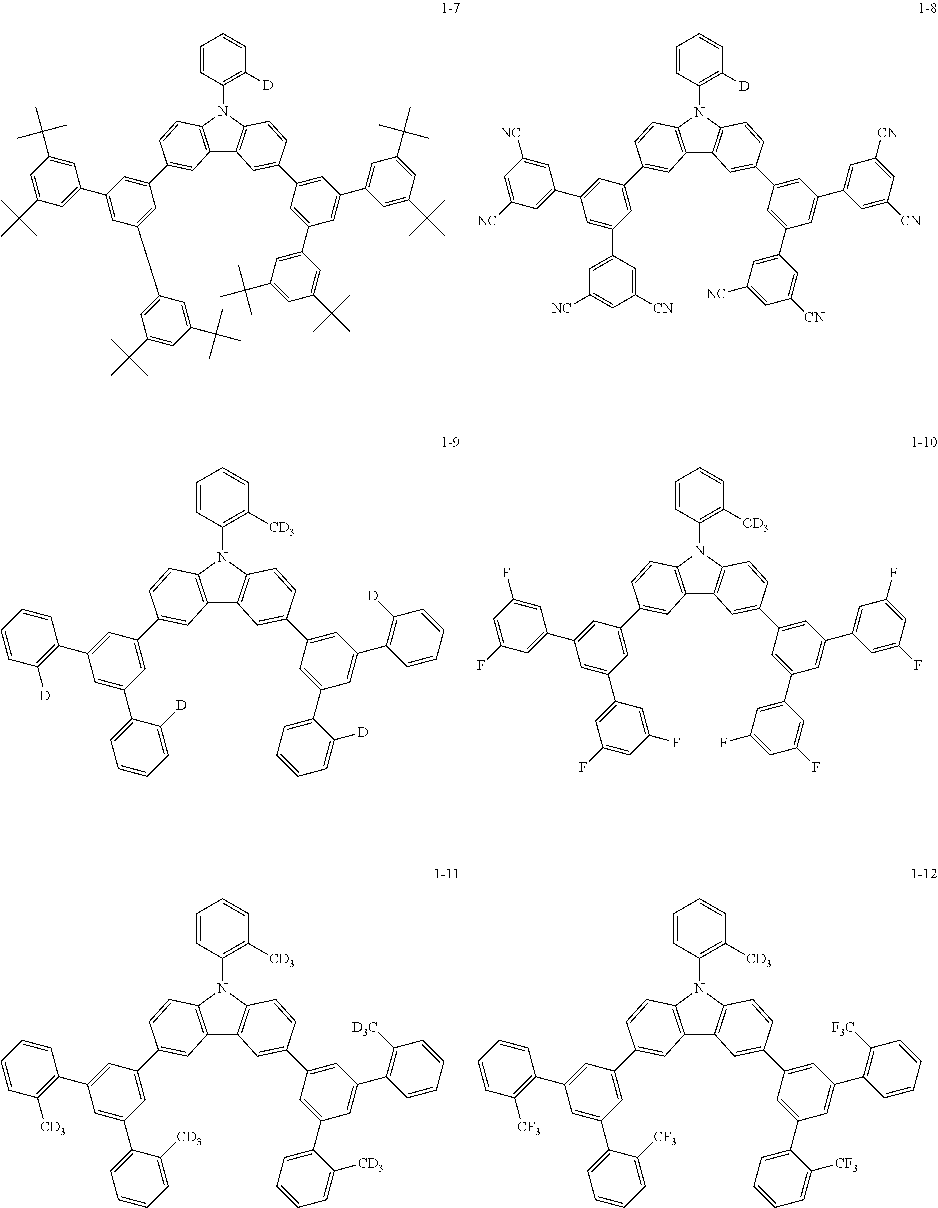

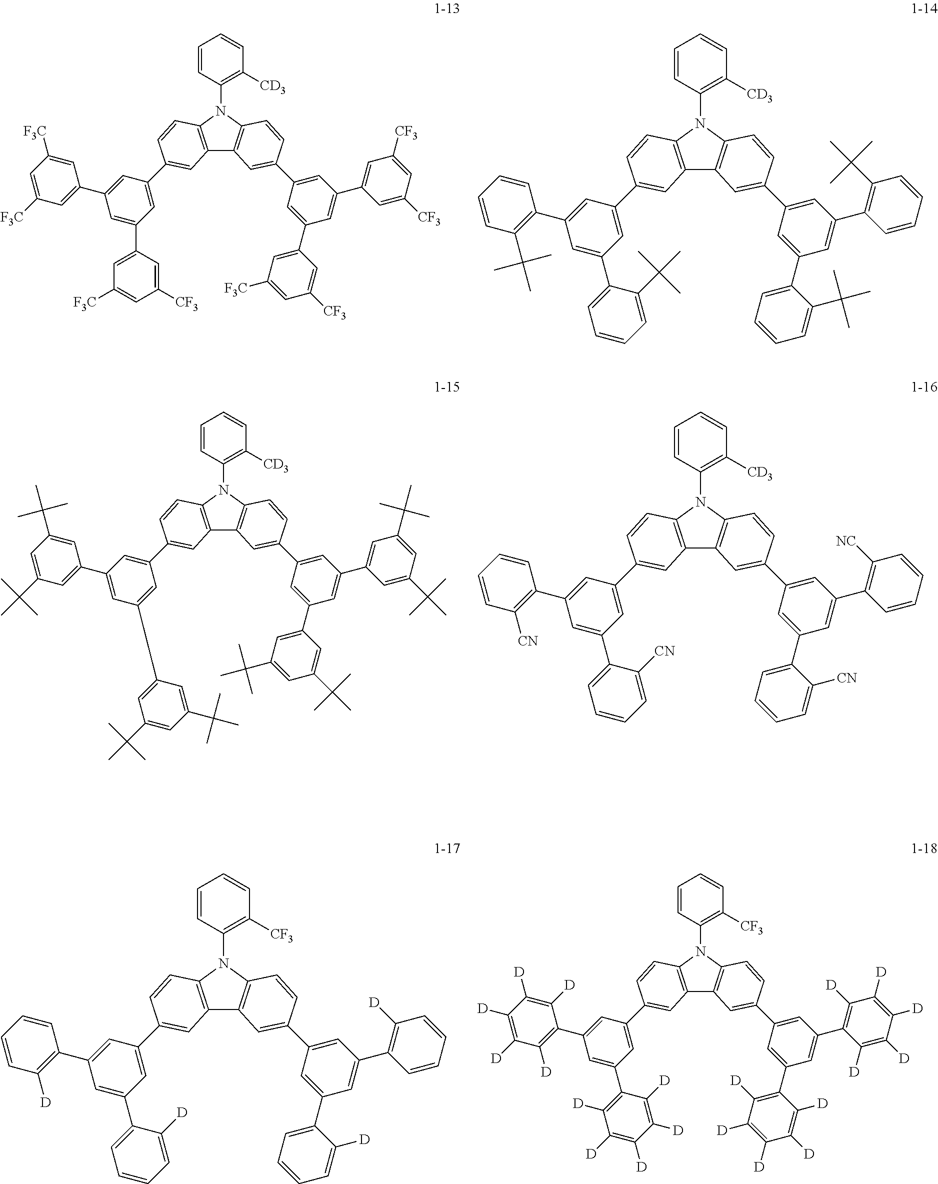

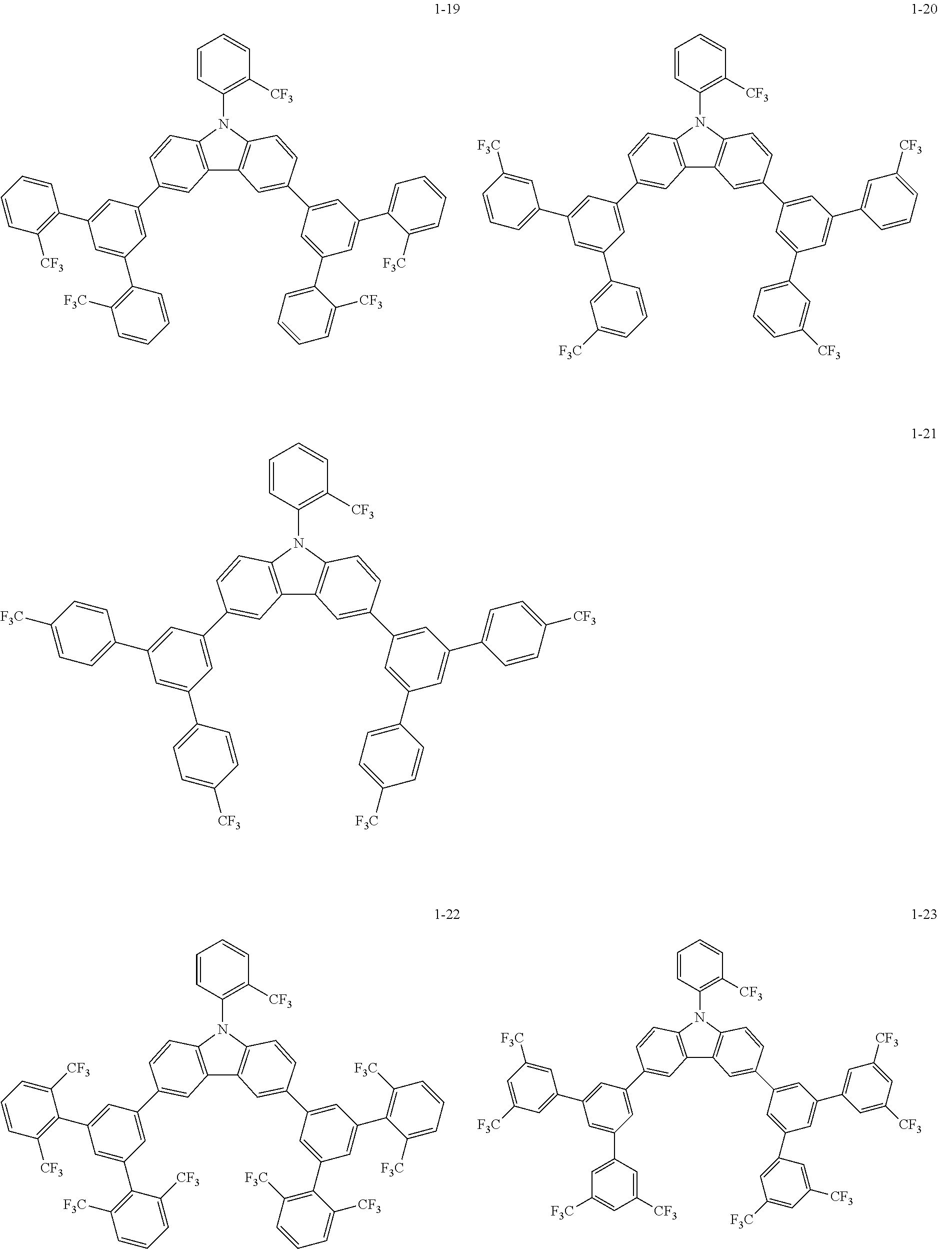

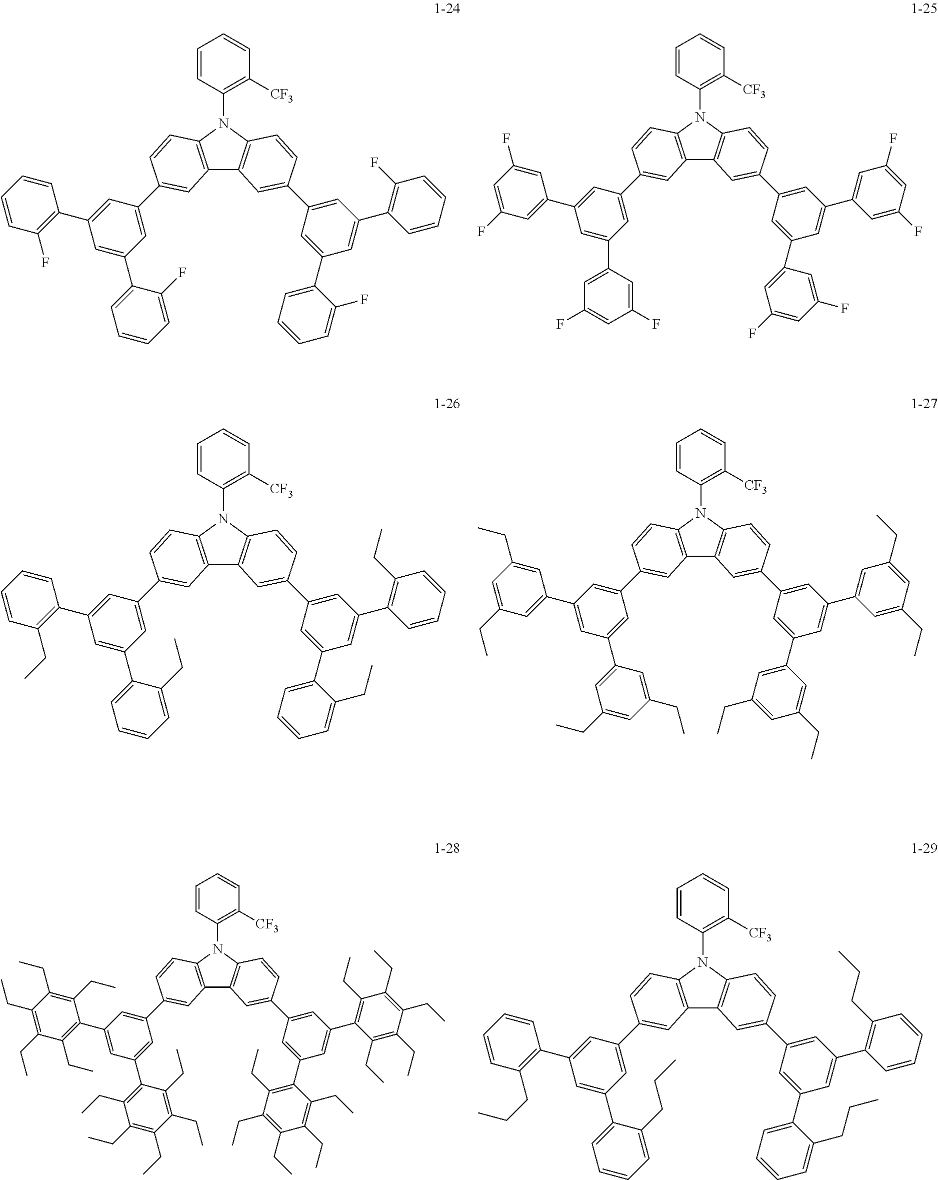

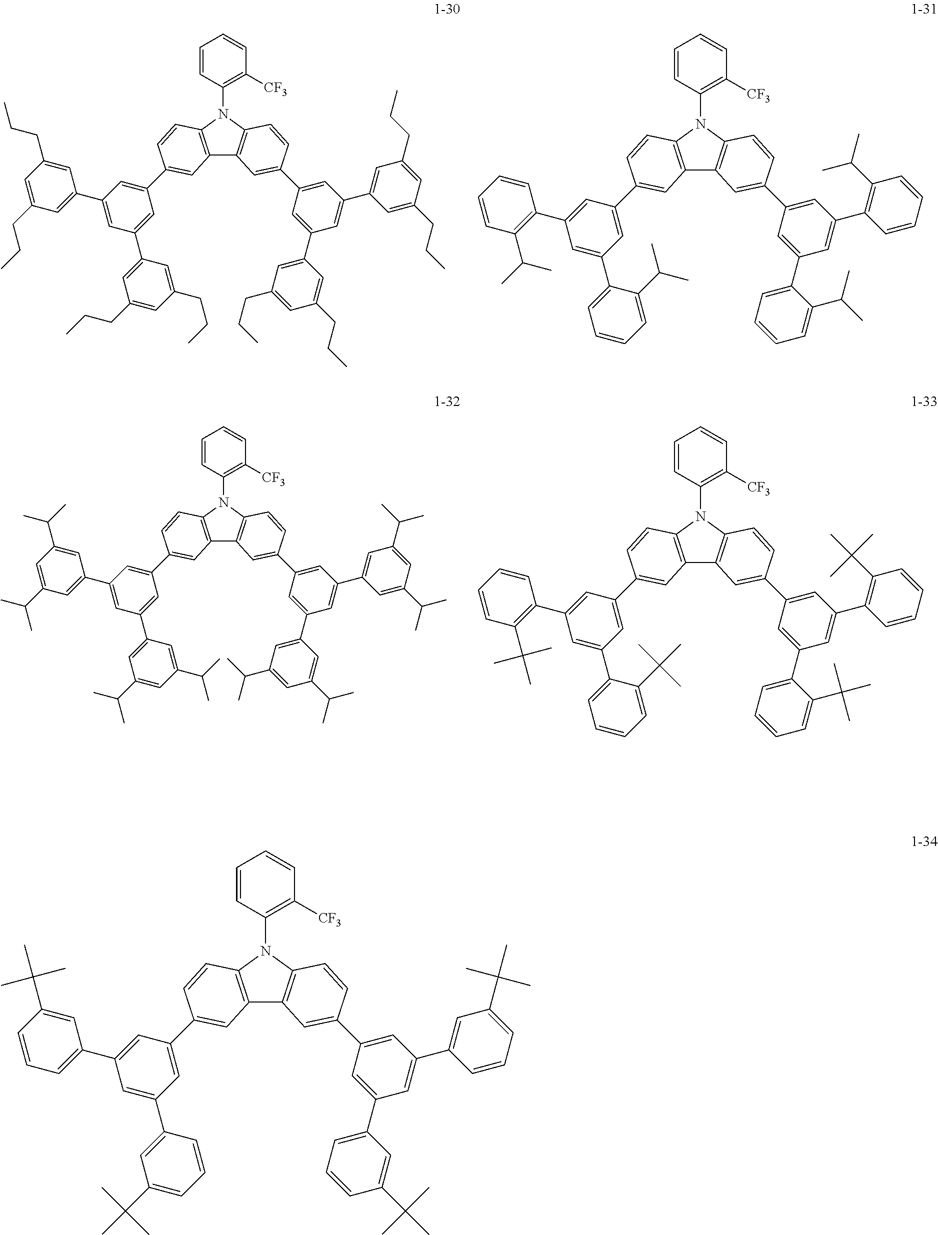

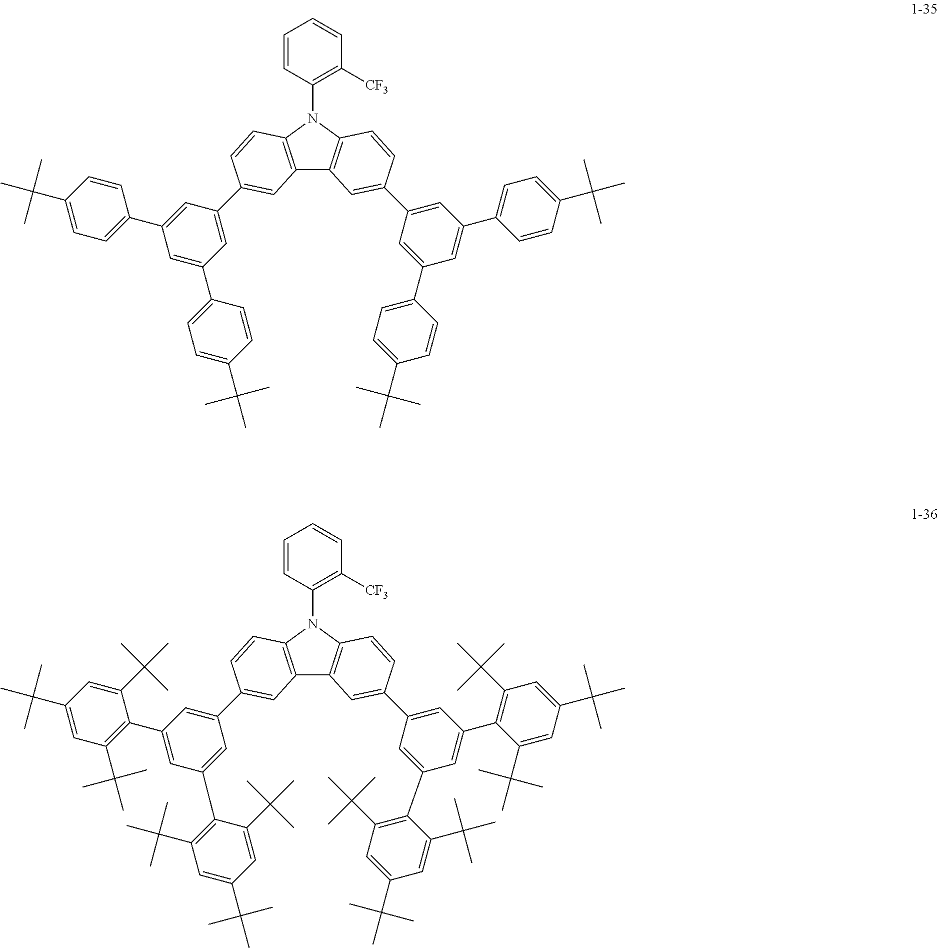

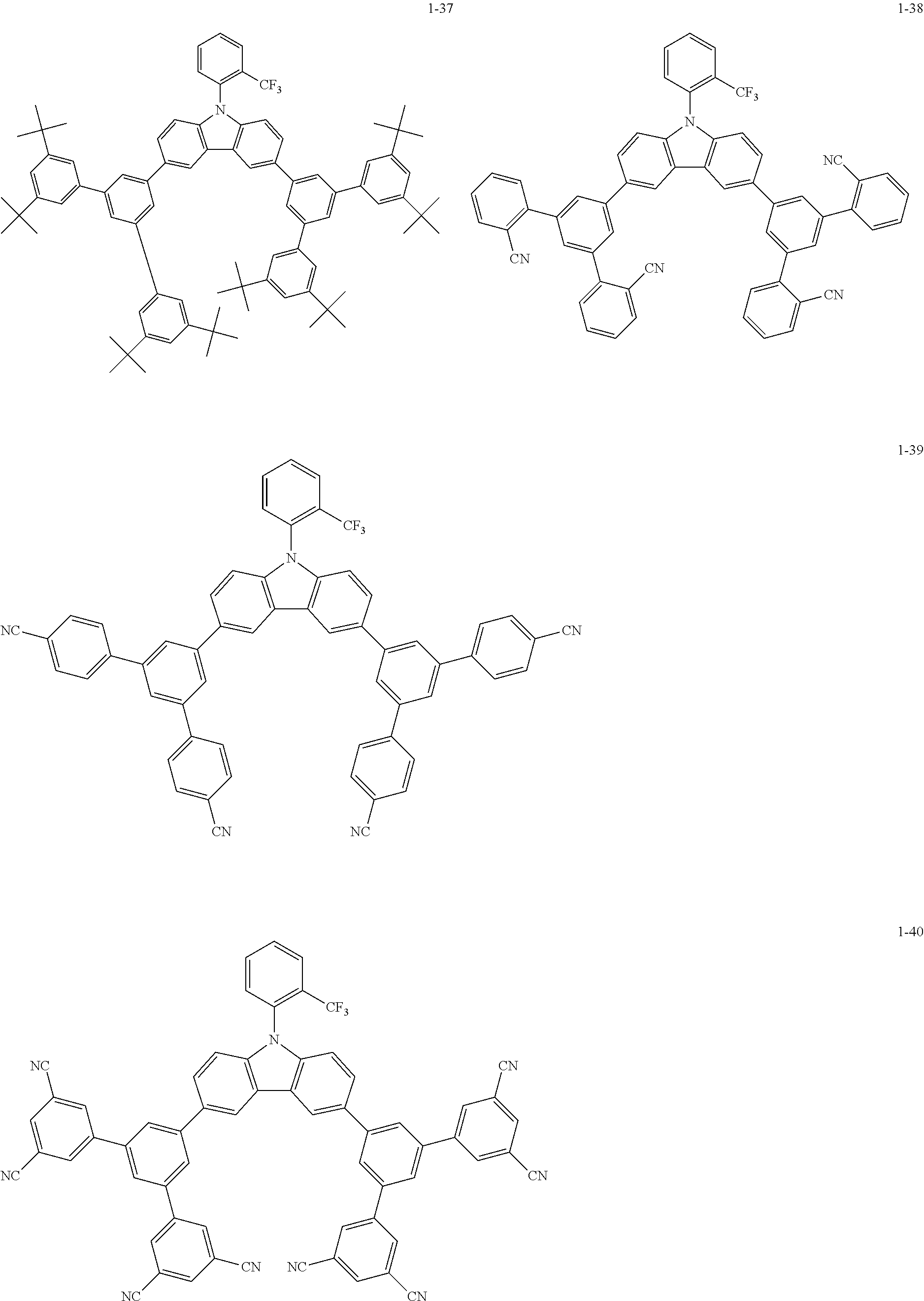

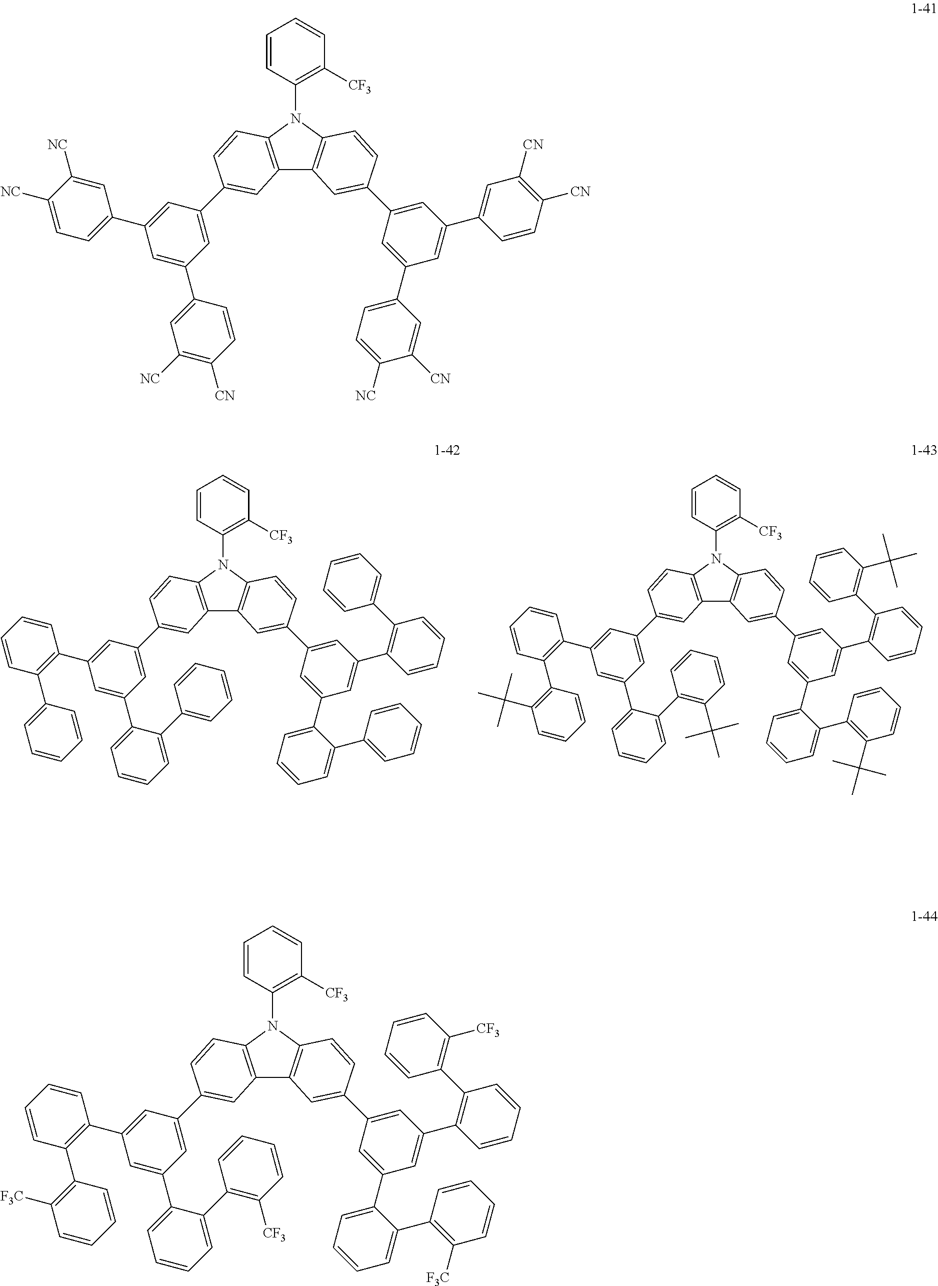

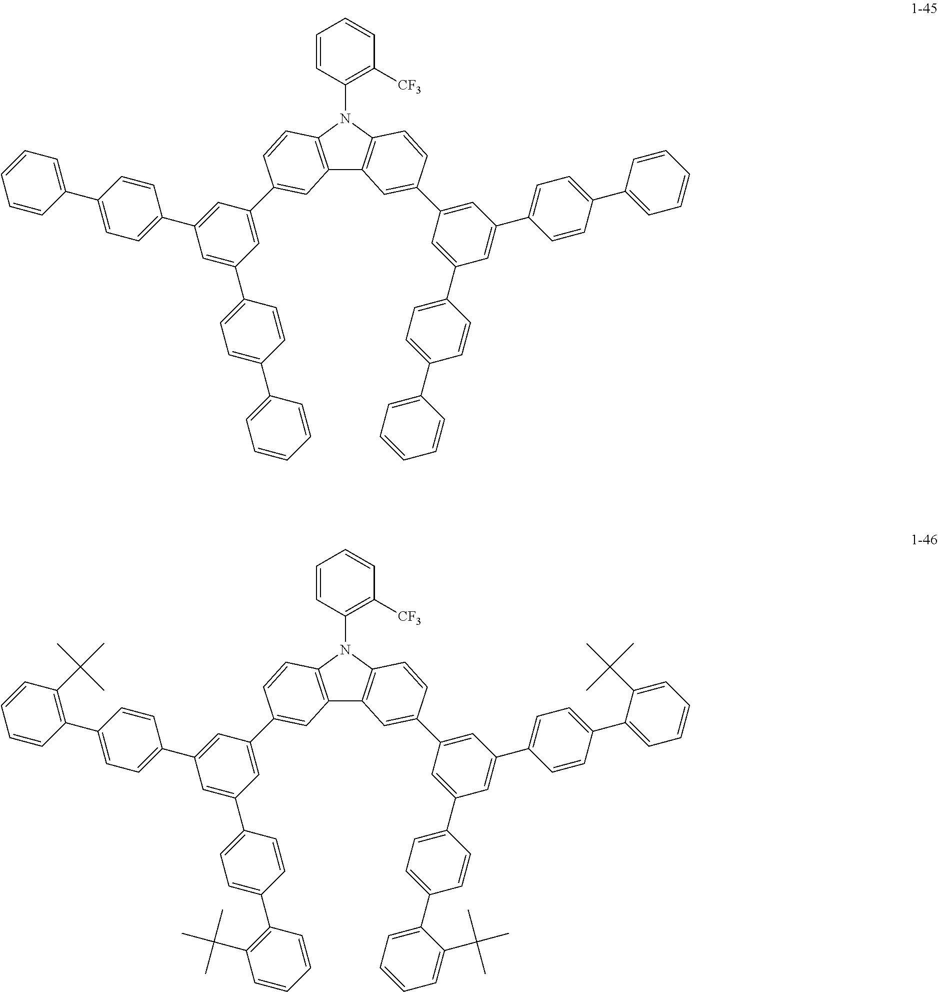 [SMILES: FC(F)(F)c1ccccc1-n1c2ccc(-c3cc(-c4ccc(-c5ccccc5)cc4)cc(-c4ccc(-c5ccccc5)cc4)c3)cc2c2cc(-c3cc(-c4ccc(-c5ccccc5)cc4)cc(-c4ccc(-c5ccccc5)cc4)c3)ccc21 ; CC(C)(C)c1ccccc1-c1ccc(-c2cc(-c3ccc(-c4ccccc4C(C)(C)C)cc3)cc(-c3ccc4c(c3)c3cc(-c5cc(-c6ccc(-c7ccccc7C(C)(C)C)cc6)cc(-c6ccc(-c7ccccc7C(C)(C)C)cc6)c5)ccc3n4-c3ccccc3C(F)(F)F)c2)cc1]

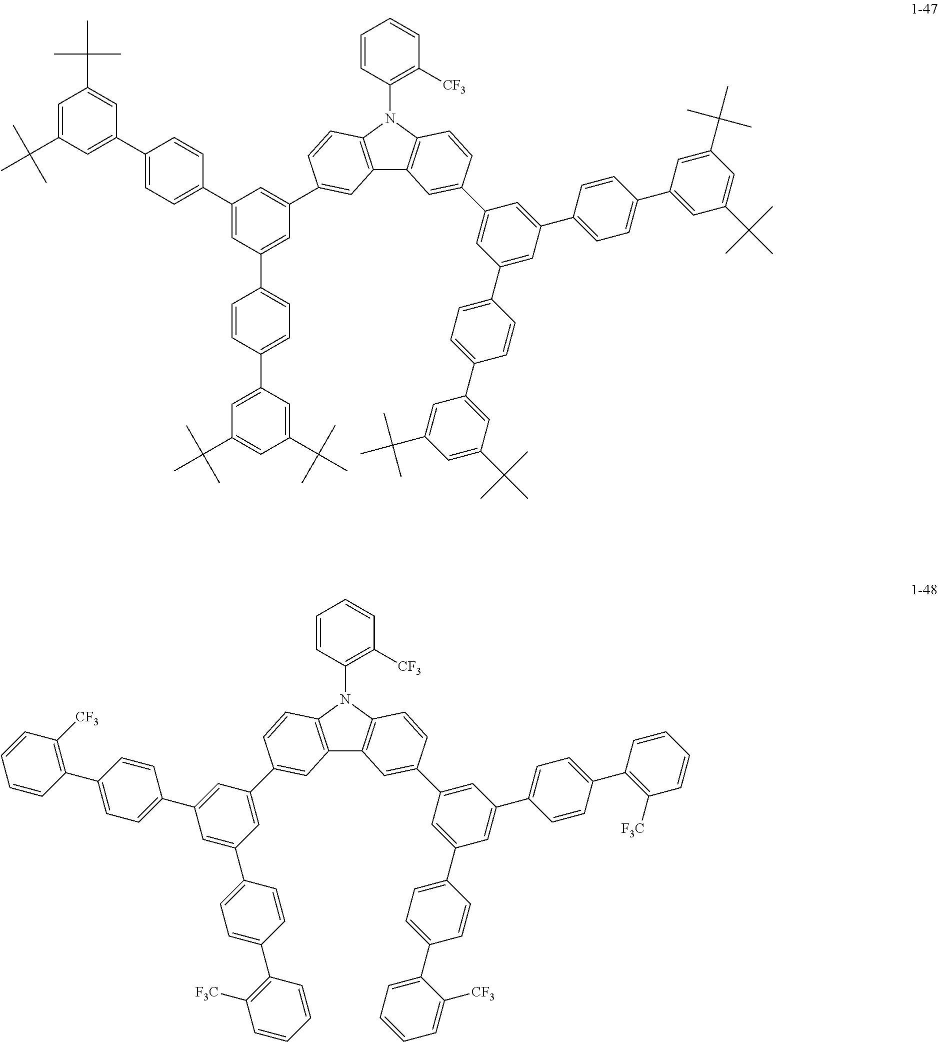 [SMILES: CC(C)(C)c1cc(-c2ccc(-c3cc(-c4ccc(-c5cc(C(C)(C)C)cc(C(C)(C)C)c5)cc4)cc(-c4ccc5c(c4)c4cc(-c6cc(-c7ccc(-c8cc(C(C)(C)C)cc(C(C)(C)C)c8)cc7)cc(-c7ccc(-c8cc(C(C)(C)C)cc(C(C)(C)C)c8)cc7)c6)ccc4n5-c4ccccc4C(F)(F)F)c3)cc2)cc(C(C)(C)C)c1 ; FC(F)(F)c1ccccc1-c1ccc(-c2cc(-c3ccc(-c4ccccc4C(F)(F)F)cc3)cc(-c3ccc4c(c3)c3cc(-c5cc(-c6ccc(-c7ccccc7C(F)(F)F)cc6)cc(-c6ccc(-c7ccccc7C(F)(F)F)cc6)c5)ccc3n4-c3ccccc3C(F)(F)F)c2)cc1]

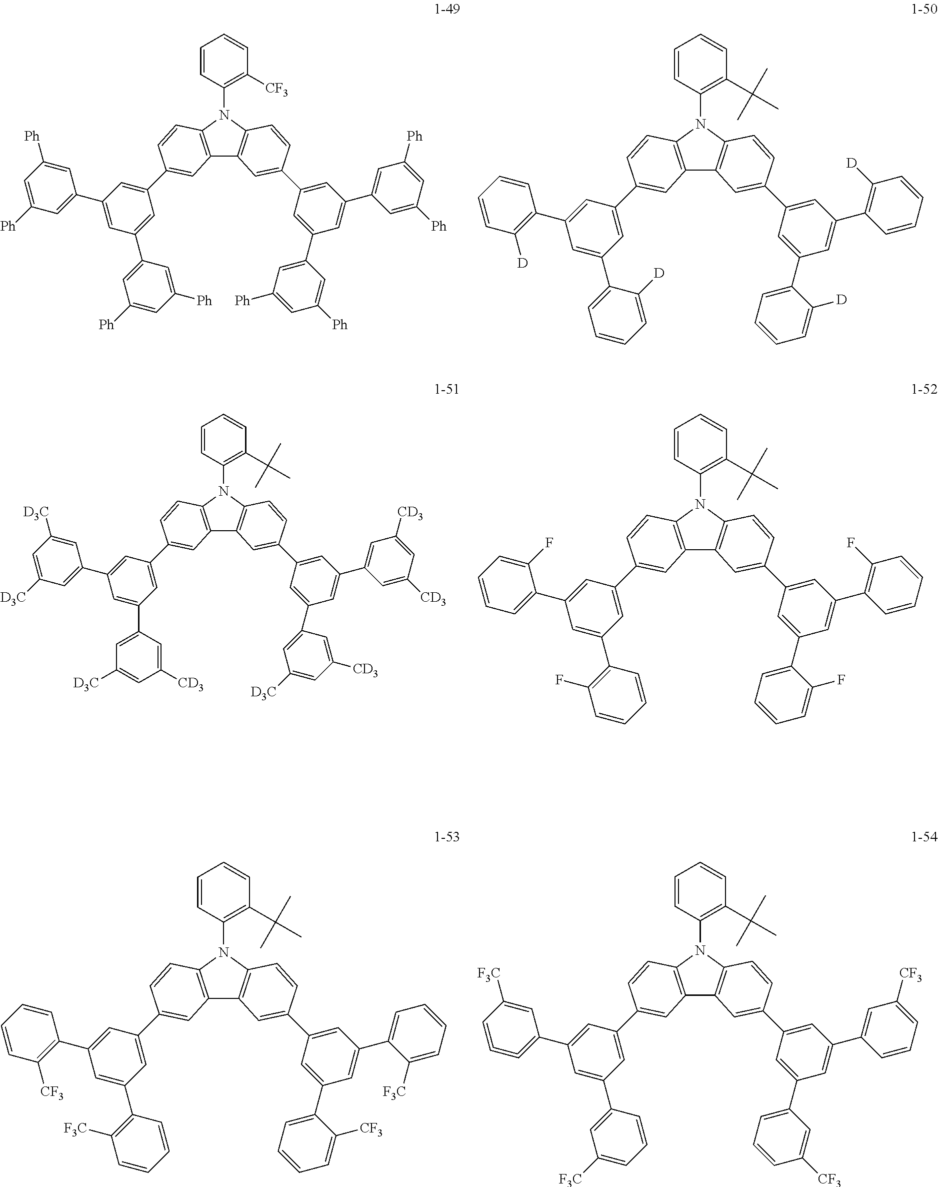

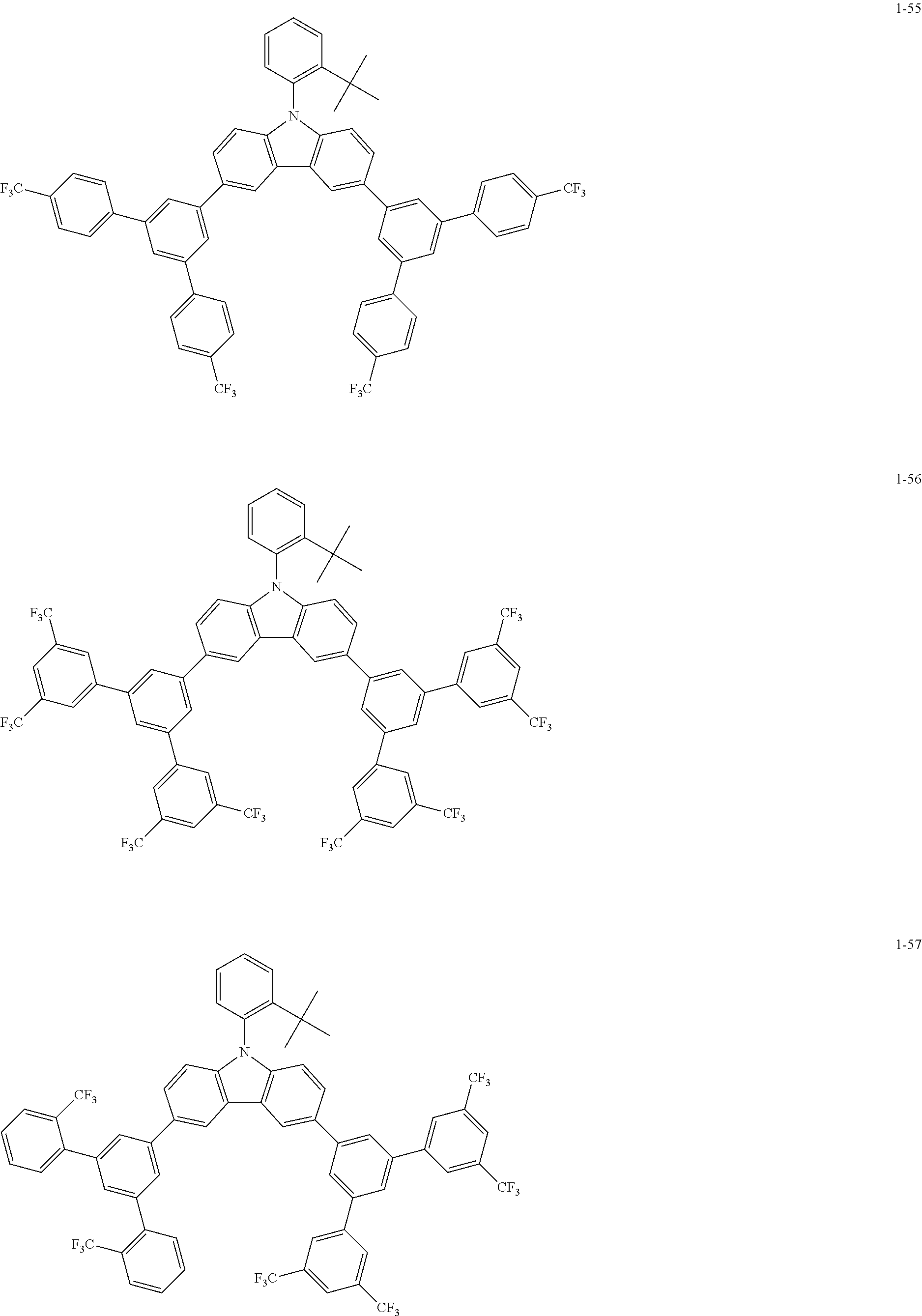 [SMILES: CC(C)(C)c1ccccc1-n1c2ccc(-c3cc(-c4ccc(C(F)(F)F)cc4)cc(-c4ccc(C(F)(F)F)cc4)c3)cc2c2cc(-c3cc(-c4ccc(C(F)(F)F)cc4)cc(-c4ccc(C(F)(F)F)cc4)c3)ccc21 ; CC(C)(C)c1ccccc1-n1c2ccc(-c3cc(-c4cc(C(F)(F)F)cc(C(F)(F)F)c4)cc(-c4cc(C(F)(F)F)cc(C(F)(F)F)c4)c3)cc2c2cc(-c3cc(-c4cc(C(F)(F)F)cc(C(F)(F)F)c4)cc(-c4cc(C(F)(F)F)cc(C(F)(F)F)c4)c3)ccc21 ; CC(C)(C)c1ccccc1-n1c2ccc(-c3cc(-c4cc(C(F)(F)F)cc(C(F)(F)F)c4)cc(-c4cc(C(F)(F)F)cc(C(F)(F)F)c4)c3)cc2c2cc(-c3cc(-c4ccccc4C(F)(F)F)cc(-c4ccccc4C(F)(F)F)c3)ccc21]

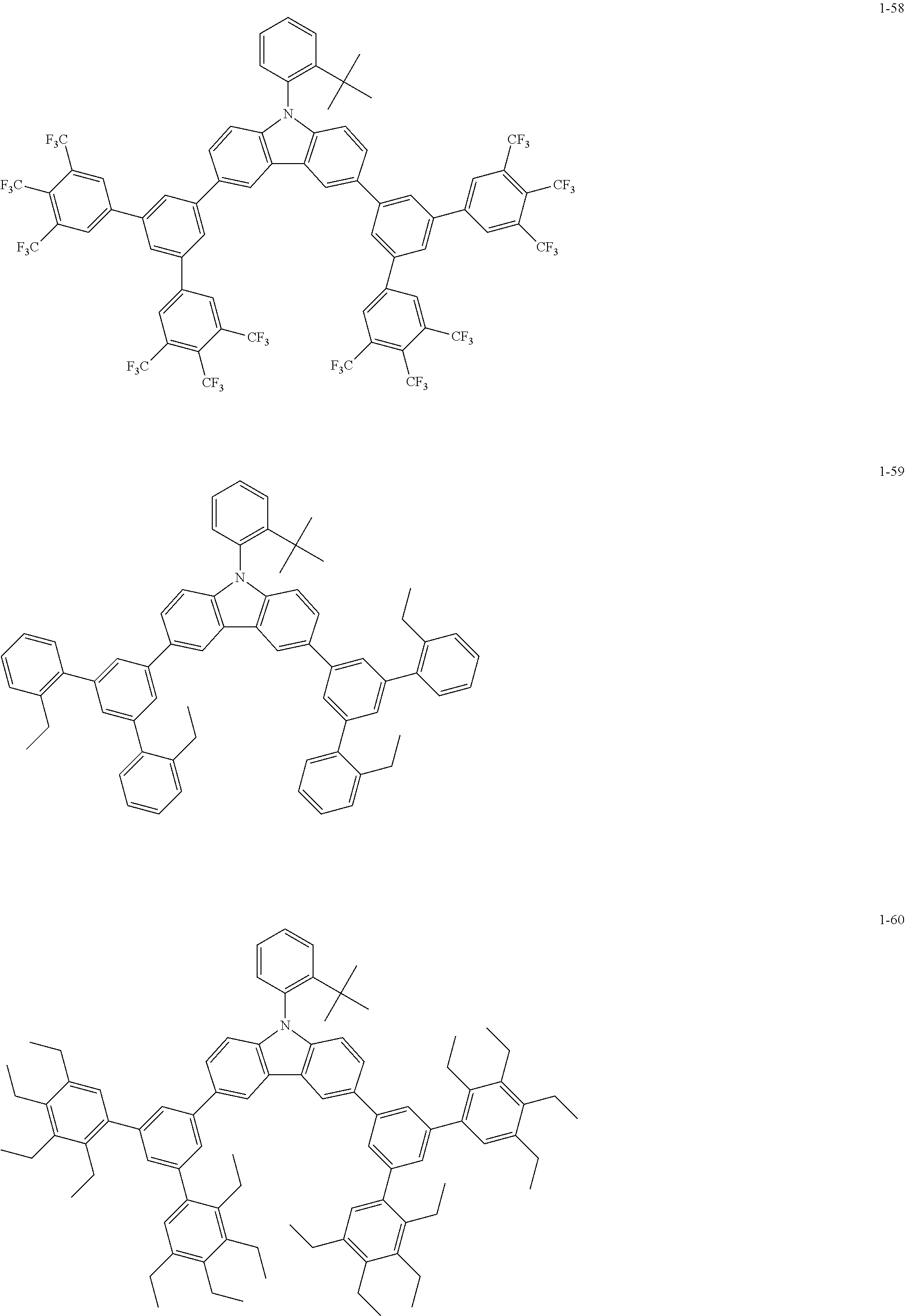 [SMILES: CC(C)(C)c1ccccc1-n1c2ccc(-c3cc(-c4cc(C(F)(F)F)c(C(F)(F)F)c(C(F)(F)F)c4)cc(-c4cc(C(F)(F)F)c(C(F)(F)F)c(C(F)(F)F)c4)c3)cc2c2cc(-c3cc(-c4cc(C(F)(F)F)c(C(F)(F)F)c(C(F)(F)F)c4)cc(-c4cc(C(F)(F)F)c(C(F)(F)F)c(C(F)(F)F)c4)c3)ccc21 ; CCc1ccccc1-c1cc(-c2ccc3c(c2)c2cc(-c4cc(-c5ccccc5CC)cc(-c5ccccc5CC)c4)ccc2n3-c2ccccc2C(C)(C)C)cc(-c2ccccc2CC)c1 ; CCc1cc(-c2cc(-c3ccc4c(c3)c3cc(-c5cc(-c6cc(CC)c(CC)c(CC)c6CC)cc(-c6cc(CC)c(CC)c(CC)c6CC)c5)ccc3n4-c3ccccc3C(C)(C)C)cc(-c3cc(CC)c(CC)c(CC)c3CC)c2)c(CC)c(CC)c1CC]

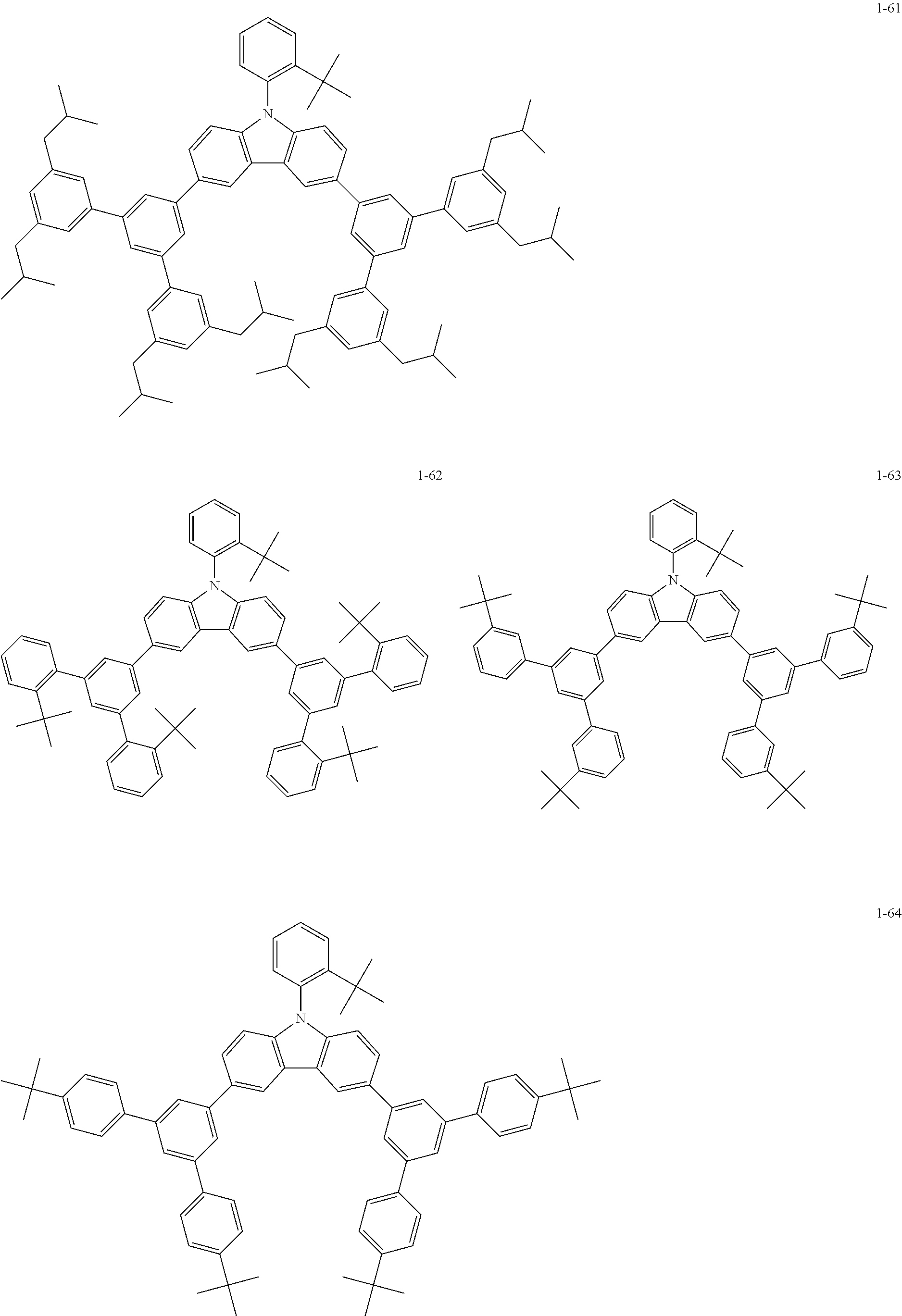

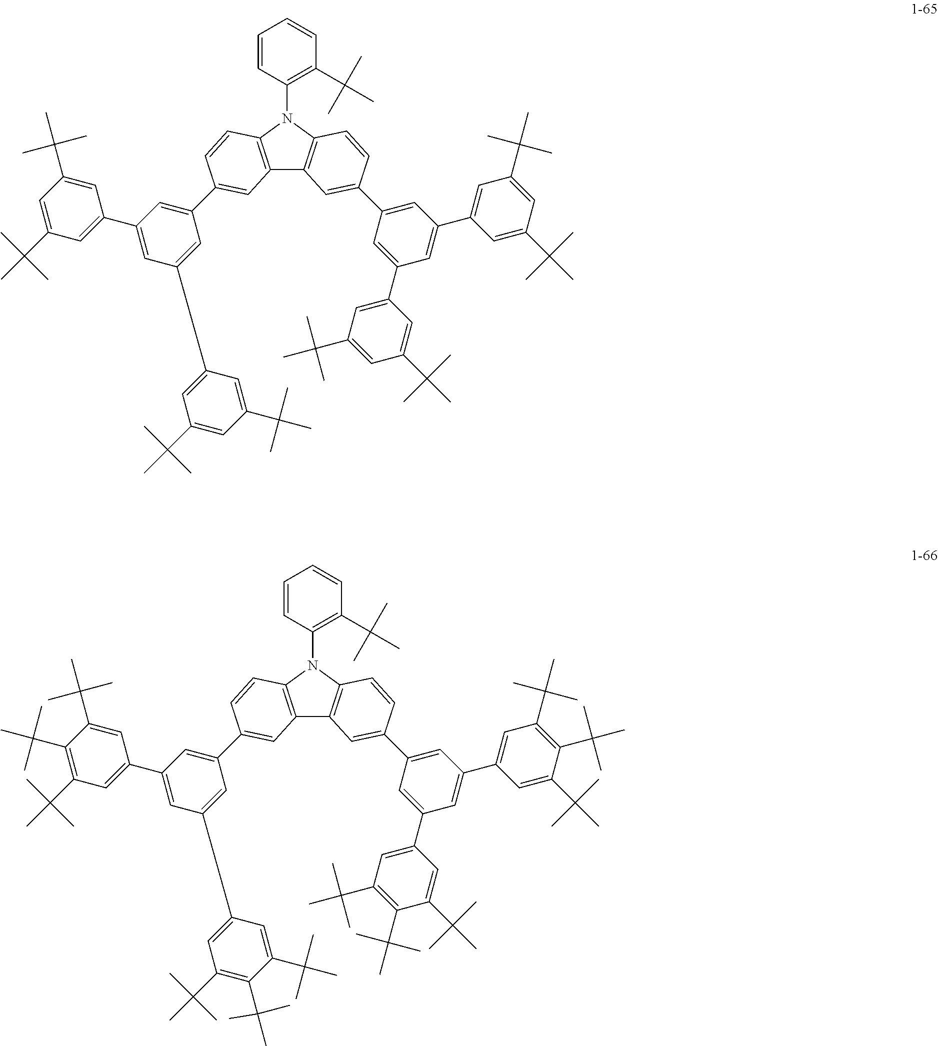

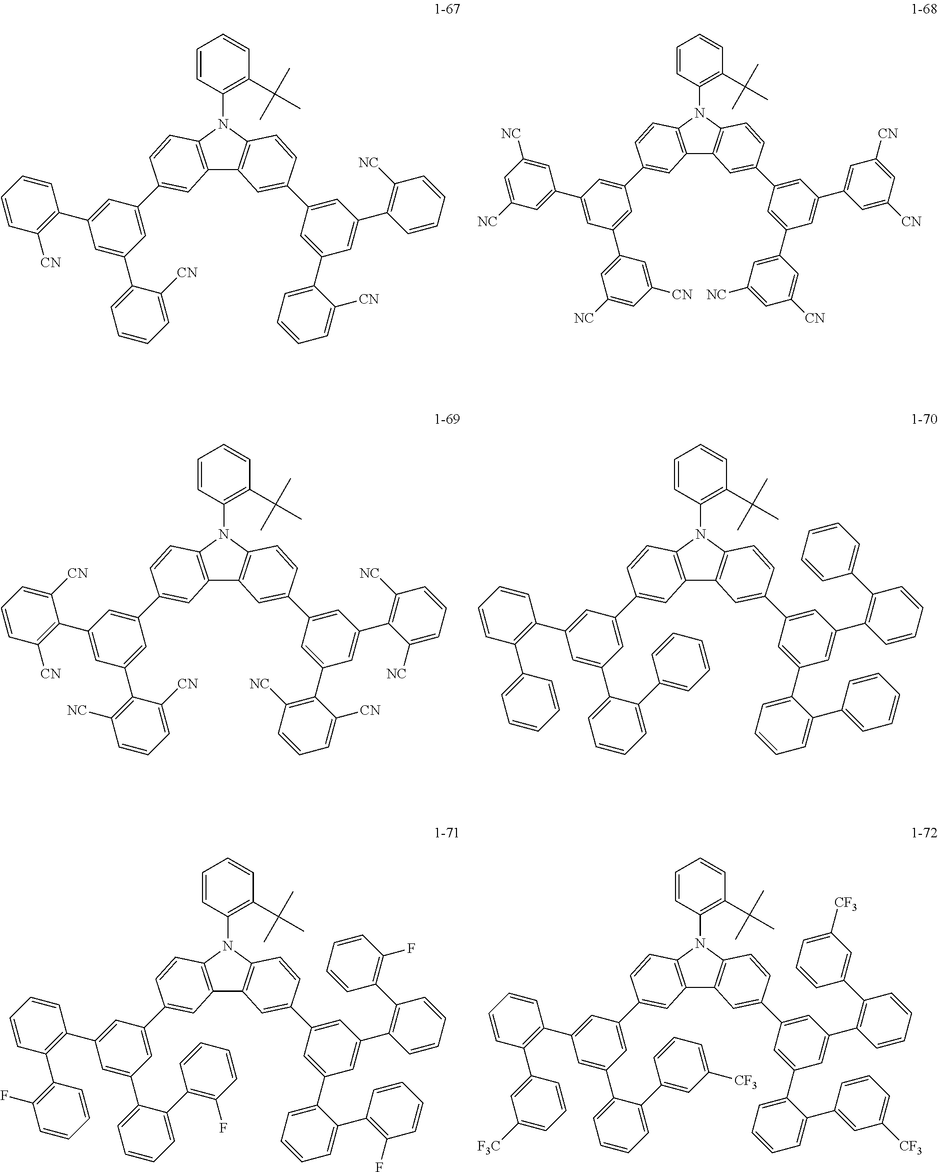

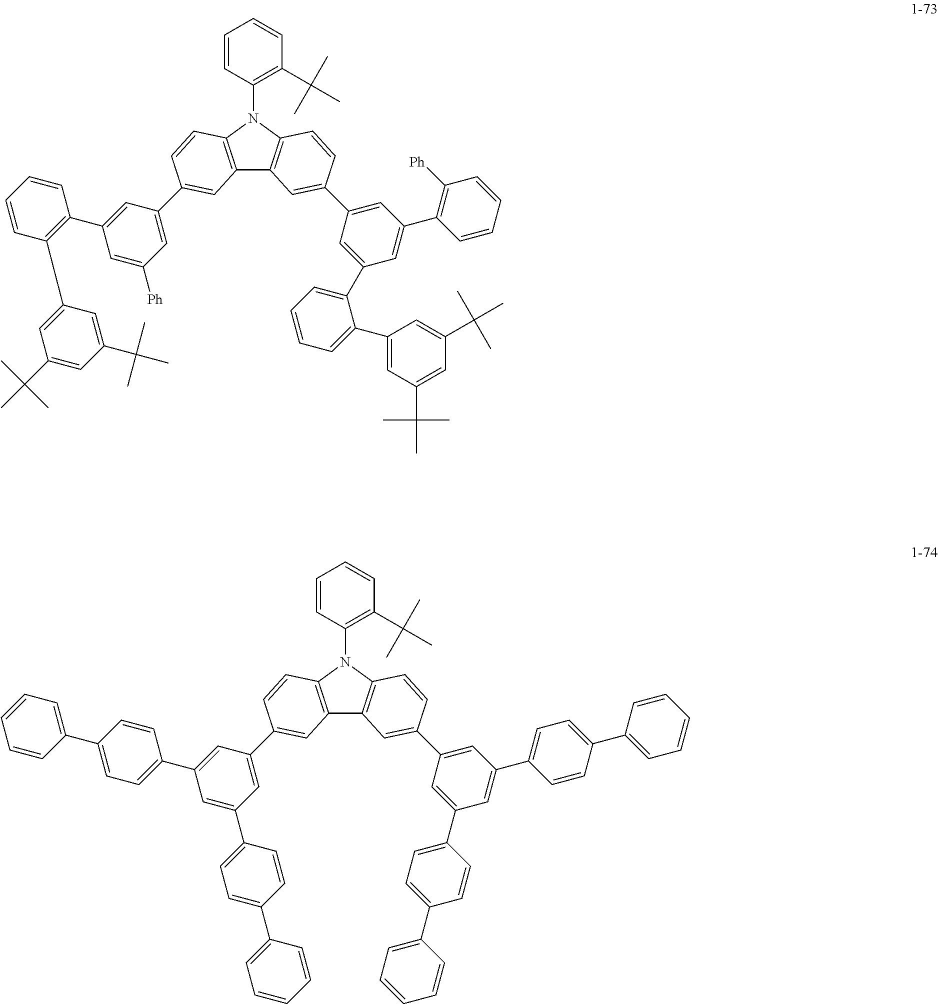 [SMILES: CC(C)(C)c1cc(-c2ccccc2-c2cc(-c3ccccc3)cc(-c3ccc4c(c3)c3cc(-c5cc(-c6ccccc6-c6ccccc6)cc(-c6ccccc6-c6cc(C(C)(C)C)cc(C(C)(C)C)c6)c5)ccc3n4-c3ccccc3C(C)(C)C)c2)cc(C(C)(C)C)c1 ; CC(C)(C)c1ccccc1-n1c2ccc(-c3cc(-c4ccc(-c5ccccc5)cc4)cc(-c4ccc(-c5ccccc5)cc4)c3)cc2c2cc(-c3cc(-c4ccc(-c5ccccc5)cc4)cc(-c4ccc(-c5ccccc5)cc4)c3)ccc21]

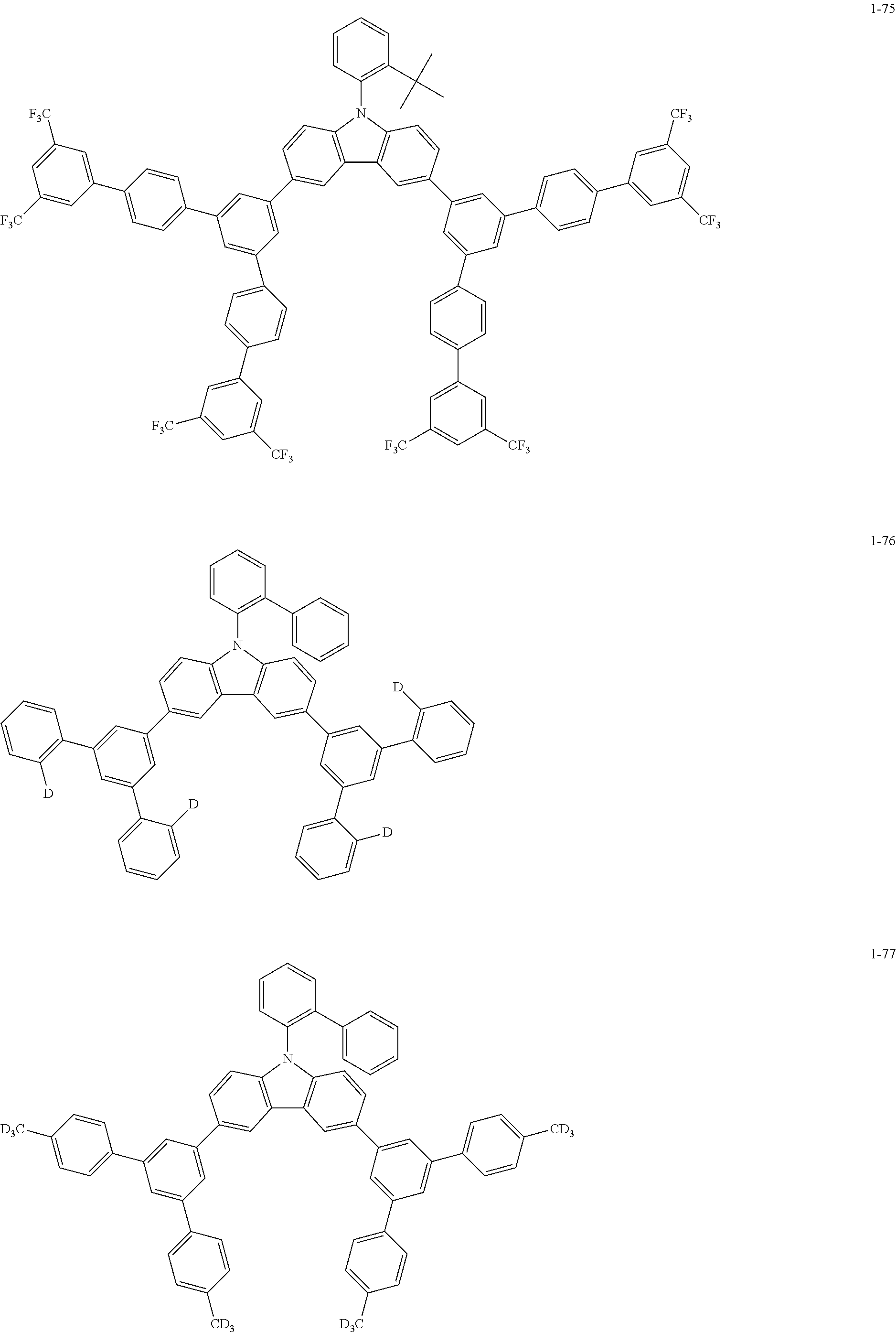

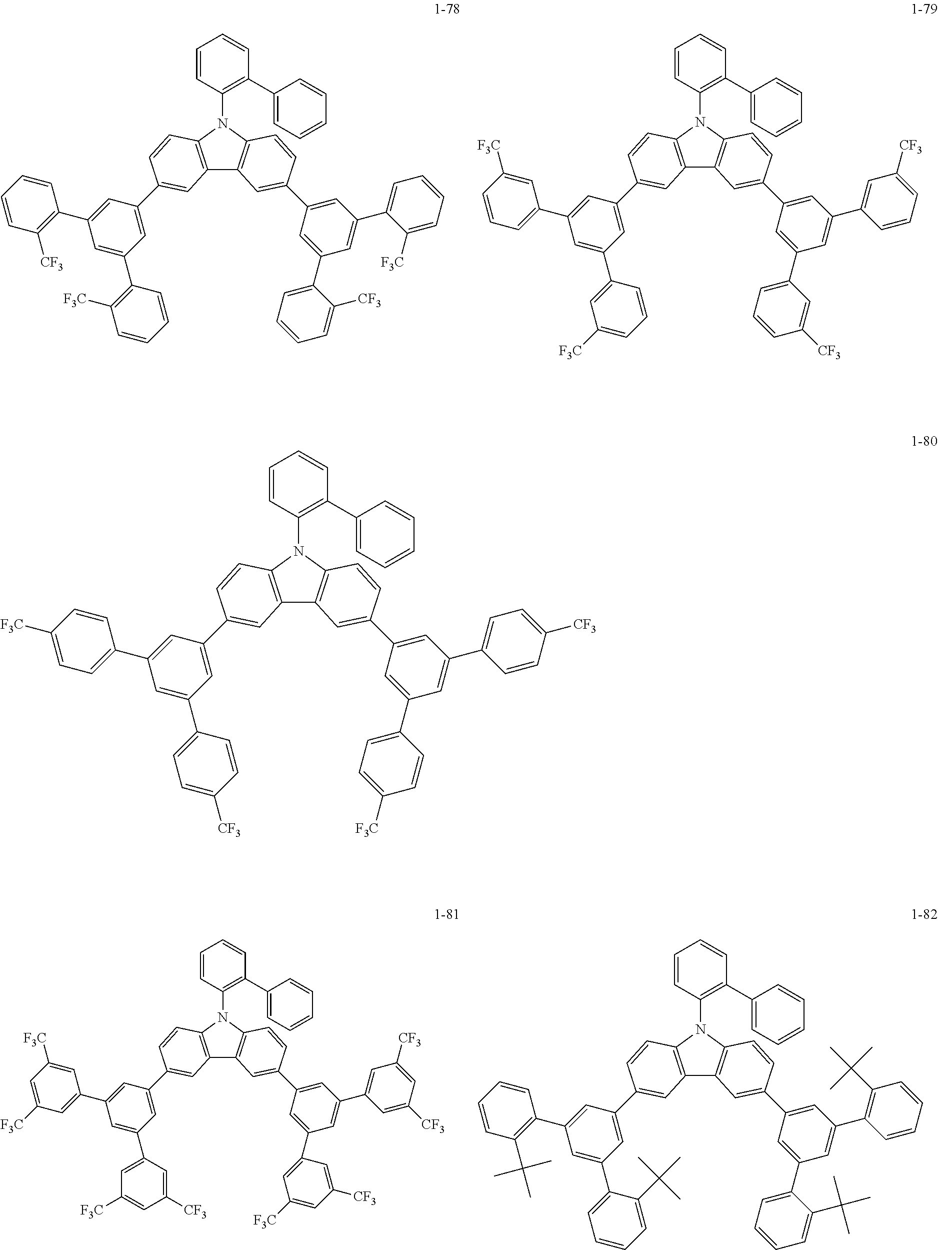

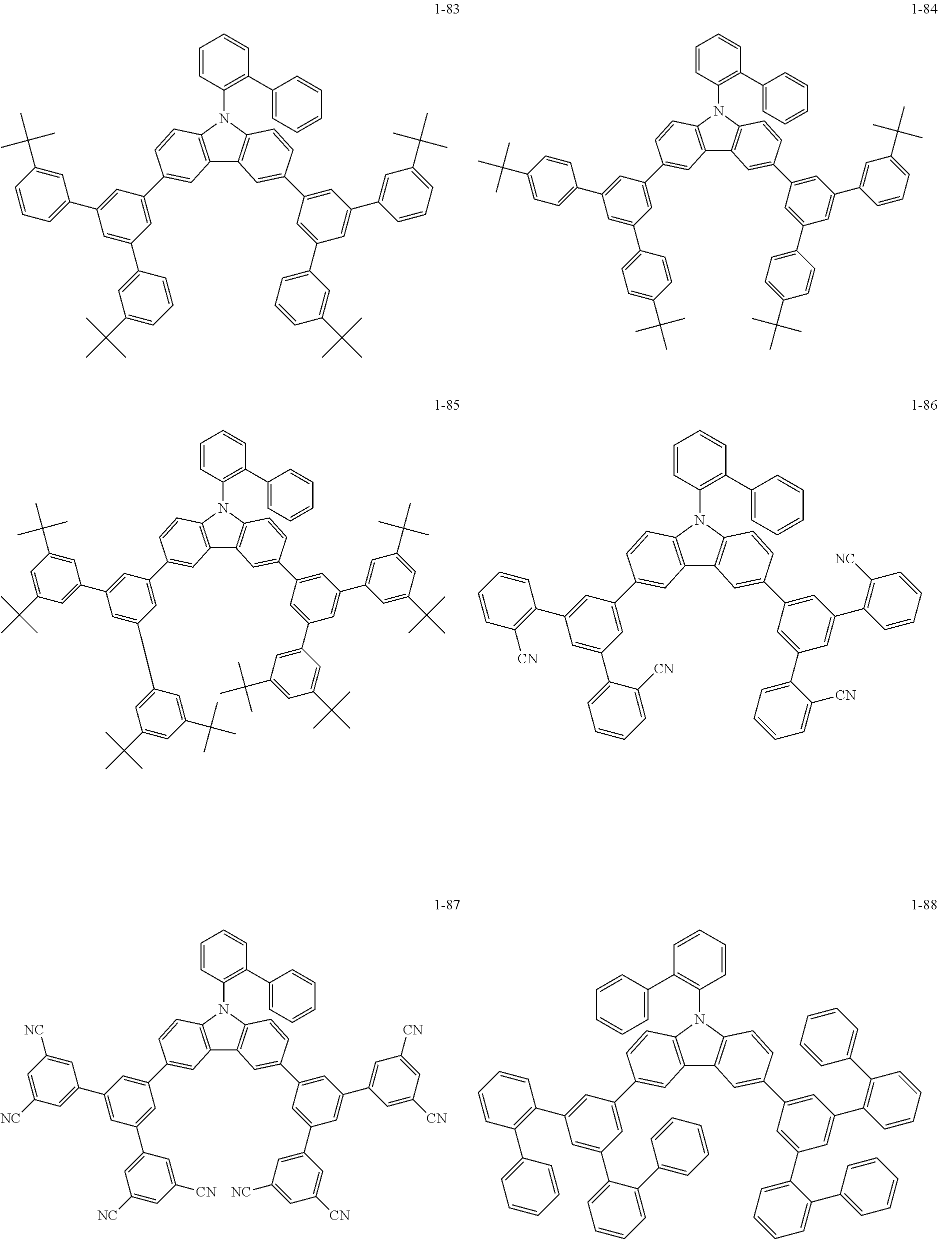

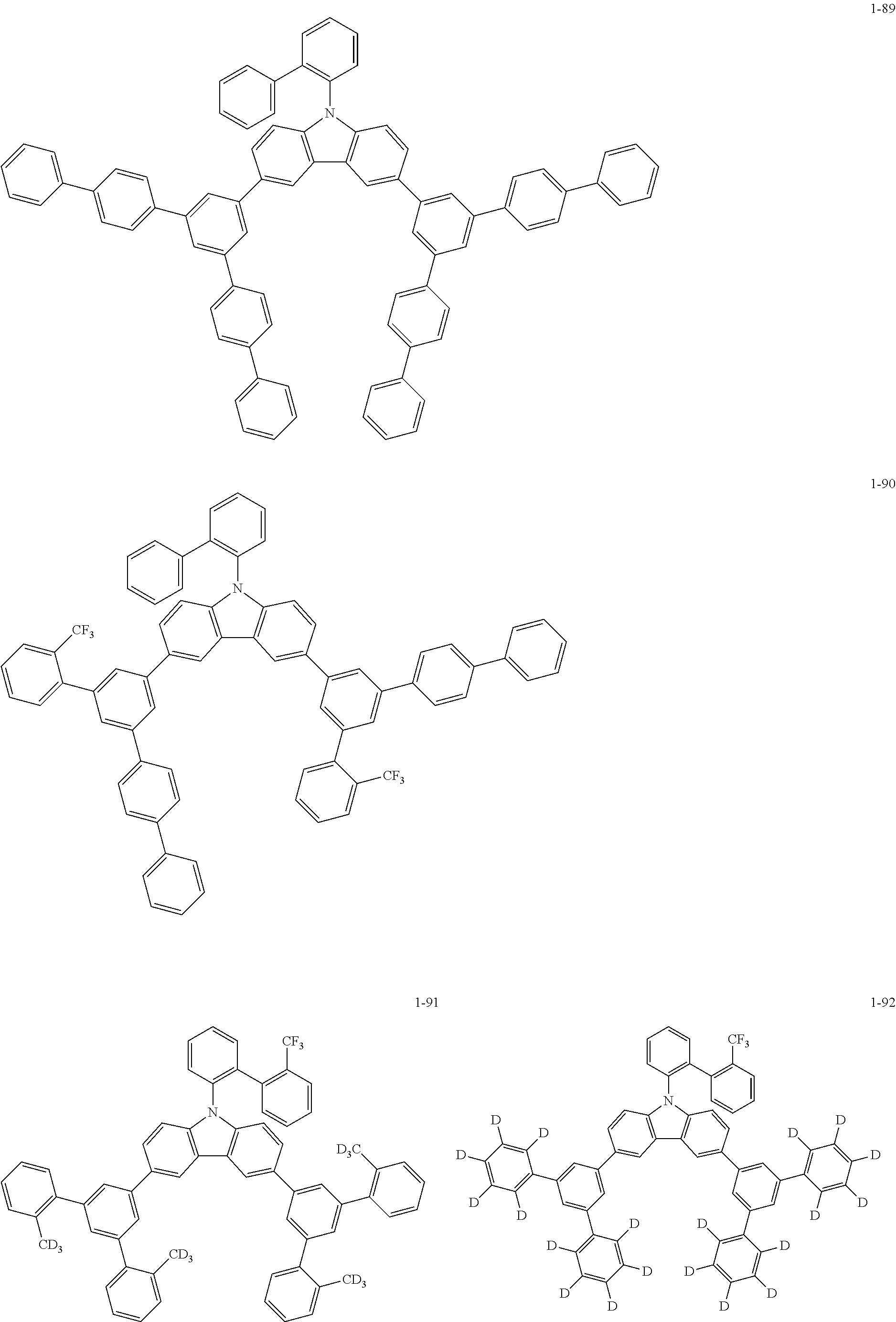

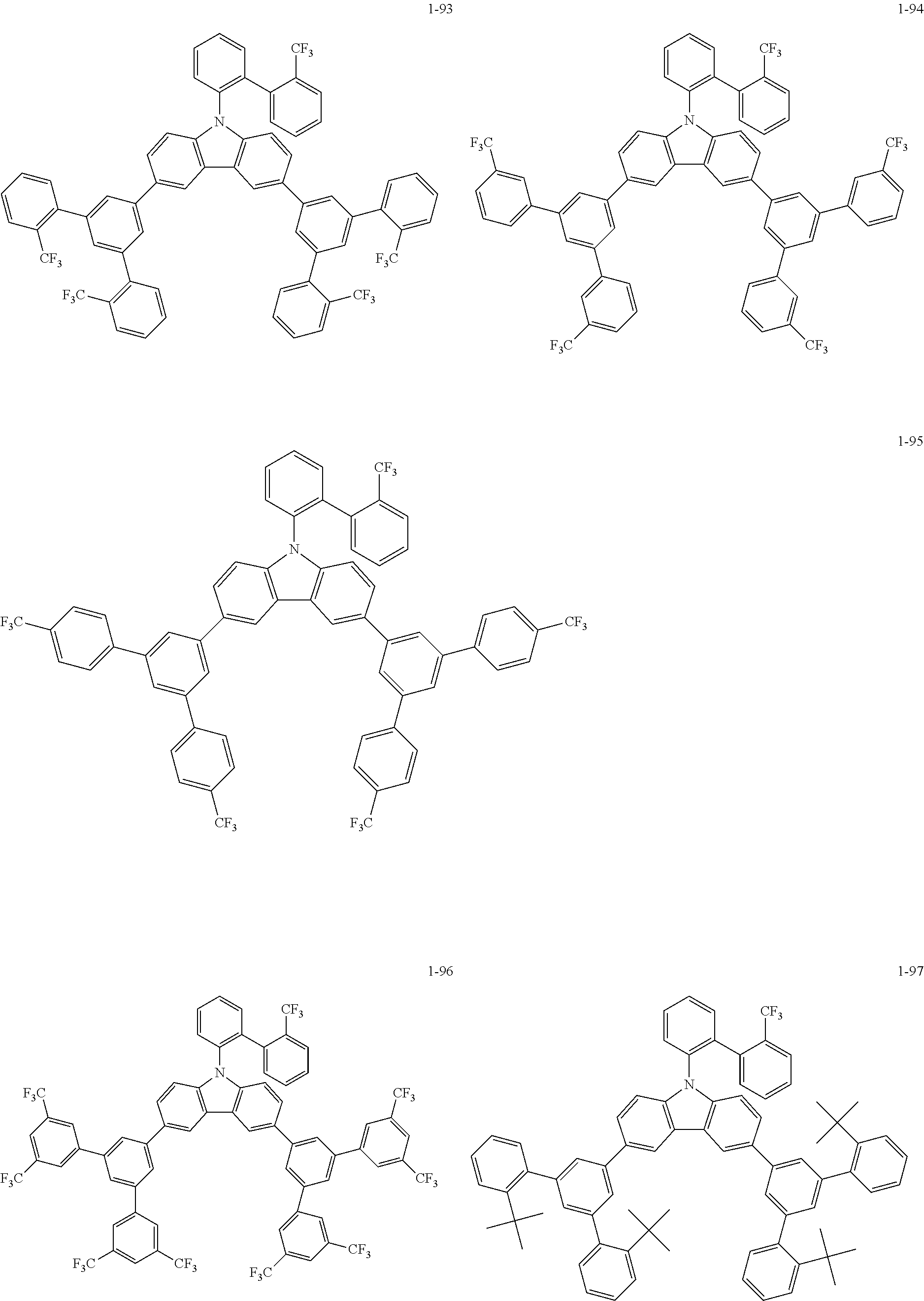

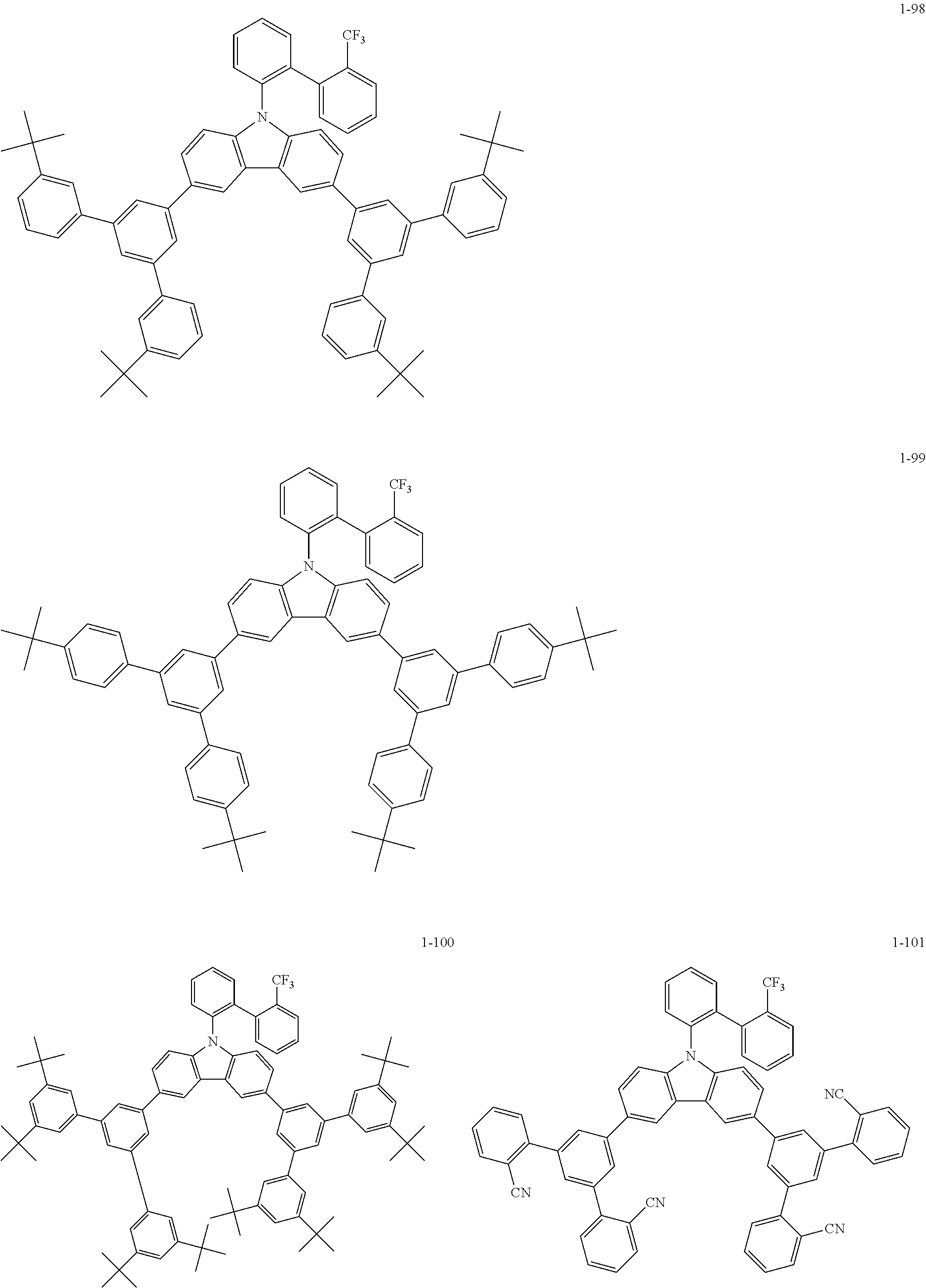

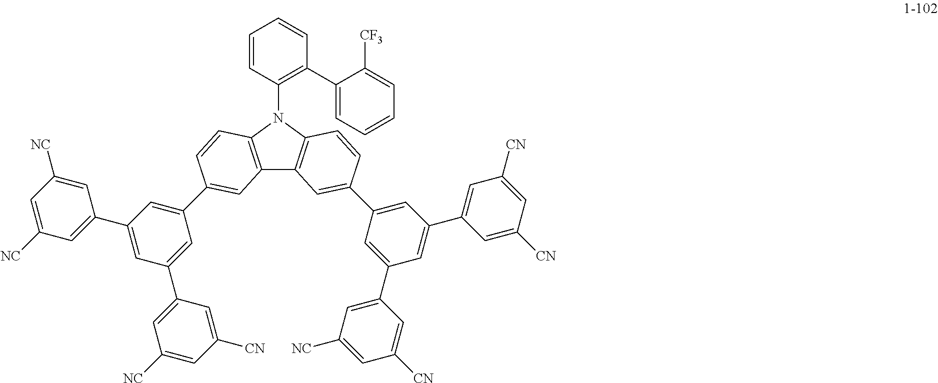 [SMILES: N#Cc1cc(C#N)cc(-c2cc(-c3cc(C#N)cc(C#N)c3)cc(-c3ccc4c(c3)c3cc(-c5cc(-c6cc(C#N)cc(C#N)c6)cc(-c6cc(C#N)cc(C#N)c6)c5)ccc3n4-c3ccccc3-c3ccccc3C(F)(F)F)c2)c1]

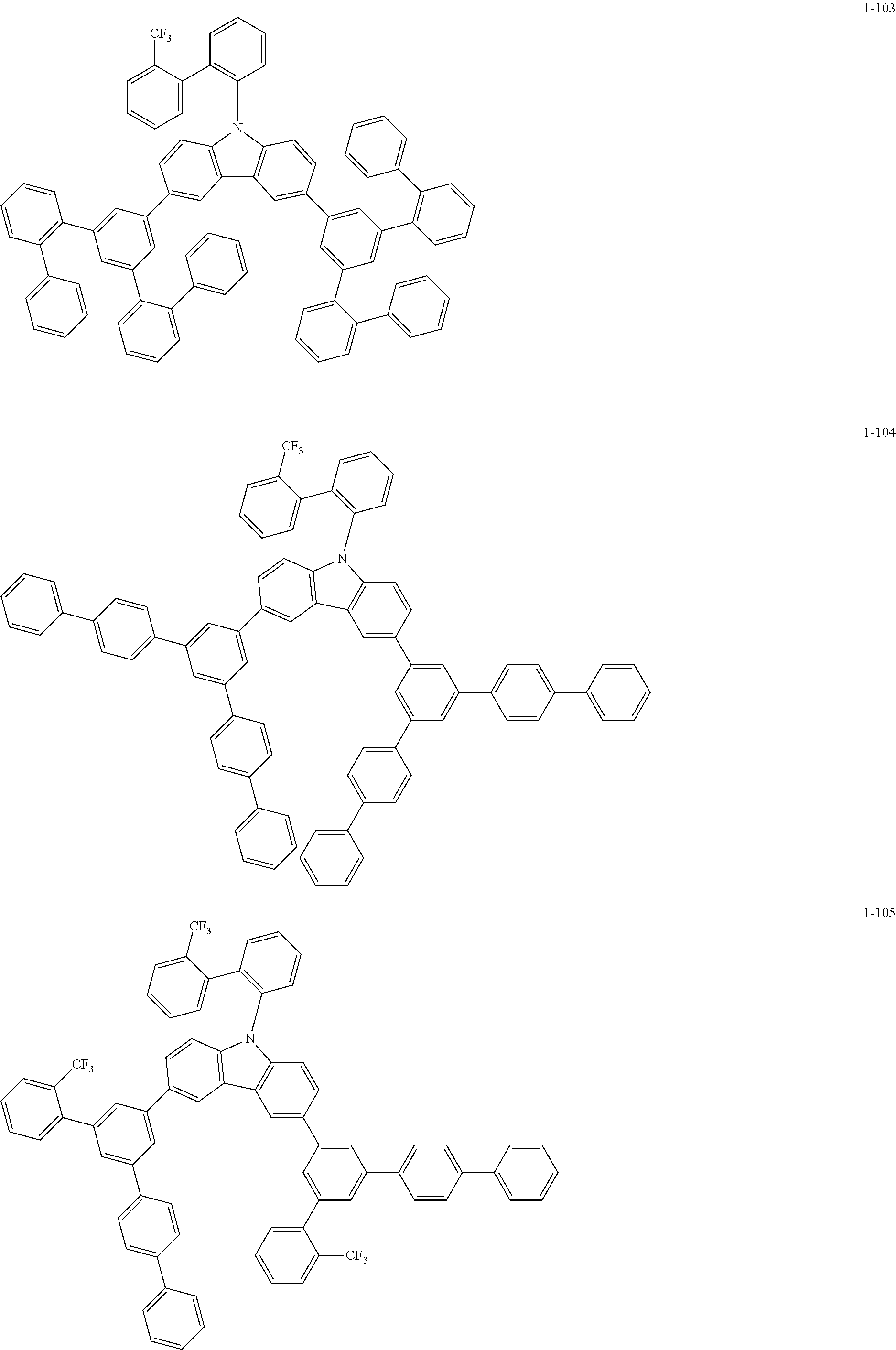 [SMILES: FC(F)(F)c1ccccc1-c1ccccc1-n1c2ccc(-c3cc(-c4ccccc4-c4ccccc4)cc(-c4ccccc4-c4ccccc4)c3)cc2c2cc(-c3cc(-c4ccccc4-c4ccccc4)cc(-c4ccccc4-c4ccccc4)c3)ccc21 ; FC(F)(F)c1ccccc1-c1ccccc1-n1c2ccc(-c3cc(-c4ccc(-c5ccccc5)cc4)cc(-c4ccc(-c5ccccc5)cc4)c3)cc2c2cc(-c3cc(-c4ccc(-c5ccccc5)cc4)cc(-c4ccc(-c5ccccc5)cc4)c3)ccc21 ; FC(F)(F)c1ccccc1-c1cc(-c2ccc(-c3ccccc3)cc2)cc(-c2ccc3c(c2)c2cc(-c4cc(-c5ccc(-c6ccccc6)cc5)cc(-c5ccccc5C(F)(F)F)c4)ccc2n3-c2ccccc2-c2ccccc2C(F)(F)F)c1]

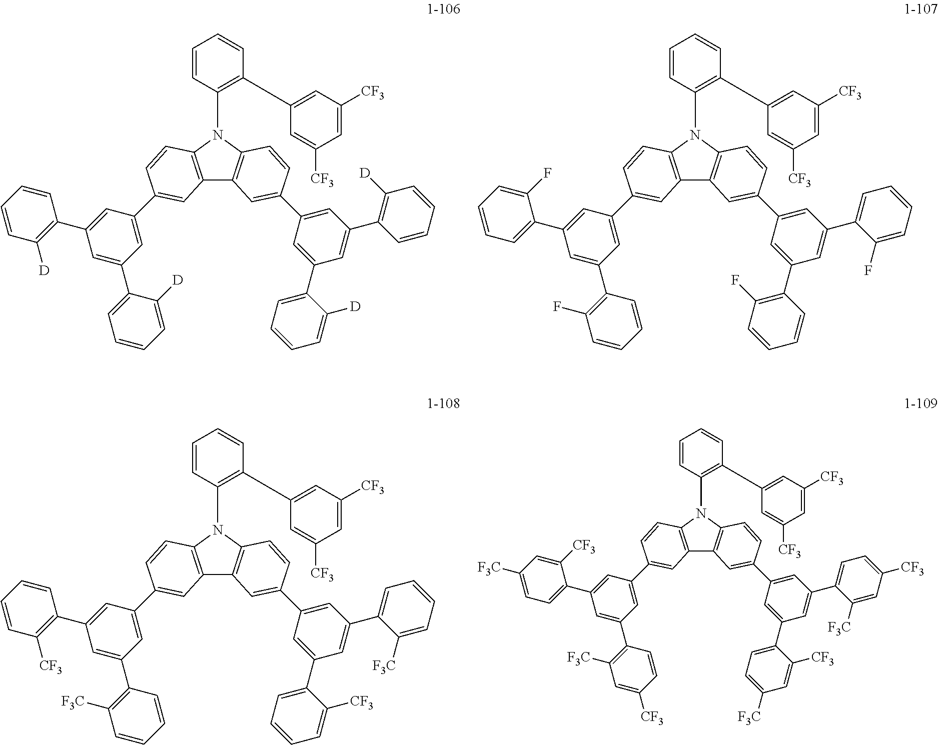

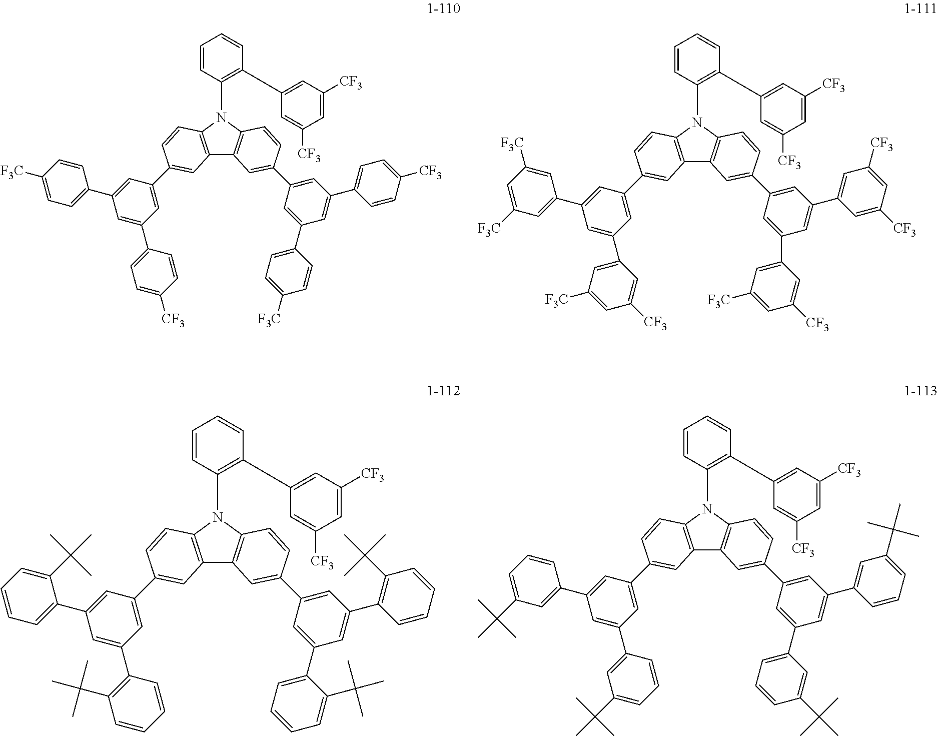

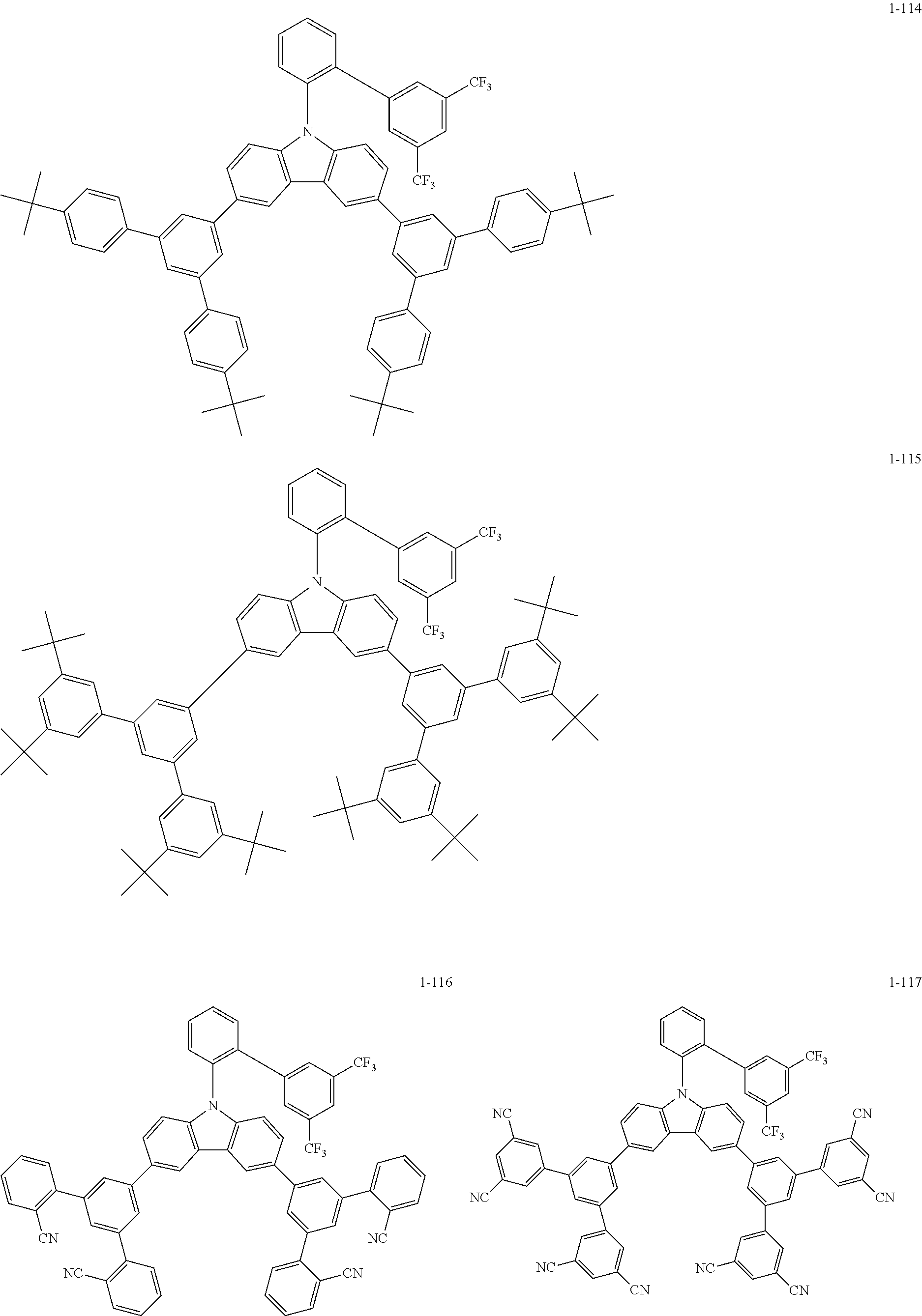

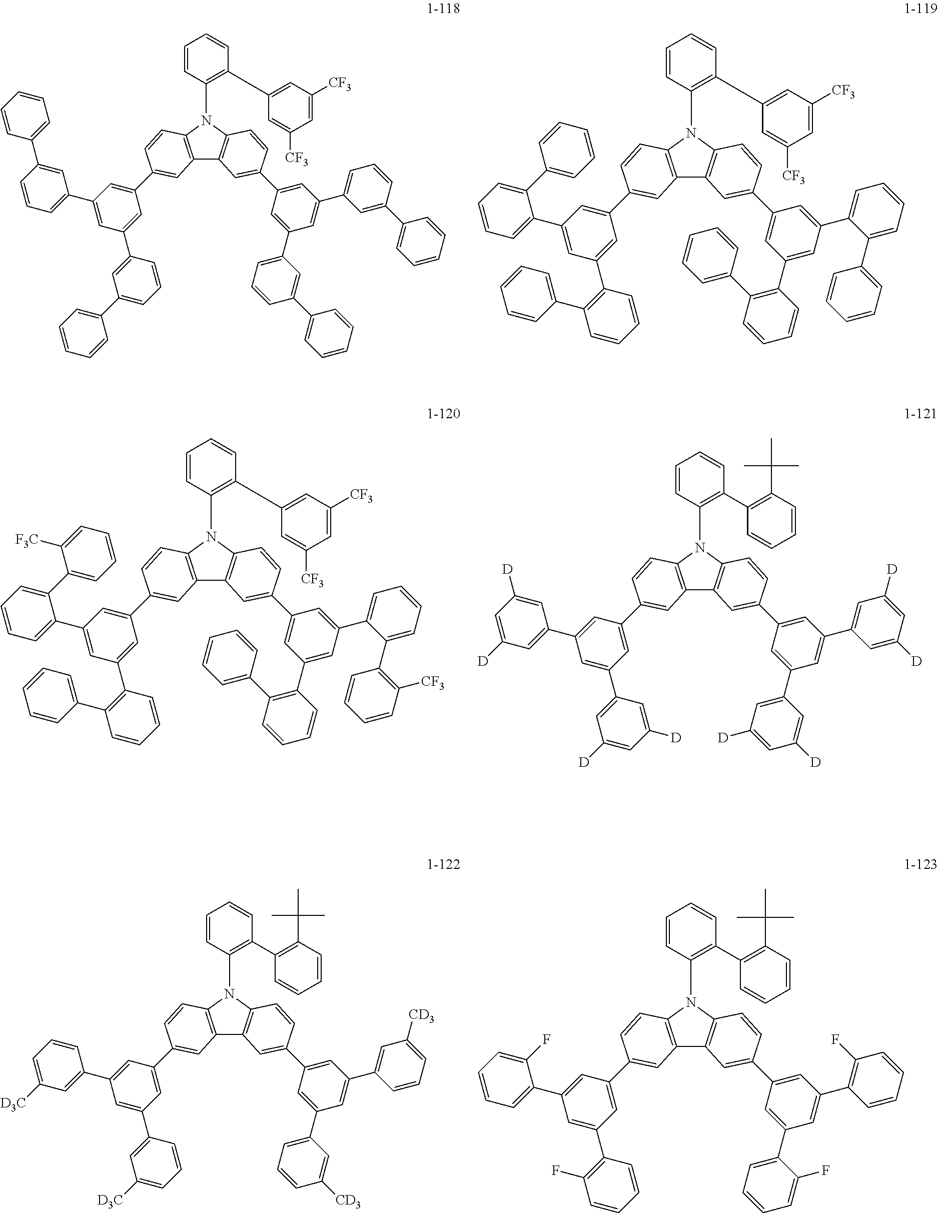

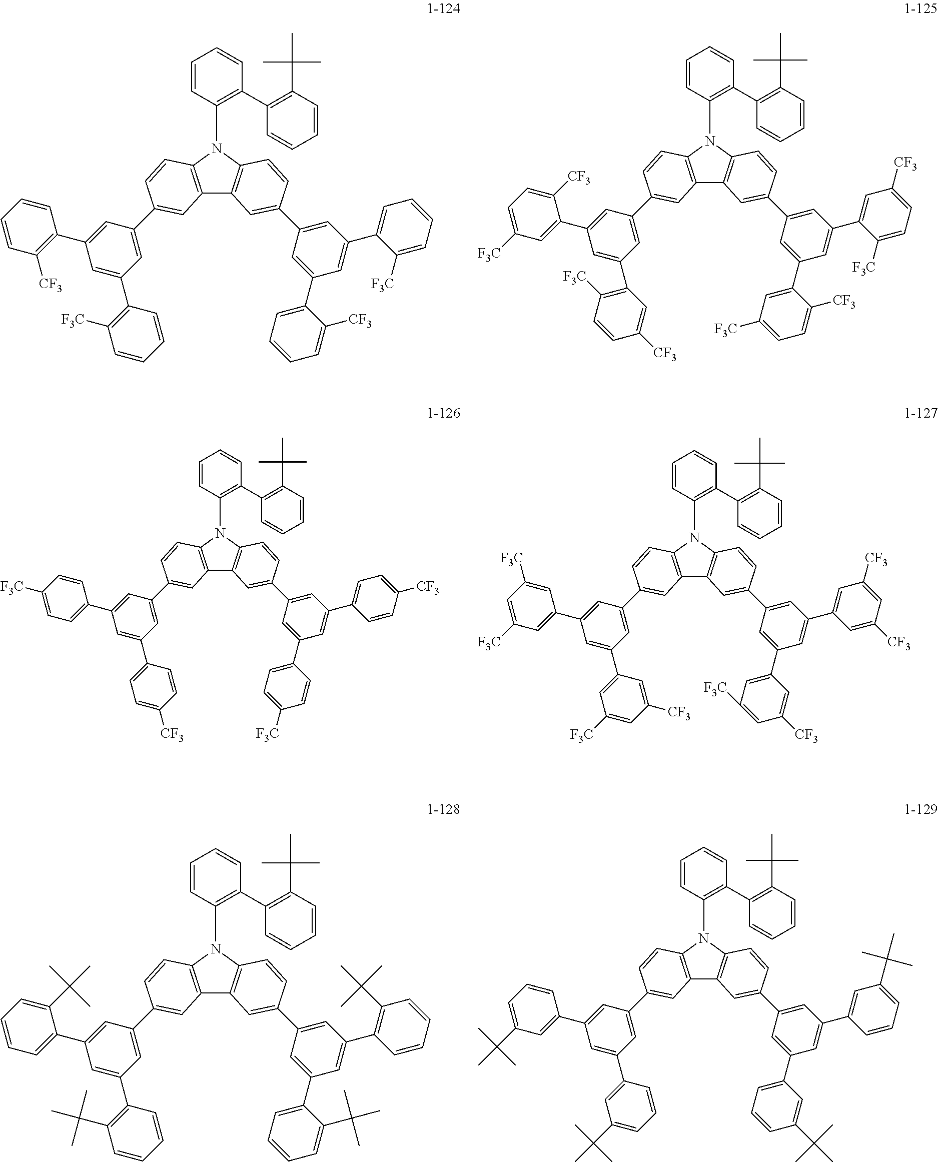

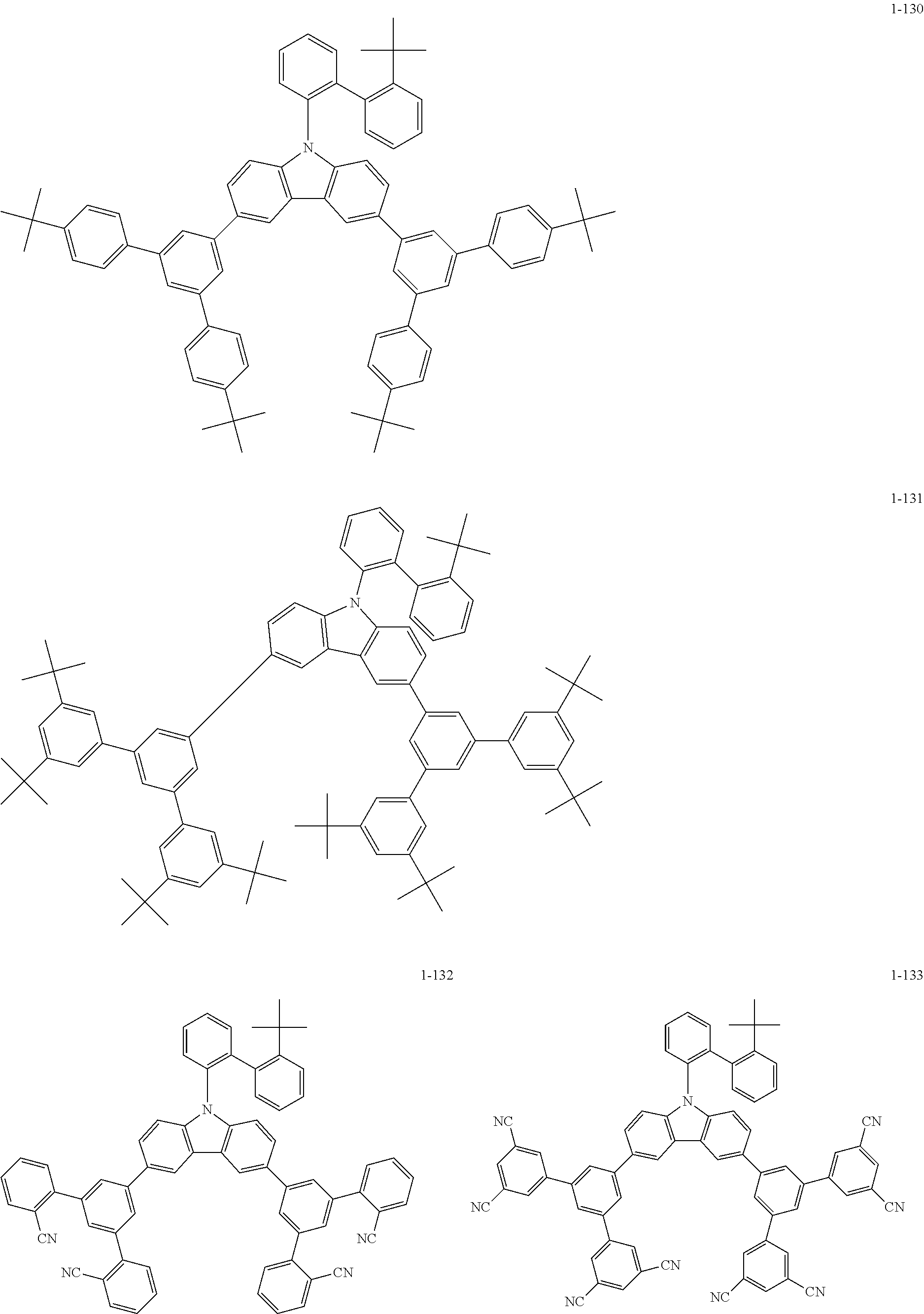

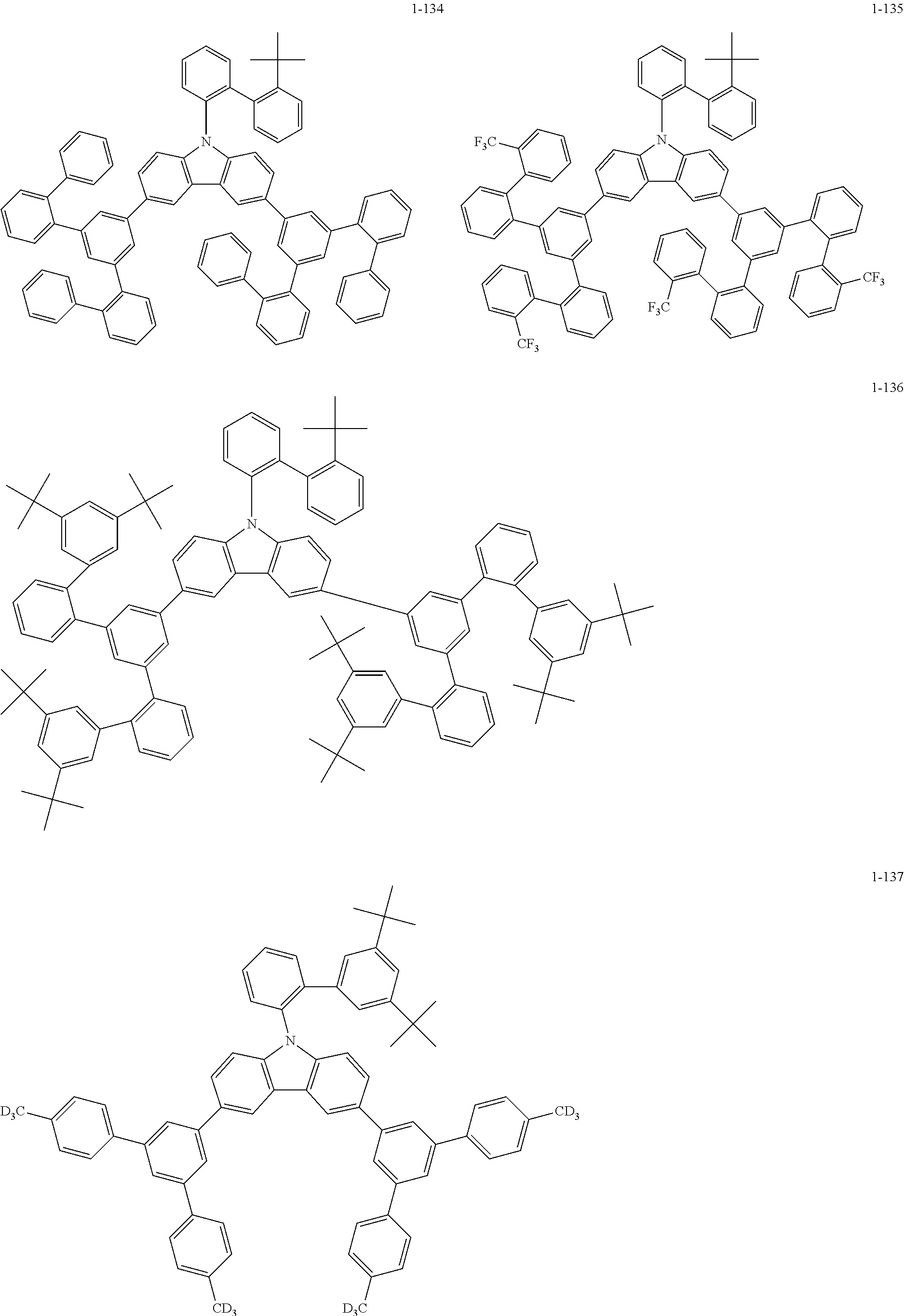

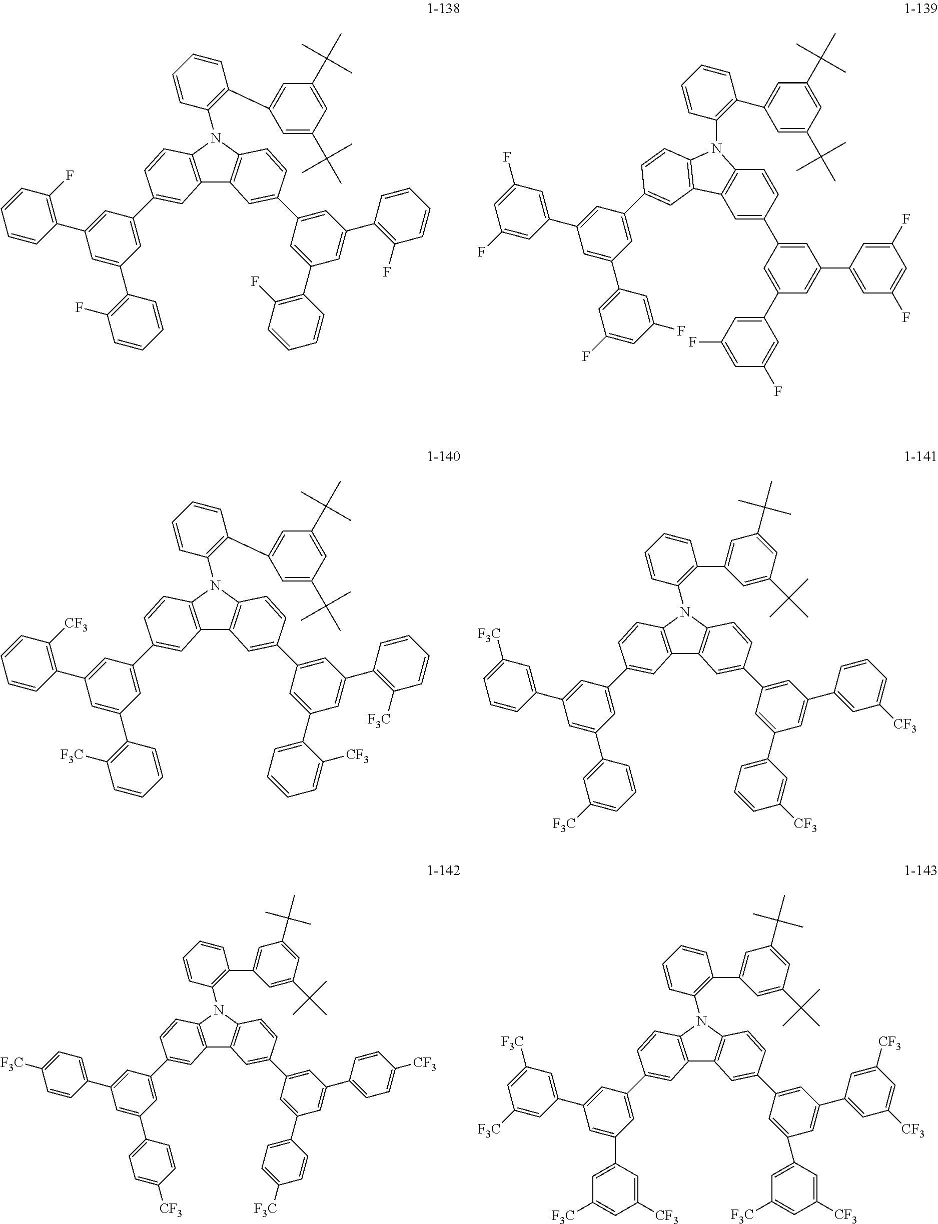

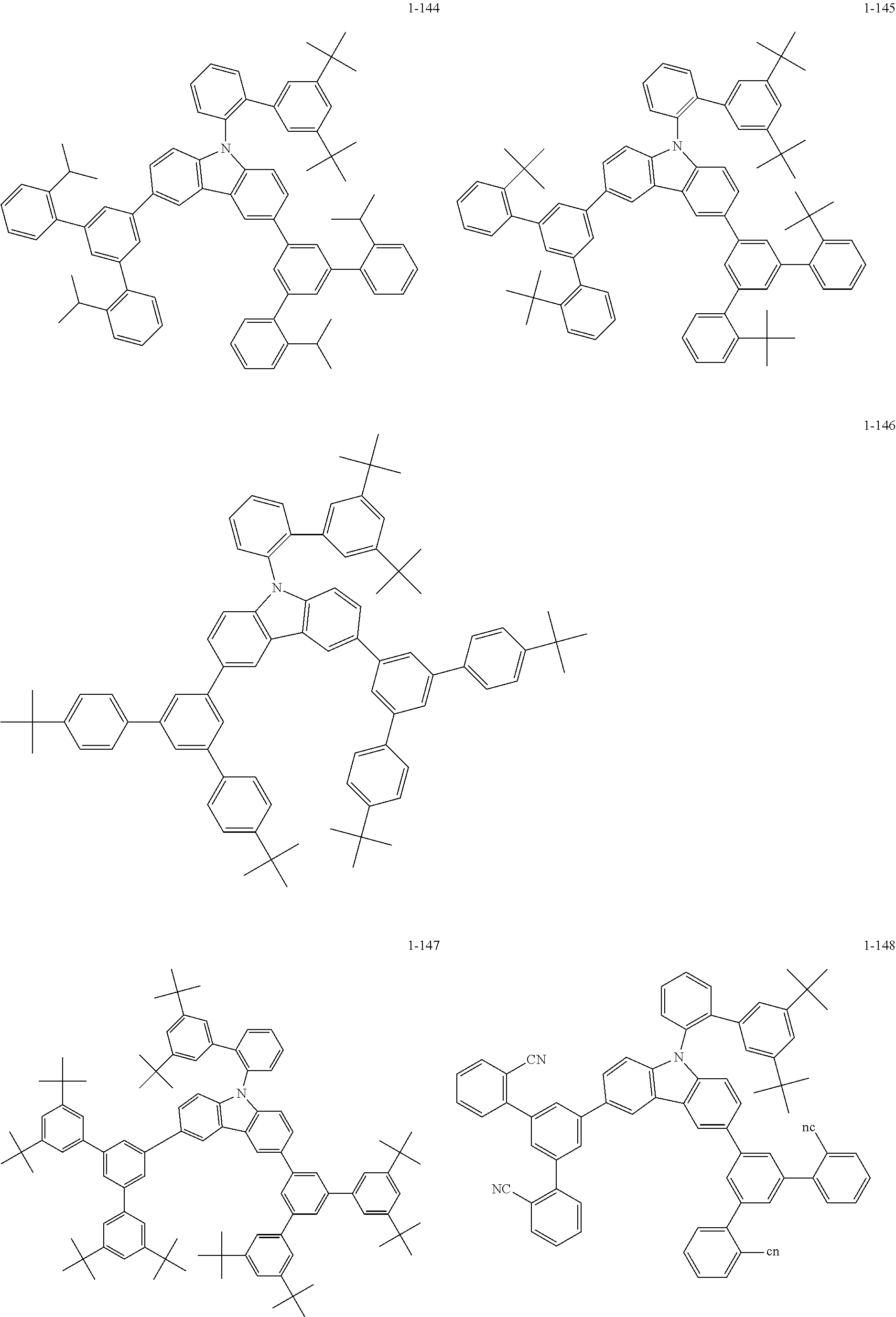

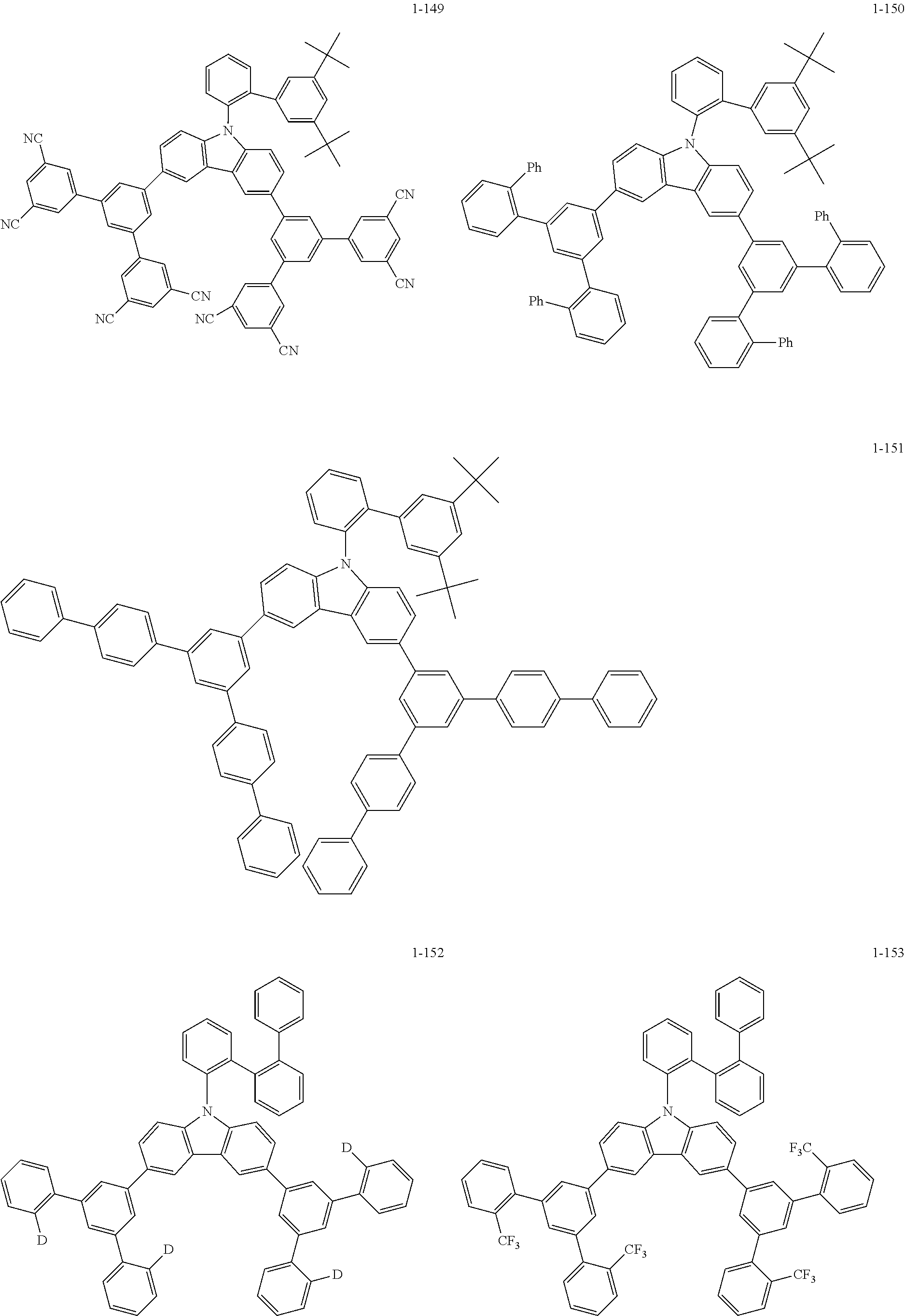

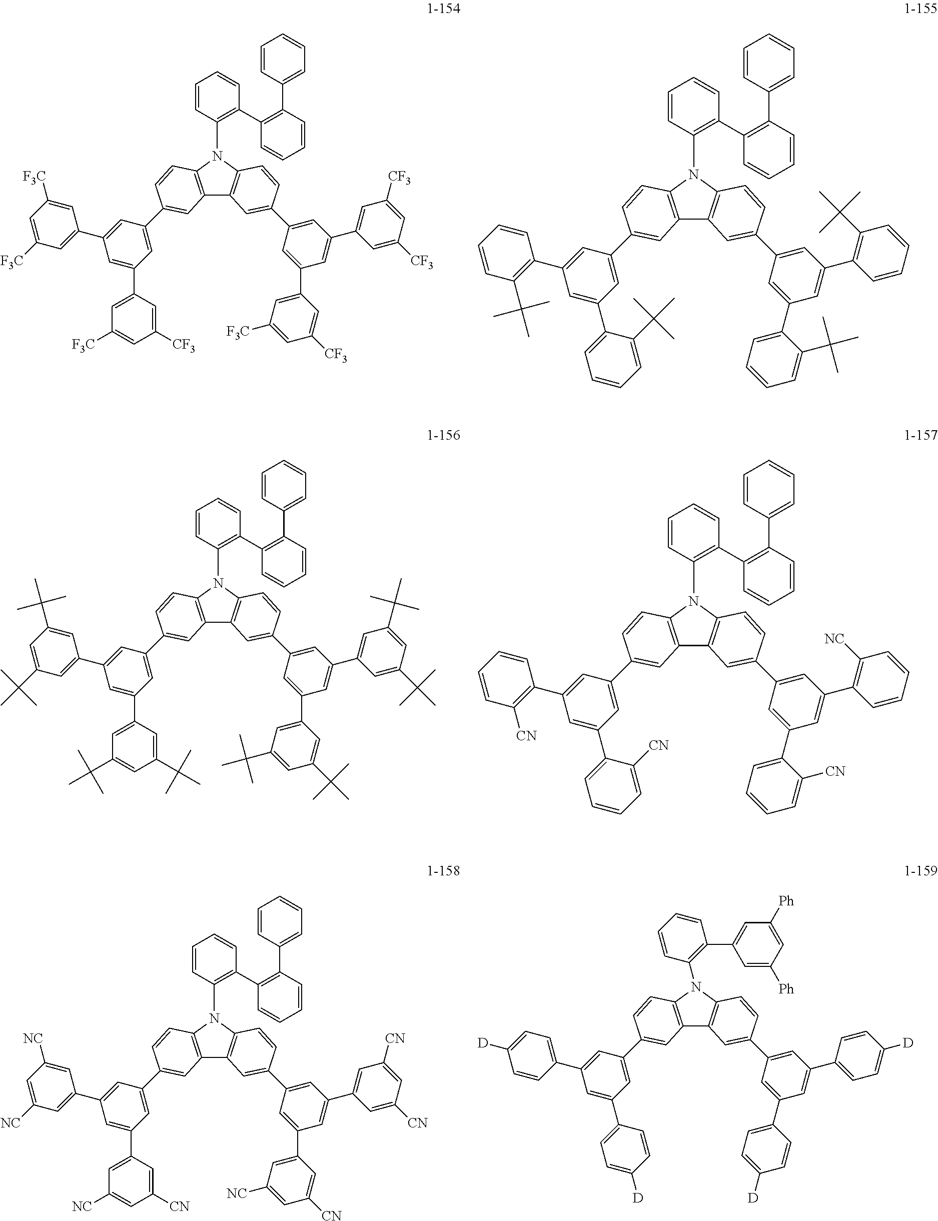

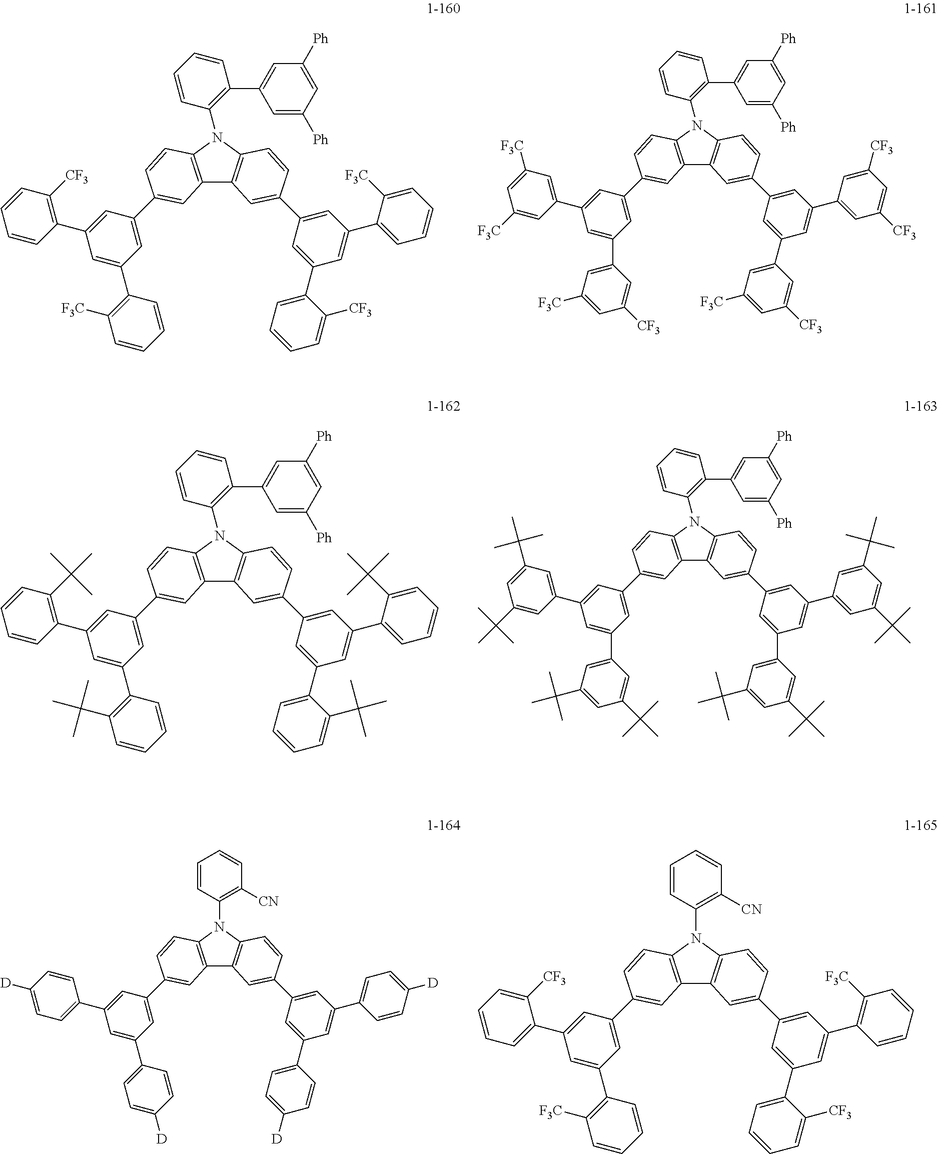

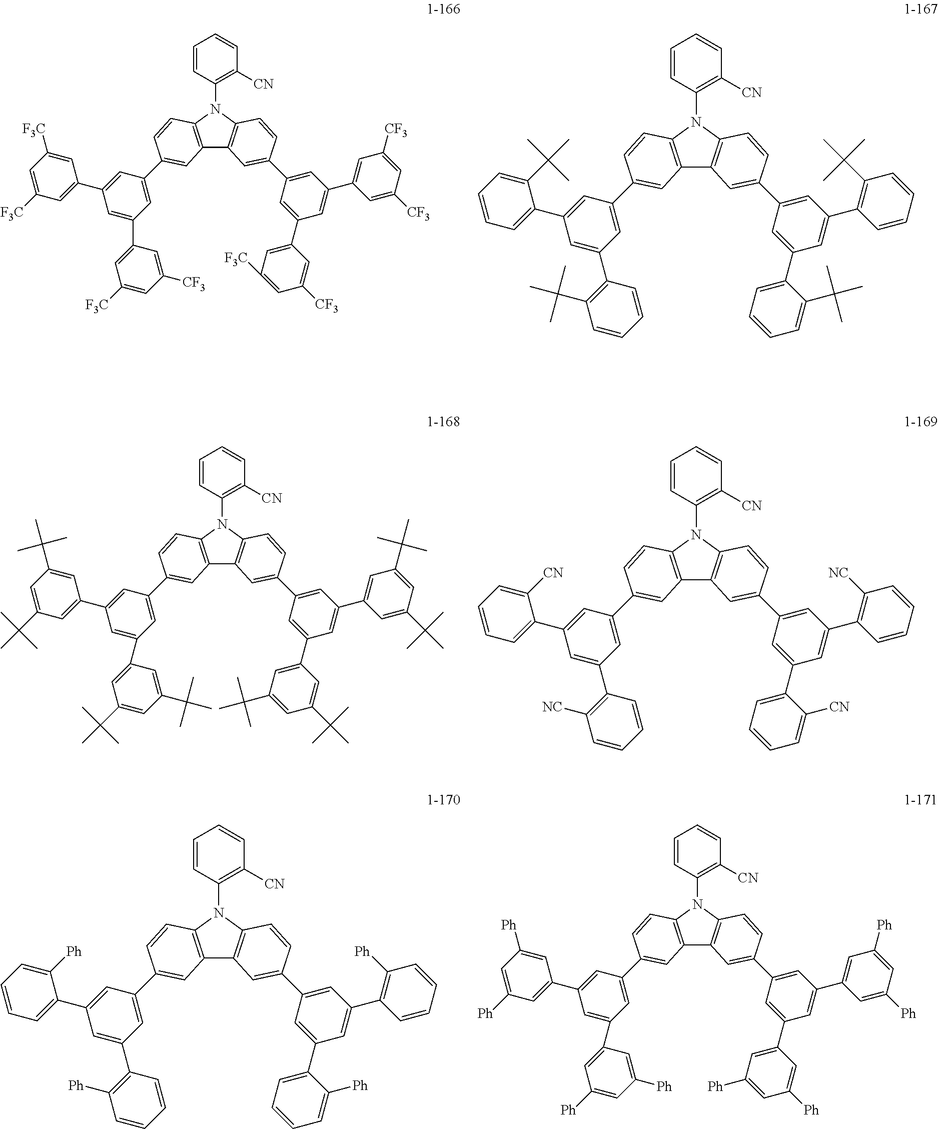

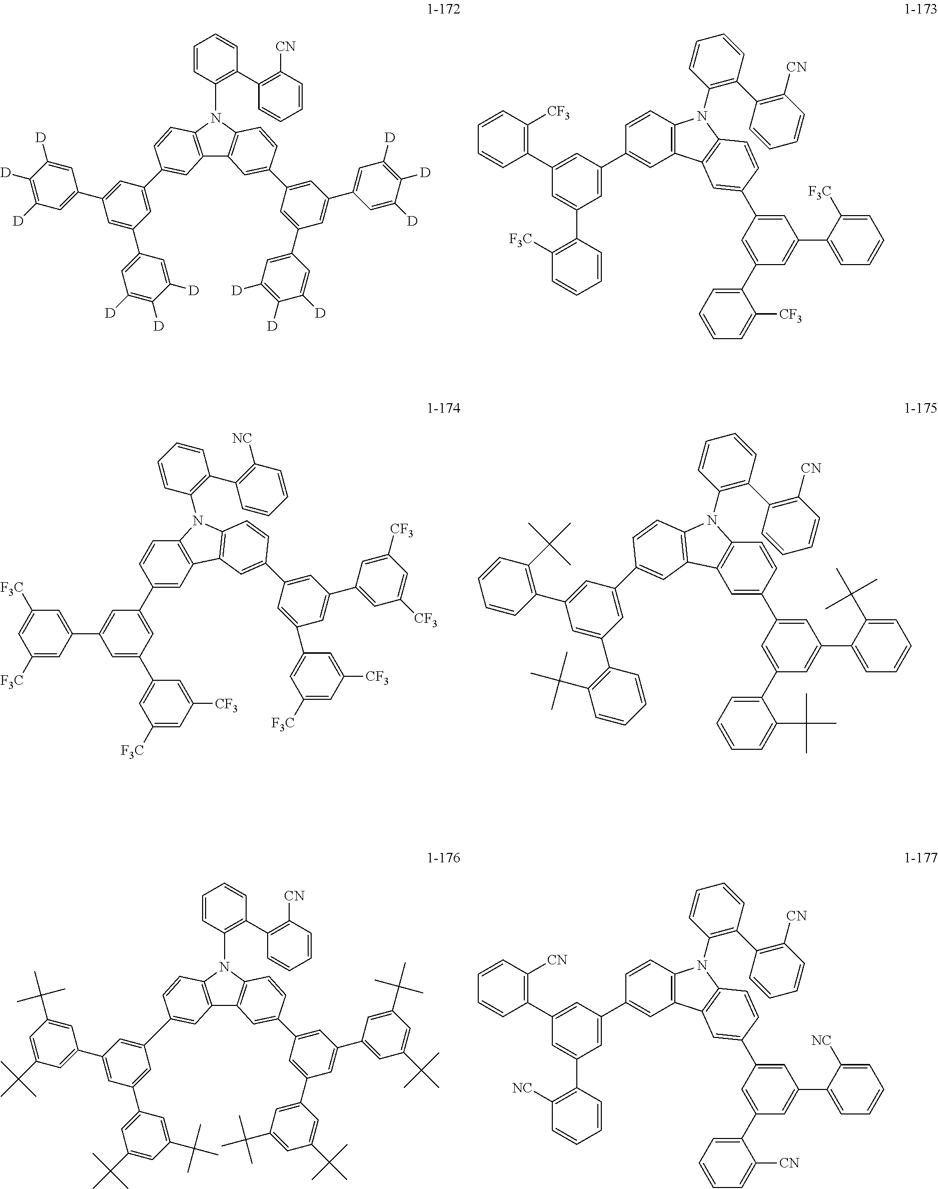

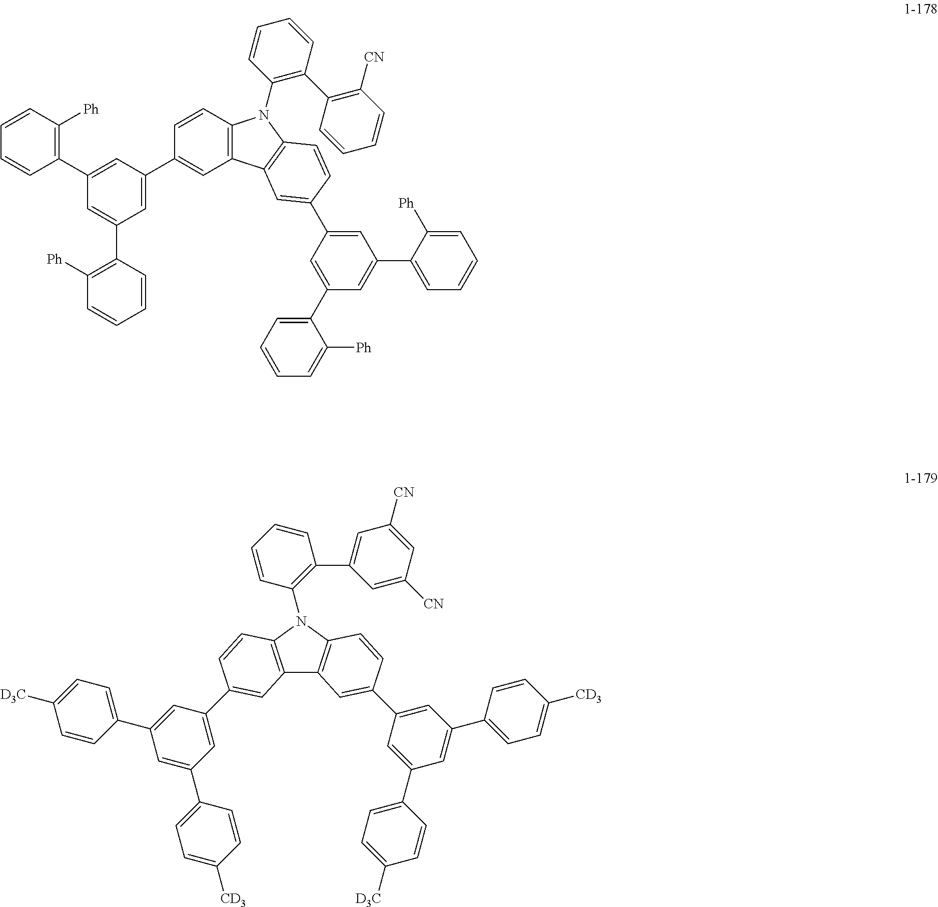 [SMILES: N#Cc1ccccc1-c1ccccc1-n1c2ccc(-c3cc(-c4ccccc4-c4ccccc4)cc(-c4ccccc4-c4ccccc4)c3)cc2c2cc(-c3cc(-c4ccccc4-c4ccccc4)cc(-c4ccccc4-c4ccccc4)c3)ccc21 ; [2H]C([2H])([2H])c1ccc(-c2cc(-c3ccc(C([2H])([2H])[2H])cc3)cc(-c3ccc4c(c3)c3cc(-c5cc(-c6ccc(C([2H])([2H])[2H])cc6)cc(-c6ccc(C([2H])([2H])[2H])cc6)c5)ccc3n4-c3ccccc3-c3cc(C#N)cc(C#N)c3)c2)cc1]

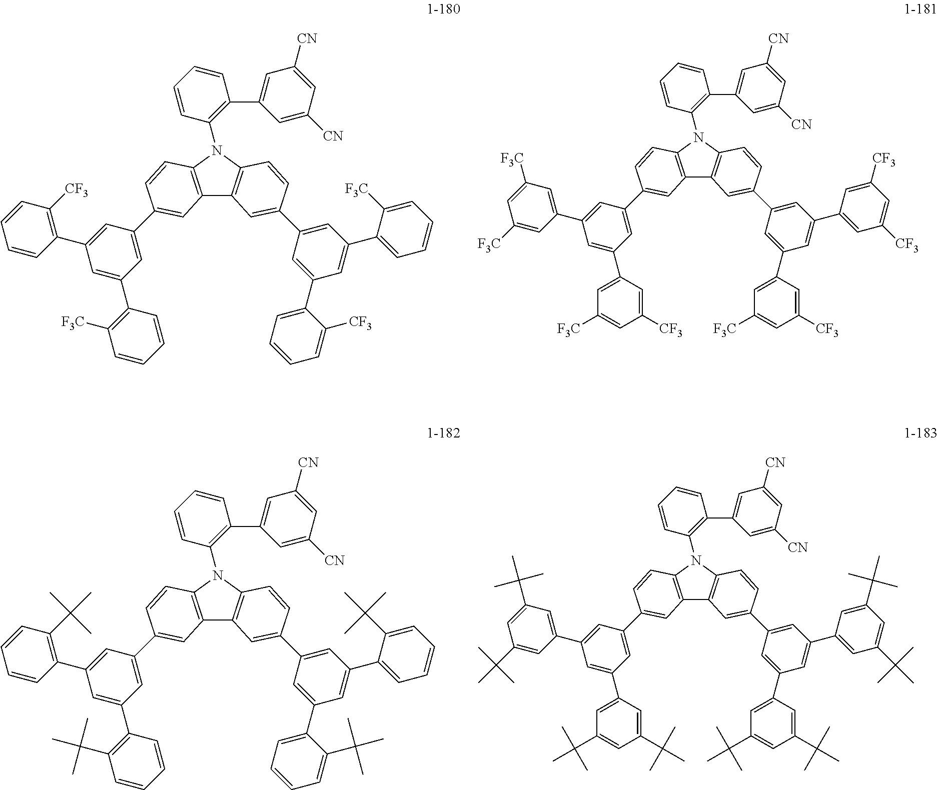

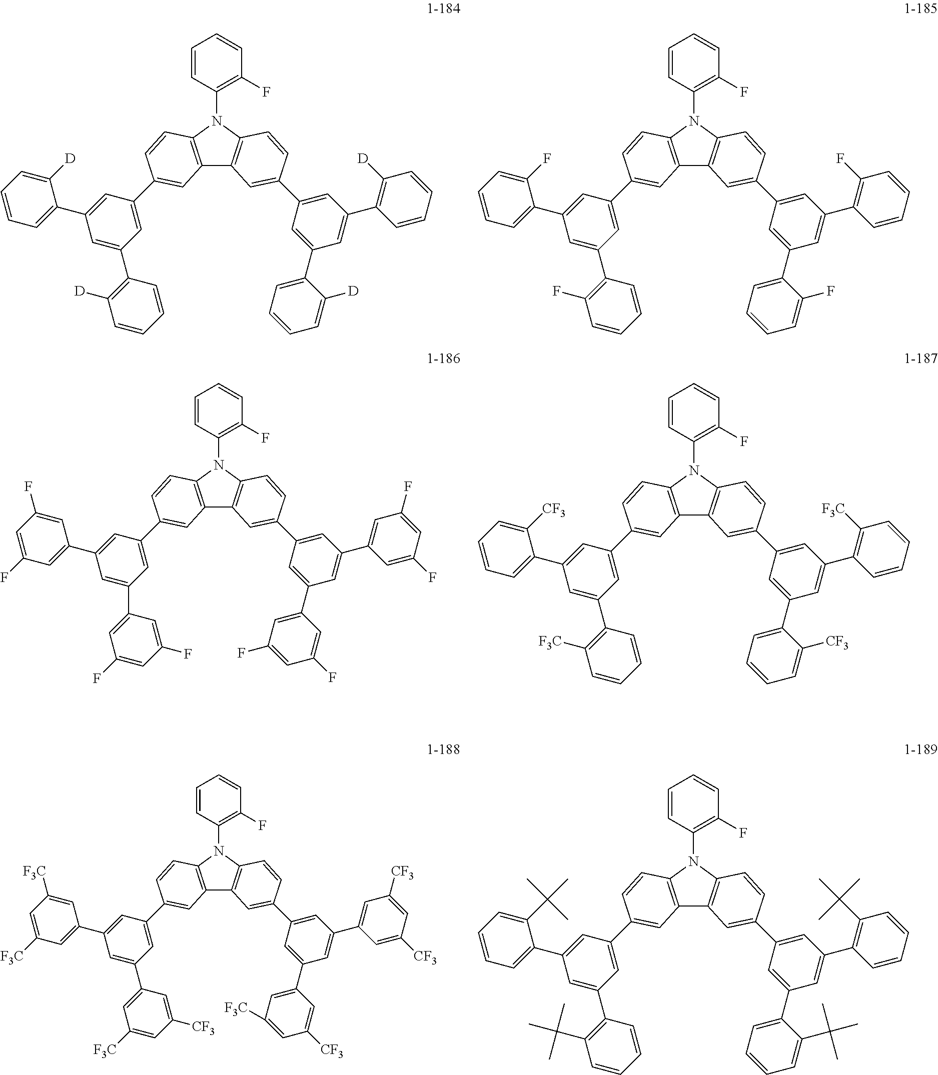

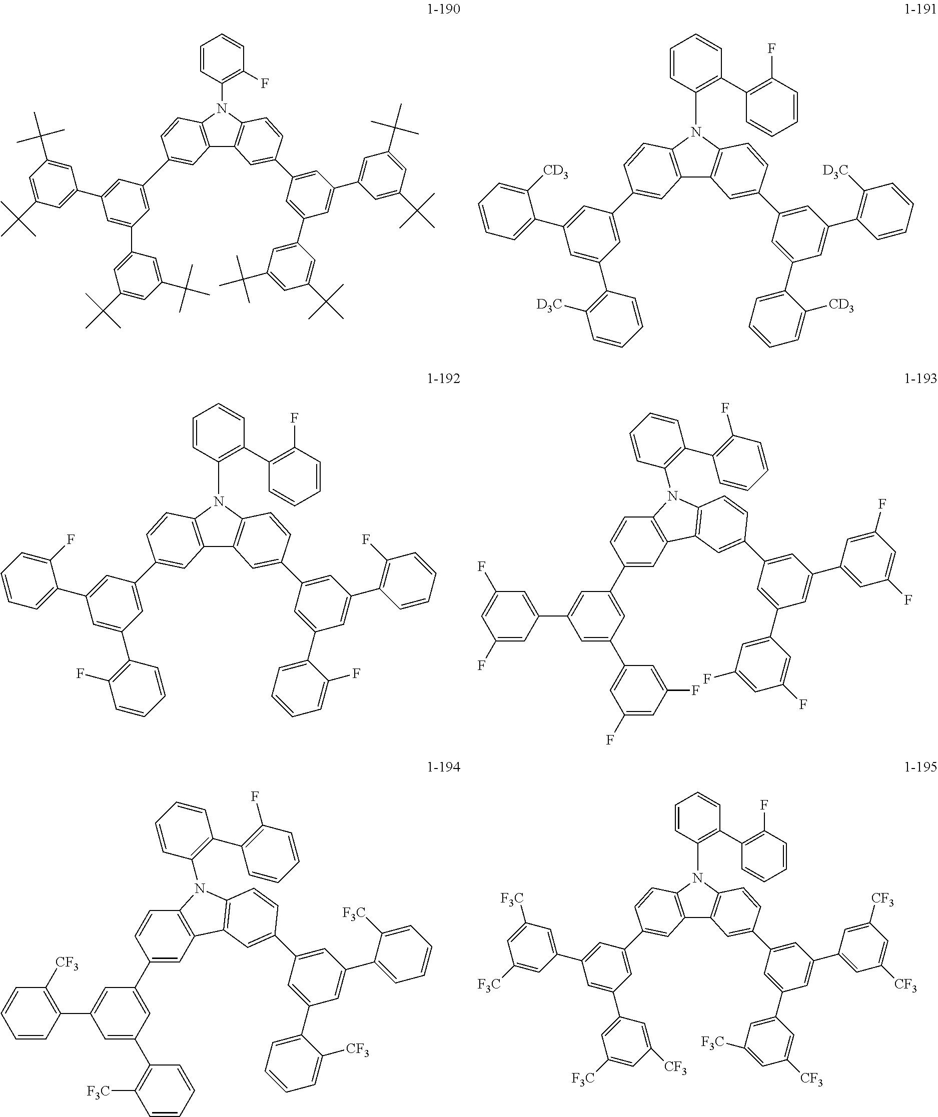

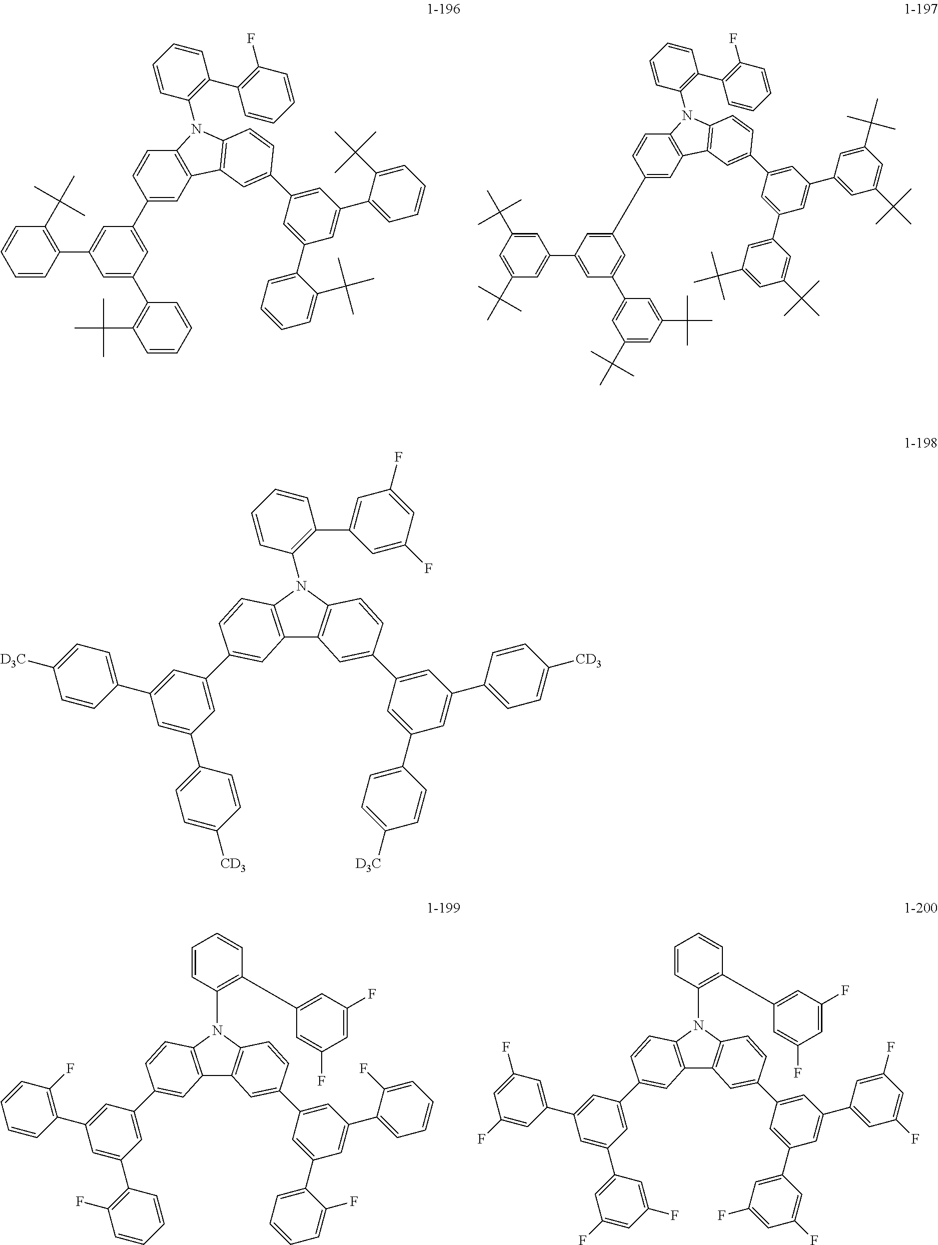

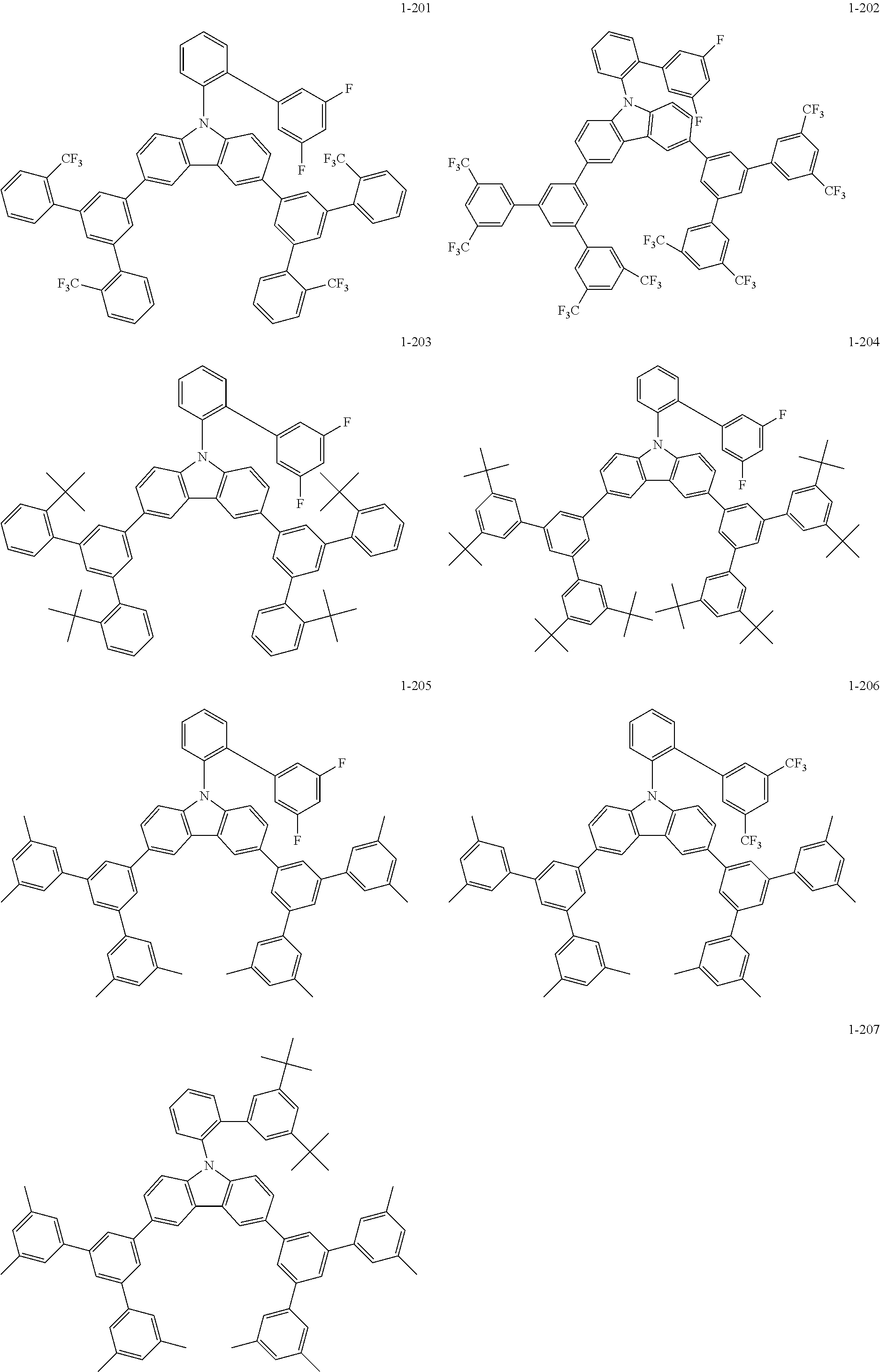

7. An organic light emitting device comprising a first electrode, a second electrode, and an organic material layer having one or more layers disposed between the first electrode and the second electrode,
wherein one or more layers of the organic layer comprise the organic light emitting compound of [Chemical Formula 1] according to claim 1.

8. The organic light emitting device of claim 7, wherein the organic layer comprises one or more selected among a hole injection layer, a hole transport layer, a layer which simultaneously injects and transports holes, an electron transport layer, an electron injection layer, a layer which simultaneously transports and injects electrons, an electron blocking layer, a hole blocking layer and a light emitting layer, and
one or more layers of the layers comprise the organic light emitting compound represented by [Chemical Formula I].

9. The organic light emitting device of claim 7, further comprising a light efficiency improving layer (capping layer) formed on at least one side opposite to the organic layer at the top or bottom part of the first electrode and the second electrode,
wherein the light efficiency improving layer comprises the organic light emitting compound represented by [Chemical Formula I].

10. The organic light emitting device of claim 7, wherein the light efficiency improving layer is formed on at least one of the bottom of the first electrode or the top of the second electrode.
